SEQUENCE LISTING (499 sequences)

SEQ ID NO: 1 - protein, 6 aa, Artificial Sequence
  Synthetic
MQLAIS

SEQ ID NO: 2 - protein, 3 aa, Artificial Sequence
  Synthetic
MFL

SEQ ID NO: 3 - protein, 3 aa, Artificial Sequence
  Synthetic
MSR

SEQ ID NO: 4 - protein, 5 aa, Artificial Sequence
  Synthetic
MFCNS

SEQ ID NO: 5 - protein, 5 aa, Artificial Sequence
  Synthetic
MSGVD

SEQ ID NO: 6 - protein, 8 aa, Artificial Sequence
  Synthetic
MRRGEREG

SEQ ID NO: 7 - protein, 32 aa, Artificial Sequence
  Synthetic
MICPFRNRQNKKKKKESWRILHSSSSSSSPIH

SEQ ID NO: 8 - protein, 23 aa, Artificial Sequence
  Synthetic
MKRETETEVSYFLLCISHILLKP

SEQ ID NO: 9 - protein, 10 aa, Artificial Sequence
  Synthetic
MFMNSSLLYS

SEQ ID NO: 10 - protein, 9 aa, Artificial Sequence
  Synthetic
MSCHFLVIS

SEQ ID NO: 11 - protein, 22 aa, Artificial Sequence
  Synthetic
MARLFPNDFFFFVVAGLGFPLL

SEQ ID NO: 12 - protein, 8 aa, Artificial Sequence
  Synthetic
MILREIAA

SEQ ID NO: 13 - protein, 5 aa, Artificial Sequence
  Synthetic
MIATQ

SEQ ID NO: 14 - protein, 16 aa, Artificial Sequence
  Synthetic
MLEIFCFYLRETDLDS

SEQ ID NO: 15 - protein, 14 aa, Artificial Sequence
  Synthetic
MRCLVQLHEQSLSR

SEQ ID NO: 16 - protein, 2 aa, Artificial Sequence
  Synthetic
MG

SEQ ID NO: 17 - protein, 9 aa, Artificial Sequence
  Synthetic
MVGLIFQSG

SEQ ID NO: 18 - protein, 1 aa, Artificial Sequence
  Synthetic
M

SEQ ID NO: 19 - protein, 4 aa, Artificial Sequence
  Synthetic
MRRS

SEQ ID NO: 20 - protein, 17 aa, Artificial Sequence
  Synthetic
MKQLFLSSMFTSKRVKG

SEQ ID NO: 21 - protein, 14 aa, Artificial Sequence
  Synthetic
MIGFSRFDFSGSCS

SEQ ID NO: 22 - protein, 2 aa, Artificial Sequence
  Synthetic
ML

SEQ ID NO: 23 - protein, 16 aa, Artificial Sequence
  Synthetic
MLLISMQISPFFSSHF

SEQ ID NO: 24 - protein, 12 aa, Artificial Sequence
  Synthetic
MPKNKNKIISVA

SEQ ID NO: 25 - protein, 10 aa, Artificial Sequence
  Synthetic
MAVLHIIFTA

SEQ ID NO: 26 - protein, 20 aa, Artificial Sequence
  Synthetic
MKPKLSNNSKLTTLFFCLCC

SEQ ID NO: 27 - protein, 7 aa, Artificial Sequence
  Synthetic
MCFFLSS

SEQ ID NO: 28 - protein, 12 aa, Artificial Sequence
  Synthetic
MSSRFDPKGFGR

SEQ ID NO: 29 - protein, 9 aa, Artificial Sequence
  Synthetic
MIETRTIFL

SEQ ID NO: 30 - protein, 6 aa, Artificial Sequence
  Synthetic
MNLLLF

SEQ ID NO: 31 - protein, 3 aa, Artificial Sequence
  Synthetic
MVL

SEQ ID NO: 32 - protein, 5 aa, Artificial Sequence
  Synthetic
MCPKR

SEQ ID NO: 33 - protein, 3 aa, Artificial Sequence
  Synthetic
MER

SEQ ID NO: 34 - protein, 15 aa, Artificial Sequence
  Synthetic
MDSRSQTFISLSLTI

SEQ ID NO: 35 - protein, 52 aa, Artificial Sequence
  Synthetic
MMESKAGNKKSSSNSSLCYEAPLGYSIEDVRPFGGIKKFKSSVYSNCAKRPS

SEQ ID NO: 36 - protein, 31 aa, Artificial Sequence
  Synthetic
METKWREKAPTLSHPKASSSSSSSSSSSSLI

SEQ ID NO: 37 - protein, 6 aa, Artificial Sequence
  Synthetic
MFQSNH

SEQ ID NO: 38 - protein, 31 aa, Artificial Sequence
  Synthetic
MSTGLSLSSNGFYYVHVGISLPLCLLHSEMT

SEQ ID NO: 39 - DNA, 21 bp, Artificial Sequence
  Synthetic
atgcagcttgcgatttcatag

SEQ ID NO: 40 - DNA, 12 bp, Artificial Sequence
  Synthetic
atgttcttgtga

SEQ ID NO: 41 - DNA, 12 bp, Artificial Sequence
  Synthetic
atgagcagatag

SEQ ID NO: 42 - DNA, 18 bp, Artificial Sequence
  Synthetic
atgttctgcaacagttga

SEQ ID NO: 43 - DNA, 18 bp, Artificial Sequence
  Synthetic
atgagtggggtggattaa

SEQ ID NO: 44 - DNA, 27 bp, Artificial Sequence
  Synthetic
atgcggcgaggagaaagagaaggttaa

SEQ ID NO: 45 - DNA, 99 bp, Artificial Sequence
  Synthetic
atgatatgtccgtttagaaacagacaaaataagaagaagaagaaagagtcgtggaggattcttcattcttcctcatcctcttcttcaccgattcattag

SEQ ID NO: 46 - DNA, 72 bp, Artificial Sequence
  Synthetic
atgaagagagagactgaaacagaggtttcttactttcttctctgtatctctcatattttgcttaaaccctaa

SEQ ID NO: 47 - DNA, 33 bp, Artificial Sequence
  Synthetic
atgtttatgaattcttcattgctctattcttag

SEQ ID NO: 48 - DNA, 30 bp, Artificial Sequence
  Synthetic
atgagctgtcacttcttagtaataagttga

SEQ ID NO: 49 - DNA, 69 bp, Artificial Sequence
  Synthetic
atggctcgtctttttccaaacgatttcttcttcttcgtcgtcgccggtttagggtttccgttgctgtag

SEQ ID NO: 50 - DNA, 27 bp, Artificial Sequence
  Synthetic
atgatcttacgtgaaattgcagcctaa

SEQ ID NO: 51 - DNA, 18 bp, Artificial Sequence
  Synthetic
atgatagctacccagtaa

SEQ ID NO: 52 - DNA, 51 bp, Artificial Sequence
  Synthetic
atgcttgagatattttgtttttacctccgagaaacggatttagattcgtaa

SEQ ID NO: 53 - DNA, 45 bp, Artificial Sequence
  Synthetic
atgcgttgtttggtacagcttcacgaacaatctctctctcgatag

SEQ ID NO: 54 - DNA, 9 bp, Artificial Sequence
  Synthetic
atgggttag

SEQ ID NO: 55 - DNA, 30 bp, Artificial Sequence
  Synthetic
atggttggtttaatatttcaatcgggataa

SEQ ID NO: 56 - DNA, 6 bp, Artificial Sequence
  Synthetic
atgtga

SEQ ID NO: 57 - DNA, 15 bp, Artificial Sequence
  Synthetic
atgcgtcgctcttaa

SEQ ID NO: 58 - DNA, 54 bp, Artificial Sequence
  Synthetic
atgaagcaattgttcctttcaagcatgtttacgagcaaaagagtgaaagggtag

SEQ ID NO: 59 - DNA, 45 bp, Artificial Sequence
  Synthetic
atgattggcttctcacgattcgatttttccggctcctgttcttaa

SEQ ID NO: 60 - DNA, 9 bp, Artificial Sequence
  Synthetic
atgttataa

SEQ ID NO: 61 - DNA, 51 bp, Artificial Sequence
  Synthetic
atgcttttaatttccatgcaaatctctcctttcttctcaagtcatttttga

SEQ ID NO: 62 - DNA, 39 bp, Artificial Sequence
  Synthetic
atgccaaaaaataagaacaaaattatatccgttgcttga

SEQ ID NO: 63 - DNA, 33 bp, Artificial Sequence
  Synthetic
atggcggttcttcacattatcttcactgcgtaa

SEQ ID NO: 64 - DNA, 63 bp, Artificial Sequence
  Synthetic
atgaagccaaaactatccaataactcgaaattgactactcttttcttttgtctctgttgttga

SEQ ID NO: 65 - DNA, 24 bp, Artificial Sequence
  Synthetic
atgtgtttcttcttgtcttcctag

SEQ ID NO: 66 - DNA, 39 bp, Artificial Sequence
  Synthetic
atgtcgtcgcggttcgaccccaagggatttggccggtaa

SEQ ID NO: 67 - DNA, 30 bp, Artificial Sequence
  Synthetic
atgatagaaacaagaactatctttttataa

SEQ ID NO: 68 - DNA, 21 bp, Artificial Sequence
  Synthetic
atgaaccttcttctcttctag

SEQ ID NO: 69 - DNA, 12 bp, Artificial Sequence
  Synthetic
atggttctgtaa

SEQ ID NO: 70 - DNA, 18 bp, Artificial Sequence
  Synthetic
atgtgccccaaaagataa

SEQ ID NO: 71 - DNA, 12 bp, Artificial Sequence
  Synthetic
atggaaagataa

SEQ ID NO: 72 - DNA, 48 bp, Artificial Sequence
  Synthetic
atggactctcgttctcagacatttatttctctcagtcttacaatataa

SEQ ID NO: 73 - DNA, 159 bp, Artificial Sequence
  Synthetic
atgatggaatcgaaagctggtaataagaagtcaagcagcaatagttccttatgttacgaagcaccccttggttacagcattgaagacgttcgtcctttcggtggaatcaagaaattcaaatcttctgtctactccaactgcgctaagaggccttcctga

SEQ ID NO: 74 - DNA, 96 bp, Artificial Sequence
  Synthetic
atggagacgaagtggagagagaaagctcccactctctcacaccccaaagcttcttcttcttcttcctcttcttcctcttcctcttctctaatctga

SEQ ID NO: 75 - DNA, 21 bp, Artificial Sequence
  Synthetic
atgtttcaatctaaccattga

SEQ ID NO: 76 - DNA, 96 bp, Artificial Sequence
  Synthetic
atgtcaactggattaagcttaagtagtaatggtttttactatgttcatgtggggatttctcttcctctctgtttacttcattccgagatgacttga

SEQ ID NO: 77 - DNA, 249 bp, Artificial Sequence
  Synthetic
gagagaggactgggtctggtctcttcgctgcaacctatagctgttgtttgctcttcgacgggattctcactactcttttgccaaaaaaaagagatcggaggttccgaaggtgaatgcagcttgcgatttcatagaaaagaagattcgtttgctggattaggcttatttgtgtatcatagctttgaggttttaactgagatttattgatagtggaacttaggttttcgagaggtgtgaacagttgggtat

SEQ ID NO: 78 - DNA, 184 bp, Artificial Sequence
  Synthetic
aaattaagagacatctgatcgaattttgttccgacgaccgtgaatcaccagcaaaggattcgtgtcaatgttcttgtgagatcgaactttctctgggttcgtgcagaagctttgcttttttgagtatcgcgtttaaggcacatcgaagaagagagaccctaatttgatattttgagttctatcg

SEQ ID NO: 79 - DNA, 564 bp, Artificial Sequence
  Synthetic
cgtggggaacgttttttcctggaagaagaagaagaagagctcaacaagctcaacgaccaaaaaacttcggacacgaagactttttaattcatttctcctcttttgtttttttcgttccaaaatattcgatactctcgatctcttcttcgtgatcctcattaaataaaaatacgatttttattctttttttgtgagtgcaccaaattttttgactttggattagcgtagaattcaagcacattctgggtttattcgtgtatgagtagacattgattttgtcaaagttgcattcttttatataaaaaaagtttaatttccttttttcttttcttttctcttttttttttttttcccccatgttatagattcttccccaaattttgaagaaaggagagaactaaagagtcctttttgagattcttttgctgcttcccttgcttgattagatcatttttgtgattctggattttgtgggggtttcgtgaagcttattgggatcttatctgattcaggattttctcaaggctgcattgccgtatgagcagatagttttatttaggcatt

SEQ ID NO: 80 - DNA, 802 bp, Artificial Sequence
  Synthetic
cttcttcttctgattctcatttcaaataagagagagagagagagaagtaagtaaaactttagcagagagaagaataaacaaataattatagcaccgtcacgtcgccgccgtatttcgttaccggaaaaaaaaaatcattcttcaacataaaaataaaaacagtctctttctttctatctttgtctatctttgattattctctgtgtacccatgttctgcaacagttgagcaagtgcatgccccatatctctctgtttctcatttcccgatctttgcattaatcatatacttcgcctgagatctcgattaagccagcttatagaagaagaaacggcaccagcttctgtcgttttagttagctcgagatctgtgtttctttttttcttggcttctgagcttttggcggtggtgggtttttctggagaaacccaaacgactatcaaagttttgttttttacaattttaagtgggagttatgagtggggtggattaagtaagttacaagtatgaaggagttgaagattcgaagaagcggttttgaagtcggcgagaccaagattgcgagcttatttggctgatgatttatttcagggaagaagaaataaatctgttttttttagggtttttagatttggttggtgaatgggtgggaggtggagggaaacagttaaaaaagttatgcttttagtgtctcttcttcataattacatttgggcatcttgaaatctttggatctttgaagaaacaaagttgtgtttttttttttgttctttgttgtttgctttttaagttagaataaaaa

SEQ ID NO: 81 - DNA, 35 bp, Artificial Sequence
  Synthetic
cgagatgcggcgaggagaaagagaaggttaaggtt

SEQ ID NO: 82 - DNA, 163 bp, Artificial Sequence
  Synthetic
attgtgtggtgacaagcaacacatgatatgtccgtttagaaacagacaaaataagaagaagaagaaagagtcgtggaggattcttcattcttcctcatcctcttcttcaccgattcattagaaaccaaattacaaaaaaaaacgtctatacacaaaaaaacaa

SEQ ID NO: 83 - DNA, 175 bp, Artificial Sequence
  Synthetic
agtgagctaatgaagagagagactgaaacagaggtttcttactttcttctctgtatctctcatattttgcttaaaccctaaaaccctttttggattaggttttctccaaatcttatccgccgtgataaaatctgattgctttttttcttcatgaaagtttgatttgtgaaactcg

SEQ ID NO: 84 - DNA, 192 bp, Artificial Sequence
  Synthetic
cctttctcttccgatcgcatcttcttcaaaaatttcccacctgtgtttcacaaattccatgtttatgaattcttcattgctctattcttagtcacctttgatttctctcgctttctatccgatccaattgtttgatgatcttctctgtaacaagctcataaggtttgagcttcatctctctggagagaatcc

SEQ ID NO: 85 - DNA, 365 bp, Artificial Sequence
  Synthetic
aagcgaacaagtctttgcctctttggtttactttcctctgttttcgatccatttagaaaatgttattcacgaggagtgttgctcggatttcttctaagtttctgagaaaccgtagcttctatggctcctctcaatctctcgcctctcatcggttcgcaatcattcccgatcagggtcactcttgttctgactctccacacaagtagggttacgtttgcagaacaacttattcattgaaatctccggtttttggtggatttagtcatcaactctatcaccagagtagctccttggttgaggaggagcttgacccattttcgcttgttgccgatgagctgtcacttcttagtaataagttgagagag

SEQ ID NO: 86 - DNA, 417 bp, Artificial Sequence
  Synthetic
caagagtagaccgccgacttagattttttcgccgacgagagaatatatataaatggctcgtctttttccaaacgatttcttcttcttcgtcgtcgccggtttagggtttccgttgctgtagcaattttctctcgcttctctctccccttttacagtttctcttatattgctcttgccttgcgtccaatctcaagagttcataagagttgacatttgtgaacatcgaagaaatacggtgacgtttcttctctgattactttttgccaacatgaatactaatgtatttatcaacaagtgctacagcctgtttttttcgaagctgttggtgagttcccatccttagtactgctagacagttcggtgtgttagttgactttatattcaaggttataggtttagtgttgttagtagagaaaa

SEQ ID NO: 87 - DNA, 240 bp, Artificial Sequence
  Synthetic
acattcatctctctctctcagtcaaattgttgttttctttcttcgaatcggtgcagaaaattcagggaagttctggggaaggttgttgcgtttgactcctttggcttagttttctttcgaattccgtgcttcctgatgatcttacgtgaaattgcagcctaaaatttcgagattgttttttttactcagaaaacgagatttgactgatatgaatcgaaaatctgtgatttaaagtgaagc

SEQ ID NO: 88 - DNA, 170 bp, Artificial Sequence
  Synthetic
aaactgctgaccgatcccaaaggttgaaagattctttggcgctaaaaaatccccagttcccaaatcggcgtcctcgtttgaaaccctaattcctgaatcgaagcagatcctgatcgaatcgaaggtgttcgaatgatagctacccagtaaattcagaaccctaattaaca

SEQ ID NO: 89 - DNA, 117 bp, Artificial Sequence
  Synthetic
gtaaagagaaaagctttgagtcttccattgacatgggcgcttagcttatgcttgagatattttgtttttacctccgagaaacggatttagattcgtaatcgtgagttttttggtgta

SEQ ID NO: 90 - DNA, 87 bp, Artificial Sequence
  Synthetic
aaataaatgcgttgtttggtacagcttcacgaacaatctctctctcgatagattcttcttacctctgaatttctcgttgttggaaca

SEQ ID NO: 91 - DNA, 483 bp, Artificial Sequence
  Synthetic
agattttttttttaaacaaagaatggaaaaaaatgaataaatttgggaaacgaggaagctttggttacccaaaaaagaaagaaagaaaaaataaaaaaaaataaaaagaaaagctttctctgggtttttcttgattggtcaattacacatctccctttctctcttctctctctcaccttcgcttgctttgcttgcttcatctctttggtctccttcttgcgttttctatttactacacagaccaaacaatagagagagactttaagctatagaaaaaaagagagagattctctcaaatatgggttagtccacaattttcactaaacctcttcttcttaggattgtttttagggttagggttttgaggtgaggagagcaagtatgcgggagtttcatcctttttgagttactctggattcctcaccctctaacgacgaccaccgtcgccgccgccgccgccgtctcgacgaatatgctctacca

SEQ ID NO: 92 - DNA, 218 bp, Artificial Sequence
  Synthetic
atgagaaaagcttggtaaaaaccctatttttcttcttctcttcaatttacagttctctgcacctttttctttcccctgttttttgatcctcaatcaccaaaccctagcttgttcttctgttgattatttcgaaaagggggtttgtttgttttctgggaatcagcaaaaatcacgaaatggttggtttaatatttcaatcgggataaaatcgatcgaaa

SEQ ID NO: 93 - DNA, 198 bp, Artificial Sequence
  Synthetic
gtcacacatgtaataaaccttggtcgacaatctcgccctttccatgtgatttctccacttcctctctctctctactgcaacttcctcctcctgcttcaacttcattcgggtaatgatgaactagcgtagagatttggatcttcttcttcgtcctctcaccaactcttcaccggttagatctctttttcacgctaacga

SEQ ID NO: 94 - DNA, 88 bp, Artificial Sequence
  Synthetic
gtgtttagcttcttcactaccacacagaaacagagtttccgtctttcatcttcctccatatgcgtcgctcttaaaaacctaattcaca

SEQ ID NO: 95 - DNA, 378 bp, Artificial Sequence
  Synthetic
tcttcttcttcgttttcaggcgggtggaggagctcagagccttccagaggtaaccaaccttttattaccgacaagattctgccacacaattattacatatttttgttcccatgaagcaattgttcctttcaagcatgtttacgagcaaaagagtgaaagggtagcttgatttttgtctactctagcttcattttctggcgatctttacttgagatttaaacattttgctctcggattgataataaagaagaatttggaatatcagtaggtttggttaggactctcggattctgttgtcggttagatatttgttttgtttaatccctagatttagcagagaaatccctcaaatctcacacaatccatgtaaggaagaag

SEQ ID NO: 96 - DNA, 303 bp, Artificial Sequence
  Synthetic
cttacttaaacacagtcaaattcattttctgccttagaaaagatttttatcgaaaatcgacgtttttgaaaaaactcaaattatcgtcgttttgttctcagatttcttctgctctcttcttcttctccttcttcttcgttccaccgcctctgttgctttatcttcttcttccttccttcgattgttgattacgtcggtggatctttgttctcctctgtgttgttttcattgctagatttcgtcaatgattggcttctcacgattcgatttttccggctcctgttcttaatttcctctgagaga

SEQ ID NO: 97 - DNA, 285 bp, Artificial Sequence
  Synthetic
atcaaaatcaatgatcaaggtaacgtagtcaagttcaattactctttgtcaaatttaagtggtctctattactaaactatacacaaccgttagatcaaataattctctaccatccaacggtccaaagtctccacttctatttattacaataaaatgagaaaataaaaacgcgcggtcaccgattctctctcgctctctctgttactaaatgaagaagagaatctctccggcgagatcaccggcgttattccgataatttcgcctgagagttgtcgcatgttataa

SEQ ID NO: 98 - DNA, 221 bp, Artificial Sequence
  Synthetic
atttttattactctctcaagtagtctcatcttcttcttaatccaaaggcccaaactttgaatcatcactatcactctctctctctctctctatctctcaagaactgcacggacaacgacatgcttttaatttccatgcaaatctctcctttcttctcaagtcatttttgaaaatcaatcaaaaaactgaaacttggtggagcttttatcattcactcatca

SEQ ID NO: 99 - DNA, 294 bp, Artificial Sequence
  Synthetic
ctttcacccactttaatatgccaaaaaataagaacaaaattatatccgttgcttgaaaatcacaagctcttcttaacttcacaagtgcttcaatggcggttcttcacattatcttcactgcgtaattgaagaagttgttctctcttcctcttaatttcgagttgtgttcttaaaaaactccagagctgattcgattctcgagaagaaactaagccgacaataaagttcagatctggaaaaaagcgagctccagattacaaaaagaaacagctcgtttttttcactttcaaaaaa

SEQ ID NO: 100 - DNA, 159 bp, Artificial Sequence
  Synthetic
gagtctggttcgaaaagactgcttcaatgaagccaaaactatccaataactcgaaattgactactcttttcttttgtctctgttgttgattcgcaaaggcgaagattatccatcttctcagttactcctactggaaccaaaagctcagaaccttaaaac

SEQ ID NO: 101 - DNA, 456 bp, Artificial Sequence
  Synthetic
gaagcaattgttgcattagcctacccatttcctccttctttctctcttctatctgtgaacaaggcacattagaactcttcttttcaacttttttaggtgtatatagatgaatctagaaatagttttatagttggaaattaattgaagagagagagatattactacaccaatcttttcaagaggtcctaacgaattacccacaatccaggaaacccttattgaaattcaattcatttctttctttctgtgtttgtgattttcccgggaaatatttttgggtatatgtctctctgtttttgctttcctttttcataggagtcatgtgtttcttcttgtcttcctagcttcttctaataaagtccttctcttgtgaaaatctctcgaattttcatttttgttccattggagctatcttatagatcacaaccagagaaaaagatcaaatctttaccgtta

SEQ ID NO: 102 - DNA, 268 bp, Artificial Sequence
  Synthetic
aattggtggatgtcgtcgcggttcgaccccaagggatttggccggtaaaattattgggagttgtctttctcttgcactctctctagttccaaaccctagcaattcctctgttttcaccattttcggagattatcaccttctccccgattcgccgccttgtgattacatctacgtaaagagtttctggtagaaattttccctcttttagctgcagattggcatcagattccgttctggatgtgtcggtgatcgattttccgcgtcggtg

SEQ ID NO: 103 - DNA, 288 bp, Artificial Sequence
  Synthetic
atttcataaatcatagagagagagagagagagagagagagagtttggaaacattccaaaaccagaactcgatattatttcaccaaagaatgatagaaacaagaactatctttttataaaactctttacaccccaaaagaaaatgtctcactcgttttgccttataatatttctttcaacaacaacccaaatctacaaaaaatcccaataaaaaaaaacttcagtctgtttgacattttgtcgaacacttggacggcatcacaaaaagctctaaactttctgactacca

SEQ ID NO: 104 - DNA, 262 bp, Artificial Sequence
  Synthetic
gaccctcttctctctctctagctagtctcaggtcagagaagccatcatcaacattcaacaagagagccgtgtttgtgtcttgactgattcttctctcaagcttttttaatctctctctcttttcccacgtaattcccccaaatccattctttctagggttcgatctccctctctcaatcatgaaccttcttctcttctagaccccacaaagtttcccccttttatttgatcggcgacggagaagcctaagtctgatcccgga

SEQ ID NO: 105 - DNA, 69 bp, Artificial Sequence
  Synthetic
atggttctgtaaccggacaacatctcaaaacttgttctgttttttttttttcatttcttagacagaaaa

SEQ ID NO: 106 - DNA, 581 bp, Artificial Sequence
  Synthetic
aagaacaaacaactaccaaacttgtaggcagtagcaggaggaagtgggtgggattaacattgtcatttctctctctttttcttttacaaatctttccgttttgttttcttttggttttccggtgagcactgttgtgtttccaattccggcactctttagggttccctttcagaagaaaacttcacattgttgtttctctcaaccgtgacatcttggattactacttctgactactttcctttttcatgtgccccaaaagataatagttactttttcaaaatctggttttgttgtttgggtttgtgtcattcattgataaagtcactaatggagaagtacaaaacaattgcaaaatttcgaatctgtgttgtcattgctgaattctgtagtggatgtttgcttgcagtttagagcttcggagtgcgaagagtgagacacaagaggattctttctggaaccgcattattccctttagaggaggaagaagaagacaactcactcacaaggaaaacaaaggttcctctggttactctgaaatattcaaaccaatggtgagcaattggtagcacttgctaaagaag

SEQ ID NO: 107 - DNA, 132 bp, Artificial Sequence
  Synthetic
aaacacaaaaaaacgaagatagccatcgttttggtgagagaagagagaagagagaagaagaaggccatggaaagataatactctgctttttttttagaaatatacagaggaaataaagagagagagaaggag

SEQ ID NO: 108 - DNA, 113 bp, Artificial Sequence
  Synthetic
tatggactctcgttctcagacatttatttctctcagtcttacaatataaattttcattcttaccatccataattttgtattgtcttctccacagatctattccagctcacgcc

SEQ ID NO: 109 - DNA, 468 bp, Artificial Sequence
  Synthetic
acaatatcacaaactcgtttgctcttttcatcattactaaatcataagcggctctcaagttctttagggtttcgagttttctcaatctcctacctgattaaggttaatttcttatcttggatcaataacaagagaattataactccggattgtaatcaatattcctctacataaaaagcgtgaatgagattatgatggaatcgaaagctggtaataagaagtcaagcagcaatagttccttatgttacgaagcaccccttggttacagcattgaagacgttcgtcctttcggtggaatcaagaaattcaaatcttctgtctactccaactgcgctaagaggccttcctgagtactagccagttccctccatagcttttcaattacaacaatctccttttctcaaagctctggttccccaaatcctctcgtcttttgtttgccctcacaacaacaacaacaacgcagag

SEQ ID NO: 110 - DNA, 134 bp, Artificial Sequence
  Synthetic
aaaaaataatccccaaataatggagacgaagtggagagagaaagctcccactctctcacaccccaaagcttcttcttcttcttcctcttcttcctcttcctcttctctaatctgaatccaaagcctctctcttt

SEQ ID NO: 111 - DNA, 152 bp, Artificial Sequence
  Synthetic
gaagatctcatttctctttctccttttcttctccgacgattcttctcagttctcagatctgatcgatttcttcatcagatgtttcaatctaaccattgagattgaatagtcaccattagtagaagcttcgagatcaatttcgaatcgggatc

SEQ ID NO: 112 - DNA, 714 bp, Artificial Sequence
  Synthetic
tctttcccttcttcttccccaataatctcgctgaaactctcttgctcttgcttctaaaaatctgttctttgagactttgatcacacagttatcaaaatcataatctcttctttcctggtttttttttttttcttcttcttcttcccgtttcacggtacgtttactctgttcgatcaccgagtgtatgataaaatgtttctgtgaaatcaaataacatatcactttctaataaacatcaaaatttctccttttttacagaaacaagaagtttttttgggaaagccgttgacttgactttttctttggggtgttgtgtgggagcttatagtatggtaccataagtgggagcttatagtttggggtgttgtgtgggagcttatagtatgaggaaaaatgttagatttgaagaatgcttcactgattttttaccataagtatgtcaactggattaagcttaagtagtaatggtttttactatgttcatgtggggatttctcttcctctctgtttacttcattccgagatgacttgagattttttcaaagtatagttcttggagttaagcttacctagtaatcactttatataacatcccttcgtttacatttgtgctttcacctggaaacactttagacttttctctcttctgccgtgtgtatttagttgtctagtcaaatttaagttgagtttaggctctagtctttggttttggtt

SEQ ID NO: 113 - DNA, 15 bp, Arabidopsis thaliana
gaaagagagagagag

SEQ ID NO: 114 - DNA, 15 bp, Arabidopsis thaliana
gagagagagagagag

SEQ ID NO: 115 - DNA, 15 bp, Arabidopsis thaliana
gagagagagagagag

SEQ ID NO: 116 - DNA, 15 bp, Arabidopsis thaliana
gagaaagaaagagag

SEQ ID NO: 117 - DNA, 15 bp, Arabidopsis thaliana
aagagagaaagagag

SEQ ID NO: 118 - DNA, 15 bp, Arabidopsis thaliana
gaagaagaagaagag

SEQ ID NO: 119 - DNA, 15 bp, Arabidopsis thaliana
gaagaagaagaagaa

SEQ ID NO: 120 - DNA, 15 bp, Arabidopsis thaliana
gaagaagaagaagaa

SEQ ID NO: 121 - DNA, 15 bp, Arabidopsis thaliana
gaagaagaagaagaa

SEQ ID NO: 122 - DNA, 15 bp, Arabidopsis thaliana
ggaagagaagaagaa

SEQ ID NO: 123 - DNA, 15 bp, Arabidopsis thaliana
aacagagaaagagag

SEQ ID NO: 124 - DNA, 15 bp, Arabidopsis thaliana
gaagcagaaagagag

SEQ ID NO: 125 - DNA, 15 bp, Arabidopsis thaliana
agaagagagagagag

SEQ ID NO: 126 - DNA, 15 bp, Arabidopsis thaliana
ggaggagaagaagaa

SEQ ID NO: 127 - DNA, 15 bp, Arabidopsis thaliana
aaaagaaagaaagaa

SEQ ID NO: 128 - DNA, 15 bp, Arabidopsis thaliana
gaaaaagaaagaaaa

SEQ ID NO: 129 - DNA, 15 bp, Arabidopsis thaliana
aaaagaaagaaagaa

SEQ ID NO: 130 - DNA, 15 bp, Arabidopsis thaliana
aagagagaagaagaa

SEQ ID NO: 131 - DNA, 15 bp, Arabidopsis thaliana
cgaggagaaagagaa

SEQ ID NO: 132 - DNA, 15 bp, Arabidopsis thaliana
taaagagagagagag

SEQ ID NO: 133 - DNA, 15 bp, Arabidopsis thaliana
aaaggaaagaaagaa

SEQ ID NO: 134 - DNA, 15 bp, Arabidopsis thaliana
cacggagaaagagaa

SEQ ID NO: 135 - DNA, 15 bp, Arabidopsis thaliana
gaaagaaaaaaaaaa

SEQ ID NO: 136 - DNA, 15 bp, Arabidopsis thaliana
cacagagagagagag

SEQ ID NO: 137 - DNA, 15 bp, Arabidopsis thaliana
aaagaaaagagagaa

SEQ ID NO: 138 - DNA, 15 bp, Arabidopsis thaliana
gagaaagaaagaaaa

SEQ ID NO: 139 - DNA, 15 bp, Arabidopsis thaliana
cagagaaagagagag

SEQ ID NO: 140 - DNA, 15 bp, Arabidopsis thaliana
ggaggaggaagagaa

SEQ ID NO: 141 - DNA, 15 bp, Arabidopsis thaliana
agaagaaagaaagaa

SEQ ID NO: 142 - DNA, 15 bp, Arabidopsis thaliana
gaaaaaaaaaaaaaa

SEQ ID NO: 143 - DNA, 15 bp, Arabidopsis thaliana
gaagaagatagagaa

SEQ ID NO: 144 - DNA, 15 bp, Arabidopsis thaliana
caaagagaaacagaa

SEQ ID NO: 145 - DNA, 15 bp, Arabidopsis thaliana
gaacgaaagagagaa

SEQ ID NO: 146 - DNA, 15 bp, Arabidopsis thaliana
gagaaagatagagag

SEQ ID NO: 147 - DNA, 15 bp, Arabidopsis thaliana
aacaaaaaaaaagaa

SEQ ID NO: 148 - DNA, 15 bp, Arabidopsis thaliana
aaaggaaaaagaaaa

SEQ ID NO: 149 - DNA, 15 bp, Arabidopsis thaliana
gcagaagaagaagag

SEQ ID NO: 150 - DNA, 15 bp, Arabidopsis thaliana
aaaagaaaaacagag

SEQ ID NO: 151 - DNA, 15 bp, Arabidopsis thaliana
caaagagatagagag

SEQ ID NO: 152 - DNA, 15 bp, Arabidopsis thaliana
aaaagaaaaaaaaaa

SEQ ID NO: 153 - DNA, 15 bp, Arabidopsis thaliana
aaagaagaaaaaaaa

SEQ ID NO: 154 - DNA, 15 bp, Arabidopsis thaliana
aaaggagataaagag

SEQ ID NO: 155 - DNA, 15 bp, Arabidopsis thaliana
gaacaagaagaagaa

SEQ ID NO: 156 - DNA, 15 bp, Arabidopsis thaliana
acaagaaaaaaagaa

SEQ ID NO: 157 - DNA, 15 bp, Arabidopsis thaliana
ggaaaagaagaaaaa

SEQ ID NO: 158 - DNA, 15 bp, Arabidopsis thaliana
ggaaaaaaaacagag

SEQ ID NO: 159 - DNA, 15 bp, Arabidopsis thaliana
tgcagagaaagagaa

SEQ ID NO: 160 - DNA, 15 bp, Arabidopsis thaliana
tagagagaggaagaa

SEQ ID NO: 161 - DNA, 15 bp, Arabidopsis thaliana
aacacagagagagaa

SEQ ID NO: 162 - DNA, 15 bp, Arabidopsis thaliana
gaagaagaaaacgaa

SEQ ID NO: 163 - DNA, 15 bp, Arabidopsis thaliana
gacaaaagaagagaa

SEQ ID NO: 164 - DNA, 15 bp, Arabidopsis thaliana
ccaggaaaaaaagag

SEQ ID NO: 165 - DNA, 15 bp, Arabidopsis thaliana
tgcagagaaaaagaa

SEQ ID NO: 166 - DNA, 15 bp, Arabidopsis thaliana
caaaaaaaaaaaaag

SEQ ID NO: 167 - DNA, 15 bp, Arabidopsis thaliana
gaaacaaaaaaaaaa

SEQ ID NO: 168 - DNA, 15 bp, Arabidopsis thaliana
aaaaaaagaggagaa

SEQ ID NO: 169 - DNA, 15 bp, Arabidopsis thaliana
aaaacagaaaaaaag

SEQ ID NO: 170 - DNA, 15 bp, Arabidopsis thaliana
agagaagaaacagag

SEQ ID NO: 171 - DNA, 15 bp, Arabidopsis thaliana
gaaaaaaaaaacgaa

SEQ ID NO: 172 - DNA, 15 bp, Arabidopsis thaliana
gaaagaaggagaaaa

SEQ ID NO: 173 - DNA, 15 bp, Arabidopsis thaliana
acagaaaaaaaagaa

SEQ ID NO: 174 - DNA, 15 bp, Arabidopsis thaliana
caaagaagagaagaa

SEQ ID NO: 175 - DNA, 15 bp, Arabidopsis thaliana
gggagaagagaagag

SEQ ID NO: 176 - DNA, 15 bp, Arabidopsis thaliana
tagagagagagaaag

SEQ ID NO: 177 - DNA, 15 bp, Arabidopsis thaliana
gaagaaaaaaacgag

SEQ ID NO: 178 - DNA, 15 bp, Arabidopsis thaliana
caagaaaaaacagag

SEQ ID NO: 179 - DNA, 15 bp, Arabidopsis thaliana
gcgagagacagagag

SEQ ID NO: 180 - DNA, 15 bp, Arabidopsis thaliana
gaaagaaataaaaag

SEQ ID NO: 181 - DNA, 15 bp, Arabidopsis thaliana
gacgaaaagaaaaag

SEQ ID NO: 182 - DNA, 15 bp, Arabidopsis thaliana
taaagaaacagagag

SEQ ID NO: 183 - DNA, 15 bp, Arabidopsis thaliana
caaaaacaaaaagaa

SEQ ID NO: 184 - DNA, 15 bp, Arabidopsis thaliana
aaaaaggaagaagag

SEQ ID NO: 185 - DNA, 15 bp, Arabidopsis thaliana
gaagaagaataaaaa

SEQ ID NO: 186 - DNA, 15 bp, Arabidopsis thaliana
tgacgagagagagag

SEQ ID NO: 187 - DNA, 15 bp, Arabidopsis thaliana
gaaacagagagagat

SEQ ID NO: 188 - DNA, 15 bp, Arabidopsis thaliana
agatgagaaggagaa

SEQ ID NO: 189 - DNA, 15 bp, Arabidopsis thaliana
tgagaagaaaaaaag

SEQ ID NO: 190 - DNA, 15 bp, Arabidopsis thaliana
cagagaaaacaagag

SEQ ID NO: 191 - DNA, 15 bp, Arabidopsis thaliana
aaaaagaaaaaagaa

SEQ ID NO: 192 - DNA, 15 bp, Arabidopsis thaliana
cgcaaagagagaaag

SEQ ID NO: 193 - DNA, 15 bp, Arabidopsis thaliana
tagagagagcgagag

SEQ ID NO: 194 - DNA, 15 bp, Arabidopsis thaliana
gagacggaaaaagag

SEQ ID NO: 195 - DNA, 15 bp, Arabidopsis thaliana
caaagagaagaacaa

SEQ ID NO: 196 - DNA, 15 bp, Arabidopsis thaliana
aaaagagagcaaaag

SEQ ID NO: 197 - DNA, 15 bp, Arabidopsis thaliana
cgagaaaatagagag

SEQ ID NO: 198 - DNA, 15 bp, Arabidopsis thaliana
ggaaaaaagaaagat

SEQ ID NO: 199 - DNA, 15 bp, Arabidopsis thaliana
gaagaagatcgagaa

SEQ ID NO: 200 - DNA, 15 bp, Arabidopsis thaliana
caaaaaaaaaaagat

SEQ ID NO: 201 - DNA, 15 bp, Arabidopsis thaliana
gaaaaaagataagaa

SEQ ID NO: 202 - DNA, 15 bp, Arabidopsis thaliana
aaaaaaaagggcgaa

SEQ ID NO: 203 - DNA, 15 bp, Arabidopsis thaliana
cacacagaaacagag

SEQ ID NO: 204 - DNA, 15 bp, Arabidopsis thaliana
tagagaaaacgagaa

SEQ ID NO: 205 - DNA, 15 bp, Arabidopsis thaliana
caccgagaaagaaaa

SEQ ID NO: 206 - DNA, 15 bp, Arabidopsis thaliana
ataaaagagagagaa

SEQ ID NO: 207 - DNA, 15 bp, Arabidopsis thaliana
cgggaaaaaaaaaaa

SEQ ID NO: 208 - DNA, 15 bp, Arabidopsis thaliana
aacacaaaaaaaaaa

SEQ ID NO: 209 - DNA, 15 bp, Arabidopsis thaliana
ccaaaaaaaacagag

SEQ ID NO: 210 - DNA, 15 bp, Arabidopsis thaliana
gaagcagatacagaa

SEQ ID NO: 211 - DNA, 15 bp, Arabidopsis thaliana
aaaagaaagtaaaaa

SEQ ID NO: 212 - DNA, 15 bp, Arabidopsis thaliana
aagggaaagagacaa

SEQ ID NO: 213 - DNA, 15 bp, Arabidopsis thaliana
aaatgaagaagagaa

SEQ ID NO: 214 - DNA, 15 bp, Arabidopsis thaliana
acggcaaaaggagaa

SEQ ID NO: 215 - DNA, 15 bp, Arabidopsis thaliana
gaaggaggtgaaaag

SEQ ID NO: 216 - DNA, 15 bp, Arabidopsis thaliana
aaagaacaaaaaaaa

SEQ ID NO: 217 - DNA, 15 bp, Arabidopsis thaliana
aaaaaaaaatcagaa

SEQ ID NO: 218 - DNA, 15 bp, Arabidopsis thaliana
agaagaaaataaaag

SEQ ID NO: 219 - DNA, 15 bp, Arabidopsis thaliana
taggaagacgaagaa

SEQ ID NO: 220 - DNA, 15 bp, Arabidopsis thaliana
aacagacgaagagaa

SEQ ID NO: 221 - DNA, 15 bp, Arabidopsis thaliana
gaagaagaatcaaaa

SEQ ID NO: 222 - DNA, 15 bp, Arabidopsis thaliana
aaaaaaaggtaagaa

SEQ ID NO: 223 - DNA, 15 bp, Arabidopsis thaliana
gccaaaaaataagaa

SEQ ID NO: 224 - DNA, 15 bp, Arabidopsis thaliana
ccagaagaaagagat

SEQ ID NO: 225 - DNA, 15 bp, Arabidopsis thaliana
agaaaagcaagagag

SEQ ID NO: 226 - DNA, 15 bp, Arabidopsis thaliana
gaacgagagagcaag

SEQ ID NO: 227 - DNA, 15 bp, Arabidopsis thaliana
agcaaaaaaaacgaa

SEQ ID NO: 228 - DNA, 15 bp, Arabidopsis thaliana
gataaaagagaagag

SEQ ID NO: 229 - DNA, 15 bp, Arabidopsis thaliana
aaaacaagtagagag

SEQ ID NO: 230 - DNA, 15 bp, Arabidopsis thaliana
taaatagagagagaa

SEQ ID NO: 231 - DNA, 15 bp, Arabidopsis thaliana
tccacaaaaagagag

SEQ ID NO: 232 - DNA, 15 bp, Arabidopsis thaliana
aagaaacacagagag

SEQ ID NO: 233 - DNA, 15 bp, Arabidopsis thaliana
agaagaaactaagaa

SEQ ID NO: 234 - DNA, 15 bp, Arabidopsis thaliana
tgaagacaaagaaag

SEQ ID NO: 235 - DNA, 15 bp, Arabidopsis thaliana
tgccaaaaaaaagag

SEQ ID NO: 236 - DNA, 15 bp, Arabidopsis thaliana
gagagaggtagcgag

SEQ ID NO: 237 - DNA, 15 bp, Arabidopsis thaliana
gccgcaaaaaaaaaa

SEQ ID NO: 238 - DNA, 15 bp, Arabidopsis thaliana
gacaaagacggagat

SEQ ID NO: 239 - DNA, 15 bp, Arabidopsis thaliana
ttaagagaggaagaa

SEQ ID NO: 240 - DNA, 15 bp, Arabidopsis thaliana
acgaaaagaagaaag

SEQ ID NO: 241 - DNA, 15 bp, Arabidopsis thaliana
cacaaaaagaaacaa

SEQ ID NO: 242 - DNA, 15 bp, Arabidopsis thaliana
ggaagaaacgcaaag

SEQ ID NO: 243 - DNA, 15 bp, Arabidopsis thaliana
tgaacggaaaaagaa

SEQ ID NO: 244 - DNA, 15 bp, Arabidopsis thaliana
gcacgaggaggaaaa

SEQ ID NO: 245 - DNA, 15 bp, Arabidopsis thaliana
tgagaagaagaacaa

SEQ ID NO: 246 - DNA, 15 bp, Arabidopsis thaliana
cactcagaagaagaa

SEQ ID NO: 247 - DNA, 15 bp, Arabidopsis thaliana
cctgaagaagaagaa

SEQ ID NO: 248 - DNA, 15 bp, Arabidopsis thaliana
acagaggaaagaaaa

SEQ ID NO: 249 - DNA, 15 bp, Arabidopsis thaliana
taataaaaaaaaaaa

SEQ ID NO: 250 - DNA, 15 bp, Arabidopsis thaliana
gaaggagaagaaaga

SEQ ID NO: 251 - DNA, 15 bp, Arabidopsis thaliana
gaaaccgaagaagaa

SEQ ID NO: 252 - DNA, 15 bp, Arabidopsis thaliana
aaaccacaagaagag

SEQ ID NO: 253 - DNA, 15 bp, Arabidopsis thaliana
aaaaaaaattgaaaa

SEQ ID NO: 254 - DNA, 15 bp, Arabidopsis thaliana
tacaaaaagaaacag

SEQ ID NO: 255 - DNA, 15 bp, Arabidopsis thaliana
caaacaaagtaaaaa

SEQ ID NO: 256 - DNA, 15 bp, Arabidopsis thaliana
tcggaaaaagcagag

SEQ ID NO: 257 - DNA, 15 bp, Arabidopsis thaliana
aagtgaaagcaagag

SEQ ID NO: 258 - DNA, 15 bp, Arabidopsis thaliana
tataaaaaaaaaaaa

SEQ ID NO: 259 - DNA, 15 bp, Arabidopsis thaliana
gtgacaaaggaagaa

SEQ ID NO: 260 - DNA, 15 bp, Arabidopsis thaliana
gaagggagggcagag

SEQ ID NO: 261 - DNA, 15 bp, Arabidopsis thaliana
ggagaagcaggaaaa

SEQ ID NO: 262 - DNA, 15 bp, Arabidopsis thaliana
gataaacaaagaaaa

SEQ ID NO: 263 - DNA, 15 bp, Arabidopsis thaliana
tgcagagagcaaaag

SEQ ID NO: 264 - DNA, 15 bp, Arabidopsis thaliana
aaaagaaacgatgag

SEQ ID NO: 265 - DNA, 15 bp, Arabidopsis thaliana
aaagaaagctgagaa

SEQ ID NO: 266 - DNA, 15 bp, Arabidopsis thaliana
cgccgaaacgaagaa

SEQ ID NO: 267 - DNA, 15 bp, Arabidopsis thaliana
agagagaagtgagag

SEQ ID NO: 268 - DNA, 15 bp, Arabidopsis thaliana
aagaaaaaaactgaa

SEQ ID NO: 269 - DNA, 15 bp, Arabidopsis thaliana
gagaaaaagtgtgag

SEQ ID NO: 270 - DNA, 15 bp, Arabidopsis thaliana
aatatagaaaaagaa

SEQ ID NO: 271 - DNA, 15 bp, Arabidopsis thaliana
gacgcaaagggcaaa

SEQ ID NO: 272 - DNA, 15 bp, Arabidopsis thaliana
catagaaaagaagat

SEQ ID NO: 273 - DNA, 15 bp, Arabidopsis thaliana
accacagaaaaacaa

SEQ ID NO: 274 - DNA, 15 bp, Arabidopsis thaliana
gaacaacaaacaaaa

SEQ ID NO: 275 - DNA, 15 bp, Arabidopsis thaliana
gacataaaacaagaa

SEQ ID NO: 276 - DNA, 15 bp, Arabidopsis thaliana
gagtgaaaaaacaaa

SEQ ID NO: 277 - DNA, 15 bp, Arabidopsis thaliana
gaaacaagtagagat

SEQ ID NO: 278 - DNA, 15 bp, Arabidopsis thaliana
caccgaggaacaaag

SEQ ID NO: 279 - DNA, 15 bp, Arabidopsis thaliana
aaccgaaaccaagag

SEQ ID NO: 280 - DNA, 15 bp, Arabidopsis thaliana
aaaacaaatcaaaag

SEQ ID NO: 281 - DNA, 15 bp, Arabidopsis thaliana
aagtaaaataaaaag

SEQ ID NO: 282 - DNA, 15 bp, Arabidopsis thaliana
gacacacacaaaaaa

SEQ ID NO: 283 - DNA, 15 bp, Arabidopsis thaliana
gagaaaggtggtgaa

SEQ ID NO: 284 - DNA, 15 bp, Arabidopsis thaliana
cggaggaatagaaaa

SEQ ID NO: 285 - DNA, 15 bp, Arabidopsis thaliana
taataagagtgaaaa

SEQ ID NO: 286 - DNA, 15 bp, Arabidopsis thaliana
caaggaaaaggcaat

SEQ ID NO: 287 - DNA, 15 bp, Arabidopsis thaliana
tcggaaaaatcagaa

SEQ ID NO: 288 - DNA, 15 bp, Arabidopsis thaliana
ctcaaagaaaaacaa

SEQ ID NO: 289 - DNA, 15 bp, Arabidopsis thaliana
aggcgaaggaaacaa

SEQ ID NO: 290 - DNA, 15 bp, Arabidopsis thaliana
tccccagaagaaaag

SEQ ID NO: 291 - DNA, 15 bp, Arabidopsis thaliana
cctgaaaagagcgaa

SEQ ID NO: 292 - DNA, 15 bp, Arabidopsis thaliana
gaaagaggtggtgat

SEQ ID NO: 293 - DNA, 25 bp, Arabidopsis thaliana
acatacacacaaaaataaaaaagac

SEQ ID NO: 294 - DNA, 115 bp, Arabidopsis thaliana
caaatccatctcatatgcttacgataacgtcccattgccaagctggttctttcactcttcaggagaaagagagagagagagagagagagagagagagttatcagagatagcaaaa

SEQ ID NO: 295 - DNA, 90 bp, Arabidopsis thaliana
gatagagattggagagcgagcgagacaaatcagaagagagagatttagatattgtagagtgagattctaaagagagagagagagagagat

SEQ ID NO: 296 - DNA, 88 bp, Arabidopsis thaliana
aggaggagaaagagaaagggggaagagaggagagagagagagagaaagagattagagagagaaagaagagaagaggagagagaaaaaa

SEQ ID NO: 297 - DNA, 66 bp, Arabidopsis thaliana
aggagattagcgaaaactcaaaacaggaacaaagttaaaagagtgagagagaaagaaagagagaag

SEQ ID NO: 298 - DNA, 104 bp, Arabidopsis thaliana
gttgtcttcagctgtgtacagaatcaagtttccaagagagaaagagagtaaaagcaaattaacaaaggaagactctgattcaccgagaaggttttggcttaaag

SEQ ID NO: 299 - DNA, 86 bp, Arabidopsis thaliana
attttggaatctttctctctctctctctctaaaaccagattcttaatagaagaagaagaagaagaagaggaaaggagaaatctgcc

SEQ ID NO: 300 - DNA, 78 bp, Arabidopsis thaliana
acgtcacgagacaaattagcatagcacgcaaagaagaagaagaagaagaagctccaagaatctgtcgcagaaatcgcc

SEQ ID NO: 301 - DNA, 77 bp, Arabidopsis thaliana
aaacaaaaccatctgacttatcaacaacaacaagaggacgaagaagaagaagaagattgttactttcttgatcgata

SEQ ID NO: 302 - DNA, 98 bp, Arabidopsis thaliana
tcagaacaacacagagccaaaggttttttgctcgcagtaaagaagaatcacactgtgaagaagaagaagaagcgaaatacaaaatcctcaggaaagaa

SEQ ID NO: 303 - DNA, 135 bp, Arabidopsis thaliana
cgtctttgaaagctaaaaagagagcaaaagcttctgtttattctccgattcgcagatcaattagctgggttttgattccgttgtgcgaaggactttaagaggttttgcagatcgaaatcggaagagaagaagaag

SEQ ID NO: 304 - DNA, 389 bp, Arabidopsis thaliana
gtcaagcagcttaaatcatctatgacttaaaattataattaagaaaaaacaatgcctaaatatgcatatatttcaaatgtatcacataacttgtgacataagaaaatataaacaaaacaaaaagggcaaaaaagacctgaaagcttagaggcacacctgcataggtcccacagttcactcgtgacaccgtaaaaggcaaaacacgaacccgccacgttatcacaaaaagcaagccacgtcaatatagtctcactgtcaactacacttaacttactattttcacatctcattttcctatctttatataaaccctccaggctcctctttaatttctttaccaccaccaacaacaaacatataaaccataaggaaaacagagaaagagagag

SEQ ID NO: 305 - DNA, 49 bp, Arabidopsis thaliana
tctttcttttgctaattctctatctcactcagctgaagcagaaagagag

SEQ ID NO: 306 - DNA, 291 bp, Arabidopsis thaliana
acaataaaggtttccagcacagagaagagagagagagattgcttaggaaacgttgtcggacttgaaaccagtttcggtaccggaatttagaaactccgttcaaatccggagccaatctctaaaggataaagcttccaactttatccattaattggagaaaattctcagagagactgaagtcgacaaagtcagagggtttcgttttttggcttctgggttttttatttcaagtgttcaatttccgaattaggtaagaaagttaggttttgagatctgtgcgaattgtgagag

SEQ ID NO: 307 - DNA, 77 bp, Arabidopsis thaliana
cttttacatttccggtaagatcaaaatcaaaaccaagttcgtttcgcggcggaggagaagaagaatcagacgggaaa

SEQ ID NO: 308 - DNA, 551 bp, Arabidopsis thaliana
ttaaattagagaaaaaaacgcagacgactaaaagatattcacacacaaaaaagaaagaaagaagaaaaattagctcacaaaataacaacaatataattaatacccaaaaaagaaaaaaaactaactgagtccatgttgaatagatctcctatagatgtaaggaaatactcggcttctacatcttaattaagcattacttcctatttctaaatagataggaagattcaagagcttctctcccagacgtgatttttgagacagccttttcatcaattttttctggcaccggtagagcgttagctcgtcggtgccaggagctagcttcttctcaccggtttcctcccataagctctctcatcggtttctctgttttttgtttcgtgttgtttcgtctcttttccctcctattagatccataaagcttcattaccgcacaaccttcgaaactactcccatctggtattagctcttctcttaccttgttcgcgattctcgtggatccctctcctcggctttccttaaagtcaagatcagcaactctttggtcctca

SEQ ID NO: 309 - DNA, 140 bp, Arabidopsis thaliana
aacgaaaaagaaagaaaaatctgtgaggacgaaaactctccgtcgttccggcgagtttctccagtgatcggcaaagtctttccggcatctattgaatttctctaaaccaattagaatattatcggtcttgataaaataaa

SEQ ID NO: 310 - DNA, 87 bp, Arabidopsis thaliana
aaaactcacactttctctctctctctctagaaaaagaaagaaagaagaaaaacttattgttattcccatttcgcccctatccgaaaa

SEQ ID NO: 311 - DNA, 289 bp, Arabidopsis thaliana
agaaacatcatgatatgatatttttctcaagtcttttggtgttggagaagaagagagaagaagaacttggtttctctctctaaaagtttattgcttggctccataaaaagtgcacctttttctctcttttctttctctctctctctctctctctctctctctctcacttctcctcggatgcactattgtccgtgagatcagagattcaccctctttagattttgcgcagaaacttttgcccacaattttgtattcgtcaaatctgagctgagatctctagagtgagaaa

SEQ ID NO: 312 - DNA, 35 bp, Arabidopsis thaliana; identical to SEQ ID NO: 81

SEQ ID NO: 313 - DNA, 98 bp, Arabidopsis thaliana
gttcttcttcattcattacaacaaactctttgagacctaaagagagagagagcgatagtgagatttagatcaacagatttgaatcgatttctgaaaac

SEQ ID NO: 314 - DNA, 240 bp, Arabidopsis thaliana
agaaaggaaaggaaagaaagaaaacaaaaggagtccaagaaaccagaagattgtctcccgacgccattatccttcaccctcggagcttttcttgaagcagggattcttctaatcattaatccctacttctttctttcttttttgtttgttctcctttgagatctatctagtactagtagtaaaaccccctcccctccattgaatttgaattgaattgaatctctgggaatcaaatctttg

SEQ ID NO: 315 - DNA, 291 bp, Arabidopsis thaliana
ttttgatatttcgacactctctctttcctctctccttgtctctgtaccgcgtcgaaatatgagaaacgaatgatttgatcatcaatcaacgagaaacacacacggagaaagagaatctcaaattagctccagctcctgatcgattccgattttcacaattctttccttggatctgctcttaccttgtcacgatttcacttccctgtgtttttgatttatacttggtcatccaataacgaaactttgatcaaactggaactacagtttattggaactccctgaagcatttag

SEQ ID NO: 316 - DNA, 102 bp, Arabidopsis thaliana
aattgaaagaaaaaaaaaaacgagaagcgttttctttctctccaaaatccattactcgcgaactttcctctgctaagtgttcactagaaagaggtggtgatt

SEQ ID NO: 317 - DNA, 140 bp, Arabidopsis thaliana
tggatgattgctgctttggtcaacgtttcaaaagaatcgttttttcttttagttccttccttctttcgctattttcgccattgattgctgaagaaaacacagagagagagagattcacttccccatttcagaaaatcaaa

SEQ ID NO: 318 - DNA, 245 bp, Arabidopsis thaliana
atgctgacacagatatttatttttgcctcttataacgaaaaaagcaaaataaaagaaaagagagaagagaaaagcattatcccttacgacgaggaagccgtcgttttgagggttcgtacaaatcctgagatcttccttcaaactctttctttgtctccttttttatctcactccgtcgtcgttttgattctttcaaagttcttcatcctctgttccgcgctgttttctggtgagtgttgattctg

SEQ ID NO: 319 - DNA, 62 bp, Arabidopsis thaliana
agaaaaatcagagaaagaaagaaaacagagcaattacttgaagaatccataggaagctgaag

SEQ ID NO: 320 - DNA, 226 bp, Arabidopsis thaliana
aataacaactatacaatgatatttttgatcaaacgtcattttccaatctttgaatctgagatgataacttgttcagcttaatctttccagtcaatttcatctccttccaattttgaagggttcatcagagaaagagagagccattcagagatccattgtaccaagctcacttcgatctacagaatcaccgagagctctctgtctctctgtcggtgatatttgtttg

SEQ ID NO: 321 - DNA, 540 bp, Arabidopsis thaliana
aacgtgctccggtgaagattaaaaaccgacgagaccctggcgccatcacaactacgcaatctcattcctccgtcttcttcggctttcaaatttaccattttacccttctctttccctgagacgtcttctttggaaatattcttctcttcttccattccaatgattttgaggttaattggaaattagagtgcaaaattgggatttagatggggattgctgatgaatctaaatgtgttttccccttgacgagtctccagatcggagacttgcaatcatatctttctgatctcagtattttcctgggaaataaaagtaaaaagatttacatattggtggataaccggccatggttgaatcctggcaccagatctgctcatttttggcaactaatggtcacaaagactctccccttttgcaaacacgaaacttcgaggggagaagaaaaatcagaatcaggacagggagaagaaaaagtcgaagcaggaggaggaagagaagcctaaagaggcttgttctcagccccagccggacgataaaaaa

SEQ ID NO: 322 - DNA, 131 bp, Arabidopsis thaliana
aaaactctactgtaactgcaaaatcttgttgttttcttaaacgaagagagaagaaagaaagaaaaaaacgttacggattctctgcttcggtttcgcgattgaagcttgagatttcatcttgaacatccgat

SEQ ID NO: 323 - DNA, 177 bp, Arabidopsis thaliana
aaaatctcacctttttgaccccaaaaatttctaaatatttcaaaatcagcctcttcgttttctttctcctcctgtctgttgatttaaagacccaaatctgacgcttctctctctctttctggtatctgcgtttgattcggagaagaaaaaaaaaaaaaaggcaaagagagagcttca

SEQ ID NO: 324 - DNA, 120 bp, Arabidopsis thaliana
acaaccctagaacaaaaaaagtatcccatttgtcatttgtcaattgtcattagcaagaacaggaagaagatagagaacagagctcttcgatcttttttcctccaaggaagaagtagaaag

SEQ ID NO: 325 - DNA, 272 bp, Arabidopsis thaliana
acacaatcgaagtcgaactctcaggattcaatcttgataccaaagagaaacagaaataaactaacatcatcgctactgtcgcctataatcttgtgagctctttatcgtcttcaatggaagttcgatgatgtaaaaactcaaataagagtgattctagaatgggaaattttctatagaaaggaaaggttttccaaaactttaatgtagtacagagctgctaccgacaaaataagcagtttaagacacgataccaaagagaacctgacctgttc

SEQ ID NO: 326 - DNA, 295 bp, Arabidopsis thaliana
aattgttttgaggtagcagctgcaaaccgctcaaacagttgcgcattaggcattacacagttccactcgttccttttgaagcttatctgtgtgactctaatctgttactataataggaacgaaagagagaactaggatctatacttgctccaaccttgctttgtttctcttctgcgatttatctctagatctactagatctggacaaggagcgaagcgaattgctggcaaattttagttttggagttttgaaacccgacgattatcgcgcttgatcgttgcttctctgatcggaa

SEQ ID NO: 327 - DNA, 70 bp, Arabidopsis thaliana
ctgaattacgaaaattctgtgaggttgaggaagcagagtgaagagaaagatagagagataagaagaagcc

SEQ ID NO: 328 - DNA, 70 bp, Arabidopsis thaliana
aacacaaacaaaaaaaaagaactctttcgtcgactaatgtgatttattgttcaccggagtattaaagaag

SEQ ID NO: 329 - DNA, 282 bp, Arabidopsis thaliana
aaaggaaaaagaaaaataaataatcgatctcaaccgtccgatcatccatcttgccatcaccgttcaccaatcttcttcgtctcctctctctttctctctttttgctgtttctagctcctctctctctggatctcgccggcgaaccgtttctcttgggtgtaaacagtagcaatcaagctatagaatctcagatatcgctgaattagctgttggattttatccgccttttcttcgttatccggggctcgggtataaggtttcatcgtcttatttcatctgtaa

SEQ ID NO: 330 - DNA, 126 bp, Arabidopsis thaliana
acttgtttccttatatattcttctccctttaaacatttaatcttttcctcttctaccatctccacaaattccaaacatctctctctctttctctctcacacacaaaattgcagaagaagaagagtc

SEQ ID NO: 331 - DNA, 30 bp, Arabidopsis thaliana
aacggaattttcccaaaagaaaaacagaga

SEQ ID NO: 332 - DNA, 37 bp, Arabidopsis thaliana
aaatcaaattcattcatatcaaagagatagagagaaa

SEQ ID NO: 333 - DNA, 398 bp, Arabidopsis thaliana
aaaagaaaaaaaaaaatctcagtcaagttcgtccgaaagttttcaacgacgacggctttttagagatttgattcgtttcactcttctgggtattgattttcttccttaaatttgcatcctttttaacgtttatccaacgatcttgctccgttactgaaactctgtttctccgttgcttctctcgtctcatttattgttcgtaacgtgattttactacttctgttactcgagtagagattacccttcttatgtccgaatctgattcgtcgtctttaagctttgtcttctcccaattagctcaaagttcgtaactttgtttacttgccaataagaaatttccagagactgaagtttccattgaatgtattgttcttggagaacttaaccggattcaggac

SEQ ID NO: 334 - DNA, 159 bp, Arabidopsis thaliana
ctcgaagactattaaaggaatatccgcaaagaagaaaaaaaaacatttttttggtaaaggactaatctttttgtttgcatcggccatctctaaccttacgattgtgtgttcttgctttgagcgaaaccctagaatcggtcttaacccatttgagcagag

SEQ ID NO: 335 - DNA, 274 bp, Arabidopsis thaliana
acattagcttcctcatttttattcttattattattattcatcagaccaacaacaaaaaggagataaagagaagaggattcatcatcatcaatcaatccttcattttatggatctactcatatcttgattcttccttctatctctcccttttcttccatctctttttctctgggtttccccggattgagttttttaatctctgattgacagatttgaagagcgtgacaaaggaagaatcttttattaaaacaaattcttctgttttaatcttggg

SEQ ID NO: 336 - DNA, 43 bp, Arabidopsis thaliana
aaacgaaaagcttttgaagaacagaggaacaagaagaagaaag

SEQ ID NO: 337 - DNA, 80 bp, Arabidopsis thaliana
agctcatattctctcactttctctctcagcttacgaacaagaaaaaaagaagaatctttagccacctttgagatcaaaag

SEQ ID NO: 338 - DNA, 203 bp, Arabidopsis thaliana
atccaaaacgtttttccttcccacaggaaaagaagaaaaacagacagcggaggactaaaacaactagccacaacacaacgcttcaaatatatattactctgccactttcttcaatcttccttcaaagattcttattacagcgacacacaactcttttccatttagatttttgattttttttggttctctaaaggaggagagaa

SEQ ID NO: 339 - DNA, 71 bp, Arabidopsis thaliana
aactttttcaaaaaaaggaaaaaaaacagagctcactcattattatctctctaaaaaccctagctttctcc

SEQ ID NO: 340 - DNA, 139 bp, Arabidopsis thaliana
aatcagctgcagagaaagagaagtcaaaacgcagctctctcttgcgttttcttcctttctcctttctcaattccccagagaacaacataactctgtaaaagggaaactctattttgttctgaatcaaaagtagttttaa

SEQ ID NO: 341 - DNA, 70 bp, Arabidopsis thaliana
atttctctttctttcttaagctttttcacaagactagactttagcttatcgttctagagagaggaagaag

SEQ ID NO: 342 - DNA, 119 bp, Arabidopsis thaliana
atagaatttctcgtttttatcacccgcttcatttgcctttctatcgccacaagaacacagagagagaacgattagcccagttccgatatcgttcggtggcttcttcatctgaagctacg

SEQ ID NO: 343 - DNA, 68 bp, Arabidopsis thaliana
gacagtcagtcactgtaacattttagatctttcccgaagaagaaaacgaagaagagacgaagagagaa

SEQ ID NO: 344 - DNA, 290 bp, Arabidopsis thaliana
cagaaacagagacaaattctaaaaaagaaacaatctttagacaaaagaagagaaatttagtcatgggttagtctgcaaaattcaattacgtcttcttcttcttcttcttcttcatctttgatttgttggcgtgtttagggtttgggatttggaggagaggcaaaatgttgaattaaataaatcgaacgactctggattcctcggcggttaacgaccgccgtcgccgccgccgtcataatccaaccaccaccaccatcaacgaccttgaatttccacaatatgcttcatca

SEQ ID NO: 345 - DNA, 209 bp, Arabidopsis thaliana
acaactttatctcagctttttcttctcaattaaaatcagtttgggattttttcgaaaacgcttttcaatcttcgtctatctgtctccacgatccacgccttgaccttcgttttttttttctcagagattagagaaaactccgataaccaatttctcaatctttttgtagatccaatttttccaggaaaaaaagaggtttcgcgaagaag

SEQ ID NO: 346 - DNA, 72 bp, Arabidopsis thaliana
agtgagtcacataaccctcttggaaagagtctcaacacttgcagagaaaaagaacaaggaagatcccggaaa

SEQ ID NO: 347 - DNA, 427 bp, Arabidopsis thaliana
ttaaacccagaaatcaccaaaaaaaaaaaaagtacatttcctttttttttgttcttaaatttttctgtggttccggtcaccgcagctctgtcatcatcttcttcttcttcatttaccaatctgaaatctactcagattctttgtgattttctccttaaaatctcgatctgtatcgtacagtgacttgtgaaattaggatcgttgtgtctgtgttttctggttacagtttgtaaaatttgaatatttgtgtgtgaagtcagattcagtttcgtgagctgttcggatttggtttgggggtatatatatagcgttgtgtgatctatttggggggttttggtttccctttttttctctcttgtgaattcgtttattgttgtatcgtcggcccgagtttatcggaactccgggtctgacgtgagttttccaa

SEQ ID NO: 348 - DNA, 96 bp, Arabidopsis thaliana
attcatcaccacaatcacctgaagagccaaagcagcaaaagaaacaaaaaaaaaacaagaagtgaagtcagatctcgaaaaagagtttacgaatcc

SEQ ID NO: 349 - DNA, 249 bp, Arabidopsis thaliana
aaacgttactgtcactaaatgaaatctatttttctttcttaaattttgctctgacaaatatttttgattgcgtcattttctactttggaaatgtctttgatttagcatttcagttcgctcaaaacatcaaatcttaccttctttagctttcacattagattctggtaattattagcacaaaaaaaagataagccagaatacgaaacaaccaaaaaaagaggagaattcttttttttttttttctttccg

SEQ ID NO: 350 - DNA, 79 bp, Arabidopsis thaliana
ctcaagaaaacaaaattactttaaaacagaaaaaaagttgataaattgcttcagtgtcaaattctgagatctgtaaaag

SEQ ID NO: 351 - DNA, 504 bp, Arabidopsis thaliana
taaaataaatgagaagaacaaaaattcagttgttaaaatcaaagtagtgtctctaccgtgatttttatttttttctatatactgtttaaacctcagtttttttgttgttgttataagatccttgtcattttttgtcgtgattagatgtaatttgtataattttagtaactcttcagtttttttttgttttaaaaatatattttctctctctctgtcttcctgcaatctatcgccggccgattcaataatttcgctttactctgccaaaaaagtttgttcttttgttttctgggattatccaaagagaagaaacagaggaaatcaatctcttttttagtttcagaccctaaatcctaggttttgaagttttgtttctttagtaattttgtcaggttttgtgtctggtgttgggatttttcggagcttggtttcttgaaccagctccattttctaaaaattccttctttaaatccccattgttgtaagtcttaaagaaaaaagaag

SEQ ID NO: 352 - DNA, 69 bp, Arabidopsis thaliana
gtcatttgctaaggaaaaaaaaaacgaaaacgtgtgtctgtctcttctcgtagcgtctctcaagctcag

SEQ ID NO: 353 - DNA, 106 bp, Arabidopsis thaliana
aaaaccaacttctaatttggaatcaaattgaaccgaatcgaaccggttgaagttgaaagaaggagaaaaggcgttgtctccgtgcgagaaaggcaaatcggagacg

SEQ ID NO: 354 - DNA, 387 bp, Arabidopsis thaliana
ttttttattttcttgacaagtctgcatttttctcctctgttttggaattttctcgtttctggttttccgatcataaaaaacaaacaaaactaccgtaaaataggctctctccacagaaaaaaaagaagacttttctttcattcttctgcaagtaactgagcagatttcggttttttcttcttcaaattgatatttttaaagttataaaaatttcttgtccataatttccgttttccttaaattcagctgtcctaacgtcaaatctcagacactcgcttgcgtgtctccctctcttaaactctctctttctctttctcttttggtttctgggttatttcaaagaaaagaatcaagaaacccctctttctctcttacaagaatcccatc

SEQ ID NO: 355 - DNA, 32 bp, Arabidopsis thaliana
aaaacctcacagccacacaaagaagagaagaa

SEQ ID NO: 356 - DNA, 165 bp, Arabidopsis thaliana
atatctgtctcatctcatctctcatcgttccgggagaagagaagagagacccatccctcacttcaaagttcaaagtctcgaaggatcttctccaactctctctaaacaagattccaaattttcaaaggtgaatttgtttgatagaatcaagaacaaacctttaaa

SEQ ID NO: 357 - DNA, 101 bp, Arabidopsis thaliana
atatttcttcccatcgtcactagtcacgaccacacaaacaaaaaaaatataacatttagagagagagaaaggtacagcagtggcaaactcgtaaataaaga

SEQ ID NO: 358 - DNA, 233 bp, Arabidopsis thaliana
aattatggtttacgaagactgagaagaaaaaaacgagcatcgtccatcgagatccaaatcctcagtttcattttcatctctctctctcgtattgatcagctactcgaaactccggtaacggattttcacaatcccggcggcgaaactcttcttcccggctaagttttcattttcttcagattcctcgtaaagttgccggtggaccaaggtccaactcttgaacaccccaaatc

SEQ ID NO: 359 - DNA, 140 bp, Arabidopsis thaliana
cattcatttgttctttcttcagagaaaaacaaaaaacagagcattttttttggtcaagagcaagaaaaaacagagcatacttttgcaaaaagcagagcttggagcgctttcttgtcatctaaaattcaaaggcagagacg

SEQ ID NO: 360 - DNA, 132 bp, Arabidopsis thaliana
gtttggaaataacgtgtaagtaggacccacttttgtgattatccgccgcacagaagtctctcctccactccacaaatagcattcccggcgagagacagagagcgaagaagaagactcaaaccaaaaaaaaaa

SEQ ID NO: 361 - DNA, 352 bp, Arabidopsis thaliana
atcgacggttagaaatgaaacgattaggagattagatcgttgaacaaaacgacgtgttttggtctatttataaagaaagaaataaaaaggagagatgaccaaacacgcctttatcatagtttctatctccgatgacacaaaacgaggaagattatttgacattttaagtaagaacagctagctttgccatctccctaaaggcaataaatctcggatccactttcacgatattttgatattttttctatttataatctttctgggttttgagtcttttgaaggctgaattgctctgaaatctcaattgtataatcatctcctgggtcgtcgttatcgtgatcatctagaaagc

SEQ ID NO: 362 - DNA, 123 bp, Arabidopsis thaliana
atccaatcatagacgaaaagaaaaaggttcctttttttgactttgtatccgtagatcatcttcttcttcttcttccagagttttatccttatccgttccatcaaattctctctctaagcaaag

SEQ ID NO: 363 - DNA, 255 bp, Arabidopsis thaliana
gtttctcatctccagctctcattttctctctcatcttcaaccttaactctcttttctctctactctttctttggacgaatctgtctattgtttgtaagttttcaaggaaggtaaagaaacagagagatctaacttcgtctgcagggtttaagcagaggttggtttgtggattcttcgatttcttcttcagatttagtctacaatgaagtgagaatttctaaagataaacaaagaaaaacttgagactttagcaag

SEQ ID NO: 364 - DNA, 109 bp, Arabidopsis thaliana
aattgtctcttcttttctttttgtacttgtcaaaaacaaaaagaacaacaaaaaaaatctcaaccgtagaaaattccgacaagagttcagttcatacaatgaactaagt

SEQ ID NO: 365 - DNA, 115 bp, Arabidopsis thaliana
atcttcggaaagtctcatttctcgatccccaattcgtggattagggttaaaagaaccatttttattctcgtcgcgcaacaacaaatccagatcgaaaaaggaagaagagatcgaa

SEQ ID NO: 366 - DNA, 589 bp, Arabidopsis thaliana
taatccaatcttcttcttacataaacacctctcctcccccaccgtttccaaaagagagaagctttctcactaacaccaaaaacaagtctttgaagaagaataaaaagattggattttgataagtttagtgaaaatgggggagcttttgtgttcttcactgtggaacccgtcacgattcattgttgcttctctcaaaaggtattttctgggtttagcttcttagaggttcttcgttcttaaaggtctgtttttttttaggttgtgatactttgaatgtaaaaaagggaagatttttagtttcgatatgtatatctctcggatgggtttgagtcggagtttcccgccgctttttgggggatttcgggaaattctagggttagggttggatattgtcttcctctagcagtctctgccacttttaaaatctcttcatctttctttgagagtgaaagaggtttttttatttgtttgtgtcttcctgggaatcgagattctggatcttaatcaatatgtgggttaattgggagatctgggatttgggagatcttgtggtggattgaagaaaaagcaaggttgtagattttgaaaa

SEQ ID NO: 367 - DNA, 85 bp, Arabidopsis thaliana
ggtagaaagaaaggatttttatttatccagaatcaatcgccggagaagaagataaacacagagagtgacgagagagagagtgaaa

SEQ ID NO: 368 - DNA, 155 bp, Arabidopsis thaliana
cctgtctagcgttgacgacaccaaaattgaaaatttggcatcatttgcgaaacagagagagatccattcaattccaaaaggattctcttttgggaaaaccctaaatcgacccaccaaatttggagactgtgattgagcatgagcgtcagaagttg

SEQ ID NO: 369 - DNA, 208 bp, Arabidopsis thaliana
attagatccctttaattttagtaattaagtaaaaagattataaaagatgagaaggagaagatagcttcttcatcgagaaacctcgaaatcaaaaagcacgtcggtgacttgtactcttcaatctcttcttcctctctttcacatctccttctctcgaacccatcgacctgcgctaattcatcatcgaccttgctcaaattcatcaacc

SEQ ID NO: 370 - DNA, 115 bp, Arabidopsis thaliana
aacttccaaatcctttatataacttctcacaagtcaccaccatttctctctagaaaatatcagaaaaacaaaaccatctcaaagtttcttgagaagaaaaaaagggtcaagaaag

SEQ ID NO: 371 - DNA, 109 bp, Arabidopsis thaliana
gagtccaagttgactccttcgagctttgattctcgttccaataatacttcctccaccatctctcctcctctcgttagatctaagaaacagagaaaacaagagagataga

SEQ ID NO: 372 - DNA, 198 bp, Arabidopsis thaliana
ccaattctaaaccaaacaacagattctcataatcatctcttcttttttcctctttacgaaaagaagaaagatcaaaccttccaagtaatcattttctttctctctctcacacacacacattcactagttttagcttcacaaaatgtgatctaacttcatttacctatatgcaggtttacacaaaaagaaaaaagaacg

SEQ ID NO: 373 - DNA, 51 bp, Arabidopsis thaliana
ctagccgcaaagagagaaagggagggaggagagtgtagcagatcggcgaaa

SEQ ID NO: 374 - DNA, 62 bp, Arabidopsis thaliana
gtccagcttctgagctcagagatagagagagcgagaggttagagataacagtagttttaccg

SEQ ID NO: 375 - DNA, 213 bp, Arabidopsis thaliana
aaattgataacttctaataaatggagggtgcaattaataaataaggagagacggaaaaagagacgccgttgaaacaccgcaaaacagagaagcgccttttgattgtctctctcccggagatctctctttctcttcttctccatccttcttctctcggcgcgcgcttcatccccaccaccttcgaattcgtgccctttgagggaagctgctagg

SEQ ID NO: 376 - DNA, 162 bp, Arabidopsis thaliana
attttatagagacgtctctggaaaaaacattcccaaaattggcttataaatactttcaaaaccacaaggccacaactcatcattcgcaccaaagagaagaacaaaacatcatcatatattctattgactagattaatttcttctaagtgcaaaagaggagaa

SEQ ID NO: 377 - DNA, 135 bp, Arabidopsis thaliana; identical to SEQ ID NO: 303

SEQ ID NO: 378 - DNA, 186 bp, Arabidopsis thaliana
tccgtgattcttctctttagcttatttttggggaagacaattccgagaaaatagagagtagagagatcctaaagagtcaaaagaggtcaggtgattgattaacccgttgaataatctccttctcccgttgaatcgggtcgaaatagttgaactttaagccaaaccctagcttgaggaggaagagga

SEQ ID NO: 379 - DNA, 76 bp, Arabidopsis thaliana
aaacaaacgcaggaggcctggaaaaaagaaagataacgggactcgagagattgagattacggagccacccactttc

SEQ ID NO: 380 - DNA, 171 bp, Arabidopsis thaliana
tatatgctttctctggacaaacgcaaaaacttttgtagaaccctaaaaattcccaaaatccgtcggagaagaagatcgagaagaatcaacaactaatctgaagaattttccaaattccgtcttcgtatcgtctacgagatccttatctctcccctgaatctggaacctttg

SEQ ID NO: 381 - DNA, 347 bp, Arabidopsis thaliana
aacaaaactcgaatcagagaattccagatattacttacataagacaattttagcaattagctttcaaatctcatctctttattctctctctctatctcttctcctcaagaaccctaaaaatctccagaaaaaagatcccaaatttcgtatttcaacgatctgaatctctctctctttcgggtttattttgtttcccgatatggtttagaatttgtgatttaaatggaagctgacgtgtcaatttcctgaaaaaacccttatcgcgaaattttccagattaccaaaaaaaaaaagattgaaacttttttcgatttgtttgaagaagaagcacggtaggaacgacgacg

SEQ ID NO: 382 - DNA, 355 bp, Arabidopsis thaliana
agagagagagagaacacaaagtgggaaaaaagataagaacccaccataaagttttaacatttttcccttcaaaaggcgaaagcttttgatttgtataaaagtcccacttaatcacctctctagcttctcattccatttccatctcctctcttttgttttctaagttgcttcaagagttttggatagtgtagcagagagattttaactaatgggtttataaaattttgttcttttgcgtgaacaagttgtcaacttctagacagattttctttttgaagtgttttcttgtcgaaattcttcttcttttggtcaaagaacgcaagattcttctgtagttcctctaaaaaaaatccta

SEQ ID NO: 383 - DNA, 508 bp, Arabidopsis thaliana
cgtccttcttatcattataatcatctttttaatcaaaaaaggtttgcacataacataagcttttttctttctctcttaatcagaaaacaatcttgtctcacaaaaatataattaatgattctaaatttccctaaccgtccgatcacaaaagatcgtgatcatcgcgtggaaactttagaccaatcttttccctaaaccggaccgtaccagattccttctctctctctctgcttagagagttttaggttcgttttcccacttaagccaaattggacaagatttggacgtttctgtatctctcttaaagctaaaaaaaagggcgaatttttccatggcgttgtcggagtttcagctagctctgagcttggtggtcttgttcttctagctgatttgatcgaaaccccatgttcttatgattttacacgacctaatccaaaactccagagcacacggagacggagtacatattgttcagcgcaagtgaaagcaagagcctttttgtctattg

SEQ ID NO: 384 - DNA, 88 bp, Arabidopsis thaliana; identical to SEQ ID NO: 94

SEQ ID NO: 385 - DNA, 231 bp, Arabidopsis thaliana
aaaggaagaaaggggtagaattggaaatatgtagagaaaacgagaataactctgacgcgaacgtttctctcctccgtctctcgatccctctcttgacgtctcgctgatctgttttgctaagattcaagcttcaaaaccctaatttctctagccattagcatcgatttcagctcaacttcagattcaaggaaacaattattagcttctcaagtgcttcagtgatccgataca

SEQ ID NO: 386 - DNA, 71 bp, Arabidopsis thaliana
gttatcctcatctagtcatcttcaccctctaactcaccgagaaagaaaagtaaagagagtttggtgtcact

SEQ ID NO: 387 - DNA, 125 bp, Arabidopsis thaliana
gagccctcacttgacagaactcagaaatttgaaagagaaataaaagagagagaagctcccagagaagaaaagccctaaaagccccactcctctttccagtttcttttgatctctcagcatcgaaa

SEQ ID NO: 388 - DNA, 121 bp, Arabidopsis thaliana
ctttggtcctacttagtacttacctgcccctctcgacaaaatttcttttgtactttcacatttctctgtaataaactcggtaggtttgcgaaaacctcgccgccgggaaaaaaaaaaatca

SEQ ID NO: 389 - DNA, 293 bp, Arabidopsis thaliana
aatctccccttggttgatcggtgaacacaaaaaaaaaaatctaaaataatcgcaaaatacatttgaagaagctacacgatcaacaacagcaaaggatttcgattgttgaaaaagttgactcttcttaatttgattcgttgtcttggtttctgggttttcttcttcttcttctgcggcgctctccaattttacaccttgcgaccagcgagaaaagaaacaaatttcacccccattgaagaaggacctttggttaagctccatggtgtggtatgcgcaaagtggacaatacctag

SEQ ID NO: 390 - DNA, 54 bp, Arabidopsis thaliana
taagagacagagagatcttaacacaaaacaaagcaaacaccaaaaaaaacagag

SEQ ID NO: 391 - DNA, 120 bp, Arabidopsis thaliana
aaacccattgctcaagaaaacttttcagacagatttgtttcgagaaaagatcgcttgcttggcttttcaggataatctgagatctatctgtagaagaagcagatacagaattcagaaacg

SEQ ID NO: 392 - DNA, 37 bp, Arabidopsis thaliana
aaaaaaagaaagtaaaaaacgcgtcagggaagagaag

SEQ ID NO: 393 - DNA, 214 bp, Arabidopsis thaliana
aataatgtgttgcaaaagaggcaaactatacaacgtgaaagtggtaggtctaccagatcccataccctcattttaatggcggagattacaagggaaagagacaactccaattcaaagctctgattttttccaccaatccccattttttcccttttacaattcttaagctagttttatacttttcttcttcctttcatttgggttaagagaagcc

SEQ ID NO: 394 - DNA, 285 bp, Arabidopsis thaliana; identical to SEQ ID NO: 97

SEQ ID NO: 395 - DNA, 347 bp, Arabidopsis thaliana
atccgacggcaaaaggagaattaagatttttaactttaaacgagagtttcgtttatttactcaaaaatttacttctgaaatctctatttgaatttcggggaaaaaaatcctaagtaagggaatgcagagagatggtcggagtatcgccggtgaagactaagctgtgtgatcggtttaaccgatccgtcggcggcaggaattgccaccggaaacacgtcgaggacgggtgatccagttttctaaactctcgtctctcgaattcttcgaagatatcgaaaaactgtaaatcttttttttcttctacttttttacaaaattctctaatcatcgttgtaaagtaaaaaacc

SEQ ID NO: 396 - DNA, 291 bp, Arabidopsis thaliana
ttctttcgtgaaatttgtcatctcttctttcagaaacttatctggattctagccaatttctgttgtgactttgacattatcttctccagaaggaggtgaaaagagaatttgtgggtcctggtaagttccgaattcgtatttgattgagctctgagtttcaagggtttgtgttggatcaatctttagattcgttggtgaaagcgtttaaatcgacgaaaaaagtgatgctttggaagatatgatcttctctatctctggttattactgggtttcgagattcttgtgcttaag

SEQ ID NO: 397 - DNA, 48 bp, Arabidopsis thaliana
taaaccaccaattctctcatccgtaccaaagaacaaaaaaaagataaa

SEQ ID NO: 398 - DNA, 85 bp, Arabidopsis thaliana
acttctcataaaaaaggtcatttcaaaaaaaaatcagaaaccgtcaaaaagccaccgttgatatttcttccttgttgcttcttca

SEQ ID NO: 399 - DNA, 285 bp, Arabidopsis thaliana
ctcctctctcttctctcttctttcgcgtttcgaaggttggggaaagctttcgcagaagaaaataaaagctagagagagaatgtcaatgtttttttgatgctccgtctggcaattagggtttcttttttctttgatttcgtccccttcgagaactgaatctcccgcctatatcgacgccgtctaattcctatcatttctcgttgctccaaaaccctaactttactaccgtcggtcattattttcactttctcggctcgatttggtgttggaggttggtaatcagtt

SEQ ID NO: 400 - DNA, 151 bp, Arabidopsis thaliana
cgagcgaccaaaacgcagagttttgacagcaattgagtggataccgaatcacaataatacagaaagacattaaaagcaacaaggaatcgcgcgattgggggcagttggagagacgaacaagtcgtggtgagattttaggaagacgaagaag

SEQ ID NO: 401 - DNA, 181 bp, Arabidopsis thaliana
aagtatcaaaaaaattacaactttacgatttgcttagaaaggagaagacatctggagcaacaggatttacaaaagttattatctttatcgatttctcttcttcctagacccaacagacgaagagaatttgttgttggttgtctctggtctcttcgtctaggttttttttgggttattaaag

SEQ ID NO: 402 - DNA, 66 bp, Arabidopsis thaliana
cttaaattatcgtttgtgacggaagaagaatcaaaacaattaatcgcgaggcttgagaatcaatca

SEQ ID NO: 403 - DNA, 69 bp, Arabidopsis thaliana
agagaggcaaataatatattcagtagcaaaaaaaaaatctgggatttctaaaaaaaggtaagaaggaaa

SEQ ID NO: 404 - DNA, 294 bp, Arabidopsis thaliana; identical to SEQ ID NO: 99

SEQ ID NO: 405 - DNA, 228 bp, Arabidopsis thaliana
tagttacgtgtttctgtttttctctaatttttctcttgttgttctcgattaacgaaaaagacttgtcgttctcaattcttatcgatttaagaacaaatcatctaacgaagattacttccgaagatcagaaacaaacacaaactgtgaatcgttgtttgttaattctctttaaaatcgccagaagaaagagatctccgttttctacagaagaaaagcaagagagtaaga

SEQ ID NO: 406 - DNA, 228 bp, Arabidopsis thaliana; identical to SEQ ID NO: 405

SEQ ID NO: 407 - DNA, 276 bp, Arabidopsis thaliana
gagaacgagagagcaagccattgcaggaaatggcgattccagtgacgagaatgatggttcctcacgcaataccatcgcttcgtctctcacatccaaaccctagtcgcgttgacttcctctgtcgctgtgctccatcagaaatccaaccacttcggcctgaactctctttatctgtcggaattcacgcaatccctcatccagataagtgtcgaaattatataggtagagaaaggtggtgaagatgctttctttgtaagtagttatagaggtggagtc

SEQ ID NO: 408 - DNA, 48 bp, Arabidopsis thaliana
aatattttcattaatcgattctcaaagtcaagcaaaaaaaacgaaaca

SEQ ID NO: 409 - DNA, 444 bp, Arabidopsis thaliana
cctttcattgatttcatcatcatcatcatccttcgttttttctctatcgatctagcagattctttcggggaccaaaatcaaaatcatggtggatcatcaatggaaggatttaatcggataaaagagaagagacggaatcacgacgggagaagagatcgggaaatcggaaaatcggagatgatggggatttctttcgccgccaaactccgtttccgatctcgatttcgaacttcttcaatcgattcttattgcttcgctcgtgaggctttctccgattgtatctcctccgtccatttcttcttcttataacctttttctttgtaataacctccgtcctcttcagctttctttcttttcatcttcaatctcaccttaaattctccacttttttcttcttctccttctgttctcgattgctttgtttgttgtgttgtgcatacatat

SEQ ID NO: 410 - DNA, 164 bp, Arabidopsis thaliana
aatcgtttccacgaaaacaagtagagagagtgattcgagttttccaatcataaaaatcagcgaagaagatcttcgttcttgttcattctgtgaggtttcattgttaaaatcgaaacgaatctcaggttggagtaatccttgggagagatccgatttccgtttcc

SEQ ID NO: 411 - DNA, 259 bp, Arabidopsis thaliana
gaaaaaaccgtatctcattattatataaatagagagagaacagccccacgtaaacaaatagcgatagagcaactgtgtcgattgtcccaaataattttaaaaataatttcacgtgtccccattttgctgacgtcattattcccctttttcctttttattgtcacatcagaattttttctaactcattcatttcaatcaatcttcttcttcttcttcttcttcttcctcagagaaattctgtgttgttgtatacagagag

SEQ ID NO: 412 - DNA, 61 bp, Arabidopsis thaliana
actcacacatccacaaaaagagagttagagattccaaggaggagagtgcgtgagcgtgaca

SEQ ID NO: 413 - DNA, 23 bp, Arabidopsis thaliana
aagaaacacagagagcaaaacac

SEQ ID NO: 414 - DNA, 77 bp, Arabidopsis thaliana
gcttctgtggctaacaaagagcaaacaaacacttagaagaaactaagaatactctcatcaaggcgatatagaaaaaa

SEQ ID NO: 415 - DNA, 134 bp, Arabidopsis thaliana
tttttttttgggttctgtcttgaagacaaagaaagctttcttctataatacatctttctctacagatcacacagaagcaaaaattccatctccgatttcggaagagagttgttctcttctctgagaagaagaag

SEQ ID NO: 416 - DNA, 249 bp, Arabidopsis thaliana; identical to SEQ ID NO: 77

SEQ ID NO: 417 - DNA, 299 bp, Arabidopsis thaliana
aaaataaacatttgtctctatttctcttataaaaattcaataattgaacctcctctctctctctctcttctctcccttcttcttctccgatttcgactttgaatcatttcttcgagagaggtagcgagaaagggatcgccttttctcactctctgcggattctcaattttgggcaagaaggcaagaacagtttttatcgcaattgagtcttgaagaccacaaggatttgatcacattggtgcttctgcctgtttatctgagtttgaggacaagaacttctggggcgtttataatttgcc

SEQ ID NO: 418 - DNA, 204 bp, Arabidopsis thaliana
atctttggcttctacatccaattatttacttgcttaattttattcatctgaattattttttggtgtaagaagaatgtttcgccgcaaaaaaaaaaatctgatccgacatcattagaacaaaaaaaaacattggcgttgaatataagctgcttctcttgttcttcttctaccttacgcttctgactgttattagagactatgtaa

SEQ ID NO: 419 - DNA, 712 bp, Arabidopsis thaliana
acacacaccaacgttgattcttcttcttcttcttcttctctctttctcatctaaaccaaaaaatggcagatcagctcaccgatgatcagatctctgagttcaaggaagcttttagccttttcgacaaagacggagatggttcttctctctcagatctttcctcttttgtataattttcattcataatagactcacttgcgttttttttggtgttttgagtatcacttagtcttggctttaggaatttgatgctcttcgttgtccataaaatctctggatattcacattaacattaaacgcgagatttgatgatatctttatcgttcgttgattataaattataatcgcaatcggatctatctcgataataatctctaacttaatcgtgttttagtcttccagattttactaattgtgattagaattgacacaaatcttagaattcaataatcgaagtagattacattgacatttgtagattttttgtttaattgattcagttatttgagtaggttacaatgaaatttgaagattttgtgttcatttgatacagttgttagagtaactaaaatgaaatttgaagattttgtgtgttattagagtaaattacaatgaaaatttgaagatttggtgttaaaatctgttactgatttgagagaaatgtgtggttttgtgtttaggttgcatcacaacgaaagagctaggaacagtg

SEQ ID NO: 420 - DNA, 78 bp, Arabidopsis thaliana
gatttcataaaccacgactgacttctcctgctcgccgatcagatctccgacgaagtttttgattaagagaggaagaag

SEQ ID NO: 421 - DNA, 198 bp, Arabidopsis thaliana; identical to SEQ ID NO: 372

SEQ ID NO: 422 - DNA, 30 bp, Arabidopsis thaliana
aagaaatagtaatacacaaaaagaaacaaa

SEQ ID NO: 423 - DNA, 176 bp, Arabidopsis thaliana
gggaacgcggaagaaacgcaaagccctctccttttgcttctggtcctctcgtcccgtttcgccgctctctataggggcaagtgagaggttactgtctctttcttctttcagacactcgagacgagaaaggctcgtatctgattttaccgccaccggaccatctgtgatagacaata

SEQ ID NO: 424 - DNA, 256 bp, Arabidopsis thaliana
acgaaaactcataaagccaaagcctttcttcttcttcttttcttccgattattcccaaacacaaaaatactgctgaggaaaagcaatccacacgattcgattcaaagttttcattttttctctaaaagtttggattttgatttcgttgctgaacggaaaaagaatcagctcctttcagtttagggttttgggtttctgtttggtctctatcagatgatgtgtgaggagattcttcctctgtttgtgtctgtttcag

SEQ ID NO: 425 - DNA, 45 bp, Arabidopsis thaliana
tttcttcggcgatctagggttttagttgtcgcacgaggaggaaaa

SEQ ID NO: 426 - DNA, 132 bp, Arabidopsis thaliana
ctcattctcaaatctctcattgtgtgtctgtgactatctctctatacaattcaaactcttcaagattacttcctcttcactttgagaagaagaacaaaccaacaaatctccaaaatacaccgaacaacatta

SEQ ID NO: 427 - DNA, 129 bp, Arabidopsis thaliana
taacggtgaaaaatcgtcatctacttcttcttgaaaccctagttccaaaatctgcacacacactcagaagaagaagacgtcatctctctatctctgtctttctgctaatttcacgaagaatctgagaat

SEQ ID NO: 428 - DNA, 79 bp, Arabidopsis thaliana
acaattaaagtgagaattttcctgaagaagaagaacttttgctttttttctgggtttgcttttttgttgtgtcaatgaa

SEQ ID NO: 429 - DNA, 76 bp, Arabidopsis thaliana
attttgttttgcgtttctgaatttgtggccattatcttctcacactctcttctcttagctcacagaggaaagaaaa

SEQ ID NO: 430 - DNA, 64 bp, Arabidopsis thaliana
gttggtgatccgatttttctgggtttggttgggttccttttttattttttaataaaaaaaaaaa

SEQ ID NO: 431 - DNA, 182 bp, Arabidopsis thaliana
aatcgtcgataatcattagggtaaagcaaaaatagtgaagcagagccgcaaaaacacttttcccaaaatcaacgaagatagattcagatcggaagcgaaagaacgattcggtctcctccacagatcgaacatcgaaggagaagaaagaccatcatcacaacaagcatcgaaagaagagcaag

SEQ ID NO: 432 - DNA, 74 bp, Arabidopsis thaliana
taaaagcagcggcgtcatcgagagaaaccgaagaagaagcagtaacaaatttggtgaagtcacgagaatcaacg

SEQ ID NO: 433 - DNA, 66 bp, Arabidopsis thaliana
atgaattaggaatctgtgattatgataacggagtctgaagcctagactcgaaaccacaagaagaga

SEQ ID NO: 434 - DNA, 51 bp, Arabidopsis thaliana
aattgatcgcactgtcaaaccaaaaaaaattgaaaaccctaaattggttga

SEQ ID NO: 435 - DNA, 294 bp, Arabidopsis thaliana; identical to SEQ ID NO: 99

SEQ ID NO: 436 - DNA, 75 bp, Arabidopsis thaliana
ttatctttctcaacgcacgccttaccattaaggagacccaaatttcctgcaacaaacaaagtaaaaaagttgaga

SEQ ID NO: 437 - DNA, 40 bp, Arabidopsis thaliana
tattttcgtgctcggaaaaagcagagtaaagctttaaaaa

SEQ ID NO: 438 - DNA, 656 bp, Arabidopsis thaliana
aaaaaagggcgaatttttccatggcgttgtcggagtttcagctagctctgagcttggtggtcttgttcttctagctgatttgatcgaaaccccatgttcttatgattttacacgacctaatccaaaactccaggtccttgattgattcttctctctctccagctccagattcttctgatttcttttgttatcatttgtttttgtaagatttgtatccgtttttgggttttgcttagctgattcttgctggatcgagagttgaataactctgcttttcttcaatctggttttttttttttgtttcatagaggagaaaggttgtggatttctcaggtggggatttgagaattagggttttctgattgggggttttcttattgatgttaccttcaccaaattgttgtcggagatctagatttggttcagttatggaataatggctcgtctcttgccatctctattcgtaattagcatcttcttcttcatccaaagactcctcctttcttcgttaatccatcgccagctattgaatctgaagcaaatctgagaatctaccgaactcacgcacctgtatattgcttacacgatacagagcacacggagacggagtacatattgttcagcgcaagtgaaagcaagagcctttttgtctattg

SEQ ID NO: 439 - DNA, 146 bp, Arabidopsis thaliana
atactcgtatcttgtagcagccactaaagcaaaattctgagatcgaaaaagctatataaaaaaaaaaaactgcttccgtttcatcgattttgtccagatcttccccttcttccggtaatcgaagcttacgagatagttgagtgaag

SEQ ID NO: 440 - DNA, 274 bp, Arabidopsis thaliana; identical to SEQ ID NO: 335

SEQ ID NO: 441 - DNA, 254 bp, Arabidopsis thaliana
ttagggacgggacactagagaagggagggcagagagcgattttgttctctctctacttctcggtcgtcttcttcgtctccactctagggttttactctatcttcttcttcatcatcatcttctacaccaatctctagcgttaatctgtttctgctggagaagatttacgcttgttcctcggttctcttacttctgctccggttcgatcgcttgctaagtgtttcgagttggttcgcacttcggtgggcgatatc

SEQ ID NO: 442 - DNA, 63 bp, Arabidopsis thaliana
caagtctacgagcttcttcttctcggaatcggagaagcaggaaaattccggaggagcaggaag

SEQ ID NO: 443 - DNA, 255 bp, Arabidopsis thaliana; identical to SEQ ID NO: 363

SEQ ID NO: 444 - DNA, 176 bp, Arabidopsis thaliana
actgacacaaaagggaatgcgcttcatgcgggtcatcctcttaatctcaaactctctaggactacactaaatctaactttttgcagagagcaaaagattcaataattgagattgatctcaaaaccaaagctctcgtgctcttgtcgttgatgttggttgtgtagactttgtataca

SEQ ID NO: 445 - DNA, 69 bp, Arabidopsis thaliana
atccaaagctctgatgtaagaaactctacacttgttcgagtttcggagaaaagaaacgatgaggaagag

SEQ ID NO: 446 - DNA, 305 bp, Arabidopsis thaliana
atacaattccaacaaaaccacaaagacgactctcttcagagagttttgagagggtgagagagccgtgctcggcgttgttagaaagaaagctgagaattgcaactgcttacaagagcaatgtcgacaagctgatcaagagtctcttggatttgtgcttctgtacttcttaagaggaaggtcccgcaagataccatcttctcaaaagtccaatcaatctacgcttttcaattcgccacgtcacagaatcctgaccgttagatacaaacgcgccaactcgtcaaactttgctttctggtacggcggcg

SEQ ID NO: 447 - DNA, 134 bp, Arabidopsis thaliana
gaaatgttaataaataaacctaaaccaatagaaccgcagtttttcctcctcgccgaaacgaagaagattctccttctctccgtcagacaaatctacgaacaagcgagcctgagcttaagaccaaactcatagag

SEQ ID NO: 448 - DNA, 161 bp, Arabidopsis thaliana
cgtaactaatccctaaatcaagagagaagtgagagacactgagactttgtagttgaccggatcattctcacttcgccggccgacgttcttccttccgccgtcggtatctatatttacgatccacgatctctcttgctgtttctgtcttcatcgtgacgaaa

SEQ ID NO: 449 - DNA, 242 bp, Arabidopsis thaliana
catctctttgtgcctctctttactcatctctttttccacaagagtcttgagttttataaaaaagacaagcttgaagctttgtttgaatggagttactgtttgatctttgtttgttcttttgtctttaaccacttggcccattctttgtctgtttctttcatcaaccacataaacaaaaaggaaacctcatctgtaaacaagtgtttatccaaggataaagaaaaaaactgaaacttgtgaac

SEQ ID NO: 450 - DNA, 25 bp, Arabidopsis thaliana
gagaaaaagtgtgagtcagagaata

SEQ ID NO: 451 - DNA, 28 bp, Arabidopsis thaliana
acaaacacaaaatatagaaaaagaaata

SEQ ID NO: 452 - DNA, 102 bp, Arabidopsis thaliana
agatccactcacacctcgtctcctaatctgtacggttcttatttcgaaagggtaaaaaccaaaagcgacgcaaagggcaaaatcggaaaaagtgttttattt

SEQ ID NO: 453 - DNA, 249 bp, Arabidopsis thaliana; identical to SEQ ID NO: 77

SEQ ID NO: 454 - DNA, 56 bp, Arabidopsis thaliana
aatcactcctcaagcaaatcactcctcacaccacagaaaaacaaataattgaagaa

SEQ ID NO: 455 - DNA, 285 bp, Arabidopsis thaliana
actctaaagcctttttcccctcttctcattctcgagctccggacttgtcttgaaaccgtgaaggaatctgtatcttttgtatgttacccattttattgtcgttaagaatcaatttagaggcaaaacgccgagaggtttgcccgggagagtgtttttacatcgatcagggtttaagcagaggttggtttgtcatttcgccagtttgcttcttcaaattcactctacgatgaagtgagaacaacaaacaaaacatagataagatagagaccttggaactgttggaag

SEQ ID NO: 456 - DNA, 43 bp, Arabidopsis thaliana
aagagacataaaacaagaatcttatcttctggtcaagagagag

SEQ ID NO: 457 - DNA, 188 bp, Arabidopsis thaliana
ataaccttcctctctatttttacaatttattttgttattagaagtggtagtggagtgaaaaaacaaatcctaagcagtcctaaccgatccccgaagctaaagattcttcaccttcccaaataaagcaaaacctagatccgacattgaaggaaaaaccttttagatccatctctgaaaaaaaaccaacc

SEQ ID NO: 458 - DNA, 112 bp, Arabidopsis thaliana
aaccttactcctcctcctcttcctctttctctaatcggcaaaattttctgctcctgagaaacaagtagagatactaaagatggaatctttgaactaaattcgaaacctttta

SEQ ID NO: 459 - DNA, 45 bp, Arabidopsis thaliana
acaacattctgaggagtgagtaatctccggcaccgaggaacaaag

SEQ ID NO: 460 - DNA, 141 bp, Arabidopsis thaliana
agagctttcaaaaaattgttgtacttcccaacggatctctgacgtttggtccagagccgacgacgacccacaaccgaaaccaagagctatctctttttcctcttctctctctccttctctacctgcgttcgtgcttaaaca

SEQ ID NO: 461 - DNA, 114 bp, Arabidopsis thaliana
acatttccttttaaattaaattgcgttaatttctcacttccctttacttcttcttcttcaccatcacaaacatcttcgtctcttgaagattccaaaaaaaacaaatcaaaagct

SEQ ID NO: 462 - DNA, 119 bp, Arabidopsis thaliana
aagtcgccgggaaaagtaaaataaaaagccgtcacgtctccgataaataatagagtatcgttagataggtagcttcaacgtaaggaatctaaattggttcagctcaaaaaacgaaaacg

SEQ ID NO: 463 - DNA, 51 bp, Arabidopsis thaliana
atcatcatcacccacagcacagagacacacacaaaaaacccataaaaaaat

SEQ ID NO: 464 - DNA, 276 bp, Arabidopsis thaliana; identical to SEQ ID NO: 407

SEQ ID NO: 465 - DNA, 98 bp, Arabidopsis thaliana
acgagccttaacgcgtagaatcttcccgtactttacttttccggaggaatagaaaattgggggctagggttcgcaattgtagttttcgagcgaagaag

SEQ ID NO: 466 - DNA, 127 bp, Arabidopsis thaliana
tctcgtaataagagtgaaaaacaagccttaacctgtaaacgcttacgctagttaaatacacaacaaagaccgattcgcttttcactctctcgttcaagatctagaattcaatttgtgaggtttggag

SEQ ID NO: 467 - DNA, 70 bp, Arabidopsis thaliana
gagagtcgacaaggaaaaggcaatgcaagaagaagcttaaatctctcttctctgctcctgaagtctgttc

SEQ ID NO: 468 - DNA, 79 bp, Arabidopsis thaliana
gcgttggttctcttcttcaaaacaagctctctctgtccctctctgtctctctctttgggtaatcggaaaaatcagaaaa

SEQ ID NO: 469 - DNA, 42 bp, Arabidopsis thaliana
atacaaatcataactcaaagaaaaacaacccctcaacggtcg

SEQ ID NO: 470 - DNA, 85 bp, Arabidopsis thaliana
accaccaccattttagggtttcttcgtgccattgatattttgagaggcgaaggaaacaatacgattcagagagagacgagtgaaa

SEQ ID NO: 471 - DNA, 20 bp, Arabidopsis thaliana
ctaattccccagaagaaaag

SEQ ID NO: 472 - DNA, 71 bp, Arabidopsis thaliana
tgactgcgtctttcttctctctctatctgtaatttgattggattttggatcgaaacctgaaaagagcgaaa

SEQ ID NO: 473 - DNA, 102 bp, Arabidopsis thaliana; identical to SEQ ID NO: 316

SEQ ID NO: 474 - DNA, 483 bp, Arabidopsis thaliana
tctagaaacagcatccgtttttataatttaattttcttacaaaggtaggaccaacatttgtgatctataaatcttcctactacgttatatagagacccttcgacataacacttaactcgtttatatatttgttttacttgttttgcacatacacacaaaaataaaaaagactttatatttatttactttttaatcacacggattagctccggcgaagtatggtcgtcgtcttcatcttcttcctccatcatcagatttttccttaaatggaagaaaccaaacgaaactccgatcttctccgttctcgtgttttcctctctggcttttattgctgggattgggaatttctcaccgctctcttgctttttagttgctgattctttttccttcgactttctatttccaatctttcttcttctctttgtgtattagattatttttagttttatttttctgtggtaaaataaaaaaagttcgccggag

SEQ ID NO: 475 - protein, 593 aa, Arabidopsis thaliana
MDTTIDGFADSYEISSTSFVATDNTDSSIVYLAAEQVLTGPDVSALQLLSNSFESVFDSPDDFYSDAKLVLSDGREVSFHRCVLSARSSFFKSALAAAKKEKDSNNTAAVKLELKEIAKDYEVGFDSVVTVLAYVYSSRVRPPPKGVSECADENCCHVACRPAVDFMLEVLYLAFIFKIPELITLYQRHLLDVVDKVVIEDTLVILKLANICGKACMKLLDRCKEIIVKSNVDMVSLEKSLPEELVKEIIDRRKELGLEVPKVKKHVSNVHKALDSDDIELVKLLLKEDHTNLDDACALHFAVAYCNVKTATDLLKLDLADVNHRNPRGYTVLHVAAMRKEPQLILSLLEKGASASEATLEGRTALMIAKQATMAVECNNIPEQCKHSLKGRLCVEILEQEDKREQIPRDVPPSFAVAADELKMTLLDLENRVALAQRLFPTEAQAAMEIAEMKGTCEFIVTSLEPDRLTGTKRTSPGVKIAPFRILEEHQSRLKALSKTVELGKRFFPRCSAVLDQIMNCEDLTQLACGEDDTAEKRLQKKQRYMEIQETLKKAFSEDNLELGNSSLTDSTSSTSKSTGGKRSNRKLSHRRR

SEQ ID NO: 476 - DNA, 483 bp, Artificial Sequence; identical to SEQ ID NO: 474
  Synthetic

SEQ ID NO: 477 - DNA, 769 bp, Artificial Sequence
  Synthetic
aagcttgcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagg

SEQ ID NO: 478 - DNA, 3071 bp, Artificial Sequence
  Synthetic
cgacgactagtttacagagaatttggaccgtccgatgtaaagcgaaaatagatctaggttttccacgtgtcccctattttaatgaaaccttctgattcatgtagaagttttactcaatttaatattttttagtatgtagttttgtgtgtgtgtgtgtgtgtgtttttatggctccacaccaacttttaaaatggtagaagcatgttgcatgtgatcgagtaaaaagccaataatgagattcagaaaaataaaaattacttatatagttttttagagaaaaaattgtattttgtttaaagccttaatccggttgttgaaagagctgtgtcacgagttaaaaatattttcttttcattttttaagtaattagtttataatgcaaaaatggtttttatttatttgtcttcgcttatagaactgcaaattgagagagaaaaaaatgaattagtggtggtgaccaaacattcaggaagctgtgattgatcatttgtttttgaggtgagtgtagtggcaacgtatgacgttaacatatggcgtacataataattacatgaacttaatcataataatcatattgcatttaattcatatatcatatcccattagttggaccacttgatttgaggtcatgagaagaacatttatgttttttttagtttgaatcggagtgatcactaaaaactagatactgaaaattttcaaactaaaatcatattaatcttcaaaaaatgtgaaatctaaaaaaaaaaaaaattttaacgcgttcattgtagccaagtagccaagtattgttaaagtagtagtaaaagaagtttagctttaagtgatataatttgacacaaatcctacttagatatggataataggatatagcttcatgtatatttttatcgttgcttctgtaaccccaaaatgtgttgatataagcatttgaatattcgtatgtataatgttttcttttcaccgtaaaacatattacaatgttagtttatattggattttgaatgtgtttatgaacagtttttgtcgactcaaaagttaagatgagaatatggaagaaagtaaagtttaaaagtcatgatgggaacaaggaatggaactcaaacattctaatactcaacaaacgcaattatattattaccatgactcatctttcaagttccatcaaaaagattcgtggaaaataatagacttacgtttcaaatccatgtttctttctttataacaaaaaaaatggatgtttcttgacgcgtgtcgagagtactcaccattactctgacttcagtgagtttggtcaagtggtctttttttttctcatgtcaccaaaggtccaaaccctagaaattagttcgaactttccatagaagaactgaataaatggtccaaaattgttttaaaaaggacctaagccattagttcattgaattcgagttaatgggtgaagatttttatgataacgaaagtcggagtaattatgcttttggtccgatagttttctaatttgttttctttccattttttttttttcaaatactacatactatataagatagtggtttgtgttaatgtcatcgatgtgttaccatccgcattatattaattatttatcccaacataaagtcagaatctgtaatttctttgttataaaatacagtaaatggttccgtttaagctgttagatgatttttgagtaaaaactaatgtaaaaaaaacaaaaaaaaaacaatgtagttcataatacatgcatgttttaaagaagtttcttgtttactatcaacttgaatagtatttcacgaagtcaaaattgttcattccgacttttctatgtggagaaaaaaaattctatcattgtgcacaatttaacagaatgtaatttcttgtaaaagaagaggaaacaattcgctgttagtaaatgtgaagtatagaagtctaaaatgagatacctcaactagcttgaattaagaaaaaaaacaaaaactctatcgacatgaaaaaggtcgcaaatatttatcatttatcaatgccaaaggagtatttggttcacaaaatactgaatcatttatatagatatataattagctctaaattctactataacttgcaaaataagtatactgactcaattatatagcgtttaaaaatagacgatttgtatgatgaggtccatatatatggagatgtgcatgcaactatcgacattttcacacgttgatatcgtctttctccaatggagacttgaatttgtgtaaactatgaatactcgtctctctaagaccttttttcttcaaccatgccaactatttaggtaagattttactgtctttgattgatattaaatacttagccgtggcgttatcaatgaatgataataaaaatgcggataaaagccaaaggtgttggaaataaatccaagaatgaagacgtagatgtcgatgggtattttaagaacttgaatttgtcacgactcacacgttaaaatatattatccgaattgtttagtctaaagacacacatatattgaaaaagaaaaggtaaatgaagctcattggtgcctaaatgtgaaatgaagccgaaatgtgttaggtgaacacatttaaatatacaaaaagaaatataatagaaacaaaactaattaacaaagtcgcaatttgtattgtataaaatatctttccgtctcccgtcatatttgaaaaaaaaaaaattacaaatctgttaattttaaaactttctagaaaaacacaagtatataattttctcttttcgtgcgtgtttgttttaaaataacattgttttgattggcgactcaacatattttagcatttacatatttctgcatatattaaatgatttataaactcaactatagattaaaatataatttgacatctaataattttaacaataatataaaatatgagatttataaattacgaatataaatattcaagggagagaaaaagtagaacataattcaaaagataagactttttagacttttttaacaatatttttgatggataaaaattattcaaaagagaagaaagtaagaagaaaagatgtttctgagaatt

SEQ ID NO: 479 - DNA, 3807 bp, Artificial Sequence
  Synthetic
aagcttgcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggccggtctagaaacagcatccgttatttaatttcttacaaaggtaggaccaacatttgtgatctataaatcttcctactacgttatatagagacccttcgacataacacttaactcgtttatatatttgttttacttgttttgcacatacacacaaaaataaaaaagactttatatttatttactttttaatcacacggattagctccggcgaagtatggtcgtcgtcttcatcttcttcctccatcatcagatttttccttaaatggaagaaaccaaacgaaactccgatcttctccgttctcgtgttttcctctctggcttttattgctgggattgggaatttctcaccgctctcttgctttttagttgctgattctttttccttcgactttctatttccaatctttcttcttctctttgtgtattagattatttttagttttatttttctgtggtaaaataaaaaaagttcgccggagggtaccttcgacgacaagaccgtaccatggacaccaccattgatggattcgccgattcttatgaaatcagcagcactagtttcgtcgctaccgataacaccgactcctctattgtttatctggccgccgaacaagtactcaccggacctgatgtatctgctctgcaattgctctccaacagcttcgaatccgtctttgactcgccggatgatttctacagcgacgctaagcttgttctctccgacggccgggaagtttctttccaccggtgcgttttgtcagcgagaagctctttcttcaagagcgctttagccgccgctaagaaggagaaagactccaacaacaccgccgccgtgaagctcgagcttaaggagattgccaaggattacgaagtcggtttcgattcggttgtgactgttttggcttatgtttacagcagcagagtgagaccgccgcctaaaggagtttctgaatgcgcagacgagaattgctgccacgtggcttgccggccggcggtggatttcatgttggaggttctctatttggctttcatcttcaagatccctgaattaattactctctatcagaggcacttattggacgttgtagacaaagttgttatagaggacacattggttatactcaagcttgctaatatatgtggtaaagcttgtatgaagctattggatagatgtaaagagattattgtcaagtctaatgtagatatggttagtcttgaaaagtcattgccggaagagcttgttaaagagataattgatagacgtaaagagcttggtttggaggtacctaaagtaaagaaacatgtctcgaatgtacataaggcacttgactcggatgatattgagttagtcaagttgcttttgaaagaggatcacaccaatctagatgatgcgtgtgctcttcatttcgctgttgcatattgcaatgtgaagaccgcaacagatcttttaaaacttgatcttgccgatgtcaaccataggaatccgaggggatatacggtgcttcatgttgctgcgatgcggaaggagccacaattgatactatctctattggaaaaaggtgcaagtgcatcagaagcaactttggaaggtagaaccgcactcatgatcgcaaaacaagccactatggcggttgaatgtaataatatcccggagcaatgcaagcattctctcaaaggccgactatgtgtagaaatactagagcaagaagacaaacgagaacaaattcctagagatgttcctccctcttttgcagtggcggccgatgaattgaagatgacgctgctcgatcttgaaaatagagttgcacttgctcaacgtctttttccaacggaagcacaagctgcaatggagatcgccgaaatgaagggaacatgtgagttcatagtgactagcctcgagcctgaccgtctcactggtacgaagagaacatcaccgggtgtaaagatagcacctttcagaatcctagaagagcatcaaagtagactaaaagcgctttctaaaaccgtggaactcgggaaacgattcttcccgcgctgttcggcagtgctcgaccagattatgaactgtgaggacttgactcaactggcttgcggagaagacgacactgctgagaaacgactacaaaagaagcaaaggtacatggaaatacaagagacactaaagaaggcctttagtgaggacaatttggaattaggaaattcgtccctgacagattcgacttcttccacatcgaaatcaaccggtggaaagaggtctaaccgtaaactctctcatcgtcgtcggtacccagctttcttgtacaaagtggtgatatcaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 480 - DNA, 6091 bp, Artificial Sequence
  Synthetic
cgacgactagtttacagagaatttggaccgtccgatgtaaagcgaaaatagatctaggttttccacgtgtcccctattttaatgaaaccttctgattcatgtagaagttttactcaatttaatattttttagtatgtagttttgtgtgtgtgtgtgtgtgtgtttttatggctccacaccaacttttaaaatggtagaagcatgttgcatgtgatcgagtaaaaagccaataatgagattcagaaaaataaaaattacttatatagttttttagagaaaaaattgtattttgtttaaagccttaatccggttgttgaaagagctgtgtcacgagttaaaaatattttcttttcattttttaagtaattagtttataatgcaaaaatggtttttatttatttgtcttcgcttatagaactgcaaattgagagagaaaaaaatgaattagtggtggtgaccaaacattcaggaagctgtgattgatcatttgtttttgaggtgagtgtagtggcaacgtatgacgttaacatatggcgtacataataattacatgaacttaatcataataatcatattgcatttaattcatatatcatatcccattagttggaccacttgatttgaggtcatgagaagaacatttatgttttttttagtttgaatcggagtgatcactaaaaactagatactgaaaattttcaaactaaaatcatattaatcttcaaaaaatgtgaaatctaaaaaaaaaaaaaattttaacgcgttcattgtagccaagtagccaagtattgttaaagtagtagtaaaagaagtttagctttaagtgatataatttgacacaaatcctacttagatatggataataggatatagcttcatgtatatttttatcgttgcttctgtaaccccaaaatgtgttgatataagcatttgaatattcgtatgtataatgttttcttttcaccgtaaaacatattacaatgttagtttatattggattttgaatgtgtttatgaacagtttttgtcgactcaaaagttaagatgagaatatggaagaaagtaaagtttaaaagtcatgatgggaacaaggaatggaactcaaacattctaatactcaacaaacgcaattatattattaccatgactcatctttcaagttccatcaaaaagattcgtggaaaataatagacttacgtttcaaatccatgtttctttctttataacaaaaaaaatggatgtttcttgacgcgtgtcgagagtactcaccattactctgacttcagtgagtttggtcaagtggtctttttttttctcatgtcaccaaaggtccaaaccctagaaattagttcgaactttccatagaagaactgaataaatggtccaaaattgttttaaaaaggacctaagccattagttcattgaattcgagttaatgggtgaagatttttatgataacgaaagtcggagtaattatgcttttggtccgatagttttctaatttgttttctttccattttttttttttcaaatactacatactatataagatagtggtttgtgttaatgtcatcgatgtgttaccatccgcattatattaattatttatcccaacataaagtcagaatctgtaatttctttgttataaaatacagtaaatggttccgtttaagctgttagatgatttttgagtaaaaactaatgtaaaaaaaacaaaaaaaaaacaatgtagttcataatacatgcatgttttaaagaagtttcttgtttactatcaacttgaatagtatttcacgaagtcaaaattgttcattccgacttttctatgtggagaaaaaaaattctatcattgtgcacaatttaacagaatgtaatttcttgtaaaagaagaggaaacaattcgctgttagtaaatgtgaagtatagaagtctaaaatgagatacctcaactagcttgaattaagaaaaaaaacaaaaactctatcgacatgaaaaaggtcgcaaatatttatcatttatcaatgccaaaggagtatttggttcacaaaatactgaatcatttatatagatatataattagctctaaattctactataacttgcaaaataagtatactgactcaattatatagcgtttaaaaatagacgatttgtatgatgaggtccatatatatggagatgtgcatgcaactatcgacattttcacacgttgatatcgtctttctccaatggagacttgaatttgtgtaaactatgaatactcgtctctctaagaccttttttcttcaaccatgccaactatttaggtaagattttactgtctttgattgatattaaatacttagccgtggcgttatcaatgaatgataataaaaatgcggataaaagccaaaggtgttggaaataaatccaagaatgaagacgtagatgtcgatgggtattttaagaacttgaatttgtcacgactcacacgttaaaatatattatccgaattgtttagtctaaagacacacatatattgaaaaagaaaaggtaaatgaagctcattggtgcctaaatgtgaaatgaagccgaaatgtgttaggtgaacacatttaaatatacaaaaagaaatataatagaaacaaaactaattaacaaagtcgcaatttgtattgtataaaatatctttccgtctcccgtcatatttgaaaaaaaaaaaattacaaatctgttaattttaaaactttctagaaaaacacaagtatataattttctcttttcgtgcgtgtttgttttaaaataacattgttttgattggcgactcaacatattttagcatttacatatttctgcatatattaaatgatttataaactcaactatagattaaaatataatttgacatctaataattttaacaataatataaaatatgagatttataaattacgaatataaatattcaagggagagaaaaagtagaacataattcaaaagataagactttttagacttttttaacaatatttttgatggataaaaattattcaaaagagaagaaagtaagaagaaaagatgtttctgagaatttctagggcgcgccttcttacaaaggtaggaccaacatttgtgatctataaatcttcctactacgttatatagagacccttcgacataacacttaactcgtttatatatttgttttacttgttttgcacatacacacaaaaataaaaaagactttatatttatttactttttaatcacacggattagctccggcgaagtatggtcgtcgtcttcatcttcttcctccatcatcagatttttccttaaatggaagaaaccaaacgaaactccgatcttctccgttctcgtgttttcctctctggcttttattgctgggattgggaatttctcaccgctctcttgctttttagttgctgattctttttccttcgactttctatttccaatctttcttcttctctttgtgtattagattatttttagttttatttttctgtggtaaaataaaaaaagttcgccggagggtaccttcgacgacaagaccgaccatggacaccaccattgatggattcgccgattcttatgaaatcagcagcactagtttcgtcgctaccgataacaccgactcctctattgtttatctggccgccgaacaagtactcaccggacctgatgtatctgctctgcaattgctctccaacagcttcgaatccgtctttgactcgccggatgatttctacagcgacgctaagcttgttctctccgacggccgggaagtttctttccaccggtgcgttttgtcagcgagaagctctttcttcaagagcgctttagccgccgctaagaaggagaaagactccaacaacaccgccgccgtgaagctcgagcttaaggagattgccaaggattacgaagtcggtttcgattcggttgtgactgttttggcttatgtttacagcagcagagtgagaccgccgcctaaaggagtttctgaatgcgcagacgagaattgctgccacgtggcttgccggccggcggtggatttcatgttggaggttctctatttggctttcatcttcaagatccctgaattaattactctctatcagaggcacttattggacgttgtagacaaagttgttatagaggacacattggttatactcaagcttgctaatatatgtggtaaagcttgtatgaagctattggatagatgtaaagagattattgtcaagtctaatgtagatatggttagtcttgaaaagtcattgccggaagagcttgttaaagagataattgatagacgtaaagagcttggtttggaggtacctaaagtaaagaaacatgtctcgaatgtacataaggcacttgactcggatgatattgagttagtcaagttgcttttgaaagaggatcacaccaatctagatgatgcgtgtgctcttcatttcgctgttgcatattgcaatgtgaagaccgcaacagatcttttaaaacttgatcttgccgatgtcaaccataggaatccgaggggatatacggtgcttcatgttgctgcgatgcggaaggagccacaattgatactatctctattggaaaaaggtgcaagtgcatcagaagcaactttggaaggtagaaccgcactcatgatcgcaaaacaagccactatggcggttgaatgtaataatatcccggagcaatgcaagcattctctcaaaggccgactatgtgtagaaatactagagcaagaagacaaacgagaacaaattcctagagatgttcctccctcttttgcagtggcggccgatgaattgaagatgacgctgctcgatcttgaaaatagagttgcacttgctcaacgtctttttccaacggaagcacaagctgcaatggagatcgccgaaatgaagggaacatgtgagttcatagtgactagcctcgagcctgaccgtctcactggtacgaagagaacatcaccgggtgtaaagatagcacctttcagaatcctagaagagcatcaaagtagactaaaagcgctttctaaaaccgtggaactcgggaaacgattcttcccgcgctgttcggcagtgctcgaccagattatgaactgtgaggacttgactcaactggcttgcggagaagacgacactgctgagaaacgactacaaaagaagcaaaggtacatggaaatacaagagacactaaagaaggcctttagtgaggacaatttggaattaggaaattcgtccctgacagattcgacttcttccacatcgaaatcaaccggtggaaagaggtctaaccgtaaactctctcatcgtcgtcggtacccagctttcttgtacaaagtggtgatatcaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 481 - RNA, 15 nt, Artificial Sequence
  Synthetic
  misc_feature (3)..(4): n is a, c, g, or u
  misc_feature (9)..(10): n is a, c, g, or u
vvnnvrvrnnvbvad

SEQ ID NO: 482 - DNA, 111 bp, Arabidopsis thaliana
atggaagaaaccaaacgaaactccgatcttctccgttctcgtgttttcctctctggcttttattgctgggattgggaatttctcaccgctctcttgctttttagttgctga

SEQ ID NO: 483 - DNA, 80 bp, Phaseolus vulgaris
atggattctgatcgtcccagaaactctaccttgtttcactctcgcttctttctcttcggtttctattgctgggactggga

SEQ ID NO: 484 - DNA, 80 bp, Glycine max
atggcttctaaaacccctaccttattccactctcgtttcttcctcttcggcttctattgctgggactgggaatttctcac

SEQ ID NO: 485 - DNA, 123 bp, Gossypium raimondii
atggaaactgatacgatcaaaccctgcgataacgtcgttcattctcgattcttccgttctggattttattgctggggttgggaattcttgactgctctgcttctctttagttgttctgcctaa

SEQ ID NO: 486 - DNA, 135 bp, Nicotiana benthamiana
atggaagaaactaagatcataatcaatcgccccaaaaacaaccttgttcattctatggtttttctattcggtttctatgtttgggattgggaattcctgactgcccttttgcttttcagttattgcttcttctaa

SEQ ID NO: 487 - DNA, 171 bp, Cicer arietinum
atggattttaattgtaacaaaaataaaccttactcttcttcttcttcttcttcttcttcttcttcttatttttttttttcttcttttcattttcgctttttcctctttggtttttattgctgggattgggaattcttaactgctcttctccttttcagttttttttcttaa

SEQ ID NO: 488 - DNA, 135 bp, Phoenix dactylifera
atggaaggaaaggggggcgagggattcggaatcggagcaagaggcgatgggtgctgccgctgggtgatcatcagcgggttctactgctggggctgggagttcttgactgctctcctgctcttctcgtgccgctga

SEQ ID NO: 489 - DNA, 147 bp, Musa acuminata subsp malaccensis
atggaagaagagagaccgataggtgttgcagggaggagcgacgttgatggcggcctcggtctgggttgctcccacagggtgatcatcaccggtttctactgctggggttgggagttcttgaccgctctcctcgtcttcgcgttttga

SEQ ID NO: 490 - DNA, 117 bp, Oryza sativa
atgggagtagaggcgggcggcggctgcggtgggagggcggtagtcaccggattctacgtctggggctgggagttcctcaccgccctcctgctcttctcggccaccacctcctactag

SEQ ID NO: 491 - protein, 36 aa, Arabidopsis thaliana
MEETKRNSDLLRSRVFLSGFYCWDWEFLTALLLFSC

SEQ ID NO: 492 - protein, 39 aa, Phaseolus vulgaris
MDSDRPRNSTLFHSRFFLFGFYCWDWEFLTALLLFSSSS

SEQ ID NO: 493 - protein, 36 aa, Glycine max
MASKTPTLFHSRFFLFGFYCWDWEFLTALLLFSSSS

SEQ ID NO: 494 - protein, 40 aa, Gossypium raimondii
METDTIKPCDNVVHSRFFRSGFYCWGWEFLTALLLFSCSA

SEQ ID NO: 495 - protein, 44 aa, Nicotiana benthamiana
MEETKIIINRPKNNLVHSMVFLFGFYVWDWEFLTALLLFSYCFF

SEQ ID NO: 496 - protein, 56 aa, Cicer arietinum
MDFNCNKNKPYSSSSSSSSSSSYFFFSSFHFRFFLFGFYCWDWEFLTALLLFSFFS

SEQ ID NO: 497 - protein, 44 aa, Phoenix dactylifera
MEGKGGEGFGIGARGDGCCRWVIISGFYCWGWEFLTALLLFSCR

SEQ ID NO: 498 - protein, 48 aa, Musa acuminata subsp malaccensis
MEEERPIGVAGRSDVDGGLGLGCSHRVIITGFYCWGWEFLTALLVFAF

SEQ ID NO: 499 - protein, 38 aa, Oryza sativa
MGVEAGGGCGGRAVVTGFYVWGWEFLTALLLFSATTSY

(19) United States
(12) Patent Application Publication
(10) Pub. No.: US 2019/0352664 A1
(43) Pub. Date: Nov. 21, 2019

(54) COMPOSITIONS AND METHODS FOR CONTROLLING GENE EXPRESSION

(71) Applicant: DUKE UNIVERSITY, DURHAM, NC (US)

(72) Inventors: XINNIAN DONG, DURHAM, NC (US); GEORGE GREENE, DURHAM, NC (US); GUOYONG XU, DURHAM, NC (US)

(21) Appl. No.: 16/482,941

(22) PCT Filed: Feb. 2, 2018
(86) PCT No.: PCT/US2018/016608

§ 371 Date: Aug. 1, 2019

Related U.S. Application Data
(60) Provisional application No. 62/453,807, filed on Feb. 2, 2017.

(51) Int. Cl. C12N 15/82

(52) U.S. Cl. CPC C12N 15/8281

(57) ABSTRACT

The invention generally relates to compositions (including constructs, vectors, and cells) and methods of using such compositions for controlling gene expression. More specifically, the invention relates to use of R-motif sequences and/or uORF sequences to control gene expression.

### CROSS-REFERENCE TO RELATED PATENT APPLICATIONS

The present application claims the benefit of priority to U.S. Provisional Patent Application No. 62/453,807, filed on Feb. 2, 2017, the content of which is incorporated herein by reference in its entirety.

### STATEMENT REGARDING FEDERALLY SPONSORED RESEARCH

This invention was made with government support under grant number 5R01 GM069594-11 awarded by the National Institute of Health. The United States government has certain rights in the invention.

### SEQUENCE LISTING

This application is being filed electronically via EFS-Web and includes an electronically submitted Sequence Listing in .txt format. The .txt file contains a sequence listing entitled “2018-02-02_5667-00424_ST25.txt” created on Feb. 2, 2018 and is 155,230 bytes in size. The Sequence Listing contained in this .txt file is part of the specification and is hereby incorporated by reference herein in its entirety.

### INTRODUCTION

Controlling plant disease has been a struggle for mankind since the advent of agriculture. Knowledge obtained through studies of plant immune mechanisms has led to the development of strategies for engineering resistant crops through ectopic expression of plants' own defense genes, such as the master immune regulator NPR1. However, enhanced resistance is often associated with a significant fitness penalty making the product undesirable for agricultural application.

To meet the demand on food production caused by the explosion in world population and at the same time the desire to limit pesticide pollution to the environment, new strategies must be developed to control crop diseases. As an alternative to the traditional chemical control and breeding methods, studies of plant immune mechanisms have made it possible to engineer resistance through ectopic expression of plants' own resistance-conferring genes. The first line of active defense in plants involves recognition of microbial-associated molecular patterns (MAMPs) or damage-associated molecular patterns (DAMPs) by the host pattern-recognizing receptors (PRRs) and is known as pattern-triggered immunity (PTI). Ectopic expression of PRRs for MAMPs, the DAMP signal, eATP, and in vivo release of the DAMP molecules, oligogalacturonides, have been shown to enhance resistance in transgenic plants. Besides PRR-mediated basal resistance, plant genomes also encode hundreds of intracellular nucleotide-binding and leucine-rich repeat (NB-LRR) immune receptors (also known as “R proteins”) to detect the presence of pathogen-specific effectors delivered inside the plant cells. Individual or stacked R genes have been transformed into plants to confer effector-triggered immunity (ETI). Besides PRR and R genes, NPR1 is another favourite gene used in engineering plant resistance because unlike R proteins that are activated by specific pathogen effectors, NPR1 is a positive regulator of broad-spectrum resistance induced by a general plant immune signal salicylic acid. While R proteins only function within the same family of plants, overexpression of the Arabidopsis NPR1 (AtNPR1) could enhance resistance in diverse plant families such as rice, wheat, tomato and cotton against a variety of pathogens.

However, a major challenge in engineering disease resistance is to overcome the associated fitness costs. In the absence of specialized immune cells, immune induction in plants involves switching from growth-related activities to defense. Plants normally avoid autoimmunity by tightly controlling transcription, mRNA nuclear export and active degradation of defense proteins. Currently predominantly transcriptional control has been used to engineer disease resistance. There thus remains a need in the art for new compositions and methods that allow more stringent pathogen-inducible expression of defense proteins so that the associated fitness costs of expressing defense proteins may be minimized.

### SUMMARY

In one aspect, DNA constructs are provided. The DNA constructs may include a heterologous promoter operably connected to a DNA polynucleotide encoding a RNA transcript including a 5′ regulatory sequence located 5′ to an insert site, wherein the 5′ regulatory sequence includes an R-motif sequence. Optionally, the DNA constructs may further include a uORF polynucleotide encoding any one of the uORF polypeptides of SEQ ID NOs: 1-38 in Table 1, or a variant thereof. Alternatively, the DNA constructs may include a heterologous promoter operably connected to a DNA polynucleotide encoding a RNA transcript including a 5′ regulatory sequence located 5′ to an insert site, wherein the 5′ regulatory sequence includes an uORF polynucleotide encoding any one of the uORF polypeptides of SEQ ID NOs: 1-38 in Table 1 or a variant thereof.

In another aspect, vectors, cells, and plants including any of the constructs described herein are provided.

In a further aspect, methods for controlling the expression of a heterologous polypeptide in a cell are provided. The methods may include introducing any one of the constructs or vectors described herein into the cell. Preferably, the constructs and vectors include a heterologous coding sequence encoding a heterologous polypeptide.

### BRIEF DESCRIPTION OF THE DRAWINGS

FIGS. 1A-1E show translational activities during elf18-induced PTI. FIG. 1A, Schematic of the 35S:uORFs_{TBF1}-LUC reporter. The reporter is a fusion between the TBF1 exon1 (uORF1/2 and sequence of the N-terminal 73 amino acids) and the firefly luciferase gene (LUC) expressed constitutively by the CaMV 35S promoter. R, R-motif. FIG. 1B, Translation of the 35S:uORFs_{TBF1}-LUC reporter in wild type (WT) and efr-1 in response to elf18 treatment. Mean±s.e.m. (n=9) after normalization to that at time 0. FIGS. 1C, 1D, Polysome profiling of global translational activity (FIG. 1C) and TBF1 mRNA translational activity calculated as ratios of polysomal/total mRNA (FIG. 1D) in WT and efr-1 in response to elf18 treatment. Lower case letters indicate fractions in polysome profiling. FIG. 1E, Schematic of RS and RF library construction using uORFs_{TBF1}-LUC/WT plants. RS, RNA-seq; RF, ribosome footprint. RNase I and Alkaline are two methods of generating RNA fragments.

FIGS. 2A-2J show global analyses of transcriptome (RSfc), translatome (RFfc) and translational efficiency (TEfc) upon elf18 treatment and identification of novel PTI regulators based on TEfc. FIG. 2A, Histogram of log₂RSfc and log₂RFfc. μ and δ are mean and standard derivation, respectively, of log₂RSfc and log₂RFfc. FIG. 2B, Pearson correlation coefficient r was shown between RS and RF as log₂RPKM for expressed genes with RPKM in CDS≥1 within either Mock or elf18. FIGS. 2C, 2D, Relationships between RSfc and RFfc (FIG. 2C) and between RSfc and TEfc (FIG. 2D). dn, down; nc, no change. FIG. 2E, Venn diagrams showing overlaps between RSfc and TEfc. FIG. 2F, RS and TE changes in known or homologues of known components of the ethylene- and the damage-associated molecular pattern Pep-mediated PTI signalling pathways. The pathway was modified from Zipfel¹⁷. In rectangular boxes: Black, RS-changed; Red, TE-up; green, TE-down. FIG. 2G, Elf18-induced resistance to Psm ES4326. Mean±s.e.m. of 12 biological replicates from 2 experiments. FIG. 2H, Schematic of the dual LUC system. Test, 5′ leader sequence (including UTR) or 3′ UTR of the gene tested; LUC, firefly luciferase; RLUC, renilla luciferase, Ter, terminator. FIG. 2I, Dual-LUC assay of EIN4 UTRs on TE upon elf18 treatment in N. benthamiana. EV, empty vector. Mean±s.e.m. (n=4). FIG. 2J, EIN4 TE changes upon elf18 treatment calculated as ratios of polysomal/total mRNA. Mean±s.d. from 2 experiments with 3 technical replicates. See FIGS. 10A-10C.

FIGS. 3A-3G shows the effects of R-motif on TE changes during PTI induction. FIG. 3A, R-motif consensus (SEQ ID NO: 481). FIG. 3B, Confirmation of TE induction of R-motif-containing genes in response to elf18. 5′ leader sequences of 20 endogenous genes were inserted as “Test” sequences. FIGS. 3C, 3D, Effects of R-motif deletion mutations (ΔR) on basal translational activities (FIG. 3C) and on translational responsiveness to elf18 (FIG. 3D). FIG. 3E, Gain of elf18-responsiveness with inclusion of GA, G[A]₃, G[A]₆ and G[A]ₙ repeats (total length of 120 nt) in the 5′ UTR of the dual luciferase reporter. FIGS. 3F, 3G, Contributions of R-motif and uORFs to TBF1 basal translational activity (FIG. 3F) and translational response to elf18 (FIG. 3G). Mean±s.e.m. of LUC/RLUC activity ratios in N. benthamiana (n=3 for FIGS. 3B, 3D-G or 3 experiments with 3 technical replicates for FIG. 3C) normalized to Mock (FIGS. 3B, 3D, 3E, 3G) or WT 5′ leader sequences (FIGS. 3C, 3F). See FIGS. 12A-12L.

FIGS. 4A-4H show R-motif controls translational responsiveness to PTI induction through interaction with PAB. FIG. 4A, Effects of co-expressing PAB2 on translation of R-motif-containing genes. Mean±s.e.m. of LUC/RLUC activity ratios (n=4) after normalized to the YFP control. FIG. 4B, RNA pull down of in vitro synthesized PAB2. 0.2 nmol GA, G[A]₃, G[A]₆ and G[A]ₙ repeats and poly(A) RNAs (120 nt) were biotinylated. Beads, control without the RNA probes. FIG. 4C, Binding of G[A]ₙ RNA with increasing amounts of PAB2. FIG. 4D, G[A]ₙ RNA pull down of in vivo synthesized PAB2 upon PTI induction. YFP, negative protein control. “−” or “+” mean PAB2 from Mock or elf18 treated tissue, respectively. FIG. 4E, TBF1 TE changes in the pab2 pab4 (pab2/4) mutant upon elf18 treatment calculated as ratios of polysomal/total mRNA (mean±s.d., n=3). FIGS. 4F, 4G, Elf18-induced resistance to Psm ES4326 in pab2 pab4 and pab2 pab8 plants (FIG. 4F, mean±s.e.m., n=8), and in primary transformants overexpressing PAB2 in the pab2 pab8 mutant background (OE-PAB2) (FIG. 4G, mean±s.e.m., n=8 for control and efr-1, and 17 and 13 for OE-PAB2 lines with Mock and elf18 treatment, respectively). Control, transgenic plants expressing YFP in the WT background. Both control and OE-PAB2 were selected for basta-resistance and further confirmed by PCR. FIG. 4H, Working model for PAB playing opposing roles in regulating basal and elf18-induced translation through differential interactions with R-motif. See FIGS. 13A-13C.

FIGS. 5A-5E show the translational activities during elf18-induced PTI, related to FIGS. 1A-1E. FIG. 5A, Translation of the 35S:uORFs_{TBF1}-LUC reporter in wild type (WT) after Mock or elf18 treatment. Mean±s.e.m. (n=12) after normalization to LUC activity at time 0. FIGS. 5B, 5C, Transcript levels of the 35S:uORFs_{TBF1}-LUC reporter in WT after Mock or elf18 treatment (FIG. 5B) and in WT or efr-1 upon elf18 treatment (FIG. 5C). Transcript levels are expressed as fold changes normalized to time 0. Mean±s.d. (n=3). FIGS. 5D, 5E, Polysome profiling of global translational activity (FIG. 5D) and TBF1 mRNA translational activity calculated as ratios of polysomal/total mRNA (FIG. 5E) in response to Mock and elf18 treatment in WT. Lower case letters indicate fractions in polysome profiling.

FIGS. 6A-6C show the improvement made in the library construction protocol. FIG. 6A, Addition of 5′ deadenylase and RecJ_{f} to remove excess 5′ pre-adenylylated linker. mRNA fragments of RS and RF were size-selected and dephosphorylated by PNK treatment, followed by 5′ pre-adenylylated linker ligation. The original method used gel purification to remove the excess linker. In the new method (pink background), 5′ deadenylase was used to remove pre-adenylylated group (Ap) from the unligated linker allowing cleavage by RecJ_{f}. The resulting sample could then be used directly for reverse transcription. FIG. 6B, The original (Original) and new (New) methods to remove excess linker were compared. 26 and 34 nt synthetic RNA markers were used for linker ligation. RNA markers without the linker were used as controls. Arrow indicates the excess linkers. DNA ladder, 10-bp. FIG. 6C, Reverse transcription (RT) showed the improvement of the new method over the original one. Half of the ligation mixture (O) was gel purified to remove excess linkers before RT (loaded 2×). The other half (N) was treated with 5′ deadenylase and RecJ_{f}, and directly used as template for RT (loaded 1×). RT primers were loaded as control. Arrow indicates excess RT primers.

FIGS. 7A-7H show the quality and reproducibility of RS and RF libraries, related to FIGS. 2A-2J. FIG. 7A, BioAnalyzer profile showed high quality of RS and RF libraries. In addition to internal standards (35 bp and 10380 bp), a single ˜170 bp peak is present for RS and RF libraries for Mock and elf18 treatments with both biological replicates (Rep1/2). FIG. 7B, Length distribution of total reads from 4 RS and 4 RF libraries. FIG. 7C, Fraction of 30 nt reads in total reads from 4 RS and 4 RF libraries. Data are shown as mean±s.e.m. (n=4) of percentage of reads with 5′ aligning to A (frame1), U (frame2) and G (frame3) of the initiation codon. FIG. 7D, Read density along 5′UTR, CDS and 3′ UTR of total reads from 4 RS and 4 RF libraries. Expressed genes with RPKM in CDS≥1 and length of UTR≥1 nt were used for box plots. The top, middle and bottom line of the box indicate the 25, 50 and 75 percentiles, respectively. FIG. 7E, Nucleotide resolution of the coverage around start and stop codons using the 15^{th} nucleotide of 30-nt reads of RF. FIG. 7F, Correlation between two replicates (Rep1/2) of RS and RF samples. Data are shown as the correlation of log₂RPKM in CDS for expressed genes with RPKM in CDS≥1. Pearson correlation coefficient r is shown. FIGS. 7G, 7H, Hierarchical clustering showing the reproducibility between RS (FIG. 7G) and RF (FIG. 7H) within two replicates (Rep1/2). Darker colour means greater correlation.

FIGS. 8A-8C show a flowchart and statistical methods for transcriptome, translatome, and TE change analyses. FIG. 8A, Flowchart for read processing and assignment. FIG. 8B, Statistical methods and criteria for transcriptome (RSfc), translatome (RFfc) and TE changes (TEfc) analyses. FIG. 8C, Definition of mORF/uORF ratio shift between Mock and elf18 treatments.

FIGS. 9A-9C show additional analyses of the RS, RF and TE data. FIG. 9A, Normal distribution of log₂TE for Mock and elf18 treatment. FIG. 9B, TE changes in the endogenous TBF1 gene. Read coverage was normalized to uniquely mapped reads with IGB. TEs for the TBF1 exon 2 in Mock and elf18 treatments were determined to calculate TEfc. FIG. 9C, Correlation between TEfc and exon length, 5′ UTR length, 3′ UTR length and GC composition.

FIGS. 10A-10C show PTI responses in mutants of novel regulators, related to FIGS. 2A-2J. FIG. 10A, MAPK activation. 12-day-old ein4-1, eicbp.b and erf7 seedlings were treated with 1 μM elf18 solution and collected at indicated time points for immunoblot analysis using the phosphospecific antibody against MAPK3 and MAPK6. FIG. 10B, Callose deposition. 3-week-old plants were infiltrated with 1 μM elf18 or Mock. Leaves were stained 20 h later in aniline blue followed by confocal microscopy. FIG. 10C, Effects of EIN4 UTRs on ratios of LUC/RLUC mRNA upon elf18 treatment in the transient assay performed in N. benthamiana. EV, empty vector. Mean±s.d. (2 experiments with 3 technical replicates).

FIGS. 11A-11F show uORF-mediated translational control. FIGS. 11A, 11B, Flowcharts of steps used to identify predicted (FIG. 11A) and translated (FIG. 11B) uORFs. FIG. 11C, Read density of uORF and mORF. For those genes with reads assigning to uORF and with RPKM in its mORF≥1, log₂RPKMs for individual uORFs and mORFs are plotted for Mock and elf18 treatment, respectively. r, Pearson correlation coefficient. FIG. 11D, Histogram of mORF/uORF shift upon elf18 treatment. The ratio of mORF/uORF for elf18 divided by that for Mock was defined as shift value. Data are shown as the distribution of log₂ transformation of shift values. uORFs with significant shift determined by z-score are coloured and whose numbers are shown. FIG. 11E, Histogram of mORF/uORF shift upon hypoxia stress¹¹. FIG. 11F, Venn diagrams showing overlapping uORFs with significant ribo-shift in responses to elf18 and hypoxia treatments.

FIGS. 12A-12L show R-motif-mediated translational control in response elf18 induction, related to FIGS. 3A-3G. FIG. 12A, Effects of R-motif containing 5′ leader sequences on basal translational activities after normalization to mRNA (mean±s.e.m., n=3). FIG. 12B, Effects of R-motif deletions (ΔR) on mRNA abundance (mean±s.d., 2 experiments with 3 technical replicates). FIGS. 12C-F, Effects of R-motif deletion and R-motif point substitution mutations on basal translation (FIGS. 12C, 12E; mean±s.e.m., n=4) and mRNA levels (FIGS. 12D, 12F, mean±s.d., 2 experiments with 3 technical replicates) for IAA18 and BET10 (FIGS. 12C, 12D) and TBF1 (FIGS. 12E, 12F). FIG. 12G, mRNA levels in WT and R-motif deletion mutants with and without elf18 treatment. Mean±s.d. from 3 biological replicates with 3 technical replicates). FIG. 12H, Effects of R-motif deletions (ΔR) on translational responsiveness to elf18 measured using the dual-LUC assay (Mean±s.e.m., n=3). FIG. 12I, Effects of GA, G[A]₃, G[A]₆ and G[A]ₙ repeats on mRNA levels when inserted into 5′ UTR of the reporter in transient assay performed in N. benthamiana. Mean±s.d. from 2 experiments with 3 technical replicates. FIGS. 12J, 12K, Effects of R-motif deletion and/or uORF mutations on TBF1 mRNA abundance (FIG. 12J) and transcriptional responsiveness to Mock and elf18 treatments (FIG. 12K). Mean±s.d. from 2 experiments with 3 technical replicates after normalization to WT (FIG. 12J) or WT with Mock treatment (FIG. 12K). FIG. 12L, Contributions of R-motif and uORFs to TBF1 translational response to elf18 in transgenic Arabidopsis plants. 1, 2, and 3 represent individual transgenic lines tested. Mean±s.e.m. from 2 experiments with 3 technical replicates after normalization to Mock.

FIGS. 13A-13C show the effects of PABs on mRNA transcription and PTI-associated phenotypes, related to FIGS. 4A-4H. FIG. 13A, Influence of coexpressing PAB2 on mRNA abundance. Data are mean±s.d. (3 biological replicates with 3 technical replicates). FIG. 13B, Elf18-induced seedling growth inhibition in WT, efr-1, pab2 pab4 (pab2/4) and pab2 pab8 (pab2/8) (mean±s.e.m., n=5). FIG. 13C, MAPK activation in WT, pab2/4, pab2/8 and efr-1 seedlings after elf18 treatment measured by immunoblotting using a phosphospecific antibody against MAPK3 and MAPK6.

FIGS. 14A-14D show the roles of GCN2 in PTI in plants. FIGS. 14A-14D, Effects of the gcn2 mutation on elf18-induced eIF2α phosphorylation (FIG. 14A), translational induction (FIG. 14B, mean±s.e.m. of LUC activity, n=8) and transcription of the uORFs_{TBF1}-LUC reporter (FIG. 14C, mean±s.d. of LUC mRNA, n=3), and resistance to Psm ES4326 (FIG. 14D, mean±s.e.m., n=8).

FIGS. 15A-15H show characterization of uORFs_{TBF1}-mediated translational control and TBF1 promoter-mediated transcriptional regulation. FIG. 15A, Schematics of the constructs used to study the translational activities of WT uORFs_{TBF1} or mutant uorfs_{TBF1} (ATG to CTG). FIGS. 15B-15D, Activity of cytosol-synthesized firefly luciferase (FIG. 15B; LUC; chemiluminescence with pseudo colour); fluorescence of ER-synthesized GFP_{ER} (FIG. 15C; under UV); and cell death induced by overexpression of TBF1-YFP fusion (FIG. 15D; cleared with ethanol) after transient expression in N. benthamiana for 2 d (FIGS. 15B, 15C) and 3 d (FIG. 15D), respectively. FIG. 15E, Schematic of the dual-luciferase system. RLUC, Renilla luciferase. FIG. 15F, Changes in translation of the reporter in transgenic Arabidopsis plants harbouring the dual luciferase construct in response to Mock, Psm ES4326, Pst DC3000, Pst DC3000 hrcC⁻ (Pst hrcC⁻), elf18 and flg22. Mean±s.e.m. of the LUC/RLUC activity ratios normalized to mock treatment at each time point (n=3). FIG. 15G, LUC/RLUC mRNA levels in (FIG. 15F). FIG. 15H, Endogenous TBF1 mRNA levels. UBQ5, internal control. Mean±s.d. of LUC/RLUC mRNA normalized to mock treatment at each time point from 2 experiments with 3 technical replicates. See FIGS. 19A-19N.

FIGS. 16A-16I shows the effects of controlling transcription and translation of snc1 on defense and fitness in Arabidopsis. FIGS. 16A, 16B, Effects of controlling transcription and translation of snc1 on vegetative (FIG. 16A) and reproductive (FIG. 16B) growth. snc1, the mutant carrying the autoactivated snc1-1 allele. #1 and #2, two independent transgenic lines carrying TBF1p:uORFs_{TBF1}-snc1. FIGS. 16C, 16D, Psm ES4326 growth in WT, snc1, #1 and #2 after inoculation by spray (FIG. 16C) or infiltration (FIG. 16D). Mean±s.e.m (n=12 and 24 from three experiments for Day 0 and Day 3, respectively). FIGS. 16E, 16F, Hpa Noco2 growth. Photos (FIG. 16E) and Hpa spores were collected from the infected plants (FIG. 16F) 7 dpi. Mean±s.e.m (n=12). FIGS. 16G-16I, Analyses of rosette radius (FIG. 16G), fresh weight (FIG. 16H) and total seed weight (FIG. 16I). Mean±s.e.m. Letters above indicate significant differences (P<0.05). See FIGS. 21A-21H for 4 lines together.

FIGS. 17A-17I shows the effects of controlling transcription and translation of AtNPR1 on defense and fitness in rice. FIG. 17A, Representative symptoms observed after Xoo inoculation in field-grown T1 AtNPR1-transgenic plants. FIG. 17B, Quantification of leaf lesion length for (FIG. 17A). FIGS. 17C, 17D, Representative symptoms observed after Xoc (FIG. 17C) and M. oryzae (FIG. 17D) in T2 plants grown in the growth chamber. FIGS. 17E, 17F, Quantification of leaf lesion length for (FIGS. 17C, 17D). FIGS. 17G-17I, Fitness parameters of T1 AtNPR1 transgenic rice under field conditions, including plant height (FIG. 17G) and grain yield determined by the number of grains per plant (FIG. 17H), and by 1000-grain weight (FIG. 17I). WT, recipient Oryza sativa cultivar ZH11. Mean±s.e.m. Different letters above indicate significant differences (P<0.05). See FIGS. 24A-24D and 25A-25L for 4 lines together and for more fitness parameters.

FIGS. 18A-18D show conservation of uORF2_{TBF1} nucleotide and peptide sequences in plant species. FIG. 18A, Schematic of TBF1 mRNA structure. The 5′ leader sequence contains two uORFs, uORF1 and uORF2. CDS, coding sequence. FIGS. 18B-18D, Alignment of uORF2 nucleotide sequences (FIG. 18B) (SEQ ID NOS: 482-490) and alignment (FIG. 18C) (SEQ ID NOS: 491-499) and phylogeny (FIG. 18D) of uORF2 peptide sequences in different plant species. The corresponding triplets encoding the conserved amino acids among these species are underlined. Identical residues (black background), similar residues (grey background) and missing residues (dashes) were identified using Clustlw2. At (Arabidopsis thaliana; AT4G36988), Pv (Phaseolus vulgaris; XP_007155927), Gm (Glycine max; XP_006600987), Gr (Gossypium raimondii; CO115325), Nb (Nicotiana benthamiana; CK286574), Ca (Cicer arietinum; XP_004509145), Pd (Phoenix dactylifera; XP_008797266), Ma (Musa acuminata subsp. Malaccensis; XP_009410098), Os (Oryza sativa; Os09g28354).

FIGS. 19A-19N shows characterization of uORFs_{TBF1} and uORFs_{bZIP11} in translational control, related to FIGS. 15A-15H. FIG. 19A, Subcellular localization of the LUC-YFP fusion (FIG. 19A) and GFP_{ER} (FIG. 19B). SP, signal peptide from Arabidopsis basic chitinase; HDEL, ER retention signal. FIGS. 19C-19E, mRNA levels of LUC in (FIG. 15B; n=3), GFP_{ER} in (FIG. 15C; n=4), and TBF1-YFP in (FIG. 15D; n=3) 2 dpi before cell death was observed in plants expressing TBF1. Mean±s.d. FIG. 19F, Schematics of the 5′ leader sequences used in studying the translational activities of WT uORFs_{bZIP11}, mutant uorf2a_{bZIP11} (ATG to CTG) or uorf2b_{bZIP11} (ATG to TAG). FIGS. 19G-19I, uORFs_{bZIP11}-mediated translational control of cytosol-synthesized LUC (FIG. 19G; chemiluminescence with pseudo colour); ER-synthesized GFP_{ER} (FIG. 19H; fluorescence under UV); and cell death induced by overexpression of TBF1 (FIG. 19I; cleared using ethanol) after transient expression in N. benthamiana for 2 d (FIGS. 19G, 19H) and 3 d (FIG. 19I), respectively. FIGS. 19J-19L, mRNA levels of LUC in (FIG. 19G), GFP_{ER} in (FIG. 19H), and TBF1-YFP in (FIG. 19I) from 2 experiments with 3 technical replicates. Mean±s.d. FIG. 19M, TE changes in LUC controlled by the 5′ leader sequence containing WT uORFs_{bZIP11}, mutant uorf2a_{bZIP11} or uorf2b_{bZIP11} in response to elf18 in N. benthamiana. Mean±s.e.m. of the LUC/RLUC activity ratios (n=4). FIG. 19N, LUC/RLUC mRNA changes in (FIG. 19M). Mean±s.d. of LUC/RLUC mRNA normalized to mock treatment from 2 experiments with 3 technical replicates.

FIG. 20 shows three developmental phenotypes observed in primary Arabidopsis transformants expressing snc1. Representative images of the three developmental phenotypes observed in T1 (i.e., the first generation) Arabidopsis transgenic lines carrying 35S:uorfs_{TBF1}-snc1, 35S:uORFs_{TBF1}-snc1, TBF1p:uorfs_{TBF1}-snc1 and TBF1p:uORFs_{TRF1}-snc1 (above). Fisher's exact test was used for the pairwise statistical analysis (below). Different letters in “Total” indicate significant differences between Type III versus Type I+Type II (P<0.01).

FIGS. 21A-21I shows the effects of controlling transcription and translation of snc1 on defense and fitness in Arabidopsis, related to FIGS. 16A-16I. FIGS. 21A, 21B, Psm ES4326 growth in WT, snc1, transgenic lines #1-4 after inoculation by spray (FIG. 21A; n=8) or infiltration (FIG. 21B; n=12 and 24 from three experiments for Day 0 and Day 3 respectively). Mean±s.e.m. FIG. 21C, Hpa Noco2 growth as measured by spore counts 7 dpi. Mean±s.e.m (n=12). FIGS. 21D-21G, Analyses of plant radius (FIG. 21D), fresh weight (FIG. 21E), silique number (FIG. 21F) and total seed weight (FIG. 21G). Mean±s.e.m. FIGS. 21H, 21I, Relative levels of Psm ES4326-induced snc1 protein (FIG. 21H; numbers below immunoblots) and mRNA (FIG. 21I). Mean±s.d. from 2 experiments with 3 technical replicates (FIG. 21I). #1-4, four independent transgenic lines carrying TBF1p:uORFs_{TBF1}-snc1 with #1 and #2 shown in FIGS. 16A-16I. hpi, hours after Psm ES4326 infection; CBB, Coomassie Brilliant Blue. Different letters above bar graphs indicate significant differences (P<0.05).

FIGS. 22A-22C show functionality of uORFs_{TBF1} in rice. FIGS. 22A, 22B, LUC activity (FIG. 22A) and mRNA levels (FIG. 22B) in three independent primary transgenic rice lines (called “T0” in rice research) carrying 35S:uorfs_{TBF1}-LUC and 35S:uORFs_{TBF1}-LUC. Mean±s.e.m. of LUC activities (RLU, relative light unit) of 3 biological replicates; and mean±s.e.m. of LUC mRNA levels of 3 technical replicates after normalization to the 35S:uorfs_{TBF1}-LUC line #1. FIG. 22C, Representative lesion mimic disease (LMD) phenotypes (above) and percentage of AtNPR1-transgenic rice plants showing LMD in the second generation (T1) grown in the growth chamber (below).

FIGS. 23A-23E shows the effects of controlling transcription and translation of AtNPR1 on defense in T0 rice, related to FIGS. 17A-17I. FIGS. 23A-23D, Lesion length measurements after infection by Xoo strain PXO347 in primary transformants (T0) for 35S:uorfs_{TBF1}-AtNPR1 (FIG. 23A), 35S:uORFs_{TBF1}-AtNPR1 (FIG. 23B), TBF1p:uorfs_{TBF1}-AtNPR1 (FIG. 23C) and TBF1p:uORFs_{TBF1}-AtNPR1 (FIG. 23D). Lines further analysed in T1 and T2 are circled. FIG. 23E, Average leaf lesion lengths. WT, recipient Oryza sativa cultivar ZH11. Mean±s.e.m. Different letters above indicate significant differences (P<0.05).

FIGS. 24A-24E shows the effects of controlling transcription and translation of AtNPR1 on defense in T1 rice, related to FIGS. 17A-17I. FIGS. 24A, 24B, Representative symptoms observed in T1 AtNPR1-transgenic rice plants grown in the greenhouse (FIG. 24A) after Xoo inoculation and corresponding leaf lesion length measurements (FIG. 24B). PCR was performed to detect the presence (+) or the absence (−) of the transgene gene. FIG. 24C, Quantification of leaf lesion length of 4 lines for Xoo inoculation in field-grown T1 AtNPR1-transgenic rice plants. Mean±s.e.m. Different letters above indicate significant differences (P<0.05). FIGS. 24D, 24E, Relative levels of AtNPR1 mRNA (FIG. 24D) and protein (FIG. 24E; numbers below immunoblots) in response to Xoo infection. Mean±s.d. (FIG. 24D; n=3 technical replicates).

FIGS. 25A-25L shows the effects of controlling transcription and translation of AtNPR1 on fitness in T1 rice under field conditions, related to FIGS. 17A-17I. Different letters above indicate significant differences among constructs (P<0.05).

### DETAILED DESCRIPTION

The inventors have demonstrated that upon pathogen challenge, plants not only reprogram their transcriptional activities, but also rapidly and transiently induce translation of key immune regulators, such as the transcription factor TBF1 (Pajerowska-Mukhtar, K. M. et al. Curr. Biol. 22, 103-112 (2012)). Here, in the non-limiting Examples, the inventors performed a global translatome profiling on Arabidopsis exposed to the microbe-associated molecular pattern (MAMP), elf18. The inventors show not only a lack of correlation between translation and transcription during this pattern-triggered immunity (PTI) response, but their studies also reveal a tighter control of translation than transcription. Moreover, further investigation of genes with altered translational efficiency (TE) has led the inventors to discover several new immune-responsive cis-elements that may be used to tightly control protein expression in, for example, an inducible manner. The new immune-responsive cis-elements include “R-motif,” Upstream Open Reading Frame (uORF), and 5′ untranslated region (UTR) sequences. R-motif sequences were found to be highly enriched in the 5′ UTR of transcripts with increased TE in response to PTI induction and define an mRNA consensus sequence consisting of mostly purines. The uORF sequences were also identified in the 5′ UTR of transcripts with altered TE and were found to be independent cis-elements controlling translation of immune-responsive transcripts. The R-motif and uORF sequences may be used separately or in combination, such as in the full-length 5′ regulatory sequence from genes with altered TE, to tightly control the translation of RNA transcripts in an immune-responsive or inducible manner.

The inventors contemplate that these new immune-responsive cis-elements may be used to more stringently control protein expression in cells in various applications. One potential use for these new cis-elements is in new constructs for controlling plant diseases. To this end, the inventors have also demonstrated that the 5′ UTR region of the TBF1 gene could be used to enhance disease resistance in plants by providing tighter control of defense protein translation while also minimizing the fitness penalty associated with defense protein expression. See, e.g., Example 2. TBF1 is an important transcription factor for the plant growth-to-defense switch upon immune induction ((Pajerowska-Mukhtar, K. M. et al. Curr. Biol. 22, 103-112 (2012)). Translation of TBF1 is normally tightly suppressed by two uORFs within the 5′ region in the absence of pathogen challenge.

Besides the uORFs of TBF1, the inventors contemplate that the additional immune-responsive cis-elements disclosed herein may be used to control defense protein expression to not only minimize the adverse effects of enhanced resistance on plant growth and development, but also help protect the environment through reduction in the use of pesticides which are a major source of pollution. Making broad-spectrum pathogen resistance inducible can also lighten the selective pressure for resistance pathogens.

While providing enhanced resistance in plants is one potential use for the compositions and methods disclosed herein, the inventors also recognize that such compositions and methods may be used in other plant and non-plant applications. For example, the ubiquitous presence of uORF sequences in mRNAs of organisms ranging from yeast (13% of all mRNA) to humans (49% of all mRNA) suggests potentially broad utility of these mRNA features in controlling transgene expression.

In one aspect of the present invention, constructs are provided. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies.

As used herein, the terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of natural or synthetic origin (which may be single-stranded or double-stranded and may represent the sense or the antisense strand).

The constructs provided herein may be prepared by methods available to those of skill in the art. Notably each of the constructs claimed are recombinant molecules and as such do not occur in nature. Generally, the nomenclature used herein and the laboratory procedures utilized in the present invention include molecular, biochemical, and recombinant DNA techniques that are well known and commonly employed in the art. Standard techniques available to those skilled in the art may be used for cloning, DNA and RNA isolation, amplification and purification. Such techniques are thoroughly explained in the literature.

The DNA constructs of the present invention may include a heterologous promoter operably connected to a DNA polynucleotide encoding a RNA transcript including a 5′ regulatory sequence located 5′ to an insert site, wherein the 5′ regulatory sequence includes an R-motif sequence. Heterologous as used herein simply indicates that the promoter, 5′ regulatory sequence and the insert site or the coding sequence inserted in the insert site are not all natively found together.

An “insert site” is a polynucleotide sequence that allows the incorporation of another polynucleotide of interest. Exemplary insert sites may include, without limitation, polynucleotides including sequences recognized by one or more restriction enzymes (i.e., multicloning site (MCS)), polynucleotides including sequences recognized by site-specific recombination systems such as the λ phage recombination system (i.e., Gateway Cloning technology), the FLP/FRT system, and the Cre/lox system or polynucleotides including sequences that may be targeted by the CRISPR/Cas system. The insert site may include a heterologous coding sequence encoding a heterologous polypeptide.

A “5′ regulatory sequence” is a polynucleotide sequence that when expressed in a cell may, when DNA, be transcribed and may or may not, when RNA, be translated. For example, a 5′ regulatory sequence may include polynucleotide sequences that are not translated (i.e., R-motif sequences) but control, for example, the translation of a downstream open reading frame (i.e., heterologous coding sequence). A 5′ regulatory sequence may also include an open reading frame (i.e., uORF) that is translated and may control the translation of a downstream open reading frame (i.e., heterologous coding sequence). In accordance with the present invention, the 5′ regulatory sequence is located 5′ to an insert site.

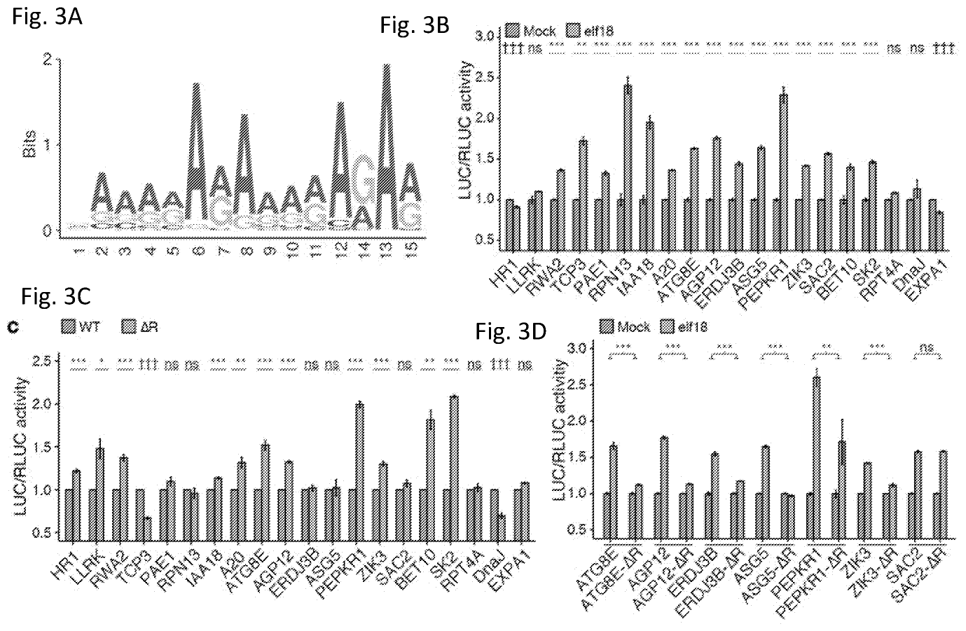

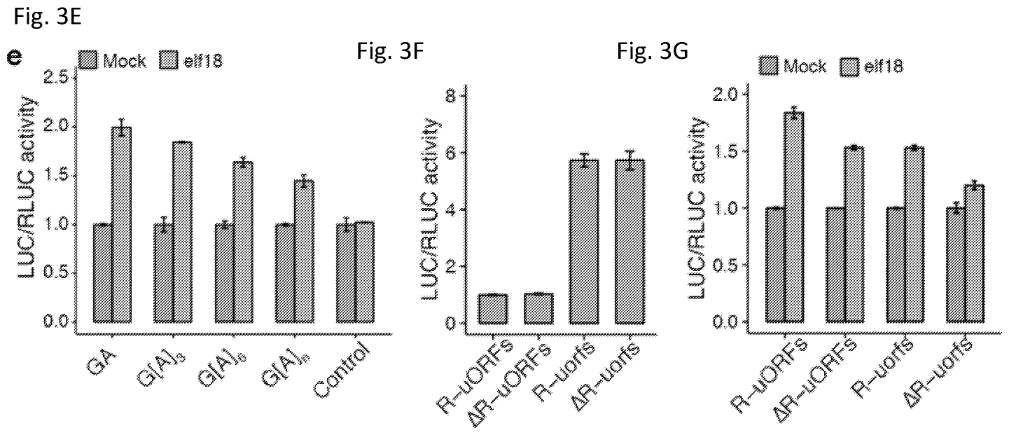

The inventors discovered a consensus sequence that is significantly enriched in the 5′ region of TE-up transcripts during PTI induction. Since the consensus sequence contains almost exclusively purines, they named it an “R-motif” in accordance with the IUPAC nucleotide code. As used herein, a “R-motif sequence” is a RNA sequence that (1) includes the consensus sequence (G/A/C)(A/G/C)(A/G/C/U)(A/G/C/U)(A/G/C)(A/G)(A/G/C)(A/G)(A/G/C/U) (A/G/C/U)(A/G/C)(A/C/U)(G/A/C)(A)(A/G/U) (See, e.g., FIG. 3A, SEQ ID NO: 481) or (2) includes 10, 11, 12, 13, 14, 15, 16, 17, 18, 19, or 20 nucleotides including G and A nucleotides in any ratio from 20G:1A to 1G:20A. In the Examples, the inventors demonstrate that R-motif sequences comprising 15 nucleotides with G[A]₃, G[A]₆ or G[A]ₙ (RNA sequences comprised of varying GA repeats having varying numbers of A nucleotides) repeats were sufficient for responsiveness to elf18. An R-motif sequence may alter the translation of an RNA transcript in an immune-responsive manner in a cell when present in the 5′ regulatory region of the transcript. An R-motif sequence may also be a DNA sequence encoding such an RNA sequence. In some embodiments, the R-motif sequence may have 40%, 60%, 80%, 90%, or 95% sequence identity to the R-motif sequences identified above. The R-motif sequence may include any one of the sequences of SEQ ID NOs: 113-293 in Table 2, a polynucleotide 10, 11, 12, 13, 14, 15, 16, 17, 18, 19, or 20 nucleotides in length comprising G and A nucleotides in any ratio from 19G:1A to 1G:19A, or a variant thereof.

Regarding polynucleotide sequences (i.e., R-motif, uORF, or 5′ regulatory polynucleotide sequences), a “variant,” “mutant,” or “derivative” may be defined as a polynucleotide sequence having at least 50% sequence identity to the particular polynucleotide over a certain length of one of the polynucleotide sequences using blastn with the “BLAST 2 Sequences” tool available at the National Center for Biotechnology Information's website. Such a pair of polynucleotides may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Regarding polynucleotide sequences, the terms “percent identity” and “% identity” and “% sequence identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent sequence identity for a polynucleotide may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 2, at least 3, at least 10, at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Polynucleotides homologous to the polynucleotides described herein are also provided. Those of skill in the art also understand the degeneracy of the genetic code and that a variety of polynucleotides can encode the same polypeptide. In some embodiments, the polynucleotides (i.e., the uORF polynucleotides) may be codon-optimized for expression in a particular cell. While particular polynucleotide sequences which are found in plants are disclosed herein any polynucleotide sequences may be used which encode a desired form of the polypeptides described herein. Thus non-naturally occurring sequences may be used. These may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

In some embodiments, the 5′ regulatory sequence lacks a TBF1 uORF sequence. A “TBF1 uORF sequence” refers to an upstream open reading frame residing in the 5′ UTR region of the TBF1 gene. The TBF1 gene is a plant transcription factor important in plant immune responses. TBF1 uORF sequences were identified in U.S. Patent Publication 2015/0113685. In some embodiments, the 5′ regulatory sequence may lack polynucleotides encoding SEQ ID NO: 102 of the US2015/0113685 publication (Met Val Val Val Phe Be Phe Phe Leu His His Gln Ile Phe Pro) or variant described therein and/or polynucleotides encoding SEQ ID NO: 103 of the US2015/0113685 publication (Met Glu Glu Thr Lys Arg Asn Ser Asp Leu Leu Arg Ser Arg Val Phe Leu Ser Gly Phe Tyr Cys Trp Asp Trp Glu Phe Leu Thr Ala Leu Leu Leu Phe Ser Cys) or variants described therein.

The 5′ regulatory sequence may include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more R-motif sequences. In some embodiments, the 5′ regulatory sequence includes between 5 and 25 R-motif sequences or any range therein. Within the 5′ regulatory sequence, each R-motif sequence may be separated by at least 0, 1, 5, 10, 15, 20, 25, 30, 35, 40, 45, 50, 55, 60, or more bases.

The 5′ regulatory sequence may include a uORF polynucleotide encoding any one of the uORF polypeptides of SEQ ID NOS: 1-38 in Table 1 or a variant thereof. In some embodiments, the 5′ regulatory sequence includes any one of the polynucleotides of SEQ ID NOs: 39-76 in Table 1 or a variant thereof. In some embodiments, the 5′ regulatory sequence includes any one of the polynucleotides of SEQ ID NOs: 77-112 in Table 1, SEQ ID NOs: 294-474 in Table 2, or a variant thereof.

The polypeptides disclosed herein (i.e., the uORF polypeptides) may include “variant” polypeptides, “mutants,” and “derivatives thereof.” As used herein the term “wild-type” is a term of the art understood by skilled persons and means the typical form of a polypeptide as it occurs in nature as distinguished from variant or mutant forms. As used herein, a “variant, “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a uORF polypeptide mutant or variant polypeptide may have one or more insertions, deletions, or substitution of at least one amino acid residue relative to the uORF “wild-type” polypeptide. The polypeptide sequences of the “wild-type” uORF polypeptides from Arabidopsis are presented in Table 1. These sequences may be used as reference sequences.

The polypeptides provided herein may be full-length polypeptides or may be fragments of the full-length polypeptide. As used herein, a “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full length polypeptide. A fragment of a uORF polypeptide may comprise or consist essentially of a contiguous portion of an amino acid sequence of the full-length uORF polypeptide (See SEQ ID NOs. in Table 1). A fragment may include an N-terminal truncation, a C-terminal truncation, or both truncations relative to the full-length uORF polypeptide.

A “deletion” in a polypeptide refers to a change in the amino acid sequence resulting in the absence of one or more amino acid residues. A deletion may remove at least 1, 2, 3, 4, 5, 10, 20, 50, 100, 200, or more amino acids residues. A deletion may include an internal deletion and/or a terminal deletion (e.g., an N-terminal truncation, a C-terminal truncation or both of a reference polypeptide).

“Insertions” and “additions” in a polypeptide refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A variant of a YTHDF polypeptide may have N-terminal insertions, C-terminal insertions, internal insertions, or any combination of N-terminal insertions, C-terminal insertions, and internal insertions.

The amino acid sequences of the polypeptide variants, mutants, or derivatives as contemplated herein may include conservative amino acid substitutions relative to a reference amino acid sequence. For example, a variant, mutant, or derivative polypeptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The DNA constructs of the present invention may also include a heterologous promoter operably connected to a DNA polynucleotide encoding a RNA transcript including a 5′ regulatory sequence located 5′ to an insert site, wherein the 5′ regulatory sequence includes a uORF polynucleotide encoding any one of the uORF polypeptides of SEQ ID NOs: 1-38 in Table 1 or a variant thereof. In some embodiments, the 5′ regulatory sequence included in the DNA construct includes any one of the polynucleotides of SEQ ID NOs: 39-76 in Table 1 or a variant thereof. In some embodiments, the 5′ regulatory sequence included in the DNA construct includes any one of the polynucleotides of SEQ ID NOs: 77-112 in Table 1, SEQ ID NOs: 294-474 in Table 2, or a variant thereof.

The constructs of the present invention may include an insert site including a heterologous coding sequence encoding a heterologous polypeptide. In some embodiments, the expression of the constructs of the present invention in a cell produces a transcript including the heterologous coding sequence and a 5′ regulatory sequence. A “heterologous coding sequence” is a region of a construct that is an identifiable segment (or segments) that is not found in association with the larger construct in nature. When the heterologous coding region encodes a gene or a portion of a gene, the gene may be flanked by DNA that does not flank the genetic DNA in the genome of the source organism. In another example, a heterologous coding region is a construct where the coding sequence itself is not found in nature.

A “heterologous polypeptide” “polypeptide” or “protein” or “peptide” may be used interchangeably to refer to a polymer of amino acids. A “polypeptide” as contemplated herein typically comprises a polymer of naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The heterologous polypeptide may include, without limitation, a plant pathogen resistance polypeptide, a therapeutic polypeptide, a transcription factor, a CAS protein (i.e. Cas9), a reporter polypeptide, a polypeptide that confers resistance to drugs or agrichemicals, or a polypeptide that is involved in the growth or development of plants.

As used herein, a “plant pathogen resistance polypeptide” refers to any polypeptide, that when expressed within a plant, makes the plant more resistant to pathogens including, without limitation, viral, bacterial, fungal pathogens, oomycete pathogens, phytoplasms, and nematodes. Suitable plant pathogen resistance polypeptides are known in the art and may include, without limitation, Pattern Recognition Receptors (PRRs) for MAMPs, intracellular nucleotide-binding and leucine-rich repeat (NB-LRR) immune receptors (also known as “R proteins”), snc-1, NPR1 such as Arabidopsis NPR1 (AtNPR1), or defense-related transcription factors such as TBF1, TGAs, WRKYs, and MYCs. NPR1 is a positive regulator of broad-spectrum resistance induced by a general plant immune signal salicylic acid. While R proteins only function within the same family of plants, overexpression of the Arabidopsis NPR1 (AtNPR1) could enhance resistance in diverse plant families such as rice, wheat, tomato and cotton against a variety of pathogens. The Arabidopsis snc1-1 (for simplicity, snc-1 herein) is an autoactivated point mutant of the NB-LRR immune receptor SNC1.

In some embodiments, the heterologous polypeptide may be a therapeutic polypeptide, industrial enzyme or other useful protein product. Exemplary therapeutic polypeptides are summarized in, for example Leader et al., Nature Review—Drug Discovery 7:21-39 (2008). Therapeutic polypeptides include but are not limited to enzymes, antibodies, hormones, cytokines, ligands, competitive inhibitors and can be naturally occurring or engineered polypeptides. The therapeutic polypeptides may include, without limitation, Insulin, Pramlintide acetate, Growth hormone (GH), somatotropin, Mecasermin, Mecasermin rinfabate, Factor VIII, Factor IX, Antithrombin III (AT-III), Protein C, beta-Gluco-cerebrosidase, Alglucosidase-alpha, Laronidase, Idursulphase, Galsulphase, Agalsidase-beta, alpha-1-Proteinase inhibitor, Lactase, Pancreatic enzymes (lipase, amylase, protease), Adenosine deaminase, immunoglobulins, Human albumin, Erythropoietin, Darbepoetin-alpha, Filgrastim, Pegfilgrastim, Sargramostim, Oprelvekin, Human follicle-stimulating hormone (FSH), Human chorionic gonadotropin (HCG), Lutropin-alpha, Type I alpha-interferon, Interferon-alpha2a, Interferon-alpha2b, Interferon-alphan3, Interferon-beta1a, Interferon-beta1b, Interferon-gamma1b, Aldesleukin, Alteplase, Reteplase, Tenecteplase, Urokinase, Factor VIIa, Drotrecogin-alpha, Salmon calcitonin, Teriparatide, Exenatide, Octreotide, Dibotermin-alpha, Recombinant human bone morphogenic protein 7 (rhBMP7), Histrelin acetate, Palifermin, Becaplermin, Trypsin, Nesiritide, Botulinumtoxin type A, Botulinum toxin type B, Collagenase, Human deoxy-ribonuclease I, dornase-alpha, Hyaluronidase (bovine, ovine), Hyaluronidase (recombinant human, Papain, L-Asparaginase, Rasburicase, Lepirudin, Bivalirudin, Streptokinase, Anistreplase, Bevacizumab, Cetuximab, Panitumumab, Alemtuzumab, Rituximab, Trastuzumab, Abatacept, Anakinra, Adalimumab, Etanercept, Infliximab, Alefacept, Efalizumab, Natalizumab, Eculizumab, Antithymocyte globulin (rabbit), Basiliximab, Daclizumab, Muromonab-CD3, Omalizumab, Palivizumab, Enfuvirtide, Abciximab, Pegvisomant, Crotalidae polyvalent immune Fab (ovine), Digoxin immune serum Fab (ovine), Ranibizumab, Denileukin diftitox, Ibritumomab tiuxetan, Gemtuzumab ozogamicin, Tositumomab, Hepatitis B surface antigen (HBsAg), HPV vaccine, OspA, Anti-Rhesus (Rh) immunoglobulin G98 Rhophylac, Recombinant purified protein derivative (DPPD), Glucagon, Growth hormone releasing hormone (GHRH), Secretin, Thyroid stimulating hormone (TSH), thyrotropin, Capromab pendetide, Satumomab pendetide, Arcitumomab, Nofetumomab, Apcitide, Imciromab pentetate, Technetium fanolesomab, HIV antigens, and Hepatitis C antigens.

The constructs of the present invention may include a heterologous promoter. The terms “heterologous promoter,” “promoter,” “promoter region,” or “promoter sequence” refer generally to transcriptional regulatory regions of a gene, which may be found at the 5′ or 3′ side of the insert site, or within the coding region of the heterologous coding sequence, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase. The heterologous promoter may be the endogenous promoter of an endogenous gene modified to include the heterologous R-motif, uORF, and/or 5′ regulatory sequences (i.e., separately or in combination) described herein using, for example, genome editing technologies. The heterologous promoter may be natively associated with the 5′UTR chosen, but be operably connected to a heterologous coding sequence.

In some embodiments, the insert site (whether including a heterologous coding sequence or not) is operably connected to the promoter. As used herein, a polynucleotide is “operably connected” or “operably linked” when it is placed into a functional relationship with a second polynucleotide sequence. For instance, a promoter is operably linked to an insert site or heterologous coding sequence within the insert site if the promoter is connected to the coding sequence or insert site such that it may affect transcription of the coding sequence. In various embodiments, the polynucleotides may be operably linked to at least 1, at least 2, at least 3, at least 4, at least 5, or at least 10 promoters.

Promoters useful in the practice of the present invention include, but are not limited to, constitutive, inducible, temporally-regulated, developmentally regulated, chemically regulated, tissue-preferred and tissue-specific promoters. Suitable promoters for expression in plants include, without limitation, the TBF1 promoter from any plant species including Arabidopsis, the 35S promoter of the cauliflower mosaic virus, ubiquitin, tCUP cryptic constitutive promoter, the Rsyn7 promoter, pathogen-inducible promoters, the maize In2-2 promoter, the tobacco PR-1a promoter, glucocorticoid-inducible promoters, estrogen-inducible promoters and tetracycline-inducible and tetracycline-repressible promoters. Other promoters include the T3, T7 and SP6 promoter sequences, which are often used for in vitro transcription of RNA. In mammalian cells, typical promoters include, without limitation, promoters for Rous sarcoma virus (RSV), human immunodeficiency virus (HIV-1), cytomegalovirus (CMV), SV40 virus, and the like as well as the translational elongation factor EF-1α promoter or ubiquitin promoter. Those of skill in the art are familiar with a wide variety of additional promoters for use in various cell types. In some embodiments, the heterologous promoter includes a plant promoter. In some embodiments, the heterologous promoter includes a plant promoter inducible by a plant pathogen or chemical inducer. The heterologous promoter may be a seed-specific or fruit-specific promoter.

The DNA constructs of the present invention may include a heterologous promoter operably connected to a DNA polynucleotide encoding a RNA transcript comprising a 5′ regulatory sequence located 5′ to a heterologous coding sequence encoding an AtNPR polypeptide comprising SEQ ID NO: 475, wherein the 5′ regulatory sequence comprises SEQ ID NO: 476 (uORFs_{TBF1}). In some embodiments, the heterologous promoter of such constructs may include SEQ ID NO: 477 (35S promoter) or SEQ ID NO: 478 (TBF1p). In some embodiments, such DNA constructs may include SEQ ID NO: 479 (35S:uORFs_{TBF1}-AtNPR1) or SEQ ID NO: 480 (TBF1p:uORFs_{TBF1}-AtNPR1).

Vectors including any of the constructs described herein are provided. The term “vector” is intended to refer to a polynucleotide capable of transporting another polynucleotide to which it has been linked. In some embodiments, the vector may be a “plasmid,” which refers to a circular double-stranded DNA loop into which additional DNA segments may be ligated. Another type of vector is a viral vector (e.g., replication defective retroviruses, herpes simplex virus, lentiviruses, adenoviruses and adeno-associated viruses), where additional polynucleotide segments may be ligated into the viral genome. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome, such as some viral vectors or transposons. Plant mini-chromosomes are also included as vectors. Vectors may carry genetic elements, such as those that confer resistance to certain drugs or chemicals.

Cells including any of the constructs or vectors described herein are provided. Suitable “cells” that may be used in accordance with the present invention include eukaryotic cells. Suitable eukaryotic cells include, without limitation, plant cells, fungal cells, and animal cells such as cells from popular model organisms including, but not limited to, Arabidopsis thaliana. In some embodiments, the cell is a plant cell such as, without limitation, a corn plant cell, a bean plant cell, a rice plant cell, a soybean plant cell, a cotton plant cell, a tobacco plant cell, a date palm cell, a wheat cell, a tomato cell, a banana plant cell, a potato plant cell, a pepper plant cell, a moss plant cell, a parsley plant cell, a citrus plant cell, an apple plant cell, a strawberry plant cell, a rapeseed plant cell, a cabbage plant cell, a cassava plant cell, and a coffee plant cell.

Plants including any of the DNA constructs, vectors, or cells described herein are provided. The plants may be transgenic or transiently-transformed with the DNA constructs or vectors described herein. In some embodiments, the plant may include, without limitation, a corn plant, a bean plant, a rice plant, a soybean plant, a cotton plant, a tobacco plant, a date palm plant, a wheat plant, a tomato plant, a banana plant, a potato plant, a pepper plant, a moss plant, a parsley plant, a citrus plant, an apple plant, a strawberry plant, a rapeseed plant, a cabbage plant, a cassava plant, and a coffee plant.

Methods for controlling the expression of a heterologous polypeptide in a cell are provided. The methods may include introducing any one of the constructs or vectors described herein into the cell. Preferably, the constructs and vectors include a heterologous coding sequence encoding a heterologous polypeptide. As used herein, “introducing” describes a process by which exogenous polynucleotides (e.g., DNA or RNA) are introduced into a recipient cell. Methods of introducing polynucleotides into a cell are known in the art and may include, without limitation, microinjection, transformation, and transfection methods. Transformation or transfection may occur under natural or artificial conditions according to various methods well known in the art, and may rely on any known method for the insertion of foreign nucleic acid sequences into a host cell. The method for transformation or transfection is selected based on the type of host cell being transformed and may include, but is not limited to, the floral dip method, Agrobacterium-mediated transformation, bacteriophage or viral infection, electroporation, heat shock, lipofection, and particle bombardment. Microinjection of polynucleotides may also be used to introduce polynucleotides and/or proteins into cells.

Conventional viral and non-viral based gene transfer methods can be used to introduce polynucleotides into cells or target tissues. Non-viral polynucleotide delivery systems include DNA plasmids, RNA, naked nucleic acid, and nucleic acid complexed with a delivery vehicle, such as a liposome. Methods of non-viral delivery of nucleic acids include the floral dip method, Agrobacterium-mediated transformation, lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™ ). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

The methods may also further include additional steps used in producing polypeptides recombinantly. For example, the methods may include purifying the heterologous polypeptide from the cell. The term “purifying” refers to the process of ensuring that the heterologous polypeptide is substantially or essentially free from cellular components and other impurities. Purification of polypeptides is typically performed using molecular biology and analytical chemistry techniques such as polyacrylamide gel electrophoresis or high performance liquid chromatography. Methods of purifying protein are well known to those skilled in the art. A “purified” heterologous polypeptide means that the heterologous polypeptide is at least 85% pure, more preferably at least 95% pure, and most preferably at least 99% pure.

The methods may also include the step of formulating the heterologous polypeptide into a therapeutic for administration to a subject. As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, mice, chickens, amphibians, reptiles, and the like. Preferably, the subject is a human patient. More preferably, the subject is a human patient in need of the heterologous polypeptide.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference in their entirety, unless explicitly indicated otherwise. The present disclosure shall control in the event there are any disparities between any definitions and/or description found in the cited references.

Unless otherwise specified or indicated by context, the terms “a”, “an”, and “the” mean “one or more.” For example, “a protein” or “an RNA” should be interpreted to mean “one or more proteins” or “one or more RNAs,” respectively. As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus≤10% of the particular term and “substantially” and “significantly” will mean plus or minus>10% of the particular term.

The following examples are meant only to be illustrative and are not meant as limitations on the scope of the invention or of the appended claims.

### EXAMPLES

### Example 1

### Revealing Global Translational Reprogramming as a Fundamental Layer of Immune Regulation in Plants

In the absence of specialized immune cells, the need for plants to reprogram transcription in order to transition from growth-related activities to defense is well understood^{1, 2}. However, little is known about translational changes that occur during immune induction. Using ribosome footprinting (RF), we performed global translatome profiling on Arabidopsis exposed to the microbe-associated molecular pattern (MAMP) elf18. We found that during the resulting pattern-triggered immunity (PTI), translation was tightly regulated and poorly correlated with transcription. Identification of genes with altered translational efficiency (TE) led to the discovery of novel regulators of this immune response. Further investigation of these genes showed that mRNA sequence features, instead of abundance, are major determinants of the observed TE changes. In the 5′ leader sequences of transcripts with increased TE, we found a highly enriched mRNA consensus sequence, R-motif, consisting of mostly purines. We showed that R-motif regulates translation in response to PTI induction through interaction with poly(A)-binding proteins. Therefore, this study provides not only strong evidence, but also a molecular mechanism for global translational reprogramming during PTI in plants.

### Results

Upon pathogen challenge, the first line of active defense in both plants and animals involves recognition of microbe-associated molecular patterns (MAMPs) by the pattern-recognition receptors (PRRs), such as the Arabidopsis FLS2 for the bacterial flagellin (epitope flg22) and EFR for the bacterial translation elongation factor EF-Tu (epitopes elf18 and elf26)³. In plants, activation of PRRs results in pattern-triggered immunity (PTI) characterized by a series of cellular changes, including an oxidative burst, MAPK activation, ethylene biosynthesis, defense gene transcription and enhanced resistance to pathogens⁴. PTI-associated transcriptional changes have been studied extensively through both molecular genetic approaches and whole genome expression profiling⁵⁻⁷. However our previous report showed that in addition to transcriptional control, translation of a key immune transcription factor (TF), TBF1, is rapidly induced during the defense response¹. TBF1 translation is regulated by two upstream open reading frames (uORFs) within the TBF1 mRNA. The inhibitory effect of the uORFs on translation of the downstream major ORF (mORF) of TBF1 was rapidly alleviated upon immune induction. Similar to TBF1, translation of the Caenorhabditis elegans immune TF, ZIP-2, was found to be regulated by 3 uORFs⁸, suggesting that de-repressing translation of pre-existing mRNAs of key immune TFs may be a common strategy for rapid response to pathogen challenge. Besides uORF-mediated TBF1 translation, perturbation of an aspartyl-tRNA synthetase by β-aminobutyric acid (BABA), a non-proteinogenic amino acid, has also been shown to prime broad-spectrum disease resistance in plants⁹. These studies suggest translational control as a major regulatory step in immune responses.

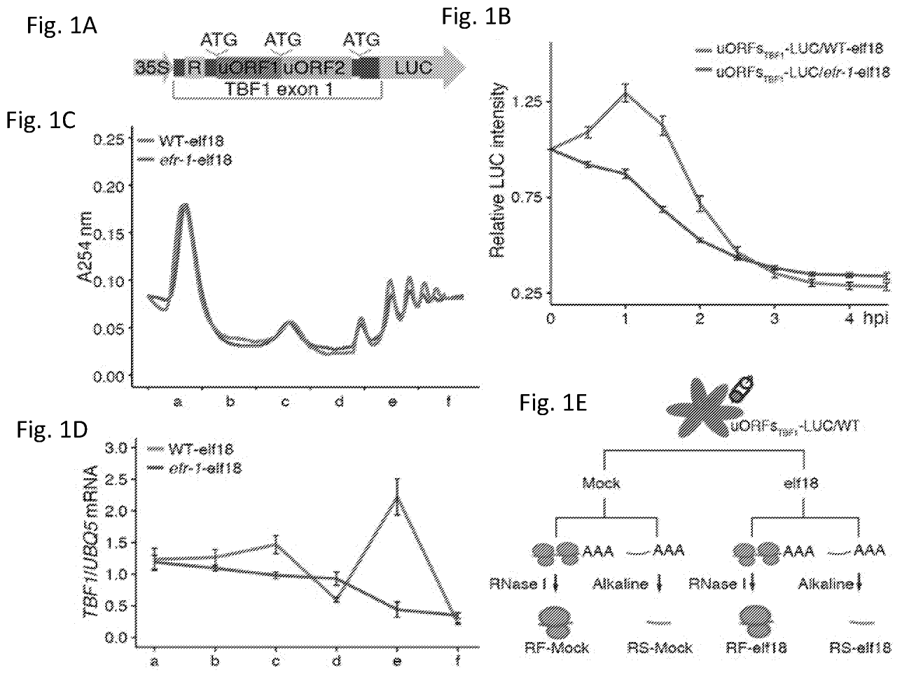

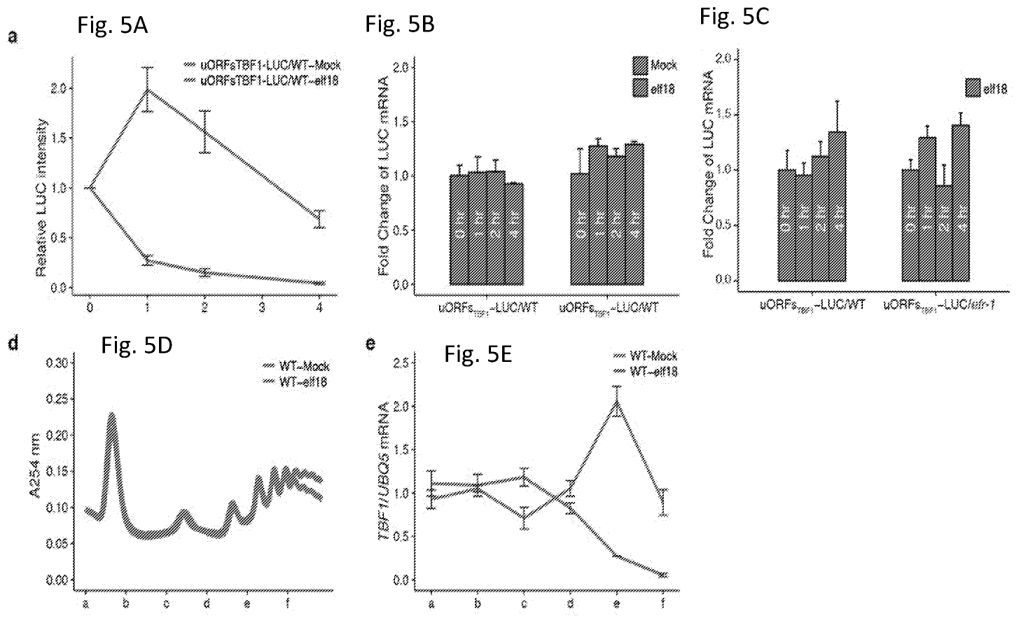

To monitor the translational changes during plant immune responses, we generated an Arabidopsis 35S:uORFs_{TBF1}-LUC reporter transgenic line (FIG. 1A). We found that in the wild type (WT) background, the reporter activity was responsive to the MAMP, elf18, with peak induction occurring one hour post-infiltration (hpi) (FIG. 1B and FIG. 5A), independent of transcriptional changes (FIG. 5B). This translational induction was compromised in the efr-1 mutant, defective in the elf18 receptor EFR⁵ (FIG. 1B and FIG. 5C), indicating that elf18 regulates the 35S:uORFs_{TBF1}-LUC reporter translation through the activity of its cell-surface receptor. Consistent with the reporter study, polysome profiling showed that in absence of overall translational activity changes (FIG. 1C and FIG. 5D), the endogenous TBF1 mRNA had a significant increase in association with the polysomal fractions after elf18 treatment in WT, but not in the efr-1 mutant (FIG. 1D and FIG. 5E).

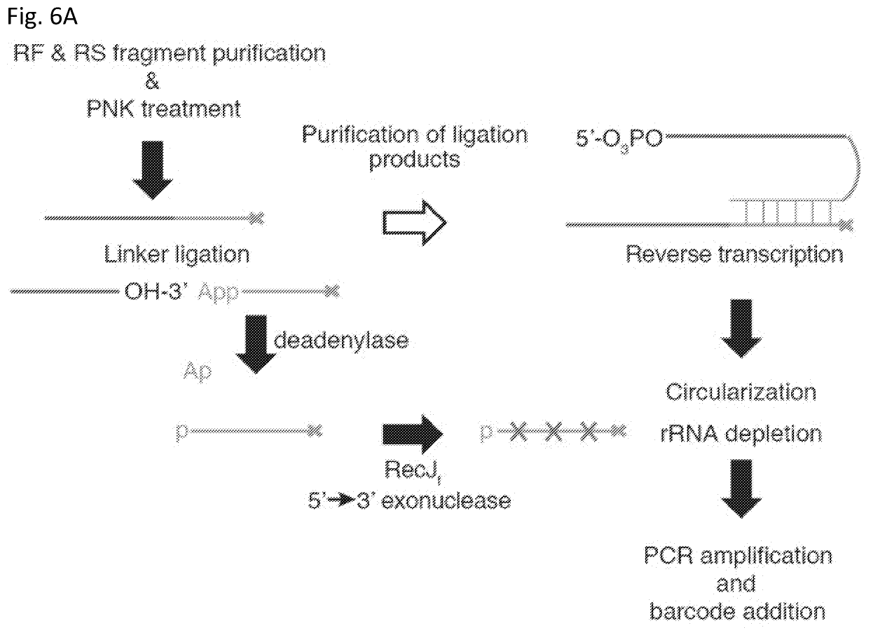

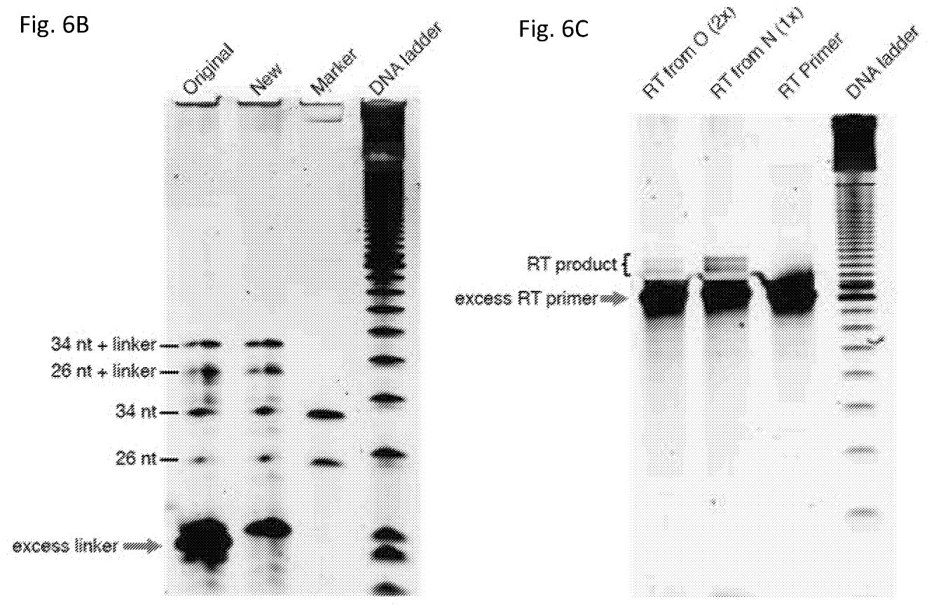

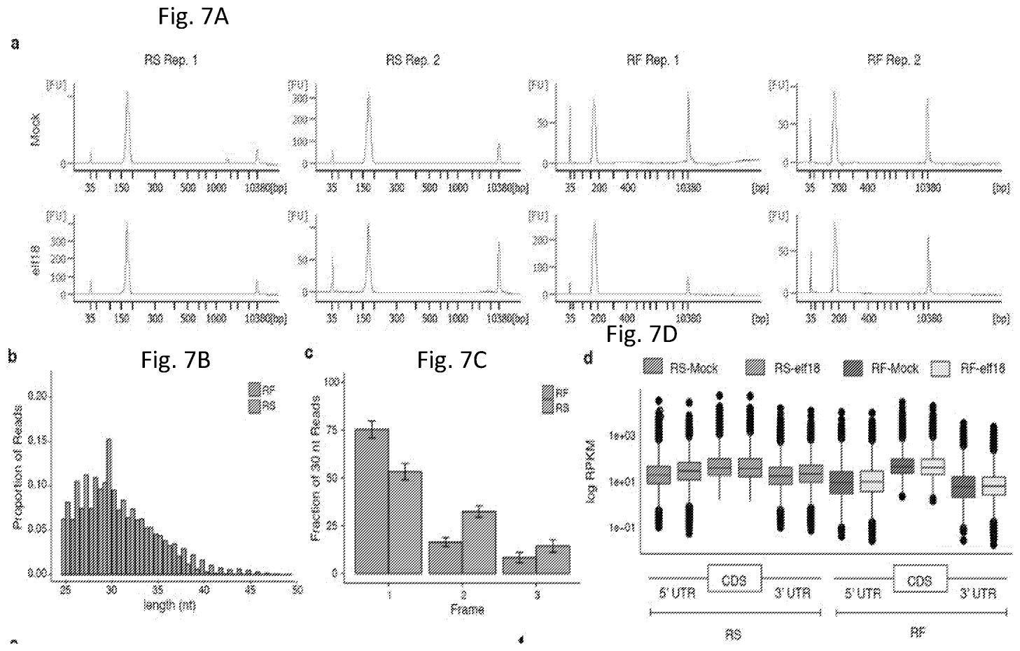

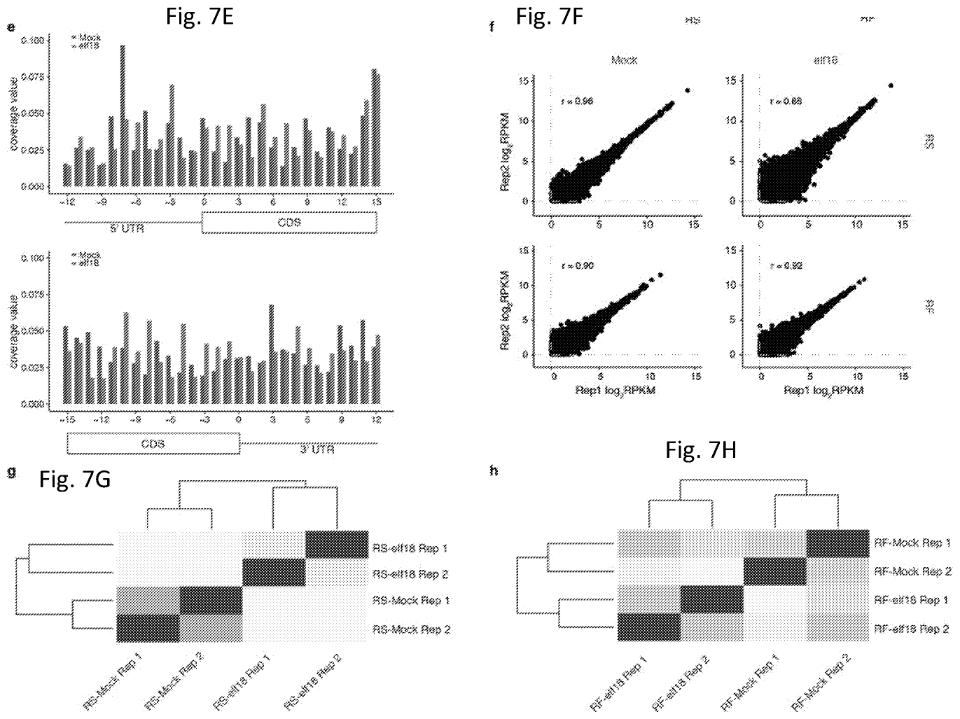

Using conditions optimized with the 35S:uORFs_{TBF1}-LUC reporter, we collected plant leaf tissues treated with either Mock or elf18 to generate libraries for ribosome footprinting-seq (RF-Mock vs RF-elf18) and RNA-seq (RS-Mock vs RS-elf18) (FIG. 1E) based on a protocol modified from previously published methods¹⁰⁻¹³ (FIGS. 6-8 all parts, Table A). Global translational status evaluation strategy, which involves counting of mRNA fragments captured by the ribosome through sequencing (Ribo-seq) versus measuring available mRNA using RNA-seq, was used to determine mRNA translational efficiency (TE). This strategy has previously been applied to study protein synthesis under different physiological conditions, such as plant responses to light, hypoxia, drought and ethylene¹¹⁻¹⁴.

TABLE A
Reads after each processing

| | | RS-Mock | RS-elf18 | RF-Mock | RF-elf18 |
|---|---|---|---|---|---|
| Raw | Rep1 | 47,085,199 | 58,742,659 | 133,768,593 | 116,236,853 |
| number | Rep2 | 47,592,232 | 58,270,271 | 113,653,155 | 125,304,695 |
| of reads | | | | | |
| Passed | Rep1 | 27,486,543 | 26,884,242 | 42,718,923 | 51,033,470 |
| reads | Rep2 | 18,843,216 | 26,721,006 | 51,905,987 | 63,096,238 |
| Unique | Rep1 | 15,576,608 | 11,988,097 | 16,809,599 | 24,748,709 |
| mapped | Rep2 | 8,463,878 | 15,824,810 | 24,866,878 | 20,900,174 |

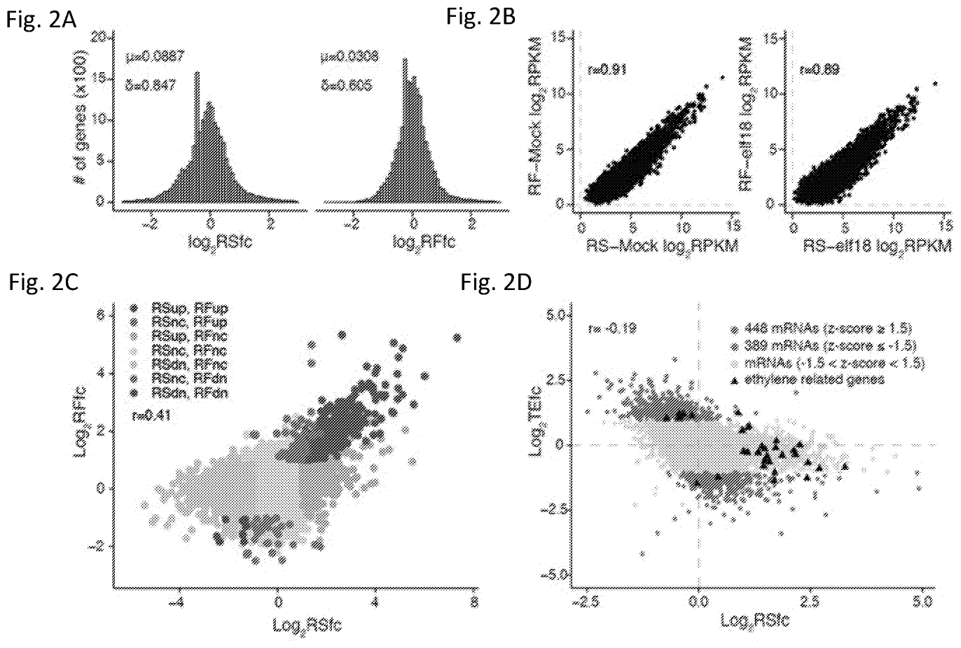

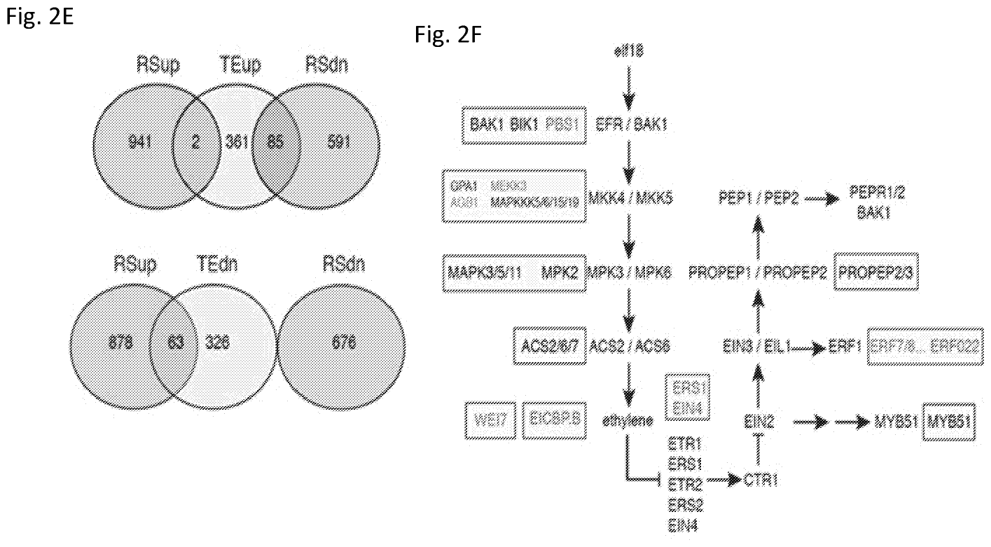

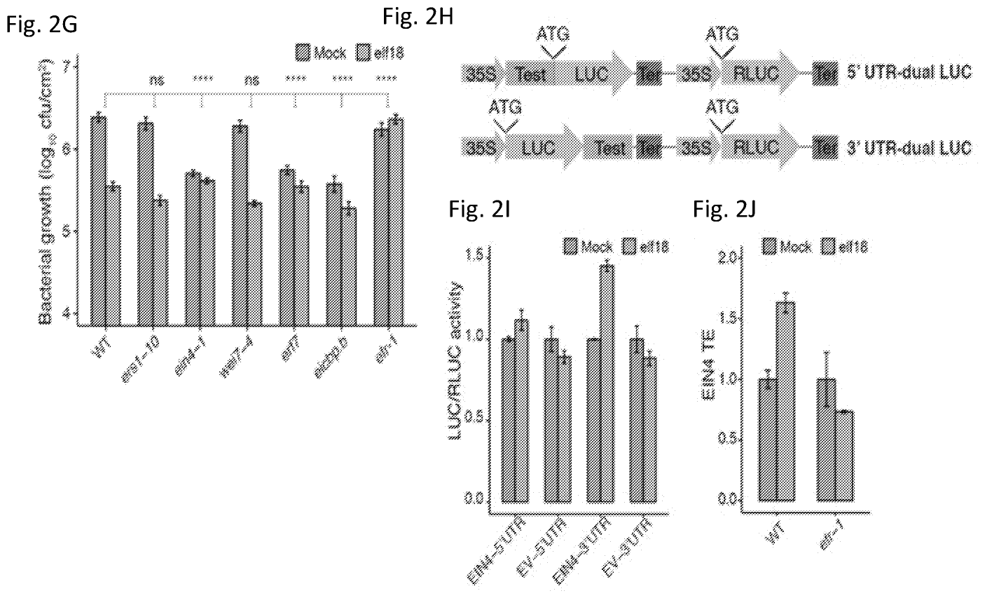

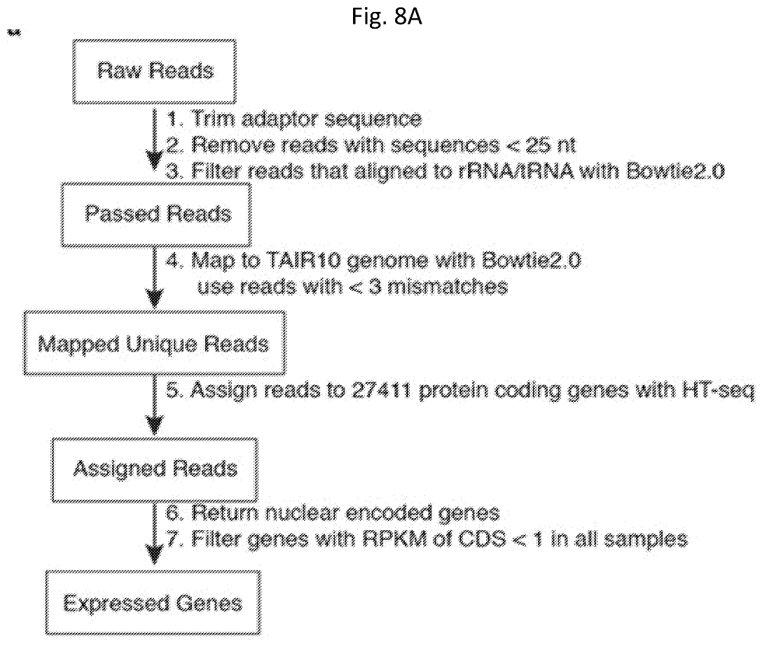

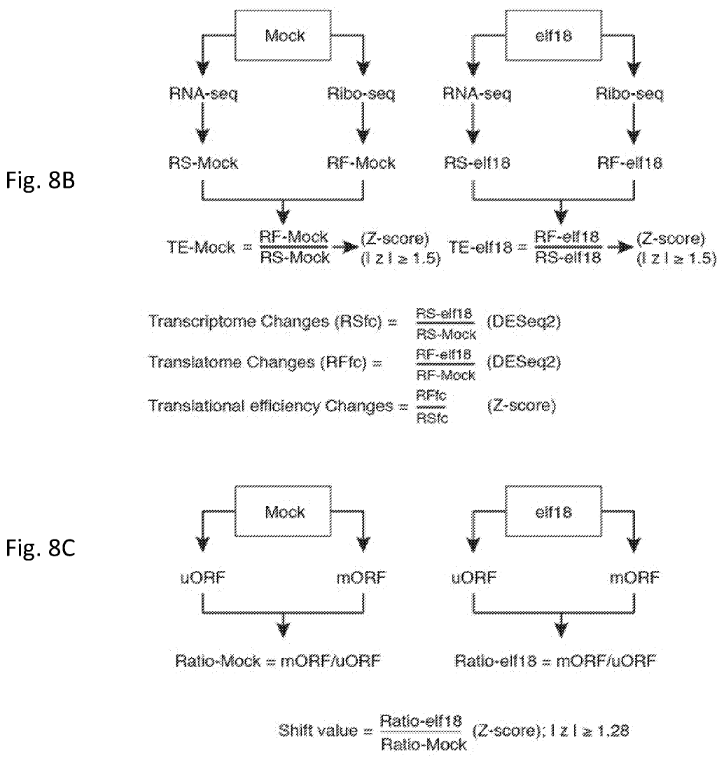

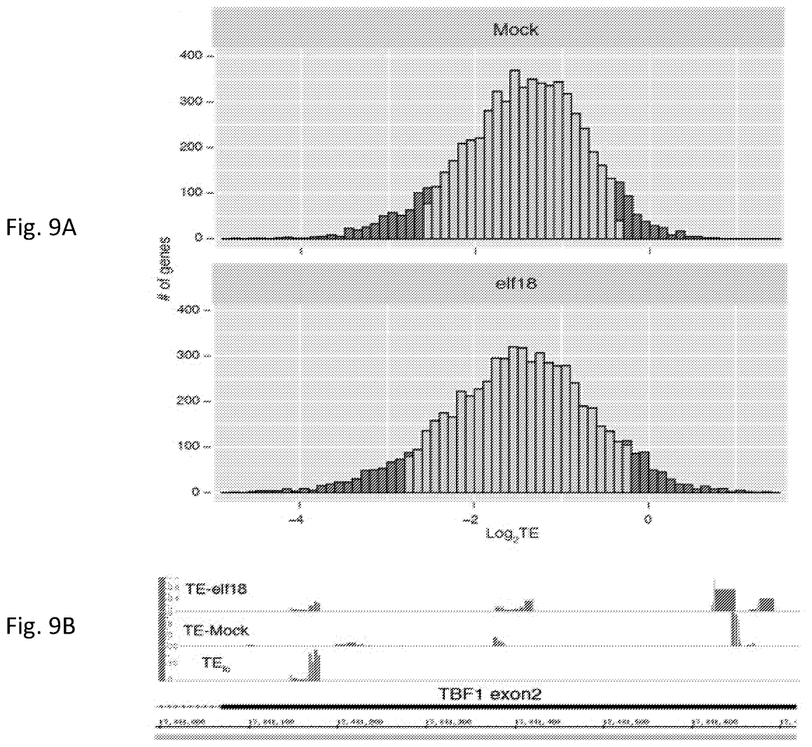

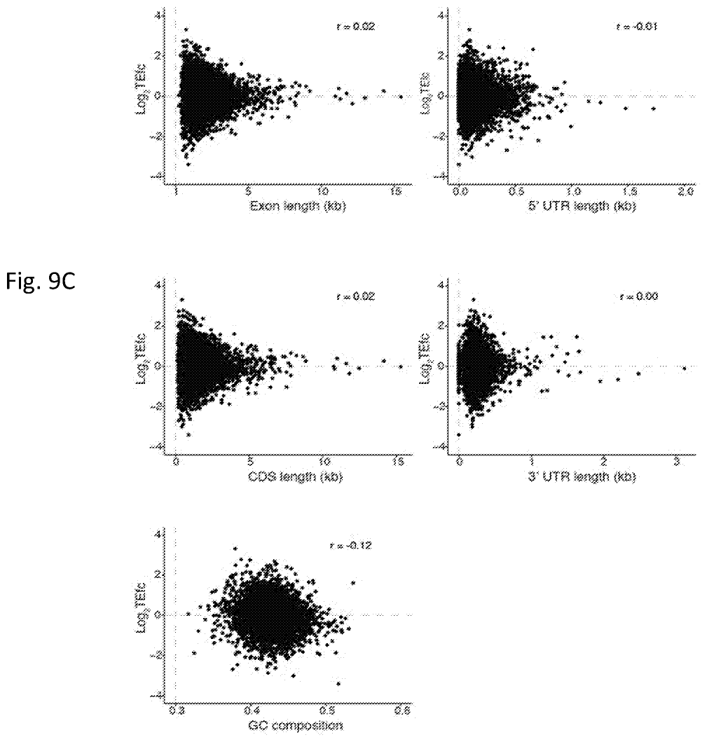

We found that upon elf18 treatment, 943 and 676 genes were transcriptionally induced (RSup) and repressed (RSdn), respectively, based on differential analysis of fold change in the transcriptome (RSfc; FIG. 8B). Gene Ontology (GO) terms enriched for RSup genes included defense response and immune response (Table B), while no GO term enrichment was found for RSdn genes. In parallel, differential analysis of the translatome (RFfc) discovered 523 genes with increased translation (RFup) and 43 genes showing decreased translation (RFdn) upon elf18 treatment (FIG. 8B). The range of RF fold changes (0.177 to 40.5) was much narrower than that of the RS fold changes (0.0232 to 160), suggesting that translation is more tightly regulated than transcription during PTI (p-value=3.22E-83; FIG. 2A). We then calculated TE values according to a previously reported formula¹⁵ (FIGS. 8B and 9B), using the endogenous TBF1 as a positive control. TE of TBF1 was determined by counting reads to its exon2 to distinguish from reads to the 35S:uORFs_{TBF}-LUC reporter containing exon1 of the TBF1 gene. Consistent with the LUC reporter assay and polysome fractionation data (FIGS. 5A and 5E), TE for the endogenous TBF1 was also increased upon elf18 treatment in our translational analysis (FIG. 9C).

TABLE B
GO term enrichment analysis for RS up-regulated genes

| | Observed | |
|---|---|---|
| GO Term | Frequency | p value |
| GO:0010200 response to chitin | 7.60% | 9.80E−46 |
| GO:0009743 response to carbohydrate stimulus | 8.40% | 2.02E−40 |
| GO:0050896 response to stimulus | 31.50% | 8.69E−31 |
| GO:0010033 response to organic substance | 14.40% | 1.57E−24 |
| GO:0042221 reponse to chemical stimulus | 18.80% | 1.45E−22 |
| GO:0006952 defense response | 10.50% | 1.77E−20 |
| GO:0006950 response to stress | 18.00% | 9.80E−19 |
| GO:0002376 immune system process | 5.80% | 2.73E−16 |
| GO:0006955 immune response | 5.20% | 1.03E−14 |
| GO:0051707 response to other organism | 7.70% | 1.42E−14 |
| GO:0045087 innate immune reponse | 5.10% | 2.58E−14 |
| GO:0051704 multi-organism process | 7.80% | 4.14E−14 |
| GO:0009607 response to biotic stimulus | 7.90% | 5.72E−14 |
| GO:0009620 reponse to fungus | 3.80% | 5.78E−12 |

In contrast to the strong correlation between levels of transcription and translation observed within the same sample (Pearson correlation values r=0.91 for Mock and 0.89 for elf18; FIG. 2B), the fold-changes (elf18/Mock) in transcription and translation were poorly correlated (r=0.41; FIG. 2C), indicating that induction of PTI involves a significant shift in global TE. Among those mRNAs with shifted TE, 448 had increased TEfc and 389 genes displayed decreased TEfc (|z|≥1.5). No correlation was found between TEfc and mRNA length or GC composition (FIG. 9D). More importantly, little correlation was found between TE changes and mRNA abundance (r=0.19; FIGS. 2D and 2E), consistent with studies performed in other systems^{13, 15}. Thus, both transcription and TE are involved in controlling protein production during PTI. Our results suggest that mRNA characteristics, apart from abundance, may be major determinants of TE.

Among the genes with increased TE (z≥1.5) upon elf18 treatment, we found moderate enrichment of genes linked to cell surface receptor signalling pathways (Table C). The lack of enrichment in immune-related GO terms is consistent with the fact that most TE-altered genes were not transcriptionally regulated and thus are unlikely to have been identified as “defense-related” in previous studies, which have primarily focused on transcriptional changes. However, by manual inspection of the TE-altered gene list, we found either a known component or a homologue of a known component of nearly every step of the ethylene- and the damage-associated molecular pattern Pep-mediated PTI signalling pathways^{7, 16, 17} (FIGS. 2D and 2F).

TABLE C
GO term enrichment found in TEup genes
in response to elf18 treatment

| | Observed | |
|---|---|---|
| GO Term | Frequency | p value |
| GO:0050896 response to stimulus | 23.50% | 9.77E−04 |
| GO:0006464 protein modification process | 10.60% | 3.43E−03 |
| GO:0007168 cell surface receptor linked signal | 2.80% | 5.08E−03 |
| GO:0009416 response to light stimulus | 5.30% | 5.08E−03 |
| GO:0007165 signal transduction | 8.40% | 5.53E−03 |
| GO:0006468 protein phosphorylation | 7.80% | 6.49E−03 |
| GO:0016310 phosphorylation | 7.80% | 7.95E−03 |
| GO:00016070 RNA metabolic process | 5.90% | 8.88E−03 |

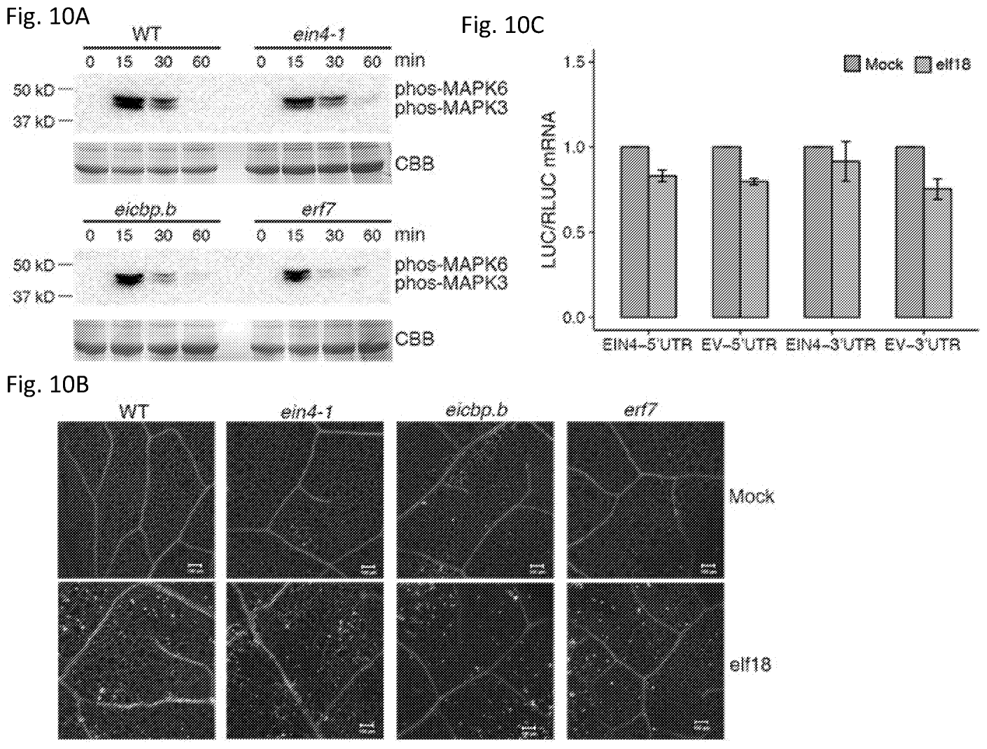

To demonstrate that TE measurement is an effective method to uncover new genes involved in the elf18 signalling pathway, we tested mutants of five TE-altered genes for elf18-induced resistance against Pseudomonas syringae pv. maculicola ES4326 (Psm ES4326). EIN4 and ERS1, which belong to the Arabidopsis ethylene receptor-related gene family¹⁸, and EICBP.B, which encodes an ethylene-induced calmodulin-binding protein, showed increased TE upon elf18 treatment. WEI7, involved in ethylene-mediated auxin increase¹⁹, and ERF7, a homologue of the ethylene responsive TF gene ERF1²⁰, showed decreased TE in response to elf18 treatment. We found that pre-treatment with elf18 induced resistance to Psm ES4326 in WT but not efr-1; among the five mutants tested, ers1-10 and wei7-4 showed responsiveness to elf18 similar to WT, whereas ein4-1, erf7, and eicbp.b displayed insensitivity to elf18-induced resistance against Psm ES4326 (FIG. 2G). The mutant phenotype of ein4-1, erf7, and eicbp.b was unlikely due to a defect in MAPK3/6 activity or callose deposition because both were found to be intact in these mutants (FIGS. 10A and 10B).

Using a dual luciferase system which allows calculation of TE using a reference Renilla luciferase (RLUC) driven by the same 35S constitutive promoter as the test gene (FIG. 2H), we found that the 3′ UTR of EIN4 was responsible for elf18-induced TE increase (FIG. 2I and FIG. 10C). Further, we confirmed that elf18-induced TE increase in EIN4 was dependent on the elf18 receptor, EFR (FIG. 2J). In contrast to EIN4, ERF7 and EICBP.B are not known to be involved in the general ethylene response and therefore may function in a defense-specific ethylene pathway. The discovery of EIN4, ERF7 and EICBP.B as new PTI components based on their TE changes suggests that there may be more novel PTI regulators to be found in the TE-altered gene list, and underscores the utility of this approach.

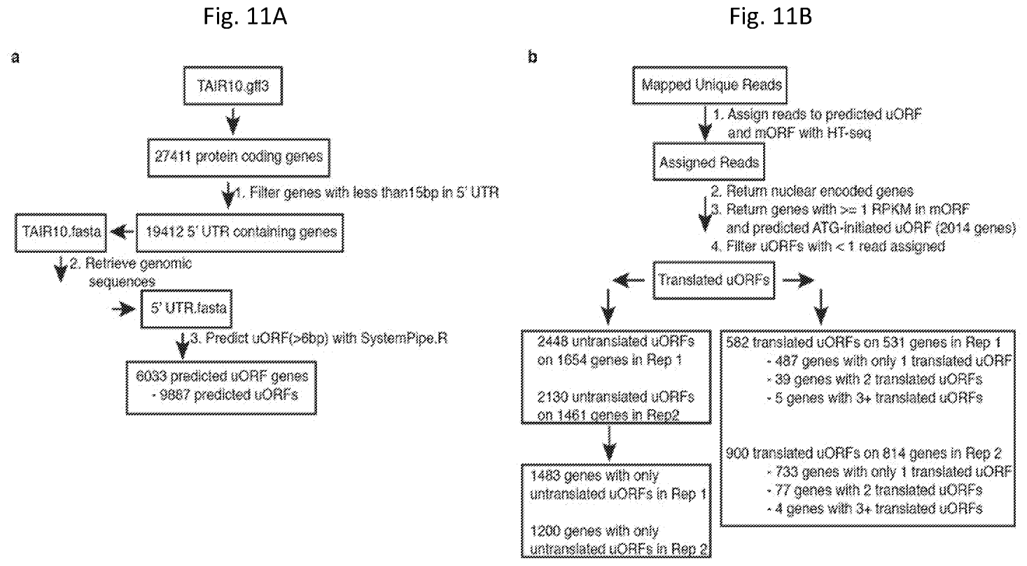

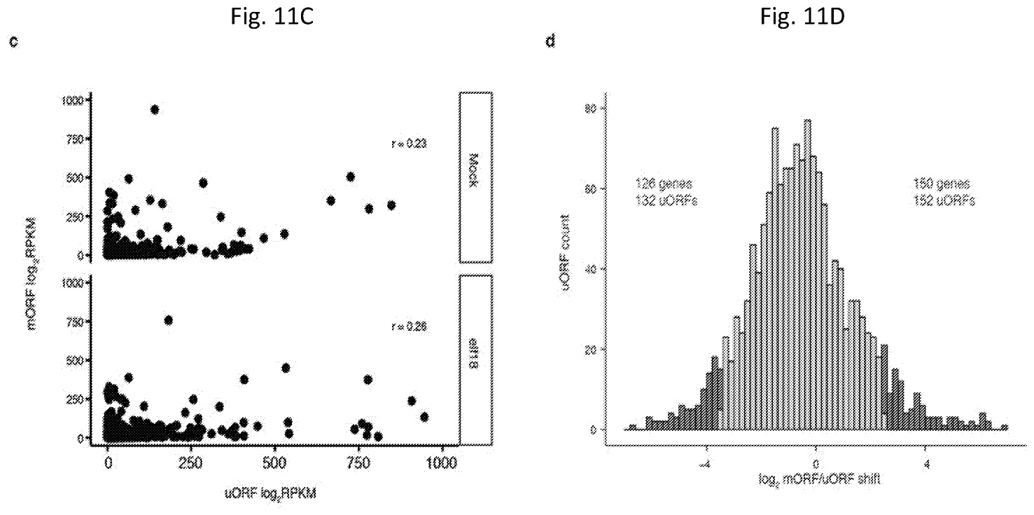

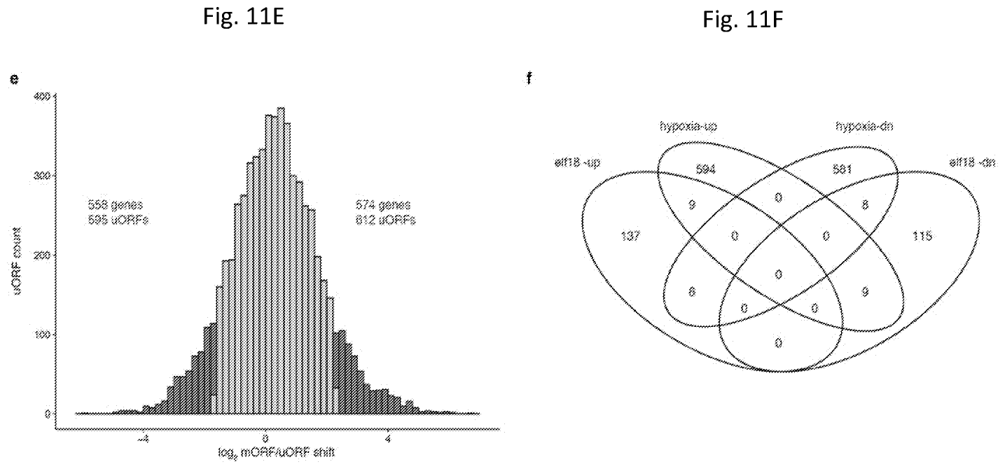

To determine the potential mechanisms governing PTI-specific translation, we studied mRNA sequence features of those transcripts with elf18-triggered TE changes. Based on our previous study of TBF1, whose translation is regulated by two uORFs¹, we first searched for the presence of uORFs (FIGS. 11A and 11B). Besides TBF1, uORFs have been associated with genes of different cellular functions in both plants²¹ and animals²². To investigate uORF-mediated translational control in response to elf18 treatment, the ratio of RF RPKM of mORFs to their cognate uORFs was calculated for all uORF-containing genes from Mock and elf18 treatments. We found no direct nor inverse overall correlation between RF reads from uORFs and mORFs (r=0.23-0.26), indicating that a uORF can have a neutral, positive or negative effect on the translation of its downstream mORF (FIG. 11C). We detected 152 uORFs belonging to 150 genes showing a ribo-shift up (i.e., increased mORF/uORF ratio) and 132 uORFs belonging to 126 genes showing a ribo-shift down (i.e., decreased mORF/uORF ratio) in response to elf18 (FIG. 11D). Interestingly, these genes with elf18-induced ribo-shift had little overlap with those found in response to hypoxia¹¹ (FIGS. 11E and 11F), suggesting that uORF-mediated translation may be signal specific. We then focused on those genes with altered TEfc in response to elf18 treatment and found 36 of them containing at least one uORF with significant ribo-shift in response to elf18 treatment. For these 36 genes, the antagonism between uORF translation and mORF translation may explain the observed TE changes in response to elf18, as observed for TBF1. The 5′ UTR and uORF sequences in several TE genes are shown in Table 1.

TABLE 1
TE UTR and uORF sequences

| transcript- | | | | | | Peptide |
|---|---|---|---|---|---|---|
| ID | alias | full name | feature | score | sequence | Seq |
| AT1G12580.1 | PEPKR1 | phospho- | 5′ | TEup | GAGAGAGGACTGGGTCTGGTCTCTTCGCTGCAA | |
| | | enolpyru- | UTR | | CCTATAGCTGTTGTTTGCTCTTCGACGGGATTCTC | |
| | | vate | | | ACTACTCTTTTGCCAAAAAAAAGAGATCGGAGGT | |
| | | carboxylase- | | | TCCGAAGGTGAATGCAGCTTGCGATTTCATAGAA | |
| | | related | | | AAGAAGATTCGTTTGCTGGATTAGGCTTATTTGT | |
| | | kinase 1 | | | GTATCATAGCTTTGAGGTTTTAACTGAGATTTATT | |
| | | | | | GATAGTGGAACTTAGGTTTTCGAGAGGTGTGAA | |
| | | | | | CAGTTGGGTAT (SEQ ID NO: 77) | |
| AT1G12580.1 | PEPKR1 | phospho- | AT1G12580. | Ribo- | ATGCAGCTTGCGATTTCATAG (SEQ ID NO: 39) | MQLAIS* |
| | | enolpyru- | 1_1 | shift | | (SEQ ID |
| | | vate | | Up | | NO: 1) |
| | | carboxylase- | | | | |
| | | related | | | | |
| | | kinase 1 | | | | |
| AT1G16700.1 | | Alpha- | 5′ | TEdown | AAATTAAGAGACATCTGATCGAATTTTGTTCCGA | |
| | | helical | UTR | | CGACCGTGAATCACCAGCAAAGGATTCGTGTCA | |
| | | ferredoxin | | | ATGTTCTTGTGAGATCGAACTTTCTCTGGGTTCG | |
| | | | | | TGCAGAAGCTTTGCTTTTTTGAGTATCGCGTTTA | |
| | | | | | AGGCACATCGAAGAAGAGAGACCCTAATTTGAT | |
| | | | | | ATTTTGAGTTCTATCG (SEQ ID NO: 78) | |
| AT1G16700.1 | | Alpha- | AT1700.1_1 | Ribo- | ATGTTCTTGTGA (SEQ ID NO: 40) | MFL* |
| | | G16 | | shift | | (SEQ ID |
| | | helical | | Down | | NO: 2) |
| | | ferredoxin | | | | |
| AT1G19270.1 | DA1 | DA1 | 5′ | TEdown | CGTGGGGAACGTTTTTTCCTGGAAGAAGAAGAA | |
| | | | UTR | | GAAGAGCTCAACAAGCTCAACGACCAAAAAACT | |
| | | | | | TCGGACACGAAGACTTTTTAATTCATTTCTCCTCT | |
| | | | | | TTTGTTTTTTTCGTTCCAAAATATTCGATACTCTC | |
| | | | | | GATCTCTTCTTCGTGATCCTCATTAAATAAAAATA | |
| | | | | | CGATTTTTATTCTTTTTTTGTGAGTGCACCAAATT | |
| | | | | | TTTTGACTTTGGATTAGCGTAGAATTCAAGCACA | |
| | | | | | TTCTGGGTTTATTCGTGTATGAGTAGACATTGAT | |
| | | | | | TTTGTCAAAGTTGCATTCTTTTATATAAAAAAAGT | |
| | | | | | TTAATTTCCTTTTTTCTTTTCTTTTCTCTTTTTTTTT | |
| | | | | | TTTTTCCCCCATGTTATAGATTCTTCCCCAAATTTT | |
| | | | | | GAAGAAAGGAGAGAACTAAAGAGTCCTTTTTGA | |
| | | | | | GATTCTTTTGCTGCTTCCCTTGCTTGATTAGATCA | |
| | | | | | TTTTTGTGATTCTGGATTTTGTGGGGGTTTCGTG | |
| | | | | | AAGCTTATTGGGATCTTATCTGATTCAGGATTTTC | |
| | | | | | TCAAGGCTGCATTGCCGTATGAGCAGATAGTTTT | |
| | | | | | ATTTAGGCATT (SEQ ID NO: 79) | |
| AT1G19270.1 | DA1 | DA1 | AT1G19270. | Ribo- | ATGAGCAGATAG (SEQ ID NO: 41) | MSR* |
| | | | 1_3 | shift | | (SEQ ID |
| | | | | Down | | NO: 3) |
| AT1G30330.1 | ARF6 | auxin | 5′ | TEdown | CTTCTTCTTCTGATTCTCATTTCAAATAAGAGAGA | |
| | | response | UTR | | GAGAGAGAGAAGTAAGTAAAACTTTAGCAGAG | |
| | | factor 6 | | | AGAAGAATAAACAAATAATTATAGCACCGTCAC | |
| | | | | | GTCGCCGCCGTATTTCGTTACCGGAAAAAAAAAA | |
| | | | | | TCATTCTTCAACATAAAAATAAAAACAGTCTCTTT | |
| | | | | | CTTTCTATCTTTGTCTATCTTTGATTATTCTCTGTG | |
| | | | | | TACCCATGTTCTGCAACAGTTGAGCAAGTGCATG | |
| | | | | | CCCCATATCTCTCTGTTTCTCATTTCCCGATCTTTG | |
| | | | | | CATTAATCATATACTTCGCCTGAGATCTCGATTAA | |
| | | | | | GCCAGCTTATAGAAGAAGAAACGGCACCAGCTT | |
| | | | | | CTGTCGTTTTAGTTAGCTCGAGATCTGTGTTTCTT | |
| | | | | | TTTTTCTTGGCTTCTGAGCTTTTGGCGGTGGTGG | |
| | | | | | GTTTTTCTGGAGAAACCCAAACGACTATCAAAGT | |
| | | | | | TTTGTTTTTTACAATTTTAAGTGGGAGTTATGAGT | |
| | | | | | GGGGTGGATTAAGTAAGTTACAAGTATGAAGGA | |
| | | | | | GTTGAAGATTCGAAGAAGCGGTTTTGAAGTCGG | |
| | | | | | CGAGACCAAGATTGCGAGCTTATTTGGCTGATG | |
| | | | | | ATTTATTTCAGGGAAGAAGAAATAAATCTGTTTT | |
| | | | | | TTTTAGGGTTTTTAGATTTGGTTGGTGAATGGGT | |
| | | | | | GGGAGGTGGAGGGAAACAGTTAAAAAAGTTAT | |
| | | | | | GCTTTTAGTGTCTCTTCTTCATAATTACATTTGGG | |
| | | | | | CATCTTGAAATCTTTGGATCTTTGAAGAAACAAA | |
| | | | | | GTTGTGTTTTTTTTTTTGTTCTTTGTTGTTTGCTTT | |
| | | | | | TTAAGTTAGAATAAAAA (SEQ ID NO: 80) | |
| AT1G30330.1 | ARF6 | auxin | AT1G30330. | Ribo- | ATGTTCTGCAACAGTTGA (SEQ ID NO: 42) | MFCNS* |
| | | response | 1_1 | shift | | (SEQ ID |
| | | factor 6 | | Up | | NO: 4) |
| AT1G30330.1 | ARF6 | auxin | AT1G30330. | Ribo- | ATGAGTGGGGTGGATTAA (SEQ ID NO: 43) | MSGVD* |
| | | response | 13 | shift | | (SEQ ID |
| | | factor 6 | | Down | | NO: 5) |
| AT1G48300.1 | | | 5′ | TEup | CGAGATGCGGCGAGGAGAAAGAGAAGGTTAAG | |
| | | | UTR | | GTT (SEQ ID NO: 81) | |
| AT1G48300.1 | | | ATG48300. | Ribo- | ATGCGGCGAGGAGAAAGAGAAGGTTAA (SEQ | MRRGERE |
| | | | 1_1 | shift | ID NO: 44) | G* (SEQ |
| | | | | Up | | ID NO: 6) |
| AT1G59700.1 | GSTU16 | glutathione | 5′ | TEdown | ATTGTGTGGTGACAAGCAACACATGATATGTCCG | |
| | | S- | UTR | | TTTAGAAACAGACAAAATAAGAAGAAGAAGAAA | |
| | | transferase | | | GAGTCGTGGAGGATTCTTCATTCTTCCTCATCCTC | |
| | | TAU16 | | | TTCTTCACCGATTCATTAGAAACCAAATTACAAA | |
| | | | | | AAAAAACGTCTATACACAAAAAAACAA (SEQ ID | |
| | | | | | NO: 82) | |
| AT1G59700.1 | GSTU16 | glutathione | AT1G59700. | Ribo- | ATGATATGTCCGTTTAGAAACAGACAAAATAAG | MICPFRN |
| | | S- | 1_1 | shift | AAGAAGAAGAAAGAGTCGTGGAGGATTCTTCAT | RQNKKKK |
| | | transferase | | Down | TCTTCCTCATCCTCTTCTTCACCGATTCATTAG | KESWRILH |
| | | TAU16 | | | (SEQ ID NO: 45) | SSSSSSSPI |
| | | | | | | H* (SEQ |
| | | | | | | ID NO: 7) |
| AT1G59990.1 | RH22 | DEA(D/H)- | 5′ | TEdown | AGTGAGCTAATGAAGAGAGAGACTGAAACAGA | |
| | | box RNA | UTR | | GGTTTCTTACTTTCTTCTCTGTATCTCTCATATTTT | |
| | | helicase | | | GCTTAAACCCTAAAACCCTTTTTGGATTAGGTTTT | |
| | | family | | | CTCCAAATCTTATCCGCCGTGATAAAATCTGATT | |
| | | protein | | | GCTTTTTTTCTTCATGAAAGTTTGATTTGTGAAAC | |
| | | | | | TCG (SEQ ID NO: 83) | |
| AT1G59990.1 | RH22 | DEA(D/H)- | AT1G59990. | Ribo- | ATGAAGAGAGAGACTGAAACAGAGGTTTCTTAC | MKRETET |
| | | box RNA | 1_1 | shift | TTTCTTCTCTGTATCTCTCATATTTTGCTTAAACCC | EVSYFLLCI |
| | | helicase | | Down | TAA (SEQ ID NO: 46) | SHILLKP* |
| | | family | | | | (SEQ ID |
| | | protein | | | | NO: 8) |
| AT1G72390.1 | | | 5′ | TEup | CCTTTCTCTTCCGATCGCATCTTCTTCAAAAATTTC | |
| | | | UTR | | CCACCTGTGTTTCACAAATTCCATGTTTATGAATT | |
| | | | | | CTTCATTGCTCTATTCTTAGTCACCTTTGATTTCTC | |
| | | | | | TCGCTTTCTATCCGATCCAATTGTTTGATGATCTT | |
| | | | | | CTCTGTAACAAGCTCATAAGGTTTGAGCTTCATC | |
| | | | | | TCTCTGGAGAGAATCC (SEQ ID NO: 84) | |
| AT1G72390.1 | | | AT1G72390. | Ribo- | ATGTTTATGAATTCTTCATTGCTCTATTCTTAG | MFMNSSL |
| | | | 1_1 | shift | (SEQ ID NO: 47) | LYS* (SEQ |
| | | | | Down | | ID NO: 9) |
| AT2G34630.1 | GPS1 | geranyl | 5′ | TEup | AAGCGAACAAGTCTTTGCCTCTTTGGTTTACTTTC | |
| | | diphosphate | UTR | | CTCTGTTTTCGATCCATTTAGAAAATGTTATTCAC | |
| | | synthase | | | GAGGAGTGTTGCTCGGATTTCTTCTAAGTTTCTG | |
| | | 1 | | | AGAAACCGTAGCTTCTATGGCTCCTCTCAATCTCT | |
| | | | | | CGCCTCTCATCGGTTCGCAATCATTCCCGATCAG | |
| | | | | | GGTCACTCTTGTTCTGACTCTCCACACAAGTAGG | |
| | | | | | GTTACGTTTGCAGAACAACTTATTCATTGAAATCT | |
| | | | | | CCGGTTTTTGGTGGATTTAGTCATCAACTCTATCA | |
| | | | | | CCAGAGTAGCTCCTTGGTTGAGGAGGAGCTTGA | |
| | | | | | CCCATTTTCGCTTGTTGCCGATGAGCTGTCACTTC | |
| | | | | | TTAGTAATAAGTTGAGAGAG (SEQ ID NO: 85) | |
| AT2G34630.1 | GPS1 | geranyl | AT2G34630. | Ribo- | ATGAGCTGTCACTTCTTAGTAATAAGTTGA (SEQ | MSCHFLVI |
| | | diphosphate | 1_2 | shift | ID NO 48) | S* (SEQ |
| | | synthase | | Up | | ID NO: 10) |
| | | 1 | | | | |
| AT2G35510.1 | SRO1 | similar to | 5′ | TEup | CAAGAGTAGACCGCCGACTTAGATTTTTTCGCCG | |
| | | RCD one | UTR | | ACGAGAGAATATATATAAATGGCTCGTCTTTTTC | |
| | | 1 | | | CAAACGATTTCTTCTTCTTCGTCGTCGCCGGTTTA | |
| | | | | | GGGTTTCCGTTGCTGTAGCAATTTTCTCTCGCTTC | |
| | | | | | TCTCTCCCCTTTTACAGTTTCTCTTATATTGCTCTT | |
| | | | | | GCCTTGCGTCCAATCTCAAGAGTTCATAAGAGTT | |
| | | | | | GACATTTGTGAACATCGAAGAAATACGGTGACG | |
| | | | | | TTTCTTCTCTGATTACTTTTTGCCAACATGAATAC | |
| | | | | | TAATGTATTTATCAACAAGTGCTACAGCCTGTTTT | |
| | | | | | TTTCGAAGCTGTTGGTGAGTTCCCATCCTTAGTA | |
| | | | | | CTGCTAGACAGTTCGGTGTGTTAGTTGACTTTAT | |
| | | | | | ATTCAAGGTTATAGGTTTAGTGTTGTTAGTAGAG | |
| | | | | | AAAA (SEQ ID NO: 86) | |
| AT2G35510.1 | SRO1 | similar to | AT2G35510. | Ribo- | ATGGCTCGTCTTTTTCCAAACGATTTCTTCTTCTTC | MARLFPN |
| | | RCD one | 1_1 | shift | GTCGTCGCCGGTTTAGGGTTTCCGTTGCTGTAG | DFFFFVVA |
| | | 1 | | Up | (SEQ ID NO: 49) | GLGFPLL* |
| | | | | | | (SEQ ID |
| | | | | | | NO: 11) |
| AT2G42950.1 | | Magnesium | 5′ | TEdown | ACATTCATCTCTCTCTCTCAGTCAAATTGTTGTTTT | |
| | | transporter | UTR | | CTTTCTTCGAATCGGTGCAGAAAATTCAGGGAAG | |
| | | CorA- | | | TTCTGGGGAAGGTTGTTGCGTTTGACTCCTTTGG | |
| | | like | | | CTTAGTTTTCTTTCGAATTCCGTGCTTCCTGATGA | |
| | | family | | | TCTTACGTGAAATTGCAGCCTAAAATTTCGAGAT | |
| | | protein | | | TGTTTTTTTTACTCAGAAAACGAGATTTGACTGAT | |
| | | | | | ATGAATCGAAAATCTGTGATTTAAAGTGAAGC | |
| | | | | | (SEQ ID NO: 87) | |
| AT2G42950.1 | | Magnesium | AT2G42950. | Ribo- | ATGATCTTACGTGAAATTGCAGCCTAA (SEQ ID | MILREIAA |
| | | transporter | 1_1 | shift | NO: 50) | * (SEQ ID |
| | | CorA- | | Down | | NO: 12) |
| | | like | | | | |
| | | family | | | | |
| | | protein | | | | |
| AT2G47210.1 | | myb-like | 5′ | TEdown | AAACTGCTGACCGATCCCAAAGGTTGAAAGATTC | |
| | | transcription | UTR | | TTTGGCGCTAAAAAATCCCCAGTTCCCAAATCGG | |
| | | factor | | | CGTCCTCGTTTGAAACCCTAATTCCTGAATCGAA | |
| | | family | | | GCAGATCCTGATCGAATCGAAGGTGTTCGAATG | |
| | | protein | | | ATAGCTACCCAGTAAATTCAGAACCCTAATTAAC | |
| | | | | | A (SEQ ID NO: 88) | |
| AT2G47210.1 | | myb-like | AT2G47210. | Ribo- | ATGATAGCTACCCAGTAA (SEQ ID NO: 51) | MIATQ* |
| | | transcription | 1_1 | shift | | (SEQ ID |
| | | factor | | Up | | NO: 13) |
| | | family | | | | |
| | | protein | | | | |
| AT3G02570.1 | PMI1 | Mannose- | 5′ | TEup | GTAAAGAGAAAAGCTTTGAGTCTTCCATTGACAT | |
| | | 6- | UTR | | GGGCGCTTAGCTTATGCTTGAGATATTTTGTTTTT | |
| | | phosphate | | | ACCTCCGAGAAACGGATTTAGATTCGTAATCGTG | |
| | | isomerase, | | | AGTTTTTTGGTGTA (SEQ ID NO: 89) | |
| | | type I | | | | |
| AT3G02570.1 | PMI1 | Mannose- | AT3G02570. | Ribo- | ATGCTTGAGATATTTTGTTTTTACCTCCGAGAAAC | MLEIFCFY |
| | | 6- | 1_2 | shift | GGATTTAGATTCGTAA (SEQ ID NO: 52) | LRETDLDS |
| | | phosphate | | Down | | * (SEQ ID |
| | | isomerase, | | | | NO: 14) |
| | | type I | | | | |
| AT3G03070.1 | | NADH- | 5′ | TEdown | AAATAAATGCGTTGTTTGGTACAGCTTCACGAAC | |
| | | ubiquinone | UTR | | AATCTCTCTCTCGATAGATTCTTCTTACCTCTGAA | |
| | | oxido- | | | TTTCTCGTTGTTGGAACA (SEQ ID NO: 90) | |
| | | reductase- | | | | |
| | | related | | | | |
| AT3G03070.1 | | NADH- | AT3G03070. | Ribo- | ATGCGTTGTTTGGTACAGCTTCACGAACAATCTC | MRCLVQL |
| | | ubiquinone | 1_1 | shift | TCTCTCGATAG (SEQ ID NO: 53) | HEQSLSR* |
| | | oxido- | | Down | | (SEQ ID |
| | | reductase- | | | | NO: 15) |
| | | related | | | | |
| AT3G15030.1 | TCP4 | TCP | 5′ | TEdown | AGATTTTTTTTTTAAACAAAGAATGGAAAAAAAT | |
| | | family | UTR | | GAATAAATTTGGGAAACGAGGAAGCTTTGGTTA | |
| | | transcription | | | CCCAAAAAAGAAAGAAAGAAAAAATAAAAAAAA | |
| | | factor | | | ATAAAAAGAAAAGCTTTCTCTGGGTTTTTCTTGA | |
| | | 4 | | | TTGGTCAATTACACATCTCCCTTTCTCTCTTCTCTC | |
| | | | | | TCTCACCTTCGCTTGCTTTGCTTGCTTCATCTCTTT | |
| | | | | | GGTCTCCTTCTTGCGTTTTCTATTTACTACACAGA | |
| | | | | | CCAAACAATAGAGAGAGACTTTAAGCTATAGAA | |
| | | | | | AAAAAGAGAGAGATTCTCTCAAATATGGGTTAG | |
| | | | | | TCCACAATTTTCACTAAACCTCTTCTTCTTAGGAT | |
| | | | | | TGTTTTTAGGGTTAGGGTTTTGAGGTGAGGAGA | |
| | | | | | GCAAGTATGCGGGAGTTTCATCCTTTTTGAGTTA | |
| | | | | | CTCTGGATTCCTCACCCTCTAACGACGACCACCG | |
| | | | | | TCGCCGCCGCCGCCGCCGTCTCGACGAATATGCT | |
| | | | | | CTACCA (SEQ ID NO: 91) | |
| AT3G15030.1 | TCP4 | TCP | AT3G15030. | Ribo- | ATGGGTTAG (SEQ ID NO: 54) | MG* (SEQ |
| | | family | 1_2 | shift | | ID NO: 16) |
| | | transcription | | Down | | |
| | | factor | | | | |
| | | 4 | | | | |
| AT3G18140.1 | | Transducin/ | 5′ | TEup | ATGAGAAAAGCTTGGTAAAAACCCTATTTTTCTT | |
| | | WD40 | UTR | | CTTCTCTTCAATTTACAGTTCTCTGCACCTTTTTCT | |
| | | repeat- | | | TTCCCCTGTTTTTTGATCCTCAATCACCAAACCCT | |
| | | like | | | AGCTTGTTCTTCTGTTGATTATTTCGAAAAGGGG | |
| | | superfamily | | | GTTTGTTTGTTTTCTGGGAATCAGCAAAAATCAC | |
| | | protein | | | GAAATGGTTGGTTTAATATTTCAATCGGGATAAA | |
| | | | | | ATCGATCGAAA (SEQ ID NO: 92) | |
| AT3G18140.1 | | Transducin/ | AT3G18140. | Ribo- | ATGGTTGGTTTAATATTTCAATCGGGATAA (SEQ | MVGLIFQS |
| | | WD40 | 1_2 | shift | ID NO: 55) | G* (SEQ |
| | | repeat- | | Down | | ID NO: 17) |
| | | like | | | | |
| | | superfamily | | | | |
| | | protein | | | | |
| AT3G55020.1 | | Ypt/Rab- | 5′ | TEup | GTCACACATGTAATAAACCTTGGTCGACAATCTC | |
| | | GAP | UTR | | GCCCTTTCCATGTGATTTCTCCACTTCCTCTCTCTC | |
| | | domain | | | TCTACTGCAACTTCCTCCTCCTGCTTCAACTTCATT | |
| | | of gyp1p | | | CGGGTAATGATGAACTAGCGTAGAGATTTGGAT | |
| | | superfamily | | | CTTCTTCTTCGTCCTCTCACCAACTCTTCACCGGTT | |
| | | protein | | | AGATCTCTTTTTCACGCTAACGA (SEQ ID NO: | |
| | | | | | 93) | |
| AT3G55020.1 | | Ypt/Rab- | AT3G55020. | Ribo- | ATGTGA (SEQ ID NO: 56) | M* (SEQ |
| | | GAP | 1_2 | shift | | ID NO: 18) |
| | | domain | | Up | | |
| | | of gyp1p | | | | |
| | | superfamily | | | | |
| | | protein | | | | |
| AT3G56010.1 | | | 5′ | TEup | GTGTTTAGCTTCTTCACTACCACACAGAAACAGA | |
| | | | UTR | | GTTTCCGTCTTTCATCTTCCTCCATATGCGTCGCT | |
| | | | | | CTTAAAAACCTAATTCACA (SEQ ID NO: 94) | |
| AT3G56010.1 | | | AT3G56010. | Ribo- | ATGCGTCGCTCTTAA (SEQ ID NO: 57) | MRRS* |
| | | | 1_1 | shift | | (SEQ ID |
| | | | | Up | | NO: 19) |
| AT3G63340.1 | | Protein | 5′ | TEdown | TCTTCTTCTTCGTTTTCAGGCGGGTGGAGGAGCT | |
| | | phosphatase | UTR | | CAGAGCCTTCCAGAGGTAACCAACCTTTTATTAC | |
| | | 2C | | | CGACAAGATTCTGCCACACAATTATTACATATTTT | |
| | | family | | | TGTTCCCATGAAGCAATTGTTCCTTTCAAGCATGT | |
| | | protein | | | TTACGAGCAAAAGAGTGAAAGGGTAGCTTGATT | |
| | | | | | TTTGTCTACTCTAGCTTCATTTTCTGGCGATCTTT | |
| | | | | | ACTTGAGATTTAAACATTTTGCTCTCGGATTGATA | |
| | | | | | ATAAAGAAGAATTTGGAATATCAGTAGGTTTGG | |
| | | | | | TTAGGACTCTCGGATTCTGTTGTCGGTTAGATAT | |
| | | | | | TTGTTTTGTTTAATCCCTAGATTTAGCAGAGAAAT | |
| | | | | | CCCTCAAATCTCACACAATCCATGTAAGGAAGAA | |
| | | | | | G (SEQ ID NO: 95) | |
| AT3G63340.1 | | Protein | AT3G63340. | Ribo- | ATGAAGCAATTGTTCCTTTCAAGCATGTTTACGA | MKQLFLSS |
| | | phosphatase | 1_1 | shift | GCAAAAGAGTGAAAGGGTAG (SEQ ID NO: 58) | MFTSKRV |
| | | 2C | | Down | | KG* (SEQ |
| | | family | | | | ID NO: 20) |
| | | protein | | | | |
| AT4G11110.1 | SPA2 | SPA1- | 5′ | TEup | CTTACTTAAACACAGTCAAATTCATTTTCTGCCTT | |
| | | related 2 | UTR | | AGAAAAGATTTTTATCGAAAATCGACGTTTTTGA | |
| | | | | | AAAAACTCAAATTATCGTCGTTTTGTTCTCAGATT | |
| | | | | | TCTTCTGCTCTCTTCTTCTTCTCCTTCTTCTTCGTTC | |
| | | | | | CACCGCCTCTGTTGCTTTATCTTCTTCTTCCTTCCT | |
| | | | | | TCGATTGTTGATTACGTCGGTGGATCTTTGTTCTC | |
| | | | | | CTCTGTGTTGTTTTCATTGCTAGATTTCGTCAATG | |
| | | | | | ATTGGCTTCTCACGATTCGATTTTTCCGGCTCCTG | |
| | | | | | TTCTTAATTTCCTCTGAGAGA (SEQ ID NO: 96) | |
| AT4G11110.1 | SPA2 | SPA1- | AT4G11110. | Ribo- | ATGATTGGCTTCTCACGATTCGATTTTTCCGGCTC | MIGFSRFD |
| | | related 2 | 1_1 | shift | CTGTTCTTAA (SEQ ID NO: 59) | FSGSCS* |
| | | | | Up | | (SEQ ID |
| | | | | | | NO: 21) |
| AT4G17840.1 | | | 5′ | TEup | ATCAAAATCAATGATCAAGGTAACGTAGTCAAGT | |
| | | | UTR | | TCAATTACTCTTTGTCAAATTTAAGTGGTCTCTAT | |
| | | | | | TACTAAACTATACACAACCGTTAGATCAAATAAT | |
| | | | | | TCTCTACCATCCAACGGTCCAAAGTCTCCACTTCT | |
| | | | | | ATTTATTACAATAAAATGAGAAAATAAAAACGCG | |
| | | | | | CGGTCACCGATTCTCTCTCGCTCTCTCTGTTACTA | |
| | | | | | AATGAAGAAGAGAATCTCTCCGGCGAGATCACC | |
| | | | | | GGCGTTATTCCGATAATTTCGCCTGAGAGTTGTC | |
| | | | | | GCATGTTATAA (SEQ ID NO: 97) | |
| AT4G17840.1 | | | AT4G17840. | Ribo- | ATGTTATAA (SEQ ID NO: 60) | ML* (SEQ |
| | | | 1_4 | shift | | ID NO: 22) |
| | | | | Up | | |
| AT4G18570.1 | | Tetratrico- | 5′ | TEdown | ATTTTTATTACTCTCTCAAGTAGTCTCATCTTCTTC | |
| | | peptide | UTR | | TTAATCCAAAGGCCCAAACTTTGAATCATCACTA | |
| | | repeat | | | TCACTCTCTCTCTCTCTCTCTATCTCTCAAGAACTG | |
| | | (TPR)-like | | | CACGGACAACGACATGCTTTTAATTTCCATGCAA | |
| | | superfamily | | | ATCTCTCCTTTCTTCTCAAGTCATTTTTGAAAATC | |
| | | protein | | | AATCAAAAAACTGAAACTTGGTGGAGCTTTTATC | |
| | | | | | ATTCACTCATCA (SEQ ID NO: 98) | |
| AT4G18570.1 | | Tetratrico- | AT4G18570. | Ribo- | ATGCTTTTAATTTCCATGCAAATCTCTCCTTTCTTC | MLLISMQI |
| | | peptide | 1_1 | shift | TCAAGTCATTTTTGA (SEQ ID NO: 61) | SPFFSSHF |
| | | repeat | | Up | | * (SEQ ID |
| | | (TPR)-like | | | | NO: 23) |
| | | superfamily | | | | |
| | | protein | | | | |
| AT4G23740.1 | | Leucine- | 5′ | TEup | CTTTCACCCACTTTAATATGCCAAAAAATAAGAA | |
| | | rich | UTR | | CAAAATTATATCCGTTGCTTGAAAATCACAAGCT | |
| | | repeat | | | CTTCTTAACTTCACAAGTGCTTCAATGGCGGTTCT | |
| | | protein | | | TCACATTATCTTCACTGCGTAATTGAAGAAGTTG | |
| | | kinase | | | TTCTCTCTTCCTCTTAATTTCGAGTTGTGTTCTTAA | |
| | | family | | | AAAACTCCAGAGCTGATTCGATTCTCGAGAAGA | |
| | | protein | | | AACTAAGCCGACAATAAAGTTCAGATCTGGAAA | |
| | | | | | AAAGCGAGCTCCAGATTACAAAAAGAAACAGCT | |
| | | | | | CGTTTTTTTCACTTTCAAAAAA (SEQ ID NO: | |
| | | | | | 99) | |
| AT4G23740.1 | | Leucine- | AT4G23740. | Ribo- | ATGCCAAAAAATAAGAACAAAATTATATCCGTTG | MPKNKNK |
| | | rich | 1_1 | shift | CTTGA (SEQ ID NO: 62) | IISVA* |
| | | repeat | | Up | | (SEQ ID |
| | | protein | | | | NO: 24) |
| | | kinase | | | | |
| | | family | | | | |
| | | protein | | | | |
| AT4G23740.1 | | Leucine- | | | ATGGCGGTTCTTCACATTATCTTCACTGCGTAA | MAVLHIIF |
| | | rich | AT4G23740. | Ribo- | (SEQ ID NO: 63) | TA* (SEQ |
| | | repeat | 1_2 | shift | | ID NO: 25) |
| | | protein | | Down | | |
| | | kinase | | | | |
| | | family | | | | |
| | | protein | | | | |
| AT4G24750.1 | | Rhodanese/ | 5′ | TEdown | GAGTCTGGTTCGAAAAGACTGCTTCAATGAAGC | |
| | | Cell | UTR | | CAAAACTATCCAATAACTCGAAATTGACTACTCTT | |
| | | cycle | | | TTCTTTTGTCTCTGTTGTTGATTCGCAAAGGCGAA | |
| | | control | | | GATTATCCATCTTCTCAGTTACTCCTACTGGAACC | |
| | | phosphatase | | | AAAAGCTCAGAACCTTAAAAC (SEQ ID NO: | |
| | | superfamily | | | 100) | |
| | | protein | | | | |
| AT4G24750.1 | | Rhodanese/ | AT4G24750. | Ribo- | ATGAAGCCAAAACTATCCAATAACTCGAAATTGA | MKPKLSN |
| | | Cell | 1_1 | shift | CTACTCTTTTCTTTTGTCTCTGTTGTTGA (SEQ ID | NSKLTTLF |
| | | cycle | | Down | NO: 64) | FCLCC* |
| | | control | | | | (SEQ ID |
| | | phosphatase | | | | NO: 26) |
| | | superfamily | | | | |
| | | protein | | | | |
| AT4G26080.1 | AtABI1 | Protein | 5′ | TEdown | GAAGCAATTGTTGCATTAGCCTACCCATTTCCTCC | |
| | | phosphatase | UTR | | TTCTTTCTCTCTTCTATCTGTGAACAAGGCACATT | |
| | | 2C | | | AGAACTCTTCTTTTCAACTTTTTTAGGTGTATATA | |
| | | family | | | GATGAATCTAGAAATAGTTTTATAGTTGGAAATT | |
| | | protein | | | AATTGAAGAGAGAGAGATATTACTACACCAATCT | |
| | | | | | TTTCAAGAGGTCCTAACGAATTACCCACAATCCA | |
| | | | | | GGAAACCCTTATTGAAATTCAATTCATTTCTTTCT | |
| | | | | | TTCTGTGTTTGTGATTTTCCCGGGAAATATTTTTG | |
| | | | | | GGTATATGTCTCTCTGTTTTTGCTTTCCTTTTTCAT | |
| | | | | | AGGAGTCATGTGTTTCTTCTTGTCTTCCTAGCTTC | |
| | | | | | TTCTAATAAAGTCCTTCTCTTGTGAAAATCTCTCG | |
| | | | | | AATTTTCATTTTTGTTCCATTGGAGCTATCTTATA | |
| | | | | | GATCACAACCAGAGAAAAAGATCAAATCTTTACC | |
| | | | | | GTTA (SEQ ID NO: 101) | |
| AT4G26080.1 | AtABI1 | Protein | AT4G26080. | Ribo- | ATGTGTTTCTTCTTGTCTTCCTAG (SEQ ID NO: | MCFFLSS* |
| | | phosphatase | 1_3 | shift | 65) | (SEQ ID |
| | | 2C | | Down | | NO: 27) |
| | | family | | | | |
| | | protein | | | | |
| AT4G32660.1 | AME3 | Protein | 5′ | TEup | AATTGGTGGATGTCGTCGCGGTTCGACCCCAAG | |
| | | kinase | UTR | | GGATTTGGCCGGTAAAATTATTGGGAGTTGTCTT | |
| | | superfamily | | | TCTCTTGCACTCTCTCTAGTTCCAAACCCTAGCAA | |
| | | protein | | | TTCCTCTGTTTTCACCATTTTCGGAGATTATCACC | |
| | | | | | TTCTCCCCGATTCGCCGCCTTGTGATTACATCTAC | |
| | | | | | GTAAAGAGTTTCTGGTAGAAATTTTCCCTCTTTTA | |
| | | | | | GCTGCAGATTGGCATCAGATTCCGTTCTGGATGT | |
| | | | | | GTCGGTGATCGATTTTCCGCGTCGGTG (SEQ ID | |
| | | | | | NO: 102) | |
| AT4G32660.1 | AME3 | Protein | AT4G32660. | Ribo- | ATGTCGTCGCGGTTCGACCCCAAGGGATTTGGCC | MSSRFDP |
| | | kinase | 1_1 | shift | GGTAA (SEQ ID NO: 66) | KGFGR* |
| | | superfamily | | Down | | (SEQ ID |
| | | protein | | | | NO: 28) |
| AT4G32800.1 | | Integrase- | 5′ | TEdown | ATTTCATAAATCATAGAGAGAGAGAGAGAGAGA | |
| | | type | UTR | | GAGAGAGAGTTTGGAAACATTCCAAAACCAGAA | |
| | | DNA- | | | CTCGATATTATTTCACCAAAGAATGATAGAAACA | |
| | | binding | | | AGAACTATCTTTTTATAAAACTCTTTACACCCCAA | |
| | | superfamily | | | AAGAAAATGTCTCACTCGTTTTGCCTTATAATATT | |
| | | protein | | | TCTTTCAACAACAACCCAAATCTACAAAAAATCC | |
| | | | | | CAATAAAAAAAAACTTCAGTCTGTTTGACATTTT | |
| | | | | | GTCGAACACTTGGACGGCATCACAAAAAGCTCT | |
| | | | | | AAACTTTCTGACTACCA (SEQ ID NO: 103) | |
| AT4G32800.1 | | Integrase- | AT4G32800. | Ribo- | ATGATAGAAACAAGAACTATCTTTTTATAA (SEQ | MIETRTIFL |
| | | type | 1_1 | shift | ID NO: 67) | * (SEQ ID |
| | | DNA- | | Down | | NO: 29) |
| | | binding | | | | |
| | | superfamily | | | | |
| | | protein | | | | |
| AT4G34460.1 | ELK4 | GTP | 5′ | TEdown | GACCCTCTTCTCTCTCTCTAGCTAGTCTCAGGTCA | |
| | | binding | UTR | | GAGAAGCCATCATCAACATTCAACAAGAGAGCC | |
| | | protein | | | GTGTTTGTGTCTTGACTGATTCTTCTCTCAAGCTT | |
| | | beta 1 | | | TTTTAATCTCTCTCTCTTTTCCCACGTAATTCCCCC | |
| | | | | | AAATCCATTCTTTCTAGGGTTCGATCTCCCTCTCT | |
| | | | | | CAATCATGAACCTTCTTCTCTTCTAGACCCCACAA | |
| | | | | | AGTTTCCCCCTTTTATTTGATCGGCGACGGAGAA | |
| | | | | | GCCTAAGTCTGATCCCGGA (SEQ ID NO: 104) | |
| AT4G34460.1 | ELK4 | GTP | AT4G34460. | Ribo- | ATGAACCTTCTTCTCTTCTAG (SEQ ID NO: 68) | MNLLLF* |
| | | binding | 1_1 | shift | | (SEQ ID |
| | | protein | | Up | | NO: 30) |
| | | beta 1 | | | | |
| AT4G37925.1 | NdhM | subunit | 5′ | TEup | ATGGTTCTGTAACCGGACAACATCTCAAAACTTG | |
| | | NDH-M | UTR | | TTCTGTTTTTTTTTTTTCATTTCTTAGACAGAAAA | |
| | | of | | | (SEQ ID NO: 105) | |
| | | NAD(P)H: | | | | |
| | | plastoquinone | | | | |
| | | dehydrogenase | | | | |
| | | complex | | | | |
| AT4G37925.1 | NdhM | subunit | AT4G37925. | | Riboshift Down ATGGTTCTGTAA (SEQ ID | MVL* |
| | | NDH-M | 1_1 | | NO: 69) | (SEQ ID |
| | | of | | | | NO: 31) |
| | | NAD(P)H: | | | | |
| | | plastoquinone | | | | |
| | | dehydrogenase | | | | |
| | | complex | | | | |
| AT4G38950.1 | | ATP | 5′ | TEdown | AAGAACAAACAACTACCAAACTTGTAGGCAGTA | |
| | | binding | UTR | | GCAGGAGGAAGTGGGTGGGATTAACATTGTCAT | |
| | | microtubule | | | TTCTCTCTCTTTTTCTTTTACAAATCTTTCCGTTTT | |
| | | motor | | | GTTTTCTTTTGGTTTTCCGGTGAGCACTGTTGTGT | |
| | | family | | | TTCCAATTCCGGCACTCTTTAGGGTTCCCTTTCAG | |
| | | protein | | | AAGAAAACTTCACATTGTTGTTTCTCTCAACCGTG | |
| | | | | | ACATCTTGGATTACTACTTCTGACTACTTTCCTTTT | |
| | | | | | TCATGTGCCCCAAAAGATAATAGTTACTTTTTCAA | |
| | | | | | AATCTGGTTTTGTTGTTTGGGTTTGTGTCATTCAT | |
| | | | | | TGATAAAGTCACTAATGGAGAAGTACAAAACAA | |
| | | | | | TTGCAAAATTTCGAATCTGTGTTGTCATTGCTGA | |
| | | | | | ATTCTGTAGTGGATGTTTGCTTGCAGTTTAGAGC | |
| | | | | | TTCGGAGTGCGAAGAGTGAGACACAAGAGGATT | |
| | | | | | CTTTCTGGAACCGCATTATTCCCTTTAGAGGAGG | |
| | | | | | AAGAAGAAGACAACTCACTCACAAGGAAAACAA | |
| | | | | | AGGTTCCTCTGGTTACTCTGAAATATTCAAACCA | |
| | | | | | ATGGTGAGCAATTGGTAGCACTTGCTAAAGAAG | |
| | | | | | (SEQ ID NO: 106) | |
| AT4G38950.1 | | ATP | AT4G38950. | Ribo- | ATGTGCCCCAAAAGATAA (SEQ ID NO: 70) | MCPKR* |
| | | binding | 1_1 | shift | | (SEQ ID |
| | | microtubule | | Down | | NO: 32) |
| | | motor | | | | |
| | | family | | | | |
| | | protein | | | | |
| AT5G11790.1 | NDL2 | N-MYC | 5′ | TEdown | AAACACAAAAAAACGAAGATAGCCATCGTTTTG | |
| | | downreg- | UTR | | GTGAGAGAAGAGAGAAGAGAGAAGAAGAAGG | |
| | | ulated- | | | CCATGGAAAGATAATACTCTGCTTTTTTTTTAGAA | |
| | | like 2 | | | ATATACAGAGGAAATAAAGAGAGAGAGAAGGA | |
| | | | | | G (SEQ ID NO: 107) | |
| AT5G11790.1 | NDL2 | N-MYC | AT5G11790. | Ribo- | ATGGAAAGATAA (SEQ ID NO: 71) | MER* |
| | | downreg- | 1_1 | shift | | (SEQ ID |
| | | ulated- | | Down | | NO: 33) |
| | | like 2 | | | | |
| AT5G14930.1 | SAG101 | senescence- | 5′ | TEdown | TATGGACTCTCGTTCTCAGACATTTATTTCTCTCA | |
| | | associated | UTR | | GTCTTACAATATAAATTTTCATTCTTACCATCCAT | |
| | | gene | | | AATTTTGTATTGTCTTCTCCACAGATCTATTCCAG | |
| | | 101 | | | CTCACGCC (SEQ ID NO: 108) | |
| AT5G14930.1 | SAG101 | senescence- | AT5G14930. | Ribo- | ATGGACTCTCGTTCTCAGACATTTATTTCTCTCAG | MDSRSQT |
| | | associated | 1_1 | shift | TCTTACAATATAA (SEQ ID NO: 72) | FISLSLTI* |
| | | gene | | Down | | (SEQ ID |
| | | 101 | | | | NO: 34) |
| AT5G15950.1 | | Adenosyl | 5′ | TEdown | ACAATATCACAAACTCGTTTGCTCTTTTCATCATT | |
| | | methionine | UTR | | ACTAAATCATAAGCGGCTCTCAAGTTCTTTAGGG | |
| | | decarboxylase | | | TTTCGAGTTTTCTCAATCTCCTACCTGATTAAGGT | |
| | | family | | | TAATTTCTTATCTTGGATCAATAACAAGAGAATT | |
| | | protein | | | ATAACTCCGGATTGTAATCAATATTCCTCTACATA | |
| | | | | | AAAAGCGTGAATGAGATTATGATGGAATCGAAA | |
| | | | | | GCTGGTAATAAGAAGTCAAGCAGCAATAGTTCC | |
| | | | | | TTATGTTACGAAGCACCCCTTGGTTACAGCATTG | |
| | | | | | AAGACGTTCGTCCTTTCGGTGGAATCAAGAAATT | |
| | | | | | CAAATCTTCTGTCTACTCCAACTGCGCTAAGAGG | |
| | | | | | CCTTCCTGAGTACTAGCCAGTTCCCTCCATAGCTT | |
| | | | | | TTCAATTACAACAATCTCCTTTTCTCAAAGCTCTG | |
| | | | | | GTTCCCCAAATCCTCTCGTCTTTTGTTTGCCCTCA | |
| | | | | | CAACAACAACAACAACGCAGAG (SEQ ID NO: | |
| | | | | | 109) | |
| AT5G15950.1 | | Adenosyl | AT5G15950. | Ribo- | ATGATGGAATCGAAAGCTGGTAATAAGAAGTCA | MMESKA |
| | | methionine | 1_1 | shift | AGCAGCAATAGTTCCTTATGTTACGAAGCACCCC | GNKKSSS |
| | | decarboxylase | | Down | TTGGTTACAGCATTGAAGACGTTCGTCCTTTCGG | NSSLCYEA |
| | | family | | | TGGAATCAAGAAATTCAAATCTTCTGTCTACTCC | PLGYSIED |
| | | protein | | | AACTGCGCTAAGAGGCCTTCCTGA (SEQ ID NO: | VRPFGGIK |
| | | | | | 73) | KFKSSVYS |
| | | | | | | NCAKRPS* |
| | | | | | | (SEQ ID |
| | | | | | | NO: 35) |
| AT5G49980.1 | AFB5 | auxin F- | 5′ | TEdown | AAAAAATAATCCCCAAATAATGGAGACGAAGTG | |
| | | box | UTR | | GAGAGAGAAAGCTCCCACTCTCTCACACCCCAAA | |
| | | protein 5 | | | GCTTCTTCTTCTTCTTCCTCTTCTTCCTCTTCCTCTT | |
| | | | | | CTCTAATCTGAATCCAAAGCCTCTCTCTTT (SEQ | |
| | | | | | ID NO: 110) | |
| AT5G49980.1 | AFB5 | auxin F- | AT5G49980. | Ribo- | ATGGAGACGAAGTGGAGAGAGAAAGCTCCCACT | METKWRE |
| | | box | 1_1 | shift | CTCTCACACCCCAAAGCTTCTTCTTCTTCTTCCTCT | KAPTLSHP |
| | | protein 5 | | Down | TCTTCCTCTTCCTCTTCTCTAATCTGA (SEQ ID | KASSSSSSS |
| | | | | | NO: 74) | SSSSSLI* |
| | | | | | | (SEQ ID |
| | | | | | | NO: 36) |
| AT5G57460.1 | | | 5′ | TEup | GAAGATCTCATTTCTCTTTCTCCTTTTCTTCTCCGA | |
| | | | UTR | | CGATTCTTCTCAGTTCTCAGATCTGATCGATTTCT | |
| | | | | | TCATCAGATGTTTCAATCTAACCATTGAGATTGA | |
| | | | | | ATAGTCACCATTAGTAGAAGCTTCGAGATCAATT | |
| | | | | | TCGAATCGGGATC (SEQ ID NO: 111) | |
| AT5G57460.1 | | | AT5G57460. | Ribo- | ATGTTTCAATCTAACCATTGA (SEQ ID NO: 75) | MFQSNH* |
| | | | 1_1 | shift | | (SEQ ID |
| | | | | Up | | NO: 37) |
| AT5G61010.1 | EXO70E2 | exocyst | 5′ | TEdown | TCTTTCCCTTCTTCTTCCCCAATAATCTCGCTGAA | |
| | | subunit | UTR | | ACTCTCTTGCTCTTGCTTCTAAAAATCTGTTCTTT | |
| | | exo70 | | | GAGACTTTGATCACACAGTTATCAAAATCATAAT | |
| | | family | | | CTCTTCTTTCCTGGTTTTTTTTTTTTTCTTCTTCTTC | |
| | | protein | | | TTCCCGTTTCACGGTACGTTTACTCTGTTCGATCA | |
| | | E2 | | | CCGAGTGTATGATAAAATGTTTCTGTGAAATCAA | |
| | | | | | ATAACATATCACTTTCTAATAAACATCAAAATTTC | |
| | | | | | TCCTTTTTTACAGAAACAAGAAGTTTTTTTGGGA | |
| | | | | | AAGCCGTTGACTTGACTTTTTCTTTGGGGTGTTG | |
| | | | | | TGTGGGAGCTTATAGTATGGTACCATAAGTGGG | |
| | | | | | AGCTTATAGTTTGGGGTGTTGTGTGGGAGCTTAT | |
| | | | | | AGTATGAGGAAAAATGTTAGATTTGAAGAATGC | |
| | | | | | TTCACTGATTTTTTACCATAAGTATGTCAACTGGA | |
| | | | | | TTAAGCTTAAGTAGTAATGGTTTTTACTATGTTCA | |
| | | | | | TGTGGGGATTTCTCTTCCTCTCTGTTTACTTCATT | |
| | | | | | CCGAGATGACTTGAGATTTTTTCAAAGTATAGTT | |
| | | | | | CTTGGAGTTAAGCTTACCTAGTAATCACTTTATAT | |
| | | | | | AACATCCCTTCGTTTACATTTGTGCTTTCACCTGG | |
| | | | | | AAACACTTTAGACTTTTCTCTCTTCTGCCGTGTGT | |
| | | | | | ATTTAGTTGTCTAGTCAAATTTAAGTTGAGTTTA | |
| | | | | | GGCTCTAGTCTTTGGTTTTGGTT (SEQ ID NO: | |
| | | | | | 112) | |
| AT5G61010.1 | EXO70E2 | exocyst | AT5G61010. | Ribo- | ATGTCAACTGGATTAAGCTTAAGTAGTAATGGTT | MSTGLSLS |
| | | subunit | 1_3 | shift | TTTACTATGTTCATGTGGGGATTTCTCTTCCTCTC | SNGFYYV |
| | | exo70 | | Down | TGTTTACTTCATTCCGAGATGACTTGA (SEQ ID | HVGISLPL |
| | | family | | | NO: 76) | CLLHSEMT |
| | | protein | | | | * (SEQ ID |
| | | E2 | | | | NO: 38) |

To further discover novel mRNA sequence features for elf18-mediated translational control, an enriched motif search was performed in 5′ leader sequences (i.e., sequences upstream of the mORF start codon) and 3′ UTRs of TE-altered genes. A consensus sequence significantly enriched in the 5′ leader sequences of TE-up transcripts was identified (38.2%, E-value=1.2e-141) (Table 2). Since this element contains almost exclusively purines (FIG. 3A), we named it “R-motif” in accordance with the IUPAC nucleotide ambiguity code. No primary sequence consensus was discovered in the 3′ UTRs of the TE-up transcripts, or in either the 5′ leader sequences or 3′ UTRs of the TE-down transcripts. We used the FIMO tool in the MEME suite²³ to find occurrences of the 15 nt R-motif in 5′ leader sequences of all Arabidopsis transcripts and found R-motif in 17.7% of transcripts, which were enriched for the GO terms: response to stimulus and biological regulation.

TABLE 2
TEUp with R motifs

| geneID | Alias | Full name | motif sequence | 5′ UTR sequence |
|---|---|---|---|---|
| AT3G56460 | | GroES-like zinc- | GAAAGAGAGAGAGA | CAAATCCATCTCATATGCTTACGATAACGTCCC |
| | | binding alcohol | G (SEQ ID NO: 113) | ATTGCCAAGCTGGTTCTTTCACTCTTCAGGAGA |
| | | dehydrogenase | | AAGAGAGAGAGAGAGAGAGAGAGAGAGAGA |
| | | family protein | | GTTATCAGAGATAGCAAAA (SEQ ID NO: 294) |
| AT3G57870 | SCE1A | sumo | GAGAGAGAGAGAGA | GATAGAGATTGGAGAGCGAGCGAGACAAATC |
| | | conjugation | G (SEQ ID NO: 114) | AGAAGAGAGAGATTTAGATATTGTAGAGTGAG |
| | | enzyme 1 | | ATTCTAAAGAGAGAGAGAGAGAGAGAT (SEQ |
| | | | | ID NO: 295) |
| AT1G20670 | | DNA-binding | GAGAGAGAGAGAGA | AGGAGGAGAAAGAGAAAGGGGGAAGAGAGG |
| | | bromodomain- | G (SEQ ID NO: 115) | AGAGAGAGAGAGAGAAAGAGATTAGAGAGAG |
| | | containing | | AAAGAAGAGAAGAGGAGAGAGAAAAAA |
| | | protein | | ID NO: 296) |
| AT1G21270 | WAK2 | wall-associated | GAGAAAGAAAGAGA | AGGAGATTAGCGAAAACTCAAAACAGGAACAA |
| | | kinase 2 | G (SEQ ID NO: 116) | AGTTAAAAGAGTGAGAGAGAAAGAAAGAGAG |
| | | | | AAG (SEQ ID NO: 297) |
| AT3G05490 | RALFL22 | ralf-like 22 | AAGAGAGAAAGAGA | GTTGTCTTCAGCTGTGTACAGAATCAAGTTTCC |
| | | | G (SEQ ID NO: 117) | AAGAGAGAAAGAGAGTAAAAGCAAATTAACA |
| | | | | AAGGAAGACTCTGATTCACCGAGAAGGTTTTG |
| | | | | GCTTAAAG (SEQ ID NO: 298) |
| AT2G46030 | UBC6 | ubiquitin- | GAAGAAGAAGAAGA | ATTTTGGAATCTTTCTCTCTCTCTCTCTCTAAAAC |
| | | conjugating | G (SEQ ID NO: 118) | CAGATTCTTAATAGAAGAAGAAGAAGAAGAAG |
| | | enzyme 6 | | AGGAAAGGAGAAATCTGCC (SEQ ID NO: 299) |
| AT4G28730 | GrxC5 | Glutaredoxin | GAAGAAGAAGAAGA | ACGTCACGAGACAAATTAGCATAGCACGCAAA |
| | | family protein | A (SEQ ID NO: 119) | GAAGAAGAAGAAGAAGAAGCTCCAAGAATCT |
| | | | | GTCGCAGAAATCGCC (SEQ ID NO: 300) |
| AT2G17660 | | RPM1- | GAAGAAGAAGAAGA | AAACAAAACCATCTGACTTATCAACAACAACAA |
| | | interacting | A (SEQ ID NO: 120) | GAGGACGAAGAAGAAGAAGAAGATTGTTACTT |
| | | protein 4 (RIN4) | | TCTTGATCGATA (SEQ ID NO: 301) |
| | | family protein | | |
| AT1G64150 | | Uncharacterized | GAAGAAGAAGAAGA | TCAGAACAACACAGAGCCAAAGGTTTTTTGCTC |
| | | protein family | A (SEQ ID NO: 121) | GCAGTAAAGAAGAATCACACTGTGAAGAAGAA |
| | | (UPF0016) | | GAAGAAGCGAAATACAAAATCCTCAGGAAAGA |
| | | | | A (SEQ ID NO: 302) |
| AT1G53850 | PAE1 | 20S | GGAAGAGAAGAAGA | CGTCTTTGAAAGCTAAAAAGAGAGCAAAAGCT |
| | | proteasome | A (SEQ ID NO: 122) | TCTGTTTATTCTCCGATTCGCAGATCAATTAGCT |
| | | alpha subunit | | GGGTTTTGATTCCGTTGTGCGAAGGACTTTAAG |
| | | E1 | | AGGTTTTGCAGATCGAAATCGGAAGAGAAGAA |
| | | | | GAAG (SEQ ID NO: 303) |
| AT3G24520 | HSFC1 | heat shock | AACAGAGAAAGAGA | GTCAAGCAGCTTAAATCATCTATGACTTAAAAT |
| | | transcription | G (SEQ ID NO: 123) | TATAATTAAGAAAAAACAATGCCTAAATATGCA |
| | | factor C1 | | TATATTTCAAATGTATCACATAACTTGTGACATA |
| | | | | AGAAAATATAAACAAAACAAAAAGGGCAAAAA |
| | | | | AGACCTGAAAGCTTAGAGGCACACCTGCATAG |
| | | | | GTCCCACAGTTCACTCGTGACACCGTAAAAGGC |
| | | | | AAAACACGAACCCGCCACGTTATCACAAAAAG |
| | | | | CAAGCCACGTCAATATAGTCTCACTGTCAACTA |
| | | | | CACTTAACTTACTATTTTCACATCTCATTTTCCTA |
| | | | | TCTTTATATAAACCCTCCAGGCTCCTCTTTAATT |
| | | | | TCTTTACCACCACCAACAACAAACATATAAACC |
| | | | | ATAAGGAAAACAGAGAAAGAGAGAG (SEQ ID |
| | | | | NO: 304) |
| AT3G46100 | HRS1 | Histidyl-tRNA | GAAGCAGAAAGAGA | TCTTTCTTTTGCTAATTCTCTATCTCACTCAGCTG |
| | | synthetase 1 | G (SEQ ID NO: 124) | AAGCAGAAAGAGAG (SEQ ID NO: 305) |
| AT1G67230 | LINC1 | little nuclei1 | AGAAGAGAGAGAGA | ACAATAAAGGTTTCCAGCACAGAGAAGAGAGA |
| | | | G (SEQ ID NO: 125) | GAGAGATTGCTTAGGAAACGTTGTCGGACTTG |
| | | | | AAACCAGTTTCGGTACCGGAATTTAGAAACTCC |
| | | | | GTTCAAATCCGGAGCCAATCTCTAAAGGATAAA |
| | | | | GCTTCCAACTTTATCCATTAATTGGAGAAAATTC |
| | | | | TCAGAGAGACTGAAGTCGACAAAGTCAGAGG |
| | | | | GTTTCGTTTTTTGGCTTCTGGGTTTTTTATTTCA |
| | | | | AGTGTTCAATTTCCGAATTAGGTAAGAAAGTTA |
| | | | | GGTTTTGAGATCTGTGCGAATTGTGAGAG |
| | | | | (SEQ ID NO: 306) |
| AT1G61690 | | phosphoinositide | GGAGGAGAAGAAGA | CTTTTACATTTCCGGTAAGATCAAAATCAAAAC |
| | | binding | A (SEQ ID NO: 126) | CAAGTTCGTTTCGCGGCGGAGGAGAAGAAGAA |
| | | | | TCAGACGGGAAA (SEQ ID NO: 307) |
| AT5G28919 | | | AAAAGAAAGAAAGA | TTAAATTAGAGAAAAAAACGCAGACGACTAAA |
| | | | A (SEQ ID NO: 127) | AGATATTCACACACAAAAAAGAAAGAAAGAAG |
| | | | | AAAAATTAGCTCACAAAATAACAACAATATAAT |
| | | | | TAATACCCAAAAAAGAAAAAAAACTAACTGAG |
| | | | | TCCATGTTGAATAGATCTCCTATAGATGTAAGG |
| | | | | AAATACTCGGCTTCTACATCTTAATTAAGCATTA |
| | | | | CTTCCTATTTCTAAATAGATAGGAAGATTCAAG |
| | | | | AGCTTCTCTCCCAGACGTGATTTTTGAGACAGC |
| | | | | CTTTTCATCAATTTTTTCTGGCACCGGTAGAGC |
| | | | | GTTAGCTCGTCGGTGCCAGGAGCTAGCTTCTTC |
| | | | | TCACCGGTTTCCTCCCATAAGCTCTCTCATCGGT |
| | | | | TTCTCTGTTTTTTGTTTCGTGTTGTTTCGTCTCTT |
| | | | | TTCCCTCCTATTAGATCCATAAAGCTTCATTACC |
| | | | | GCACAACCTTCGAAACTACTCCCATCTGGTATT |
| | | | | AGCTCTTCTCTTACCTTGTTCGCGATTCTCGTGG |
| | | | | ATCCCTCTCCTCGGCTTTCCTTAAAGTCAAGATC |
| | | | | AGCAACTCTTTGGTCCTCA (SEQ ID NO: 308) |
| AT2G03390 | | uyrB/uyrC | GAAAAAGAAAGAAA | AACGAAAAAGAAAGAAAAATCTGTGAGGACG |
| | | motif- | A (SEQ ID NO: 128) | AAAACTCTCCGTCGTTCCGGCGAGTTTCTCCAG |
| | | containing | | TGATCGGCAAAGTCTTTCCGGCATCTATTGAAT |
| | | protein | | TTCTCTAAACCAATTAGAATATTATCGGTCTTGA |
| | | | | TAAAATAAA (SEQ ID NO: 309) |
| AT1G12500 | | Nucleotide- | AAAAGAAAGAAAGA | AAAACTCACACTTTCTCTCTCTCTCTCTAGAAAA |
| | | sugar | A (SEQ ID NO: 129) | AGAAAGAAAGAAGAAAAACTTATTGTTATTCCC |
| | | transporter | | ATTTCGCCCCTATCCGAAAA (SEQ ID NO: 310) |
| | | family protein | | |
| AT1G55840 | | Sec14p-like | AAGAGAGAAGAAGA | AGAAACATCATGATATGATATTTTTCTCAAGTCT |
| | | phosphatidylino- | A (SEQ ID NO: 130) | TTTGGTGTTGGAGAAGAAGAGAGAAGAAGAA |
| | | sitol transfer | | CTTGGTTTCTCTCTCTAAAAGTTTATTGCTTGGC |
| | | family protein | | TCCATAAAAAGTGCACCTTTTTCTCTCTTTTCTTT |
| | | | | CTCTCTCTCTCTCTCTCTCTCTCTCTCTCTCACTTC |
| | | | | TCCTCGGATGCACTATTGTCCGTGAGATCAGAG |
| | | | | ATTCACCCTCTTTAGATTTTGCGCAGAAACTTTT |
| | | | | GCCCACAATTTTGTATTCGTCAAATCTGAGCTG |
| | | | | AGATCTCTAGAGTGAGAAA (SEQ ID NO: 311) |
| AT1G48300 | | | CGAGGAGAAAGAGA | CGAGATGCGGCGAGGAGAAAGAGAAGGTTAA |
| | | | A (SEQ ID NO: 131) | GGTT (SEQ ID NO: 312) |
| AT1G04690 | KV- | potassium | TAAAGAGAGAGAGA | GTTCTTCTTCATTCATTACAACAAACTCTTTGAG |
| | BETA1 | channel beta | G (SEQ ID NO: 132) | ACCTAAAGAGAGAGAGAGCGATAGTGAGATTT |
| | | subunit 1 | | AGATCAACAGATTTGAATCGATTTCTGAAAAC |
| | | | | (SEQ ID NO: 313) |
| AT1G02000 | GAE2 | UDP-D- | AAAGGAAAGAAAGA | AGAAAGGAAAGGAAAGAAAGAAAACAAAAGG |
| | | glucuronate 4- | A (SEQ ID NO: 133) | AGTCCAAGAAACCAGAAGATTGTCTCCCGACG |
| | | epimerase 2 | | CCATTATCCTTCACCCTCGGAGCTTTTCTTGAAG |
| | | | | CAGGGATTCTTCTAATCATTAATCCCTACTTCTT |
| | | | | TCTTTCTTTTTTGTTTGTTCTCCTTTGAGATCTAT |
| | | | | CTAGTACTAGTAGTAAAACCCCCTCCCCTCCATT |
| | | | | GAATTTGAATTGAATTGAATCTCTGGGAATCAA |
| | | | | ATCTTTG (SEQ ID NO: 314) |
| AT5G50430 | UBC33 | ubiquitin- | CACGGAGAAAGAGA | TTTTGATATTTCGACACTCTCTCTTTCCTCTCTCC |
| | | conjugating | A (SEQ ID NO: 134) | TTGTCTCTGTACCGCGTCGAAATATGAGAAACG |
| | | enzyme 33 | | AATGATTTGATCATCAATCAACGAGAAACACAC |
| | | | | ACGGAGAAAGAGAATCTCAAATTAGCTCCAGC |
| | | | | TCCTGATCGATTCCGATTTTCACAATTCTTTCCT |
| | | | | TGGATCTGCTCTTACCTTGTCACGATTTCACTTC |
| | | | | CCTGTGTTTTTGATTTATACTTGGTCATCCAATA |
| | | | | ACGAAACTTTGATCAAACTGGAACTACAGTTTA |
| | | | | TTGGAACTCCCTGAAGCATTTAG (SEQ ID NO: |
| | | | | 315) |
| AT2G26590 | RPN13 | regulatory | GAAAGAAAAAAAAA | AATTGAAAGAAAAAAAAAAACGAGAAGCGTTT |
| | | particle non- | A (SEQ ID NO: 135) | TCTTTCTCTCCAAAATCCATTACTCGCGAACTTT |
| | | ATPase 13 | | CCTCTGCTAAGTGTTCACTAGAAAGAGGTGGT |
| | | | | GATT (SEQ ID NO: 316) |
| AT2G21230 | | Basic-leucine | CACAGAGAGAGAGA | TGGATGATTGCTGCTTTGGTCAACGTTTCAAAA |
| | | zipper (bZIP) | G (SEQ ID NO: 136) | GAATCGTTTTTTCTTTTAGTTCCTTCCTTCTTTCG |
| | | transcription | | CTATTTTCGCCATTGATTGCTGAAGAAAACACA |
| | | factor family | | GAGAGAGAGAGATTCACTTCCCCATTTCAGAA |
| | | protein | | AATCAAA (SEQ ID NO: 317) |
| AT2G04865 | | Aminotransferase- | AAAGAAAAGAGAGA | ATGCTGACACAGATATTTATTTTTGCCTCTTATA |
| | | like, plant | A (SEQ ID NO: 137) | ACGAAAAAAGCAAAATAAAAGAAAAGAGAGA |
| | | mobile domain | | AGAGAAAAGCATTATCCCTTACGACGAGGAAG |
| | | family protein | | CCGTCGTTTTGAGGGTTCGTACAAATCCTGAGA |
| | | | | TCTTCCTTCAAACTCTTTCTTTGTCTCCTTTTTTA |
| | | | | TCTCACTCCGTCGTCGTTTTGATTCTTTCAAAGT |
| | | | | TCTTCATCCTCTGTTCCGCGCTGTTTTCTGGTGA |
| | | | | GTGTTGATTCTG (SEQ ID NO: 318) |
| AT5G24165 | | | GAGAAAGAAAGAAA | AGAAAAATCAGAGAAAGAAAGAAAACAGAGC |
| | | | A (SEQ ID NO: 138) | AATTACTTGAAGAATCCATAGGAAGCTGAAG |
| | | | | (SEQ ID NO: 319) |
| AT3G19553 | | Amino acid | CAGAGAAAGAGAGA | AATAACAACTATACAATGATATTTTTGATCAAA |
| | | permease | G (SEQ ID NO: 139) | CGTCATTTTCCAATCTTTGAATCTGAGATGATAA |
| | | family protein | | CTTGTTCAGCTTAATCTTTCCAGTCAATTTCATC |
| | | | | TCCTTCCAATTTTGAAGGGTTCATCAGAGAAAG |
| | | | | AGAGAGCCATTCAGAGATCCATTGTACCAAGCT |
| | | | | CACTTCGATCTACAGAATCACCGAGAGCTCTCT |
| | | | | GTCTCTCTGTCGGTGATATTTGTTTG (SEQ ID |
| | | | | NO: 320) |
| AT2G32970 | | | GGAGGAGGAAGAGA | AACGTGCTCCGGTGAAGATTAAAAACCGACGA |
| | | | A (SEQ ID NO: 140) | GACCCTGGCGCCATCACAACTACGCAATCTCAT |
| | | | | TCCTCCGTCTTCTTCGGCTTTCAAATTTACCATTT |
| | | | | TACCCTTCTCTTTCCCTGAGACGTCTTCTTTGGA |
| | | | | AATATTCTTCTCTTCTTCCATTCCAATGATTTTGA |
| | | | | GGTTAATTGGAAATTAGAGTGCAAAATTGGGA |
| | | | | TTTAGATGGGGATTGCTGATGAATCTAAATGTG |
| | | | | TTTTCCCCTTGACGAGTCTCCAGATCGGAGACT |
| | | | | TGCAATCATATCTTTCTGATCTCAGTATTTTCCT |
| | | | | GGGAAATAAAAGTAAAAAGATTTACATATTGG |
| | | | | TGGATAACCGGCCATGGTTGAATCCTGGCACC |
| | | | | AGATCTGCTCATTTTTGGCAACTAATGGTCACA |
| | | | | AAGACTCTCCCCTTTTGCAAACACGAAACTTCG |
| | | | | AGGGGAGAAGAAAAATCAGAATCAGGACAGG |
| | | | | GAGAAGAAAAAGTCGAAGCAGGAGGAGGAAG |
| | | | | AGAAGCCTAAAGAGGCTTGTTCTCAGCCCCAG |
| | | | | CCGGACGATAAAAAA (SEQ ID NO: 321) |
| AT2G18230 | PPa2 | pyrophosphorylase | AGAAGAAAGAAAGA | AAAACTCTACTGTAACTGCAAAATCTTGTTGTTT |
| | | 2 | A (SEQ ID NO: 141) | TCTTAAACGAAGAGAGAAGAAAGAAAGAAAA |
| | | | | AAACGTTACGGATTCTCTGCTTCGGTTTCGCGA |
| | | | | TTGAAGCTTGAGATTTCATCTTGAACATCCGAT |
| | | | | (SEQ ID NO: 322) |
| AT4G14420 | | HR-like lesion- | GAAAAAAAAAAAAA | AAAATCTCACCTTTTTGACCCCAAAAATTTCTAA |
| | | inducing | A (SEQ ID NO: 142) | ATATTTCAAAATCAGCCTCTTCGTTTTCTTTCTCC |
| | | protein-related | | TCCTGTCTGTTGATTTAAAGACCCAAATCTGAC |
| | | | | GCTTCTCTCTCTCTTTCTGGTATCTGCGTTTGAT |
| | | | | TCGGAGAAGAAAAAAAAAAAAAAGGCAAAGA |
| | | | | GAGAGCTTCA (SEQ ID NO: 323) |
| AT3G25470 | | bacterial | GAAGAAGATAGAGA | ACAACCCTAGAACAAAAAAAGTATCCCATTTGT |
| | | hemolysin- | A (SEQ ID NO: 143) | CATTTGTCAATTGTCATTAGCAAGAACAGGAAG |
| | | related | | AAGATAGAGAACAGAGCTCTTCGATCTTTTTTC |
| | | | | CTCCAAGGAAGAAGTAGAAAG (SEQ ID NO: |
| | | | | 324) |
| AT5G17530 | | phosphoglucosa- | CAAAGAGAAACAGA | ACACAATCGAAGTCGAACTCTCAGGATTCAATC |
| | | mine mutase | A (SEQ ID NO: 144) | TTGATACCAAAGAGAAACAGAAATAAACTAAC |
| | | family protein | | ATCATCGCTACTGTCGCCTATAATCTTGTGAGCT |
| | | | | CTTTATCGTCTTCAATGGAAGTTCGATGATGTA |
| | | | | AAAACTCAAATAAGAGTGATTCTAGAATGGGA |
| | | | | AATTTTCTATAGAAAGGAAAGGTTTTCCAAAAC |
| | | | | TTTAATGTAGTACAGAGCTGCTACCGACAAAAT |
| | | | | AAGCAGTTTAAGACACGATACCAAAGAGAACC |
| | | | | TGACCTGTTC (SEQ ID NO: 325) |
| AT3G06550 | RWA2 | O- | GAACGAAAGAGAGA | AATTGTTTTGAGGTAGCAGCTGCAAACCGCTCA |
| | | acetyltransferase | A (SEQ ID NO: 145) | AACAGTTGCGCATTAGGCATTACACAGTTCCAC |
| | | family protein | | TCGTTCCTTTTGAAGCTTATCTGTGTGACTCTAA |
| | | | | TCTGTTACTATAATAGGAACGAAAGAGAGAAC |
| | | | | TAGGATCTATACTTGCTCCAACCTTGCTTTGTTT |
| | | | | CTCTTCTGCGATTTATCTCTAGATCTACTAGATC |
| | | | | TGGACAAGGAGCGAAGCGAATTGCTGGCAAAT |
| | | | | TTTAGTTTTGGAGTTTTGAAACCCGACGATTAT |
| | | | | CGCGCTTGATCGTTGCTTCTCTGATCGGAA |
| | | | | (SEQ ID NO: 326) |
| AT4G34090 | | | GAGAAAGATAGAGA | CTGAATTACGAAAATTCTGTGAGGTTGAGGAA |
| | | | G (SEQ ID NO: 146) | GCAGAGTGAAGAGAAAGATAGAGAGATAAGA |
| | | | | AGAAGCC (SEQ ID NO: 327) |
| AT4G29190 | OZF2 | Zinc finger C-x8- | AACAAAAAAAAAGAA | AACACAAACAAAAAAAAAGAACTCTTTCGTCGA |
| | | C-x5-C-x3-H | (SEQ ID NO: 147) | CTAATGTGATTTATTGTTCACCGGAGTATTAAA |
| | | type family | | GAAG (SEQ ID NO: 328) |
| | | protein | | |
| AT4G17615 | SCABP5 | calcineurin B- | AAAGGAAAAAGAAA | AAAGGAAAAAGAAAAATAAATAATCGATCTCA |
| | | like protein 1 | A (SEQ ID NO: 148) | ACCGTCCGATCATCCATCTTGCCATCACCGTTCA |
| | | | | CCAATCTTCTTCGTCTCCTCTCTCTTTCTCTCTTT |
| | | | | TTGCTGTTTCTAGCTCCTCTCTCTCTGGATCTCG |
| | | | | CCGGCGAACCGTTTCTCTTGGGTGTAAACAGTA |
| | | | | GCAATCAAGCTATAGAATCTCAGATATCGCTGA |
| | | | | ATTAGCTGTTGGATTTTATCCGCCTTTTCTTCGT |
| | | | | TATCCGGGGCTCGGGTATAAGGTTTCATCGTCT |
| | | | | TATTTCATCTGTAA (SEQ ID NO: 329) |
| AT3G22420 | ZIK3 | with no lysine | GCAGAAGAAGAAGA | ACTTGTTTCCTTATATATTCTTCTCCCTTTAAACA |
| | | (K) kinase 2 | G (SEQ ID NO: 149) | TTTAATCTTTTCCTCTTCTACCATCTCCACAAATT |
| | | | | CCAAACATCTCTCTCTCTTTCTCTCTCACACACA |
| | | | | AAATTGCAGAAGAAGAAGAGTC (SEQ ID NO: |
| | | | | 330) |
| AT3G09210 | PTAC13 | plastid | AAAAGAAAAACAGA | AACGGAATTTTCCCAAAAGAAAAACAGAGA |
| | | transcriptionally | G (SEQ ID NO: 150) | (SEQ ID NO: 331) |
| | | active 13 | | |
| AT2G25610 | | ATPase, F0/V0 | CAAAGAGATAGAGA | AAATCAAATTCATTCATATCAAAGAGATAGAGA |
| | | complex, | G (SEQ ID NO: 151) | GAAA (SEQ ID NO: 332) |
| | | subunit C | | |
| | | protein | | |
| AT1G13000 | | Protein of | AAAAGAAAAAAAAA | AAAAGAAAAAAAAAAATCTCAGTCAAGTTCGT |
| | | unknown | A (SEQ ID NO: 152) | CCGAAAGTTTTCAACGACGACGGCTTTTTAGAG |
| | | function | | ATTTGATTCGTTTCACTCTTCTGGGTATTGATTT |
| | | (DUF707) | | TCTTCCTTAAATTTGCATCCTTTTTAACGTTTATC |
| | | | | CAACGATCTTGCTCCGTTACTGAAACTCTGTTTC |
| | | | | TCCGTTGCTTCTCTCGTCTCATTTATTGTTCGTA |
| | | | | ACGTGATTTTACTACTTCTGTTACTCGAGTAGA |
| | | | | GATTACCCTTCTTATGTCCGAATCTGATTCGTCG |
| | | | | TCTTTAAGCTTTGTCTTCTCCCAATTAGCTCAAA |
| | | | | GTTCGTAACTTTGTTTACTTGCCAATAAGAAATT |
| | | | | TCCAGAGACTGAAGTTTCCATTGAATGTATTGT |
| | | | | TCTTGGAGAACTTAACCGGATTCAGGAC (SEQ |
| | | | | ID NO: 333) |
| AT5G13440 | | Ubiquinol- | AAAGAAGAAAAAAA | CTCGAAGACTATTAAAGGAATATCCGCAAAGA |
| | | cytochrome C | A (SEQ ID NO: 153) | AGAAAAAAAAACATTTTTTTGGTAAAGGACTAA |
| | | reductase iron- | | TCTTTTTGTTTGCATCGGCCATCTCTAACCTTAC |
| | | sulfur subunit | | GATTGTGTGTTCTTGCTTTGAGCGAAACCCTAG |
| | | | | AATCGGTCTTAACCCATTTGAGCAGAG (SEQ ID |
| | | | | NO: 334) |
| AT3G05840 | ATSK12 | Protein kinase | AAAGGAGATAAAGA | ACATTAGCTTCCTCATTTTTATTCTTATTATTATT |
| | | superfamily | G (SEQ ID NO: 154) | ATTCATCAGACCAACAACAAAAAGGAGATAAA |
| | | protein | | GAGAAGAGGATTCATCATCATCAATCAATCCTT |
| | | | | CATTTTATGGATCTACTCATATCTTGATTCTTCC |
| | | | | TTCTATCTCTCCCTTTTCTTCCATCTCTTTTTCTCT |
| | | | | GGGTTTCCCCGGATTGAGTTTTTTAATCTCTGAT |
| | | | | TGACAGATTTGAAGAGCGTGACAAAGGAAGAA |
| | | | | TCTTTTATTAAAACAAATTCTTCTGTTTTAATCTT |
| | | | | GGG (SEQ ID NO: 335) |
| AT1G47250 | PAF2 | 20S | GAACAAGAAGAAGA | AAACGAAAAGCTTTTGAAGAACAGAGGAACAA |
| | | proteasome | A (SEQ ID NO: 155) | GAAGAAGAAAG (SEQ ID NO: 336) |
| | | alpha subunit | | |
| | | F2 | | |
| AT1G21460 | SWEET1 | Nodulin MtN3 | ACAAGAAAAAAAGA | AGCTCATATTCTCTCACTTTCTCTCTCAGCTTAC |
| | | family protein | A (SEQ ID NO: 156) | GAACAAGAAAAAAAGAAGAATCTTTAGCCACC |
| | | | | TTTGAGATCAAAAG (SEQ ID NO: 337) |
| AT4G27450 | | Aluminium | GGAAAAGAAGAAAA | ATCCAAAACGTTTTTCCTTCCCACAGGAAAAGA |
| | | induced protein | A (SEQ ID NO: 157) | AGAAAAACAGACAGCGGAGGACTAAAACAACT |
| | | with YGL and | | AGCCACAACACAACGCTTCAAATATATATTACT |
| | | LRDR motifs | | CTGCCACTTTCTTCAATCTTCCTTCAAAGATTCT |
| | | | | TATTACAGCGACACACAACTCTTTTCCATTTAGA |
| | | | | TTTTTGATTTTTTTTGGTTCTCTAAAGGAGGAGA |
| | | | | GAA (SEQ ID NO: 338) |
| AT3G61460 | BRH1 | brassinosteroid- | GGAAAAAAAACAGA | AACTTTTTCAAAAAAAGGAAAAAAAACAGAGC |
| | | responsive | G (SEQ ID NO: 158) | TCACTCATTATTATCTCTCTAAAAACCCTAGCTT |
| | | RING-H2 | | TCTCC (SEQ ID NO: 339) |
| AT2G35060 | KUP11 | K + uptake | TGCAGAGAAAGAGA | AATCAGCTGCAGAGAAAGAGAAGTCAAAACGC |
| | | permease 11 | A (SEQ ID NO: 159) | AGCTCTCTCTTGCGTTTTCTTCCTTTCTCCTTTCT |
| | | | | CAATTCCCCAGAGAACAACATAACTCTGTAAAA |
| | | | | GGGAAACTCTATTTTGTTCTGAATCAAAAGTAG |
| | | | | TTTTAA (SEQ ID NO: 340) |
| AT1G53730 | SRF6 | STRUBBELIG- | TAGAGAGAGGAAGA | ATTTCTCTTTCTTTCTTAAGCTTTTTCACAAGACT |
| | | receptor family | A (SEQ ID NO: 160) | AGACTTTAGCTTATCGTTCTAGAGAGAGGAAG |
| | | 6 | | AAG (SEQ ID NO: 341) |
| AT5G02250 | RNR1 | Ribo-nuclease | AACACAGAGAGAGA | ATAGAATTTCTCGTTTTTATCACCCGCTTCATTT |
| | | II/R family | A (SEQ ID NO: 161) | GCCTTTCTATCGCCACAAGAACACAGAGAGAG |
| | | protein | | AACGATTAGCCCAGTTCCGATATCGTTCGGTGG |
| | | | | CTTCTTCATCTGAAGCTACG (SEQ ID NO: 342) |
| AT4G13520 | SMAP1 | small acidic | GAAGAAGAAAACGA | GACAGTCAGTCACTGTAACATTTTAGATCTTTCC |
| | | protein 1 | A (SEQ ID NO: 162) | CGAAGAAGAAAACGAAGAAGAGACGAAGAGA |
| | | | | GAA (SEQ ID NO: 343) |
| AT1G53230 | TCP3 | TEOSINTE | GACAAAAGAAGAGA | CAGAAACAGAGACAAATTCTAAAAAAGAAACA |
| | | BRANCHED 1, | A (SEQ ID NO: 163) | ATCTTTAGACAAAAGAAGAGAAATTTAGTCATG |
| | | cycloidea and | | GGTTAGTCTGCAAAATTCAATTACGTCTTCTTCT |
| | | PCF | | TCTTCTTCTTCTTCATCTTTGATTTGTTGGCGTGT |
| | | transcription | | TTAGGGTTTGGGATTTGGAGGAGAGGCAAAAT |
| | | factor 3 | | GTTGAATTAAATAAATCGAACGACTCTGGATTC |
| | | | | CTCGGCGGTTAACGACCGCCGTCGCCGCCGCC |
| | | | | GTCATAATCCAACCACCACCACCATCAACGACC |
| | | | | TTGAATTTCCACAATATGCTTCATCA (SEQ ID |
| | | | | NO: 344) |
| AT5G46860 | VAM3 | Syntaxin/t- | CCAGGAAAAAAAGA | ACAACTTTATCTCAGCTTTTTCTTCTCAATTAAA |
| | | SNARE family | G (SEQ ID NO: 164) | ATCAGTTTGGGATTTTTTCGAAAACGCTTTTCAA |
| | | protein | | TCTTCGTCTATCTGTCTCCACGATCCACGCCTTG |
| | | | | ACCTTCGTTTTTTTTTTCTCAGAGATTAGAGAAA |
| | | | | ACTCCGATAACCAATTTCTCAATCTTTTTGTAGA |
| | | | | TCCAATTTTTCCAGGAAAAAAAGAGGTTTCGCG |
| | | | | AAGAAG (SEQ ID NO: 345) |
| AT4G37830 | | cytochrome c | TGCAGAGAAAAAGA | AGTGAGTCACATAACCCTCTTGGAAAGAGTCTC |
| | | oxidase-related | A (SEQ ID NO: 165) | AACACTTGCAGAGAAAAAGAACAAGGAAGATC |
| | | | | CCGGAAA (SEQ ID NO: 346) |
| AT3G14205 | | Phosphoinositide | CAAAAAAAAAAAAAG | TTAAACCCAGAAATCACCAAAAAAAAAAAAAG |
| | | phosphatase | (SEQ ID NO: 166) | TACATTTCCTTTTTTTTTGTTCTTAAATTTTTCTG |
| | | family protein | | TGGTTCCGGTCACCGCAGCTCTGTCATCATCTT |
| | | | | CTTCTTCTTCATTTACCAATCTGAAATCTACTCA |
| | | | | GATTCTTTGTGATTTTCTCCTTAAAATCTCGATC |
| | | | | TGTATCGTACAGTGACTTGTGAAATTAGGATCG |
| | | | | TTGTGTCTGTGTTTTCTGGTTACAGTTTGTAAAA |
| | | | | TTTGAATATTTGTGTGTGAAGTCAGATTCAGTT |
| | | | | TCGTGAGCTGTTCGGATTTGGTTTGGGGGTATA |
| | | | | TATATAGCGTTGTGTGATCTATTTGGGGGGTTT |
| | | | | TGGTTTCCCTTTTTTTCTCTCTTGTGAATTCGTTT |
| | | | | ATTGTTGTATCGTCGGCCCGAGTTTATCGGAAC |
| | | | | TCCGGGTCTGACGTGAGTTTTCCAA (SEQ ID |
| | | | | NO: 347) |
| AT5G38700 | | | GAAACAAAAAAAAAA | ATTCATCACCACAATCACCTGAAGAGCCAAAGC |
| | | | (SEQ ID NO: 167) | AGCAAAAGAAACAAAAAAAAAACAAGAAGTG |
| | | | | AAGTCAGATCTCGAAAAAGAGTTTACGAATCC |
| | | | | (SEQ ID NO: 348) |
| AT2G18670 | | RING/U-box | AAAAAAAGAGGAGA | AAACGTTACTGTCACTAAATGAAATCTATTTTTC |
| | | superfamily | A (SEQ ID NO: 168) | TTTCTTAAATTTTGCTCTGACAAATATTTTTGAT |
| | | protein | | TGCGTCATTTTCTACTTTGGAAATGTCTTTGATT |
| | | | | TAGCATTTCAGTTCGCTCAAAACATCAAATCTTA |
| | | | | CCTTCTTTAGCTTTCACATTAGATTCTGGTAATT |
| | | | | ATTAGCACAAAAAAAAGATAAGCCAGAATACG |
| | | | | AAACAACCAAAAAAAGAGGAGAATTCTTTTTTT |
| | | | | TTTTTTTCTTTCCG (SEQ ID NO: 349) |
| AT1G45000 | | AAA-type | AAAACAGAAAAAAA | CTCAAGAAAACAAAATTACTTTAAAACAGAAAA |
| | | ATPase family | G (SEQ ID NO: 169) | AAAGTTGATAAATTGCTTCAGTGTCAAATTCTG |
| | | protein | | AGATCTGTAAAAG (SEQ ID NO: 350) |
| AT1G20650 | ASG5 | Protein kinase | AGAGAAGAAACAGA | TAAAATAAATGAGAAGAACAAAAATTCAGTTG |
| | | superfamily | G (SEQ ID NO: 170) | TTAAAATCAAAGTAGTGTCTCTACCGTGATTTTT |
| | | protein | | ATTTTTTTCTATATACTGTTTAAACCTCAGTTTTT |
| | | | | TTGTTGTTGTTATAAGATCCTTGTCATTTTTTGT |
| | | | | CGTGATTAGATGTAATTTGTATAATTTTAGTAA |
| | | | | CTCTTCAGTTTTTTTTTGTTTTAAAAATATATTTT |
| | | | | CTCTCTCTCTGTCTTCCTGCAATCTATCGCCGGC |
| | | | | CGATTCAATAATTTCGCTTTACTCTGCCAAAAAA |
| | | | | GTTTGTTCTTTTGTTTTCTGGGATTATCCAAAGA |
| | | | | GAAGAAACAGAGGAAATCAATCTCTTTTTTAGT |
| | | | | TTCAGACCCTAAATCCTAGGTTTTGAAGTTTTGT |
| | | | | TTCTTTAGTAATTTTGTCAGGTTTTGTGTCTGGT |
| | | | | GTTGGGATTTTTCGGAGCTTGGTTTCTTGAACC |
| | | | | AGCTCCATTTTCTAAAAATTCCTTCTTTAAATCC |
| | | | | CCATTGTTGTAAGTCTTAAAGAAAAAAGAAG |
| | | | | (SEQ ID NO: 351) |
| AT4G29070 | | Phospholipase | GAAAAAAAAAACGA | GTCATTTGCTAAGGAAAAAAAAAACGAAAACG |
| | | A2 family | A (SEQ ID NO: 171) | TGTGTCTGTCTCTTCTCGTAGCGTCTCTCAAGCT |
| | | protein | | CAG (SEQ ID NO: 352) |
| AT4G26410 | | Uncharacterised | GAAAGAAGGAGAAA | AAAACCAACTTCTAATTTGGAATCAAATTGAAC |
| | | conserved | A (SEQ ID NO: 172) | CGAATCGAACCGGTTGAAGTTGAAAGAAGGAG |
| | | protein | | AAAAGGCGTTGTCTCCGTGCGAGAAAGGCAAA |
| | | UCP022280 | | TCGGAGACG (SEQ ID NO: 353) |
| AT4G03420 | | Protein of | ACAGAAAAAAAAGA | TTTTTTATTTTCTTGACAAGTCTGCATTTTTCTCC |
| | | unknown | A (SEQ ID NO: 173) | TCTGTTTTGGAATTTTCTCGTTTCTGGTTTTCCG |
| | | function | | ATCATAAAAAACAAACAAAACTACCGTAAAATA |
| | | (DUF789) | | GGCTCTCTCCACAGAAAAAAAAGAAGACTTTTC |
| | | | | TTTCATTCTTCTGCAAGTAACTGAGCAGATTTCG |
| | | | | GTTTTTTCTTCTTCAAATTGATATTTTTAAAGTTA |
| | | | | TAAAAATTTCTTGTCCATAATTTCCGTTTTCCTTA |
| | | | | AATTCAGCTGTCCTAACGTCAAATCTCAGACAC |
| | | | | TCGCTTGCGTGTCTCCCTCTCTTAAACTCTCTCT |
| | | | | TTCTCTTTCTCTTTTGGTTTCTGGGTTATTTCAAA |
| | | | | GAAAAGAATCAAGAAACCCCTCTTTCTCTCTTA |
| | | | | CAAGAATCCCATC (SEQ ID NO: 354) |
| AT2G31410 | | | CAAAGAAGAGAAGA | AAAACCTCACAGCCACACAAAGAAGAGAAGAA |
| | | | A (SEQ ID NO: 174) | (SEQ ID NO: 355) |
| AT4G33030 | SQD1 | sulfoquinovosyl | GGGAGAAGAGAAGA | ATATCTGTCTCATCTCATCTCTCATCGTTCCGGG |
| | | diacylglycerol 1 | G (SEQ ID NO: 175) | AGAAGAGAAGAGAGACCCATCCCTCACTTCAA |
| | | | | AGTTCAAAGTCTCGAAGGATCTTCTCCAACTCT |
| | | | | CTCTAAACAAGATTCCAAATTTTCAAAGGTGAA |
| | | | | TTTGTTTGATAGAATCAAGAACAAACCTTTAAA |
| | | | | (SEQ ID NO: 356) |
| AT3G52470 | | Late | TAGAGAGAGAGAAA | ATATTTCTTCCCATCGTCACTAGTCACGACCACA |
| | | embryogenesis | G (SEQ ID NO: 176) | CAAACAAAAAAAATATAACATTTAGAGAGAGA |
| | | abundant (LEA) | | GAAAGGTACAGCAGTGGCAAACTCGTAAATAA |
| | | hydroxyproline- | | AGA (SEQ ID NO: 357) |
| | | rich | | |
| | | glycoprotein | | |
| | | family | | |
| AT1G23900 | Gamma- | gamma-adaptin | GAAGAAAAAAACGA | AATTATGGTTTACGAAGACTGAGAAGAAAAAA |
| | ADR | 1 | G (SEQ ID NO: 177) | ACGAGCATCGTCCATCGAGATCCAAATCCTCAG |
| | | | | TTTCATTTTCATCTCTCTCTCTCGTATTGATCAGC |
| | | | | TACTCGAAACTCCGGTAACGGATTTTCACAATC |
| | | | | CCGGCGGCGAAACTCTTCTTCCCGGCTAAGTTT |
| | | | | TCATTTTCTTCAGATTCCTCGTAAAGTTGCCGGT |
| | | | | GGACCAAGGTCCAACTCTTGAACACCCCAAATC |
| | | | | (SEQ ID NO: 358) |
| AT1G02610 | | RING/FYVE/PHD | CAAGAAAAAACAGA | CATTCATTTGTTCTTTCTTCAGAGAAAAACAAAA |
| | | zinc finger | G (SEQ ID NO: 178) | AACAGAGCATTTTTTTTGGTCAAGAGCAAGAAA |
| | | superfamily | | AAACAGAGCATACTTTTGCAAAAAGCAGAGCT |
| | | protein | | TGGAGCGCTTTCTTGTCATCTAAAATTCAAAGG |
| | | | | CAGAGACG (SEQ ID NO: 359) |
| AT5G48220 | | Aldolase-type | GCGAGAGACAGAGA | GTTTGGAAATAACGTGTAAGTAGGACCCACTTT |
| | | TIM barrel | G (SEQ ID NO: 179) | TGTGATTATCCGCCGCACAGAAGTCTCTCCTCC |
| | | family protein | | ACTCCACAAATAGCATTCCCGGCGAGAGACAG |
| | | | | AGAGCGAAGAAGAAGACTCAAACCAAAAAAA |
| | | | | AAA (SEQ ID NO: 360) |
| AT3G61070 | PEX11E | peroxin 11E | GAAAGAAATAAAAA | ATCGACGGTTAGAAATGAAACGATTAGGAGAT |
| | | | G (SEQ ID NO: 180) | TAGATCGTTGAACAAAACGACGTGTTTTGGTCT |
| | | | | ATTTATAAAGAAAGAAATAAAAAGGAGAGATG |
| | | | | ACCAAACACGCCTTTATCATAGTTTCTATCTCCG |
| | | | | ATGACACAAAACGAGGAAGATTATTTGACATTT |
| | | | | TAAGTAAGAACAGCTAGCTTTGCCATCTCCCTA |
| | | | | AAGGCAATAAATCTCGGATCCACTTTCACGATA |
| | | | | TTTTGATATTTTTTCTATTTATAATCTTTCTGGGT |
| | | | | TTTGAGTCTTTTGAAGGCTGAATTGCTCTGAAA |
| | | | | TCTCAATTGTATAATCATCTCCTGGGTCGTCGTT |
| | | | | ATCGTGATCATCTAGAAAGC (SEQ ID NO: 361) |
| AT2G45170 | ATG8E | AUTOPHAGY 8E | GACGAAAAGAAAAA | ATCCAATCATAGACGAAAAGAAAAAGGTTCCTT |
| | | | G (SEQ ID NO: 181) | TTTTTGACTTTGTATCCGTAGATCATCTTCTTCTT |
| | | | | CTTCTTCCAGAGTTTTATCCTTATCCGTTCCATC |
| | | | | AAATTCTCTCTCTAAGCAAAG (SEQ ID NO: |
| | | | | 362) |
| AT1G53380 | | Plant protein of | TAAAGAAACAGAGA | GTTTCTCATCTCCAGCTCTCATTTTCTCTCTCATC |
| | | unknown | G (SEQ ID NO: 182) | TTCAACCTTAACTCTCTTTTCTCTCTACTCTTTCT |
| | | function | | TTGGACGAATCTGTCTATTGTTTGTAAGTTTTCA |
| | | (DUF641) | | AGGAAGGTAAAGAAACAGAGAGATCTAACTTC |
| | | | | GTCTGCAGGGTTTAAGCAGAGGTTGGTTTGTG |
| | | | | GATTCTTCGATTTCTTCTTCAGATTTAGTCTACA |
| | | | | ATGAAGTGAGAATTTCTAAAGATAAACAAAGA |
| | | | | AAAACTTGAGACTTTAGCAAG (SEQ ID NO: |
| | | | | 363) |
| AT5G07240 | IQD24 | IQ-domain 24 | CAAAAACAAAAAGAA | AATTGTCTCTTCTTTTCTTTTTGTACTTGTCAAAA |
| | | | (SEQ ID NO: 183) | ACAAAAAGAACAACAAAAAAAATCTCAACCGT |
| | | | | AGAAAATTCCGACAAGAGTTCAGTTCATACAAT |
| | | | | GAACTAAGT (SEQ ID NO: 364) |
| AT4G30010 | | | AAAAAGGAAGAAGA | ATCTTCGGAAAGTCTCATTTCTCGATCCCCAATT |
| | | | G (SEQ ID NO: 184) | CGTGGATTAGGGTTAAAAGAACCATTTTTATTC |
| | | | | TCGTCGCGCAACAACAAATCCAGATCGAAAAA |
| | | | | GGAAGAAGAGATCGAA (SEQ ID NO: 365) |
| AT5G02480 | | HSP20-like | GAAGAAGAATAAAA | TAATCCAATCTTCTTCTTACATAAACACCTCTCC |
| | | chaperones | A (SEQ ID NO: 185) | TCCCCCACCGTTTCCAAAAGAGAGAAGCTTTCT |
| | | superfamily | | CACTAACACCAAAAACAAGTCTTTGAAGAAGA |
| | | protein | | ATAAAAAGATTGGATTTTGATAAGTTTAGTGAA |
| | | | | AATGGGGGAGCTTTTGTGTTCTTCACTGTGGAA |
| | | | | CCCGTCACGATTCATTGTTGCTTCTCTCAAAAG |
| | | | | GTATTTTCTGGGTTTAGCTTCTTAGAGGTTCTTC |
| | | | | GTTCTTAAAGGTCTGTTTTTTTTTAGGTTGTGAT |
| | | | | ACTTTGAATGTAAAAAAGGGAAGATTTTTAGTT |
| | | | | TCGATATGTATATCTCTCGGATGGGTTTGAGTC |
| | | | | GGAGTTTCCCGCCGCTTTTTGGGGGATTTCGGG |
| | | | | AAATTCTAGGGTTAGGGTTGGATATTGTCTTCC |
| | | | | TCTAGCAGTCTCTGCCACTTTTAAAATCTCTTCA |
| | | | | TCTTTCTTTGAGAGTGAAAGAGGTTTTTTTATTT |
| | | | | GTTTGTGTCTTCCTGGGAATCGAGATTCTGGAT |
| | | | | CTTAATCAATATGTGGGTTAATTGGGAGATCTG |
| | | | | GGATTTGGGAGATCTTGTGGTGGATTGAAGAA |
| | | | | AAAGCAAGGTTGTAGATTTTGAAAA (SEQ ID |
| | | | | NO: 366) |
| AT3G09860 | | | TGACGAGAGAGAGA | GGTAGAAAGAAAGGATTTTTATTTATCCAGAAT |
| | | | G (SEQ ID NO: 186) | CAATCGCCGGAGAAGAAGATAAACACAGAGA |
| | | | | GTGACGAGAGAGAGAGTGAAA (SEQ ID NO: |
| | | | | 367) |
| AT2G30530 | | | GAAACAGAGAGAGA | CCTGTCTAGCGTTGACGACACCAAAATTGAAAA |
| | | | T (SEQ ID NO: 187) | TTTGGCATCATTTGCGAAACAGAGAGAGATCC |
| | | | | ATTCAATTCCAAAAGGATTCTCTTTTGGGAAAA |
| | | | | CCCTAAATCGACCCACCAAATTTGGAGACTGTG |
| | | | | ATTGAGCATGAGCGTCAGAAGTTG (SEQ ID |
| | | | | NO: 368) |
| AT4G35860 | GB2 | GTP-binding 2 | AGATGAGAAGGAGA | ATTAGATCCCTTTAATTTTAGTAATTAAGTAAAA |
| | | | A (SEQ ID NO: 188) | AGATTATAAAAGATGAGAAGGAGAAGATAGCT |
| | | | | TCTTCATCGAGAAACCTCGAAATCAAAAAGCAC |
| | | | | GTCGGTGACTTGTACTCTTCAATCTCTTCTTCCT |
| | | | | CTCTTTCACATCTCCTTCTCTCGAACCCATCGAC |
| | | | | CTGCGCTAATTCATCATCGACCTTGCTCAAATTC |
| | | | | ATCAACC (SEQ ID NO: 369) |
| AT3G53990 | | Adenine | TGAGAAGAAAAAAA | AACTTCCAAATCCTTTATATAACTTCTCACAAGT |
| | | nucleotide | G (SEQ ID NO: 189) | CACCACCATTTCTCTCTAGAAAATATCAGAAAA |
| | | alpha | | ACAAAACCATCTCAAAGTTTCTTGAGAAGAAAA |
| | | hydrolases-like | | AAAGGGTCAAGAAAG (SEQ ID NO: 370) |
| | | superfamily | | |
| | | protein | | |
| AT3G17650 | YSL5 | YELLOW STRIPE | CAGAGAAAACAAGA | GAGTCCAAGTTGACTCCTTCGAGCTTTGATTCT |
| | | like 5 | G (SEQ ID NO: 190) | CGTTCCAATAATACTTCCTCCACCATCTCTCCTC |
| | | | | CTCTCGTTAGATCTAAGAAACAGAGAAAACAA |
| | | | | GAGAGATAGA (SEQ ID NO: 371) |
| AT1G69530 | EXPA1 | expansin A1 | AAAAAGAAAAAAGA | CCAATTCTAAACCAAACAACAGATTCTCATAAT |
| | | | A (SEQ ID NO: 191) | CATCTCTTCTTTTTTCCTCTTTACGAAAAGAAGA |
| | | | | AAGATCAAACCTTCCAAGTAATCATTTTCTTTCT |
| | | | | CTCTCTCACACACACACATTCACTAGTTTTAGCT |
| | | | | TCACAAAATGTGATCTAACTTCATTTACCTATAT |
| | | | | GCAGGTTTACACAAAAAGAAAAAAGAACG |
| | | | | (SEQ ID NO: 372) |
| AT1G70600 | | Ribosomal | CGCAAAGAGAGAAA | CTAGCCGCAAAGAGAGAAAGGGAGGGAGGAG |
| | | protein | G (SEQ ID NO: 192) | AGTGTAGCAGATCGGCGAAA (SEQ ID NO: |
| | | L18e/L15 | | 373) |
| | | superfamily | | |
| | | protein | | |
| AT3G49140 | | Pentatricopeptide | TAGAGAGAGCGAGA | GTCCAGCTTCTGAGCTCAGAGATAGAGAGAGC |
| | | repeat (PPR) | G (SEQ ID NO: 193) | GAGAGGTTAGAGATAACAGTAGTTTTACCG |
| | | superfamily | | (SEQ ID NO: 374) |
| | | protein | | |
| AT3G22290 | | Endoplasmic | GAGACGGAAAAAGA | AAATTGATAACTTCTAATAAATGGAGGGTGCA |
| | | reticulum | G (SEQ ID NO: 194) | ATTAATAAATAAGGAGAGACGGAAAAAGAGAC |
| | | vesicle | | GCCGTTGAAACACCGCAAAACAGAGAAGCGCC |
| | | transporter | | TTTTGATTGTCTCTCTCCCGGAGATCTCTCTTTC |
| | | protein | | TCTTCTTCTCCATCCTTCTTCTCTCGGCGCGCGC |
| | | | | TTCATCCCCACCACCTTCGAATTCGTGCCCTTTG |
| | | | | AGGGAAGCTGCTAGG (SEQ ID NO: 375) |
| AT3G13520 | ATAGP12 | arabinogalactan | CAAAGAGAAGAACA | ATTTTATAGAGACGTCTCTGGAAAAAACATTCC |
| | | protein 12 | A (SEQ ID NO: 195) | CAAAATTGGCTTATAAATACTTTCAAAACCACA |
| | | | | AGGCCACAACTCATCATTCGCACCAAAGAGAA |
| | | | | GAACAAAACATCATCATATATTCTATTGACTAG |
| | | | | ATTAATTTCTTCTAAGTGCAAAAGAGGAGAA |
| | | | | (SEQ ID NO: 376) |
| AT1G53850 | PAE1 | 20S | AAAAGAGAGCAAAA | CGTCTTTGAAAGCTAAAAAGAGAGCAAAAGCT |
| | | proteasome | G (SEQ ID NO: 196) | TCTGTTTATTCTCCGATTCGCAGATCAATTAGCT |
| | | alpha subunit | | GGGTTTTGATTCCGTTGTGCGAAGGACTTTAAG |
| | | E1 | | AGGTTTTGCAGATCGAAATCGGAAGAGAAGAA |
| | | | | GAAG (SEQ ID NO: 377) |
| AT1G22200 | | Endoplasmic | CGAGAAAATAGAGA | TCCGTGATTCTTCTCTTTAGCTTATTTTTGGGGA |
| | | reticulum | G (SEQ ID NO: 197) | AGACAATTCCGAGAAAATAGAGAGTAGAGAGA |
| | | vesicle | | TCCTAAAGAGTCAAAAGAGGTCAGGTGATTGA |
| | | transporter | | TTAACCCGTTGAATAATCTCCTTCTCCCGTTGAA |
| | | protein | | TCGGGTCGAAATAGTTGAACTTTAAGCCAAACC |
| | | | | CTAGCTTGAGGAGGAAGAGGA (SEQ ID NO: |
| | | | | 378) |
| AT4G33520 | PAA1 | P-type ATP-ase | GGAAAAAAGAAAGA | AAACAAACGCAGGAGGCCTGGAAAAAAGAAA |
| | | 1 | T (SEQ ID NO: 198) | GATAACGGGACTCGAGAGATTGAGATTACGGA |
| | | | | GCCACCCACTTTC (SEQ ID NO: 379) |
| AT2G15560 | | Putative | GAAGAAGATCGAGA | TATATGCTTTCTCTGGACAAACGCAAAAACTTTT |
| | | endonuclease | A (SEQ ID NO: 199) | GTAGAACCCTAAAAATTCCCAAAATCCGTCGGA |
| | | or glycosyl | | GAAGAAGATCGAGAAGAATCAACAACTAATCT |
| | | hydrolase | | GAAGAATTTTCCAAATTCCGTCTTCGTATCGTCT |
| | | | | ACGAGATCCTTATCTCTCCCCTGAATCTGGAAC |
| | | | | CTTTG (SEQ ID NO: 380) |
| AT1G71980 | | Protease- | CAAAAAAAAAAAGAT | AACAAAACTCGAATCAGAGAATTCCAGATATTA |
| | | associated (PA) | (SEQ ID NO: 200) | CTTACATAAGACAATTTTAGCAATTAGCTTTCAA |
| | | RING/U-box | | ATCTCATCTCTTTATTCTCTCTCTCTATCTCTTCT |
| | | zinc finger | | CCTCAAGAACCCTAAAAATCTCCAGAAAAAAGA |
| | | family protein | | TCCCAAATTTCGTATTTCAACGATCTGAATCTCT |
| | | | | CTCTCTTTCGGGTTTATTTTGTTTCCCGATATGG |
| | | | | TTTAGAATTTGTGATTTAAATGGAAGCTGACGT |
| | | | | GTCAATTTCCTGAAAAAACCCTTATCGCGAAAT |
| | | | | TTTCCAGATTACCAAAAAAAAAAAGATTGAAAC |
| | | | | TTTTTTCGATTTGTTTGAAGAAGAAGCACGGTA |
| | | | | GGAACGACGACG (SEQ ID NO: 381) |
| AT1G51950 | IAA18 | indole-3-acetic | GAAAAAAGATAAGA | AGAGAGAGAGAGAACACAAAGTGGGAAAAAA |
| | | acid inducible | A (SEQ ID NO: 201) | GATAAGAACCCACCATAAAGTTTTAACATTTTT |
| | | 18 | | CCCTTCAAAAGGCGAAAGCTTTTGATTTGTATA |
| | | | | AAAGTCCCACTTAATCACCTCTCTAGCTTCTCAT |
| | | | | TCCATTTCCATCTCCTCTCTTTTGTTTTCTAAGTT |
| | | | | GCTTCAAGAGTTTTGGATAGTGTAGCAGAGAG |
| | | | | ATTTTAACTAATGGGTTTATAAAATTTTGTTCTT |
| | | | | TTGCGTGAACAAGTTGTCAACTTCTAGACAGAT |
| | | | | TTTCTTTTTGAAGTGTTTTCTTGTCGAAATTCTTC |
| | | | | TTCTTTTGGTCAAAGAACGCAAGATTCTTCTGT |
| | | | | AGTTCCTCTAAAAAAAATCCTA (SEQ ID NO: |
| | | | | 382) |
| AT3G58030 | | RING/U-box | AAAAAAAAGGGCGA | CGTCCTTCTTATCATTATAATCATCTTTTTAATCA |
| | | superfamily | A (SEQ ID NO: 202) | AAAAAGGTTTGCACATAACATAAGCTTTTTTCTT |
| | | protein | | TCTCTCTTAATCAGAAAACAATCTTGTCTCACAA |
| | | | | AAATATAATTAATGATTCTAAATTTCCCTAACCG |
| | | | | TCCGATCACAAAAGATCGTGATCATCGCGTGG |
| | | | | AAACTTTAGACCAATCTTTTCCCTAAACCGGAC |
| | | | | CGTACCAGATTCCTTCTCTCTCTCTCTGCTTAGA |
| | | | | GAGTTTTAGGTTCGTTTTCCCACTTAAGCCAAAT |
| | | | | TGGACAAGATTTGGACGTTTCTGTATCTCTCTT |
| | | | | AAAGCTAAAAAAAAGGGCGAATTTTTCCATGG |
| | | | | CGTTGTCGGAGTTTCAGCTAGCTCTGAGCTTGG |
| | | | | TGGTCTTGTTCTTCTAGCTGATTTGATCGAAACC |
| | | | | CCATGTTCTTATGATTTTACACGACCTAATCCAA |
| | | | | AACTCCAGAGCACACGGAGACGGAGTACATAT |
| | | | | TGTTCAGCGCAAGTGAAAGCAAGAGCCTTTTTG |
| | | | | TCTATTG (SEQ ID NO: 383) |
| AT3G56010 | | | CACACAGAAACAGAG | GTGTTTAGCTTCTTCACTACCACACAGAAACAG |
| | | | (SEQ ID NO: 203) | AGTTTCCGTCTTTCATCTTCCTCCATATGCGTCG |
| | | | | CTCTTAAAAACCTAATTCACA (SEQ ID NO: 384) |
| AT5G20165 | | | TAGAGAAAACGAGA | AAAGGAAGAAAGGGGTAGAATTGGAAATATG |
| | | | A (SEQ ID NO: 204) | TAGAGAAAACGAGAATAACTCTGACGCGAACG |
| | | | | TTTCTCTCCTCCGTCTCTCGATCCCTCTCTTGAC |
| | | | | GTCTCGCTGATCTGTTTTGCTAAGATTCAAGCTT |
| | | | | CAAAACCCTAATTTCTCTAGCCATTAGCATCGAT |
| | | | | TTCAGCTCAACTTCAGATTCAAGGAAACAATTA |
| | | | | TTAGCTTCTCAAGTGCTTCAGTGATCCGATACA |
| | | | | (SEQ ID NO: 385) |
| AT4G21445 | | | CACCGAGAAAGAAAA | GTTATCCTCATCTAGTCATCTTCACCCTCTAACT |
| | | | (SEQ ID NO: 205) | CACCGAGAAAGAAAAGTAAAGAGAGTTTGGTG |
| | | | | TCACT (SEQ ID NO: 386) |
| AT3G02530 | | TCP-1/cpn60 | ATAAAAGAGAGAGA | GAGCCCTCACTTGACAGAACTCAGAAATTTGAA |
| | | chaperonin | A (SEQ ID NO: 206) | AGAGAAATAAAAGAGAGAGAAGCTCCCAGAG |
| | | family protein | | AAGAAAAGCCCTAAAAGCCCCACTCCTCTTTCC |
| | | | | AGTTTCTTTTGATCTCTCAGCATCGAAA (SEQ ID |
| | | | | NO: 387) |
| AT1G43700 | VIP1 | VIRE2- | CGGGAAAAAAAAAA | CTTTGGTCCTACTTAGTACTTACCTGCCCCTCTC |
| | | interacting | A (SEQ ID NO: 207) | GACAAAATTTCTTTTGTACTTTCACATTTCTCTG |
| | | protein 1 | | TAATAAACTCGGTAGGTTTGCGAAAACCTCGCC |
| | | | | GCCGGGAAAAAAAAAAATCA (SEQ ID NO: |
| | | | | 388) |
| AT4G32600 | | RING/U-box | AACACAAAAAAAAAA | AATCTCCCCTTGGTTGATCGGTGAACACAAAAA |
| | | superfamily | (SEQ ID NO: 208) | AAAAAATCTAAAATAATCGCAAAATACATTTGA |
| | | protein | | AGAAGCTACACGATCAACAACAGCAAAGGATT |
| | | | | TCGATTGTTGAAAAAGTTGACTCTTCTTAATTTG |
| | | | | ATTCGTTGTCTTGGTTTCTGGGTTTTCTTCTTCTT |
| | | | | CTTCTGCGGCGCTCTCCAATTTTACACCTTGCGA |
| | | | | CCAGCGAGAAAAGAAACAAATTTCACCCCCATT |
| | | | | GAAGAAGGACCTTTGGTTAAGCTCCATGGTGT |
| | | | | GGTATGCGCAAAGTGGACAATACCTAG (SEQ |
| | | | | ID NO: 389) |
| AT1G56580 | SVB | Protein of | CCAAAAAAAACAGAG | TAAGAGACAGAGAGATCTTAACACAAAACAAA |
| | | unknown | (SEQ ID NO: 209) | GCAAACACCAAAAAAAACAGAG (SEQ ID NO: |
| | | function, | | 390) |
| | | DUF538 | | |
| AT5G43010 | RPT4A | regulatory | GAAGCAGATACAGAA | AAACCCATTGCTCAAGAAAACTTTTCAGACAGA |
| | | particle triple-A | (SEQ ID NO: 210) | TTTGTTTCGAGAAAAGATCGCTTGCTTGGCTTT |
| | | ATPase 4A | | TCAGGATAATCTGAGATCTATCTGTAGAAGAA |
| | | | | GCAGATACAGAATTCAGAAACG (SEQ ID NO: |
| | | | | 391) |
| AT3G01640 | GLCAK | glucuronokinase | AAAAGAAAGTAAAAA | AAAAAAAGAAAGTAAAAAACGCGTCAGGGAA |
| | | G | (SEQ ID NO: 211) | GAGAAG (SEQ ID NO: 392) |
| AT5G17770 | CBR1 | NADH: cytochrome | AAGGGAAAGAGACA | AATAATGTGTTGCAAAAGAGGCAAACTATACA |
| | | B5 | A (SEQ ID NO: 212) | ACGTGAAAGTGGTAGGTCTACCAGATCCCATA |
| | | reductase 1 | | CCCTCATTTTAATGGCGGAGATTACAAGGGAA |
| | | | | AGAGACAACTCCAATTCAAAGCTCTGATTTTTT |
| | | | | CCACCAATCCCCATTTTTTCCCTTTTACAATTCTT |
| | | | | AAGCTAGTTTTATACTTTTCTTCTTCCTTTCATTT |
| | | | | GGGTTAAGAGAAGCC (SEQ ID NO: 393) |
| AT4G17840 | | | AAATGAAGAAGAGA | ATCAAAATCAATGATCAAGGTAACGTAGTCAA |
| | | | A (SEQ ID NO: 213) | GTTCAATTACTCTTTGTCAAATTTAAGTGGTCTC |
| | | | | TATTACTAAACTATACACAACCGTTAGATCAAA |
| | | | | TAATTCTCTACCATCCAACGGTCCAAAGTCTCCA |
| | | | | CTTCTATTTATTACAATAAAATGAGAAAATAAA |
| | | | | AACGCGCGGTCACCGATTCTCTCTCGCTCTCTCT |
| | | | | GTTACTAAATGAAGAAGAGAATCTCTCCGGCG |
| | | | | AGATCACCGGCGTTATTCCGATAATTTCGCCTG |
| | | | | AGAGTTGTCGCATGTTATAA (SEQ ID NO: 394) |
| AT4G30960 | SNRK3.14 | SOS3- | ACGGCAAAAGGAGA | ATCCGACGGCAAAAGGAGAATTAAGATTTTTA |
| | | interacting | A (SEQ ID NO: 214) | ACTTTAAACGAGAGTTTCGTTTATTTACTCAAAA |
| | | protein 3 | | ATTTACTTCTGAAATCTCTATTTGAATTTCGGGG |
| | | | | AAAAAAATCCTAAGTAAGGGAATGCAGAGAGA |
| | | | | TGGTCGGAGTATCGCCGGTGAAGACTAAGCTG |
| | | | | TGTGATCGGTTTAACCGATCCGTCGGCGGCAG |
| | | | | GAATTGCCACCGGAAACACGTCGAGGACGGGT |
| | | | | GATCCAGTTTTCTAAACTCTCGTCTCTCGAATTC |
| | | | | TTCGAAGATATCGAAAAACTGTAAATCTTTTTTT |
| | | | | TCTTCTACTTTTTTACAAAATTCTCTAATCATCGT |
| | | | | TGTAAAGTAAAAAACC (SEQ ID NO: 395) |
| AT4G16580 | | Protein | GAAGGAGGTGAAAA | TTCTTTCGTGAAATTTGTCATCTCTTCTTTCAGA |
| | | phosphatase 2C | G (SEQ ID NO: 215) | AACTTATCTGGATTCTAGCCAATTTCTGTTGTGA |
| | | family protein | | CTTTGACATTATCTTCTCCAGAAGGAGGTGAAA |
| | | | | AGAGAATTTGTGGGTCCTGGTAAGTTCCGAATT |
| | | | | CGTATTTGATTGAGCTCTGAGTTTCAAGGGTTT |
| | | | | GTGTTGGATCAATCTTTAGATTCGTTGGTGAAA |
| | | | | GCGTTTAAATCGACGAAAAAAGTGATGCTTTG |
| | | | | GAAGATATGATCTTCTCTATCTCTGGTTATTACT |
| | | | | GGGTTTCGAGATTCTTGTGCTTAAG (SEQ ID |
| | | | | NO: 396) |
| AT4G12830 | | alpha/beta- | AAAGAACAAAAAAAA | TAAACCACCAATTCTCTCATCCGTACCAAAGAA |
| | | Hydrolases | (SEQ ID NO: 216) | CAAAAAAAAGATAAA (SEQ ID NO: 397) |
| | | superfamily | | |
| | | protein | | |
| AT4G10040 | CYTC- | cytochrome c-2 | AAAAAAAAATCAGAA | ACTTCTCATAAAAAAGGTCATTTCAAAAAAAAA |
| | 2 | | (SEQ ID NO: 217) | TCAGAAACCGTCAAAAAGCCACCGTTGATATTT |
| | | | | CTTCCTTGTTGCTTCTTCA (SEQ ID NO: 398) |
| AT3G06670 | | binding | AGAAGAAAATAAAA | CTCCTCTCTCTTCTCTCTTCTTTCGCGTTTCGAAG |
| | | | G (SEQ ID NO: 218) | GTTGGGGAAAGCTTTCGCAGAAGAAAATAAAA |
| | | | | GCTAGAGAGAGAATGTCAATGTTTTTTTGATGC |
| | | | | TCCGTCTGGCAATTAGGGTTTCTTTTTTCTTTGA |
| | | | | TTTCGTCCCCTTCGAGAACTGAATCTCCCGCCTA |
| | | | | TATCGACGCCGTCTAATTCCTATCATTTCTCGTT |
| | | | | GCTCCAAAACCCTAACTTTACTACCGTCGGTCA |
| | | | | TTATTTTCACTTTCTCGGCTCGATTTGGTGTTGG |
| | | | | AGGTTGGTAATCAGTT (SEQ ID NO: 399) |
| AT2G29700 | PH1 | pleckstrin | TAGGAAGACGAAGA | CGAGCGACCAAAACGCAGAGTTTTGACAGCAA |
| | | homologue 1 | A (SEQ ID NO: 219) | TTGAGTGGATACCGAATCACAATAATACAGAA |
| | | | | AGACATTAAAAGCAACAAGGAATCGCGCGATT |
| | | | | GGGGGCAGTTGGAGAGACGAACAAGTCGTGG |
| | | | | TGAGATTTTAGGAAGACGAAGAAG (SEQ ID |
| | | | | NO: 400) |
| AT2G20740 | | Tetraspanin | AACAGACGAAGAGA | AAGTATCAAAAAAATTACAACTTTACGATTTGC |
| | | family protein | A (SEQ ID NO: 220) | TTAGAAAGGAGAAGACATCTGGAGCAACAGG |
| | | | | ATTTACAAAAGTTATTATCTTTATCGATTTCTCTT |
| | | | | CTTCCTAGACCCAACAGACGAAGAGAATTTGTT |
| | | | | GTTGGTTGTCTCTGGTCTCTTCGTCTAGGTTTTT |
| | | | | TTTGGGTTATTAAAG (SEQ ID NO: 401) |
| AT5G40930 | TOM20-4 | translocase of | GAAGAAGAATCAAAA | CTTAAATTATCGTTTGTGACGGAAGAAGAATCA |
| | | outer | (SEQ ID NO: 221) | AAACAATTAATCGCGAGGCTTGAGAATCAATC |
| | | membrane 20-4 | | A (SEQ ID NO: 402) |
| AT5G21274 | CAM6 | calmodulin 6 | AAAAAAAGGTAAGA | AGAGAGGCAAATAATATATTCAGTAGCAAAAA |
| | | | A (SEQ ID NO: 222) | AAAAATCTGGGATTTCTAAAAAAAGGTAAGAA |
| | | | | GGAAA (SEQ ID NO: 403) |
| AT4G23740 | | Leucine-rich | GCCAAAAAATAAGAA | CTTTCACCCACTTTAATATGCCAAAAAATAAGA |
| | | repeat protein | (SEQ ID NO: 223) | ACAAAATTATATCCGTTGCTTGAAAATCACAAG |
| | | kinase family | | CTCTTCTTAACTTCACAAGTGCTTCAATGGCGGT |
| | | protein | | TCTTCACATTATCTTCACTGCGTAATTGAAGAA |
| | | | | GTTGTTCTCTCTTCCTCTTAATTTCGAGTTGTGT |
| | | | | TCTTAAAAAACTCCAGAGCTGATTCGATTCTCG |
| | | | | AGAAGAAACTAAGCCGACAATAAAGTTCAGAT |
| | | | | CTGGAAAAAAGCGAGCTCCAGATTACAAAAAG |
| | | | | AAACAGCTCGTTTTTTTCACTTTCAAAAAA (SEQ |
| | | | | ID NO: 404) |
| AT4G22820 | | A20/AN1-like | CCAGAAGAAAGAGAT | TAGTTACGTGTTTCTGTTTTTCTCTAATTTTTCTC |
| | | zinc finger | (SEQ ID NO: 224) | TTGTTGTTCTCGATTAACGAAAAAGACTTGTCG |
| | | family protein | | TTCTCAATTCTTATCGATTTAAGAACAAATCATC |
| | | | | TAACGAAGATTACTTCCGAAGATCAGAAACAA |
| | | | | ACACAAACTGTGAATCGTTGTTTGTTAATTCTCT |
| | | | | TTAAAATCGCCAGAAGAAAGAGATCTCCGTTTT |
| | | | | CTACAGAAGAAAAGCAAGAGAGTAAGA (SEQ |
| | | | | ID NO: 405) |
| AT4G22820 | | A20/AN1-like | AGAAAAGCAAGAGA | TAGTTACGTGTTTCTGTTTTTCTCTAATTTTTCTC |
| | | zinc finger | G (SEQ ID NO: 225) | TTGTTGTTCTCGATTAACGAAAAAGACTTGTCG |
| | | family protein | | TTCTCAATTCTTATCGATTTAAGAACAAATCATC |
| | | | | TAACGAAGATTACTTCCGAAGATCAGAAACAA |
| | | | | ACACAAACTGTGAATCGTTGTTTGTTAATTCTCT |
| | | | | TTAAAATCGCCAGAAGAAAGAGATCTCCGTTTT |
| | | | | CTACAGAAGAAAAGCAAGAGAGTAAGA (SEQ |
| | | | | ID NO: 406) |
| AT2G30170 | | Protein | GAACGAGAGAGCAA | GAGAACGAGAGAGCAAGCCATTGCAGGAAAT |
| | | phosphatase 2C | G (SEQ ID NO: 226) | GGCGATTCCAGTGACGAGAATGATGGTTCCTC |
| | | family protein | | ACGCAATACCATCGCTTCGTCTCTCACATCCAA |
| | | | | ACCCTAGTCGCGTTGACTTCCTCTGTCGCTGTG |
| | | | | CTCCATCAGAAATCCAACCACTTCGGCCTGAAC |
| | | | | TCTCTTTATCTGTCGGAATTCACGCAATCCCTCA |
| | | | | TCCAGATAAGTGTCGAAATTATATAGGTAGAG |
| | | | | AAAGGTGGTGAAGATGCTTTCTTTGTAAGTAGT |
| | | | | TATAGAGGTGGAGTC (SEQ ID NO: 407) |
| AT5G47120 | B11 | BAX inhibitor 1 | AGCAAAAAAAACGAA | AATATTTTCATTAATCGATTCTCAAAGTCAAGCA |
| | | | (SEQ ID NO: 227) | AAAAAAACGAAACA (SEQ ID NO: 408) |
| AT5G41990 | WNK8 | with no lysine | GATAAAAGAGAAGA | CCTTTCATTGATTTCATCATCATCATCATCCTTC |
| | | (K) kinase 8 | G (SEQ ID NO: 228) | GTTTTTTCTCTATCGATCTAGCAGATTCTTTCGG |
| | | | | GGACCAAAATCAAAATCATGGTGGATCATCAA |
| | | | | TGGAAGGATTTAATCGGATAAAAGAGAAGAGA |
| | | | | CGGAATCACGACGGGAGAAGAGATCGGGAAA |
| | | | | TCGGAAAATCGGAGATGATGGGGATTTCTTTC |
| | | | | GCCGCCAAACTCCGTTTCCGATCTCGATTTCGA |
| | | | | ACTTCTTCAATCGATTCTTATTGCTTCGCTCGTG |
| | | | | AGGCTTTCTCCGATTGTATCTCCTCCGTCCATTT |
| | | | | CTTCTTCTTATAACCTTTTTCTTTGTAATAACCTC |
| | | | | CGTCCTCTTCAGCTTTCTTTCTTTTCATCTTCAAT |
| | | | | CTCACCTTAAATTCTCCACTTTTTTCTTCTTCTCC |
| | | | | TTCTGTTCTCGATTGCTTTGTTTGTTGTGTTGTG |
| | | | | CATACATAT (SEQ ID NO: 409) |
| AT3G62600 | ERDJ3B | DNAJ heat | AAAACAAGTAGAGA | AATCGTTTCCACGAAAACAAGTAGAGAGAGTG |
| | | shock family | G (SEQ ID NO: 229) | ATTCGAGTTTTCCAATCATAAAAATCAGCGAAG |
| | | protein | | AAGATCTTCGTTCTTGTTCATTCTGTGAGGTTTC |
| | | | | ATTGTTAAAATCGAAACGAATCTCAGGTTGGA |
| | | | | GTAATCCTTGGGAGAGATCCGATTTCCGTTTCC |
| | | | | (SEQ ID NO: 410) |
| AT3G52060 | | Core-2/I- | TAAATAGAGAGAGAA | GAAAAAACCGTATCTCATTATTATATAAATAGA |
| | | branching beta- | (SEQ ID NO: 230) | GAGAGAACAGCCCCACGTAAACAAATAGCGAT |
| | | 1,6-N- | | AGAGCAACTGTGTCGATTGTCCCAAATAATTTT |
| | | acetylglucosa- | | AAAAATAATTTCACGTGTCCCCATTTTGCTGAC |
| | | minyltransferase | | GTCATTATTCCCCTTTTTCCTTTTTATTGTCACAT |
| | | family protein | | CAGAATTTTTTCTAACTCATTCATTTCAATCAAT |
| | | | | CTTCTTCTTCTTCTTCTTCTTCTTCCTCAGAGAAA |
| | | | | TTCTGTGTTGTTGTATACAGAGAG (SEQ ID NO: |
| | | | | 411) |
| AT5G06060 | | NAD(P)-binding | TCCACAAAAAGAGAG | ACTCACACATCCACAAAAAGAGAGTTAGAGAT |
| | | Rossmann-fold | (SEQ ID NO: 231) | TCCAAGGAGGAGAGTGCGTGAGCGTGACA |
| | | superfamily | | (SEQ ID NO: 412) |
| | | protein | | |
| AT1G14210 | | Ribo-nuclease | AAGAAACACAGAGA | AAGAAACACAGAGAGCAAAACAC (SEQ ID NO: |
| | | T2 family | G (SEQ ID NO: 232) | 413) |
| | | protein | | |
| AT2G26690 | | Major facilitator | AGAAGAAACTAAGAA | GCTTCTGTGGCTAACAAAGAGCAAACAAACAC |
| | | superfamily | (SEQ ID NO: 233) | TTAGAAGAAACTAAGAATACTCTCATCAAGGC |
| | | protein | | GATATAGAAAAAA (SEQ ID NO: 414) |
| AT2G05840 | PAA2 | 20S | TGAAGACAAAGAAA | TTTTTTTTTGGGTTCTGTCTTGAAGACAAAGAA |
| | | proteasome | G (SEQ ID NO: 234) | AGCTTTCTTCTATAATACATCTTTCTCTACAGAT |
| | | subunit PAA2 | | CACACAGAAGCAAAAATTCCATCTCCGATTTCG |
| | | | | GAAGAGAGTTGTTCTCTTCTCTGAGAAGAAGA |
| | | | | AG (SEQ ID NO: 415) |
| AT1G12580 | PEPKR1 | phosphoenol- | TGCCAAAAAAAAGAG | GAGAGAGGACTGGGTCTGGTCTCTTCGCTGCA |
| | | pyruvate | (SEQ ID NO: 235) | ACCTATAGCTGTTGTTTGCTCTTCGACGGGATT |
| | | carboxylase- | | CTCACTACTCTTTTGCCAAAAAAAAGAGATCGG |
| | | related kinase 1 | | AGGTTCCGAAGGTGAATGCAGCTTGCGATTTC |
| | | | | ATAGAAAAGAAGATTCGTTTGCTGGATTAGGC |
| | | | | TTATTTGTGTATCATAGCTTTGAGGTTTTAACTG |
| | | | | AGATTTATTGATAGTGGAACTTAGGTTTTCGAG |
| | | | | AGGTGTGAACAGTTGGGTAT (SEQ ID NO: |
| | | | | 416) |
| AT5G05080 | UBC22 | ubiquitin- | GAGAGAGGTAGCGA | AAAATAAACATTTGTCTCTATTTCTCTTATAAAA |
| | | conjugating | G (SEQ ID NO: 236) | ATTCAATAATTGAACCTCCTCTCTCTCTCTCTCTT |
| | | enzyme 22 | | CTCTCCCTTCTTCTTCTCCGATTTCGACTTTGAAT |
| | | | | CATTTCTTCGAGAGAGGTAGCGAGAAAGGGAT |
| | | | | CGCCTTTTCTCACTCTCTGCGGATTCTCAATTTT |
| | | | | GGGCAAGAAGGCAAGAACAGTTTTTATCGCAA |
| | | | | TTGAGTCTTGAAGACCACAAGGATTTGATCACA |
| | | | | TTGGTGCTTCTGCCTGTTTATCTGAGTTTGAGG |
| | | | | ACAAGAACTTCTGGGGCGTTTATAATTTGCC |
| | | | | (SEQ ID NO: 417) |
| AT2G30270 | | Protein of | GCCGCAAAAAAAAAA | ATCTTTGGCTTCTACATCCAATTATTTACTTGCT |
| | | unknown | (SEQ ID NO: 237) | TAATTTTATTCATCTGAATTATTTTTTGGTGTAA |
| | | function | | GAAGAATGTTTCGCCGCAAAAAAAAAAATCTG |
| | | (DUF567) | | ATCCGACATCATTAGAACAAAAAAAAACATTGG |
| | | | | CGTTGAATATAAGCTGCTTCTCTTGTTCTTCTTC |
| | | | | TACCTTACGCTTCTGACTGTTATTAGAGACTATG |
| | | | | TAA (SEQ ID NO: 418) |
| AT2G27030 | CAM5 | calmodulin 5 | GACAAAGACGGAGA | ACACACACCAACGTTGATTCTTCTTCTTCTTCTT |
| | | | T (SEQ ID NO: 238) | CTTCTCTCTTTCTCATCTAAACCAAAAAATGGCA |
| | | | | GATCAGCTCACCGATGATCAGATCTCTGAGTTC |
| | | | | AAGGAAGCTTTTAGCCTTTTCGACAAAGACGG |
| | | | | AGATGGTTCTTCTCTCTCAGATCTTTCCTCTTTT |
| | | | | GTATAATTTTCATTCATAATAGACTCACTTGCGT |
| | | | | TTTTTTTGGTGTTTTGAGTATCACTTAGTCTTGG |
| | | | | CTTTAGGAATTTGATGCTCTTCGTTGTCCATAAA |
| | | | | ATCTCTGGATATTCACATTAACATTAAACGCGA |
| | | | | GATTTGATGATATCTTTATCGTTCGTTGATTATA |
| | | | | AATTATAATCGCAATCGGATCTATCTCGATAAT |
| | | | | AATCTCTAACTTAATCGTGTTTTAGTCTTCCAGA |
| | | | | TTTTACTAATTGTGATTAGAATTGACACAAATCT |
| | | | | TAGAATTCAATAATCGAAGTAGATTACATTGAC |
| | | | | ATTTGTAGATTTTTTGTTTAATTGATTCAGTTAT |
| | | | | TTGAGTAGGTTACAATGAAATTTGAAGATTTTG |
| | | | | TGTTCATTTGATACAGTTGTTAGAGTAACTAAA |
| | | | | ATGAAATTTGAAGATTTTGTGTGTTATTAGAGT |
| | | | | AAATTACAATGAAAATTTGAAGATTTGGTGTTA |
| | | | | AAATCTGTTACTGATTTGAGAGAAATGTGTGGT |
| | | | | TTTGTGTTTAGGTTGCATCACAACGAAAGAGCT |
| | | | | AGGAACAGTG (SEQ ID NO: 419) |
| AT1G12470 | | zinc ion binding | TTAAGAGAGGAAGA | GATTTCATAAACCACGACTGACTTCTCCTGCTC |
| | | | A (SEQ ID NO: 239) | GCCGATCAGATCTCCGACGAAGTTTTTGATTAA |
| | | | | GAGAGGAAGAAG (SEQ ID NO: 420) |
| AT1G69530 | EXPA1 | expansin A1 | ACGAAAAGAAGAAA | CCAATTCTAAACCAAACAACAGATTCTCATAAT |
| | | | G (SEQ ID NO: 240) | CATCTCTTCTTTTTTCCTCTTTACGAAAAGAAGA |
| | | | | AAGATCAAACCTTCCAAGTAATCATTTTCTTTCT |
| | | | | CTCTCTCACACACACACATTCACTAGTTTTAGCT |
| | | | | TCACAAAATGTGATCTAACTTCATTTACCTATAT |
| | | | | GCAGGTTTACACAAAAAGAAAAAAGAACG |
| | | | | (SEQ ID NO: 421) |
| AT1G14280 | PKS2 | phytochrome | CACAAAAAGAAACAA | AAGAAATAGTAATACACAAAAAGAAACAAA |
| | | kinase | (SEQ ID NO: 241) | (SEQ ID NO: 422) |
| | | substrate 2 | | |
| AT1G13560 | ATAAPT1 | aminoalcoholphos- | GGAAGAAACGCAAA | GGGAACGCGGAAGAAACGCAAAGCCCTCTCCT |
| | | photransferase 1 | G (SEQ ID NO: 242) | TTTGCTTCTGGTCCTCTCGTCCCGTTTCGCCGCT |
| | | | | CTCTATAGGGGCAAGTGAGAGGTTACTGTCTCT |
| | | | | TTCTTCTTTCAGACACTCGAGACGAGAAAGGCT |
| | | | | CGTATCTGATTTTACCGCCACCGGACCATCTGT |
| | | | | GATAGACAATA (SEQ ID NO: 423) |
| AT5G16650 | | Chaperone | TGAACGGAAAAAGA | ACGAAAACTCATAAAGCCAAAGCCTTTCTTCTT |
| | | DnaJ-domain | A (SEQ ID NO: 243) | CTTCTTTTCTTCCGATTATTCCCAAACACAAAAA |
| | | superfamily | | TACTGCTGAGGAAAAGCAATCCACACGATTCG |
| | | protein | | ATTCAAAGTTTTCATTTTTTCTCTAAAAGTTTGG |
| | | | | ATTTTGATTTCGTTGCTGAACGGAAAAAGAATC |
| | | | | AGCTCCTTTCAGTTTAGGGTTTTGGGTTTCTGTT |
| | | | | TGGTCTCTATCAGATGATGTGTGAGGAGATTCT |
| | | | | TCCTCTGTTTGTGTCTGTTTCAG (SEQ ID NO: |
| | | | | 424) |
| AT1G09690 | | Translation | GCACGAGGAGGAAA | TTTCTTCGGCGATCTAGGGTTTTAGTTGTCGCA |
| | | protein SH3-like | A (SEQ ID NO: 244) | CGAGGAGGAAAA (SEQ ID NO: 425) |
| | | family protein | | |
| AT3G46110 | | Domain of | TGAGAAGAAGAACA | CTCATTCTCAAATCTCTCATTGTGTGTCTGTGAC |
| | | unknown | A (SEQ ID NO: 245) | TATCTCTCTATACAATTCAAACTCTTCAAGATTA |
| | | function | | CTTCCTCTTCACTTTGAGAAGAAGAACAAACCA |
| | | (DUF966) | | ACAAATCTCCAAAATACACCGAACAACATTA |
| | | | | (SEQ ID NO: 426) |
| AT1G72550 | | tRNA | CACTCAGAAGAAGAA | TAACGGTGAAAAATCGTCATCTACTTCTTCTTG |
| | | synthetase beta | (SEQ ID NO: 246) | AAACCCTAGTTCCAAAATCTGCACACACACTCA |
| | | subunit family | | GAAGAAGAAGACGTCATCTCTCTATCTCTGTCT |
| | | protein | | TTCTGCTAATTTCACGAAGAATCTGAGAAT |
| | | | | (SEQ ID NO: 427) |
| AT5G53280 | PDV1 | plastid division1 | CCTGAAGAAGAAGAA | ACAATTAAAGTGAGAATTTTCCTGAAGAAGAA |
| | | | (SEQ ID NO: 247) | GAACTTTTGCTTTTTTTCTGGGTTTGCTTTTTTGT |
| | | | | TGTGTCAATGAA (SEQ ID NO: 428) |
| AT5G42070 | | | ACAGAGGAAAGAAA | ATTTTGTTTTGCGTTTCTGAATTTGTGGCCATTA |
| | | | A (SEQ ID NO: 248) | TCTTCTCACACTCTCTTCTCTTAGCTCACAGAGG |
| | | | | AAAGAAAA (SEQ ID NO: 429) |
| AT4G32180 | PANK2 | pantothenate | TAATAAAAAAAAAAA | GTTGGTGATCCGATTTTTCTGGGTTTGGTTGGG |
| | | kinase 2 | (SEQ ID NO: 249) | TTCCTTTTTTATTTTTTAATAAAAAAAAAAA |
| | | | | (SEQ ID NO: 430) |
| AT2G18040 | PIN1AT | peptidylpro- | GAAGGAGAAGAAAG | AATCGTCGATAATCATTAGGGTAAAGCAAAAA |
| | | lylcis/trans | A (SEQ ID NO: 250) | TAGTGAAGCAGAGCCGCAAAAACACTTTTCCCA |
| | | isomerase, | | AAATCAACGAAGATAGATTCAGATCGGAAGCG |
| | | NIMA- | | AAAGAACGATTCGGTCTCCTCCACAGATCGAAC |
| | | interacting 1 | | ATCGAAGGAGAAGAAAGACCATCATCACAACA |
| | | | | AGCATCGAAAGAAGAGCAAG (SEQ ID NO: |
| | | | | 431) |
| AT5G16970 | AT- | alkenal | GAAACCGAAGAAGA | TAAAAGCAGCGGCGTCATCGAGAGAAACCGAA |
| | AER | reductase | A (SEQ ID NO: 251) | GAAGAAGCAGTAACAAATTTGGTGAAGTCACG |
| | | | | AGAATCAACG (SEQ ID NO: 432) |
| AT5G09410 | EICBP. | ethylene | AAACCACAAGAAGAG | ATGAATTAGGAATCTGTGATTATGATAACGGA |
| | B | induced | (SEQ ID NO: 252) | GTCTGAAGCCTAGACTCGAAACCACAAGAAGA |
| | | calmodulin | | GA (SEQ ID NO: 433) |
| | | binding protein | | |
| AT5G05360 | | | AAAAAAAATTGAAAA | AATTGATCGCACTGTCAAACCAAAAAAAATTGA |
| | | | (SEQ ID NO: 253) | AAACCCTAAATTGGTTGA (SEQ ID NO: 434) |
| AT4G23740 | | Leucine-rich | TACAAAAAGAAACAG | CTTTCACCCACTTTAATATGCCAAAAAATAAGA |
| | | repeat protein | (SEQ ID NO: 254) | ACAAAATTATATCCGTTGCTTGAAAATCACAAG |
| | | kinase family | | CTCTTCTTAACTTCACAAGTGCTTCAATGGCGGT |
| | | protein | | TCTTCACATTATCTTCACTGCGTAATTGAAGAA |
| | | | | GTTGTTCTCTCTTCCTCTTAATTTCGAGTTGTGT |
| | | | | TCTTAAAAAACTCCAGAGCTGATTCGATTCTCG |
| | | | | AGAAGAAACTAAGCCGACAATAAAGTTCAGAT |
| | | | | CTGGAAAAAAGCGAGCTCCAGATTACAAAAAG |
| | | | | AAACAGCTCGTTTTTTTCACTTTCAAAAAA (SEQ |
| | | | | ID NO: 435) |
| AT3G47560 | | alpha/beta - | CAAACAAAGTAAAAA | TTATCTTTCTCAACGCACGCCTTACCATTAAGGA |
| | | Hydrolases | (SEQ ID NO: 255) | GACCCAAATTTCCTGCAACAAACAAAGTAAAAA |
| | | superfamily | | AGTTGAGA (SEQ ID NO: 436) |
| | | protein | | |
| AT3G13740 | | Ribo-nuclease III | TCGGAAAAAGCAGA | TATTTTCGTGCTCGGAAAAAGCAGAGTAAAGCT |
| | | family protein | G (SEQ ID NO: 256) | TTAAAAA (SEQ ID NO: 437) |
| AT3G58030 | | RING/U-box | AAGTGAAAGCAAGA | AAAAAAGGGCGAATTTTTCCATGGCGTTGTCG |
| | | superfamily | G (SEQ ID NO: 257) | GAGTTTCAGCTAGCTCTGAGCTTGGTGGTCTTG |
| | | protein | | TTCTTCTAGCTGATTTGATCGAAACCCCATGTTC |
| | | | | TTATGATTTTACACGACCTAATCCAAAACTCCA |
| | | | | GGTCCTTGATTGATTCTTCTCTCTCTCCAGCTCC |
| | | | | AGATTCTTCTGATTTCTTTTGTTATCATTTGTTTT |
| | | | | TGTAAGATTTGTATCCGTTTTTGGGTTTTGCTTA |
| | | | | GCTGATTCTTGCTGGATCGAGAGTTGAATAACT |
| | | | | CTGCTTTTCTTCAATCTGGTTTTTTTTTTTTGTTT |
| | | | | CATAGAGGAGAAAGGTTGTGGATTTCTCAGGT |
| | | | | GGGGATTTGAGAATTAGGGTTTTCTGATTGGG |
| | | | | GGTTTTCTTATTGATGTTACCTTCACCAAATTGT |
| | | | | TGTCGGAGATCTAGATTTGGTTCAGTTATGGAA |
| | | | | TAATGGCTCGTCTCTTGCCATCTCTATTCGTAAT |
| | | | | TAGCATCTTCTTCTTCATCCAAAGACTCCTCCTT |
| | | | | TCTTCGTTAATCCATCGCCAGCTATTGAATCTGA |
| | | | | AGCAAATCTGAGAATCTACCGAACTCACGCACC |
| | | | | TGTATATTGCTTACACGATACAGAGCACACGGA |
| | | | | GACGGAGTACATATTGTTCAGCGCAAGTGAAA |
| | | | | GCAAGAGCCTTTTTGTCTATTG (SEQ ID NO: |
| | | | | 438) |
| AT3G07230 | | wound- | TATAAAAAAAAAAAA | ATACTCGTATCTTGTAGCAGCCACTAAAGCAAA |
| | | responsive | (SEQ ID NO: 258) | ATTCTGAGATCGAAAAAGCTATATAAAAAAAA |
| | | protein-related | | AAAACTGCTTCCGTTTCATCGATTTTGTCCAGAT |
| | | | | CTTCCCCTTCTTCCGGTAATCGAAGCTTACGAG |
| | | | | ATAGTTGAGTGAAG (SEQ ID NO: 439) |
| AT3G05840 | ATSK12 | Protein kinase | GTGACAAAGGAAGA | ACATTAGCTTCCTCATTTTTATTCTTATTATTATT |
| | | superfamily | A (SEQ ID NO: 259) | ATTCATCAGACCAACAACAAAAAGGAGATAAA |
| | | protein | | GAGAAGAGGATTCATCATCATCAATCAATCCTT |
| | | | | CATTTTATGGATCTACTCATATCTTGATTCTTCC |
| | | | | TTCTATCTCTCCCTTTTCTTCCATCTCTTTTTCTCT |
| | | | | GGGTTTCCCCGGATTGAGTTTTTTAATCTCTGAT |
| | | | | TGACAGATTTGAAGAGCGTGACAAAGGAAGAA |
| | | | | TCTTTTATTAAAACAAATTCTTCTGTTTTAATCTT |
| | | | | GGG (SEQ ID NO: 440) |
| AT3G01770 | BET10 | bromodomain | GAAGGGAGGGCAGA | TTAGGGACGGGACACTAGAGAAGGGAGGGCA |
| | | and | G (SEQ ID NO: 260) | GAGAGCGATTTTGTTCTCTCTCTACTTCTCGGTC |
| | | extraterminal | | GTCTTCTTCGTCTCCACTCTAGGGTTTTACTCTA |
| | | domain protein | | TCTTCTTCTTCATCATCATCTTCTACACCAATCTC |
| | | 10 | | TAGCGTTAATCTGTTTCTGCTGGAGAAGATTTA |
| | | | | CGCTTGTTCCTCGGTTCTCTTACTTCTGCTCCGG |
| | | | | TTCGATCGCTTGCTAAGTGTTTCGAGTTGGTTC |
| | | | | GCACTTCGGTGGGCGATATC (SEQ ID NO: 441) |
| AT3G12300 | | | GGAGAAGCAGGAAA | CAAGTCTACGAGCTTCTTCTTCTCGGAATCGGA |
| | | | A (SEQ ID NO: 261) | GAAGCAGGAAAATTCCGGAGGAGCAGGAAG |
| | | | | (SEQ ID NO: 442) |
| AT1G53380 | | Plant protein of | GATAAACAAAGAAAA | GTTTCTCATCTCCAGCTCTCATTTTCTCTCTCATC |
| | | unknown | (SEQ ID NO: 262) | TTCAACCTTAACTCTCTTTTCTCTCTACTCTTTCT |
| | | function | | TTGGACGAATCTGTCTATTGTTTGTAAGTTTTCA |
| | | (DUF641) | | AGGAAGGTAAAGAAACAGAGAGATCTAACTTC |
| | | | | GTCTGCAGGGTTTAAGCAGAGGTTGGTTTGTG |
| | | | | GATTCTTCGATTTCTTCTTCAGATTTAGTCTACA |
| | | | | ATGAAGTGAGAATTTCTAAAGATAAACAAAGA |
| | | | | AAAACTTGAGACTTTAGCAAG (SEQ ID NO: |
| | | | | 443) |
| AT1G25440 | | B-box type zinc | TGCAGAGAGCAAAA | ACTGACACAAAAGGGAATGCGCTTCATGCGGG |
| | | finger protein | G (SEQ ID NO: 263) | TCATCCTCTTAATCTCAAACTCTCTAGGACTACA |
| | | with CCT | | CTAAATCTAACTTTTTGCAGAGAGCAAAAGATT |
| | | domain | | CAATAATTGAGATTGATCTCAAAACCAAAGCTC |
| | | | | TCGTGCTCTTGTCGTTGATGTTGGTTGTGTAGA |
| | | | | CTTTGTATACA (SEQ ID NO: 444) |
| AT3G26950 | | | AAAAGAAACGATGA | ATCCAAAGCTCTGATGTAAGAAACTCTACACTT |
| | | | G (SEQ ID NO: 264) | GTTCGAGTTTCGGAGAAAAGAAACGATGAGGA |
| | | | | AGAG (SEQ ID NO: 445) |
| AT2G06025 | | Acyl-CoA N- | AAAGAAAGCTGAGA | ATACAATTCCAACAAAACCACAAAGACGACTCT |
| | | acyltransferases | A (SEQ ID NO: 265) | CTTCAGAGAGTTTTGAGAGGGTGAGAGAGCCG |
| | | (NAT) | | TGCTCGGCGTTGTTAGAAAGAAAGCTGAGAAT |
| | | superfamily | | TGCAACTGCTTACAAGAGCAATGTCGACAAGCT |
| | | protein | | GATCAAGAGTCTCTTGGATTTGTGCTTCTGTAC |
| | | | | TTCTTAAGAGGAAGGTCCCGCAAGATACCATCT |
| | | | | TCTCAAAAGTCCAATCAATCTACGCTTTTCAATT |
| | | | | CGCCACGTCACAGAATCCTGACCGTTAGATACA |
| | | | | AACGCGCCAACTCGTCAAACTTTGCTTTCTGGT |
| | | | | ACGGCGGCG (SEQ ID NO: 446) |
| AT5G43460 | | HR-like lesion- | CGCCGAAACGAAGAA | GAAATGTTAATAAATAAACCTAAACCAATAGAA |
| | | inducing | (SEQ ID NO: 266) | CCGCAGTTTTTCCTCCTCGCCGAAACGAAGAAG |
| | | protein-related | | ATTCTCCTTCTCTCCGTCAGACAAATCTACGAAC |
| | | | | AAGCGAGCCTGAGCTTAAGACCAAACTCATAG |
| | | | | AG (SEQ ID NO: 447) |
| AT2G01720 | | Ribophorin I | AGAGAGAAGTGAGA | CGTAACTAATCCCTAAATCAAGAGAGAAGTGA |
| | | | G (SEQ ID NO: 267) | GAGACACTGAGACTTTGTAGTTGACCGGATCAT |
| | | | | TCTCACTTCGCCGGCCGACGTTCTTCCTTCCGCC |
| | | | | GTCGGTATCTATATTTACGATCCACGATCTCTCT |
| | | | | TGCTGTTTCTGTCTTCATCGTGACGAAA (SEQ ID |
| | | | | NO: 448) |
| AT5G41050 | | Pollen Ole e 1 | AAGAAAAAAACTGAA | CATCTCTTTGTGCCTCTCTTTACTCATCTCTTTTT |
| | | allergen and | (SEQ ID NO: 268) | CCACAAGAGTCTTGAGTTTTATAAAAAAGACAA |
| | | extensin family | | GCTTGAAGCTTTGTTTGAATGGAGTTACTGTTT |
| | | protein | | GATCTTTGTTTGTTCTTTTGTCTTTAACCACTTG |
| | | | | GCCCATTCTTTGTCTGTTTCTTTCATCAACCACA |
| | | | | TAAACAAAAAGGAAACCTCATCTGTAAACAAGT |
| | | | | GTTTATCCAAGGATAAAGAAAAAAACTGAAAC |
| | | | | TTGTGAAC (SEQ ID NO: 449) |
| AT1G76020 | | Thioredoxin | GAGAAAAAGTGTGA | GAGAAAAAGTGTGAGTCAGAGAATA (SEQ ID |
| | | superfamily | G (SEQ ID NO: 269) | NO: 450) |
| | | protein | | |
| AT1G58270 | ZW9 | TRAF-like family | AATATAGAAAAAGAA | ACAAACACAAAATATAGAAAAAGAAATA (SEQ |
| | | protein | (SEQ ID NO: 270) | ID NO: 451) |
| AT1G19000 | | Homeodomain- | GACGCAAAGGGCAA | AGATCCACTCACACCTCGTCTCCTAATCTGTACG |
| | | like superfamily | A (SEQ ID NO: 271) | GTTCTTATTTCGAAAGGGTAAAAACCAAAAGC |
| | | protein | | GACGCAAAGGGCAAAATCGGAAAAAGTGTTTT |
| | | | | ATTT (SEQ ID NO: 452) |
| AT1G12580 | PEPKR1 | phosphoenolpy- | CATAGAAAAGAAGAT | GAGAGAGGACTGGGTCTGGTCTCTTCGCTGCA |
| | | ruvate | (SEQ ID NO: 272) | ACCTATAGCTGTTGTTTGCTCTTCGACGGGATT |
| | | carboxylase- | | CTCACTACTCTTTTGCCAAAAAAAAGAGATCGG |
| | | related kinase 1 | | AGGTTCCGAAGGTGAATGCAGCTTGCGATTTC |
| | | | | ATAGAAAAGAAGATTCGTTTGCTGGATTAGGC |
| | | | | TTATTTGTGTATCATAGCTTTGAGGTTTTAACTG |
| | | | | AGATTTATTGATAGTGGAACTTAGGTTTTCGAG |
| | | | | AGGTGTGAACAGTTGGGTAT (SEQ ID NO: |
| | | | | 453) |
| AT5G38980 | | | ACCACAGAAAAACAA | AATCACTCCTCAAGCAAATCACTCCTCACACCA |
| | | | (SEQ ID NO: 273) | CAGAAAAACAAATAATTGAAGAA (SEQ ID NO: |
| | | | | 454) |
| AT3G14870 | | Plant protein of | GAACAACAAACAAAA | ACTCTAAAGCCTTTTTCCCCTCTTCTCATTCTCG |
| | | unknown | (SEQ ID NO: 274) | AGCTCCGGACTTGTCTTGAAACCGTGAAGGAA |
| | | function | | TCTGTATCTTTTGTATGTTACCCATTTTATTGTC |
| | | (DUF641) | | GTTAAGAATCAATTTAGAGGCAAAACGCCGAG |
| | | | | AGGTTTGCCCGGGAGAGTGTTTTTACATCGATC |
| | | | | AGGGTTTAAGCAGAGGTTGGTTTGTCATTTCGC |
| | | | | CAGTTTGCTTCTTCAAATTCACTCTACGATGAAG |
| | | | | TGAGAACAACAAACAAAACATAGATAAGATAG |
| | | | | AGACCTTGGAACTGTTGGAAG (SEQ ID NO: |
| | | | | 455) |
| AT1G49975 | | | GACATAAAACAAGAA | AAGAGACATAAAACAAGAATCTTATCTTCTGGT |
| | | | (SEQ ID NO: 275) | CAAGAGAGAG (SEQ ID NO: 456) |
| AT1G14920 | RGA2 | GRAS family | GAGTGAAAAAACAAA | ATAACCTTCCTCTCTATTTTTACAATTTATTTTGT |
| | | transcription | (SEQ ID NO: 276) | TATTAGAAGTGGTAGTGGAGTGAAAAAACAAA |
| | | factor family | | TCCTAAGCAGTCCTAACCGATCCCCGAAGCTAA |
| | | protein | | AGATTCTTCACCTTCCCAAATAAAGCAAAACCT |
| | | | | AGATCCGACATTGAAGGAAAAACCTTTTAGATC |
| | | | | CATCTCTGAAAAAAAACCAACC (SEQ ID NO: |
| | | | | 457) |
| AT5G51020 | CRL | crumpled leaf | GAAACAAGTAGAGAT | AACCTTACTCCTCCTCCTCTTCCTCTTTCTCTAAT |
| | | | (SEQ ID NO: 277) | CGGCAAAATTTTCTGCTCCTGAGAAACAAGTAG |
| | | | | AGATACTAAAGATGGAATCTTTGAACTAAATTC |
| | | | | GAAACCTTTTA (SEQ ID NO: 458) |
| AT4G27990 | YLMG | YGGT family | CACCGAGGAACAAAG | ACAACATTCTGAGGAGTGAGTAATCTCCGGCA |
| | 1-2 | protein | (SEQ ID NO: 278) | CCGAGGAACAAAG (SEQ ID NO: 459) |
| AT5G17630 | | Nucleotide/sugar | AACCGAAACCAAGAG | AGAGCTTTCAAAAAATTGTTGTACTTCCCAACG |
| | | transporter | (SEQ ID NO: 279) | GATCTCTGACGTTTGGTCCAGAGCCGACGACG |
| | | family protein | | ACCCACAACCGAAACCAAGAGCTATCTCTTTTT |
| | | | | CCTCTTCTCTCTCTCCTTCTCTACCTGCGTTCGTG |
| | | | | CTTAAACA (SEQ ID NO: 460) |
| AT2G27260 | | Late | AAAACAAATCAAAAG | ACATTTCCTTTTAAATTAAATTGCGTTAATTTCT |
| | | embryogenesis | (SEQ ID NO: 280) | CACTTCCCTTTACTTCTTCTTCTTCACCATCACAA |
| | | abundant (LEA) | | ACATCTTCGTCTCTTGAAGATTCCAAAAAAAAC |
| | | hydroxyproline- | | AAATCAAAAGCT (SEQ ID NO: 461) |
| | | rich | | |
| | | glycoprotein | | |
| | | family | | |
| AT2G02040 | PTR2- | peptide | AAGTAAAATAAAAAG | AAGTCGCCGGGAAAAGTAAAATAAAAAGCCGT |
| | B | transporter 2 | (SEQ ID NO: 281) | CACGTCTCCGATAAATAATAGAGTATCGTTAGA |
| | | | | TAGGTAGCTTCAACGTAAGGAATCTAAATTGGT |
| | | | | TCAGCTCAAAAAACGAAAACG (SEQ ID NO: |
| | | | | 462) |
| AT1G75040 | PR5 | pathogenesis- | GACACACACAAAAAA | ATCATCATCACCCACAGCACAGAGACACACACA |
| | | related gene 5 | (SEQ ID NO: 282) | AAAAACCCATAAAAAAAT (SEQ ID NO: 463) |
| AT2G30170 | | Protein | GAGAAAGGTGGTGA | GAGAACGAGAGAGCAAGCCATTGCAGGAAAT |
| | | phosphatase 2C | A (SEQ ID NO: 283) | GGCGATTCCAGTGACGAGAATGATGGTTCCTC |
| | | family protein | | ACGCAATACCATCGCTTCGTCTCTCACATCCAA |
| | | | | ACCCTAGTCGCGTTGACTTCCTCTGTCGCTGTG |
| | | | | CTCCATCAGAAATCCAACCACTTCGGCCTGAAC |
| | | | | TCTCTTTATCTGTCGGAATTCACGCAATCCCTCA |
| | | | | TCCAGATAAGTGTCGAAATTATATAGGTAGAG |
| | | | | AAAGGTGGTGAAGATGCTTTCTTTGTAAGTAGT |
| | | | | TATAGAGGTGGAGTC (SEQ ID NO: 464) |
| AT5G42300 | UBL5 | ubiquitin-like | CGGAGGAATAGAAA | ACGAGCCTTAACGCGTAGAATCTTCCCGTACTT |
| | | protein 5 | A (SEQ ID NO: 284) | TACTTTTCCGGAGGAATAGAAAATTGGGGGCT |
| | | | | AGGGTTCGCAATTGTAGTTTTCGAGCGAAGAA |
| | | | | G (SEQ ID NO: 465) |
| AT3G62830 | UXS2 | NAD(P)-binding | TAATAAGAGTGAAAA | TCTCGTAATAAGAGTGAAAAACAAGCCTTAACC |
| | | Rossmann-fold | (SEQ ID NO: 285) | TGTAAACGCTTACGCTAGTTAAATACACAACAA |
| | | superfamily | | AGACCGATTCGCTTTTCACTCTCTCGTTCAAGAT |
| | | protein | | CTAGAATTCAATTTGTGAGGTTTGGAG (SEQ ID |
| | | | | NO: 466) |
| AT1G06190 | | Rho | CAAGGAAAAGGCAAT | GAGAGTCGACAAGGAAAAGGCAATGCAAGAA |
| | | termination | (SEQ ID NO: 286) | GAAGCTTAAATCTCTCTTCTCTGCTCCTGAAGTC |
| | | factor | | TGTTC (SEQ ID NO: 467) |
| AT1G47420 | SDH5 | succinate | TCGGAAAAATCAGAA | GCGTTGGTTCTCTTCTTCAAAACAAGCTCTCTCT |
| | | dehydrogenase | (SEQ ID NO: 287) | GTCCCTCTCTGTCTCTCTCTTTGGGTAATCGGAA |
| | | 5 | | AAATCAGAAAA (SEQ ID NO: 468) |
| AT1G06360 | | Fatty acid | CTCAAAGAAAAACAA | ATACAAATCATAACTCAAAGAAAAACAACCCCT |
| | | desaturase | (SEQ ID NO: 288) | CAACGGTCG (SEQ ID NO: 469) |
| | | family protein | | |
| AT5G04280 | RZ-1c | RNA-binding | AGGCGAAGGAAACA | ACCACCACCATTTTAGGGTTTCTTCGTGCCATTG |
| | | (RRM/RBD/RNP | A (SEQ ID NO: 289) | ATATTTTGAGAGGCGAAGGAAACAATACGATT |
| | | motifs) family | | CAGAGAGAGACGAGTGAAA (SEQ ID NO: 470) |
| | | protein with | | |
| | | retrovirus zinc | | |
| | | finger-like | | |
| | | domain | | |
| AT1G18440 | | Peptidyl-tRNA | TCCCCAGAAGAAAAG | CTAATTCCCCAGAAGAAAAG (SEQ ID NO: 471) |
| | | hydrolase | (SEQ ID NO: 290) | |
| | | family protein | | |
| AT5G47570 | | | CCTGAAAAGAGCGAA | TGACTGCGTCTTTCTTCTCTCTCTATCTGTAATTT |
| | | | (SEQ ID NO: 291) | GATTGGATTTTGGATCGAAACCTGAAAAGAGC |
| | | | | GAAA (SEQ ID NO: 472) |
| AT2G26590 | RPN13 | regulatory | GAAAGAGGTGGTGA | AATTGAAAGAAAAAAAAAAACGAGAAGCGTTT |
| | | particle non- | T (SEQ ID NO: 292) | TCTTTCTCTCCAAAATCCATTACTCGCGAACTTT |
| | | ATPase 13 | | CCTCTGCTAAGTGTTCACTAGAAAGAGGTGGT |
| | | | | GATT (SEQ ID NO: 473) |
| AT4G36990 | TBF1 | | ACATACACACAAAAA | TCTAGAAACAGCATCCGTTTTTATAATTTAATTT |
| | | | TAAAAAAGAC (SEQ | TCTTACAAAGGTAGGACCAACATTTGTGATCTA |
| | | | ID NO: 293) | TAAATCTTCCTACTACGTTATATAGAGACCCTTC |
| | | | | GACATAACACTTAACTCGTTTATATATTTGTTTT |
| | | | | ACTTGTTTTGCACATACACACAAAAATAAAAAA |
| | | | | GACTTTATATTTATTTACTTTTTAATCACACGGA |
| | | | | TTAGCTCCGGCGAAGTATGGTCGTCGTCTTCAT |
| | | | | CTTCTTCCTCCATCATCAGATTTTTCCTTAAATG |
| | | | | GAAGAAACCAAACGAAACTCCGATCTTCTCCGT |
| | | | | TCTCGTGTTTTCCTCTCTGGCTTTTATTGCTGGG |
| | | | | ATTGGGAATTTCTCACCGCTCTCTTGCTTTTTAG |
| | | | | TTGCTGATTCTTTTTCCTTCGACTTTCTATTTCCA |
| | | | | ATCTTTCTTCTTCTCTTTGTGTATTAGATTATTTT |
| | | | | TAGTTTTATTTTTCTGTGGTAAAATAAAAAAAG |
| | | | | TTCGCCGGAG (SEQ ID NO: 474) |

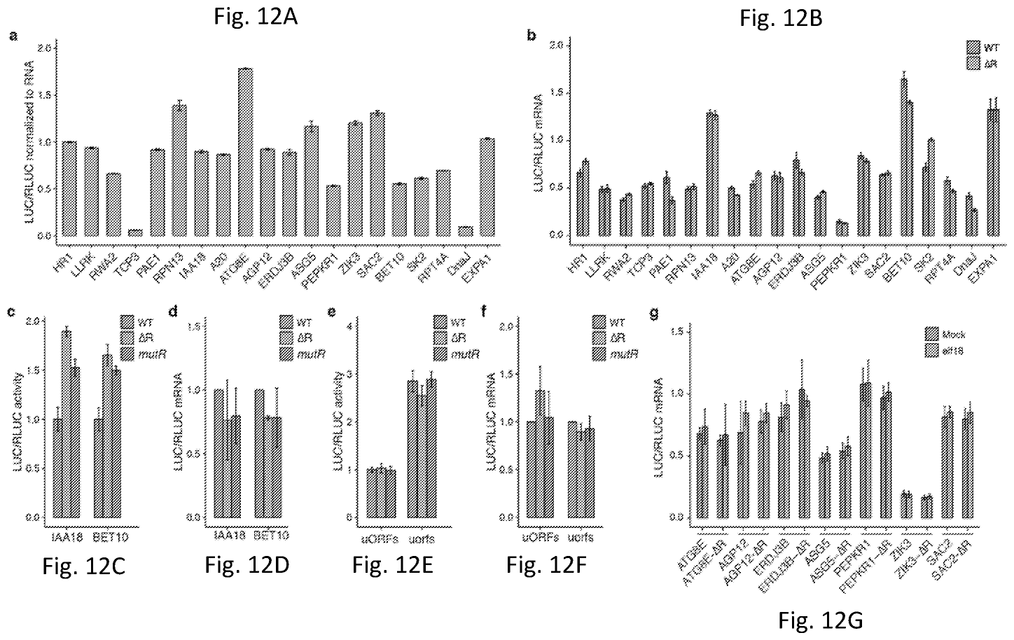

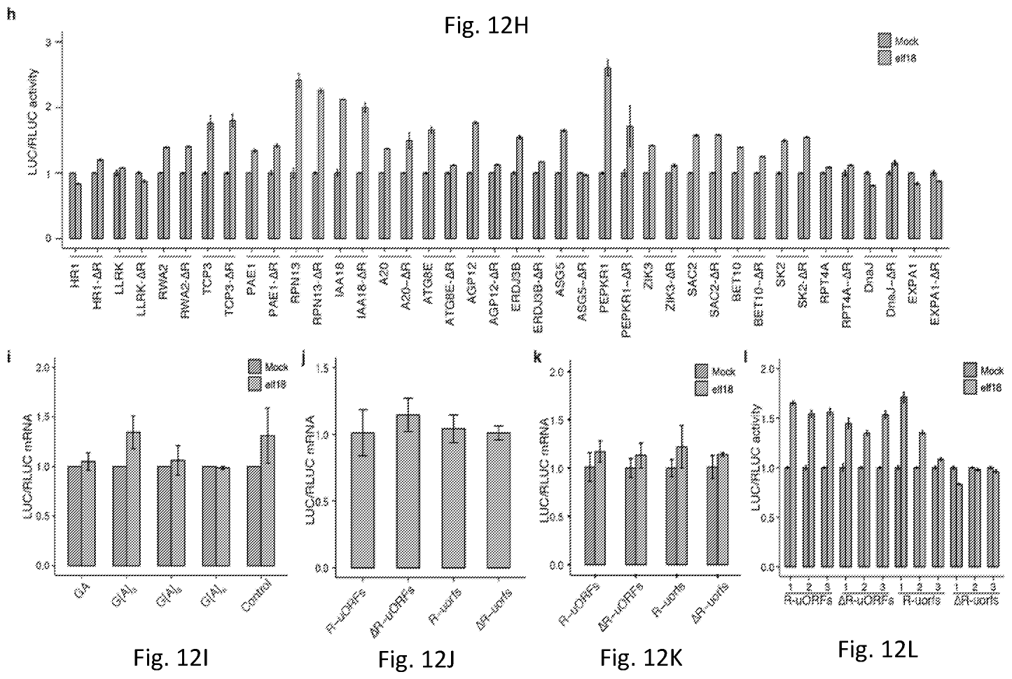

To examine the effect of R-motif on elf18-induced translation, we tested 5′ leader sequences of 20 R-motif-containing TE-up genes using the dual-luciferase system. Consistent with their known importance in controlling translation²⁴, the different 5′ leader sequences showed distinct basal translational activities after normalization to mRNA levels (FIG. 12A). In 15 of the 20 tested 5′ leader sequences, elf18-mediated TE increase was confirmed (FIG. 3B). We then generated R-motif deletion mutant reporters and found that 11 of them showed with increased TE while only two displayed decreased TE compared to their corresponding WT controls (FIG. 3C and FIG. 12B). The translational changes observed in these deletion mutants, were unlikely due to shortening of the transcripts because similar effects were observed when the R-motifs in IAA8, BET10 and TBF1 were mutated through multi-base pair substitutions (FIGS. 12C-F). These results suggest a predominantly negative role for R-motif in basal translational activity. We subsequently examined the R-motif deletion mutant reporters for responsiveness to elf18 induction and found six to have abolished or decreased responses compared to the controls (FIG. 3D and FIGS. 12G and 12H), indicating that releasing R-motif mediated repression may b an activation mechanism for these genes during PTI. To demonstrate that R-motif is sufficient for responsiveness to elf18, repeats of GA, G[A]₃, G[A]₆ and mixed G[A]ₙ, which are core sequence patterns found in R-motifs of endogenous genes, were inserted into the 5′ leader sequence of the reporter. We found that translation of resulting reporters indeed became responsive to elf18 induction (FIG. 3E and FIG. 12I). However, R-motif in some genes may have a less or more complex role in regulating translation because deleting R-motif in these genes did not affect their translation upon elf18 treatment (FIG. 12H). Other mRNA sequence features in these transcripts may influence R-motif activity.

The relationship between R-motif and uORFs during PTI-mediated translation was then conveniently studied in TBF 1 because both features were found in its transcript (FIG. 1A). TE assessment using the dual-luciferase system showed that deletion of R-motif had no significant effect on basal translation of TBF1, in contrast to the uORFs_{TBF1} mutant (ATG to CTG mutation for both uORFs start codons; FIG. 3F and FIG. 12J). However, both R-motif and uORFs mutant reporters showed compromised responses to elf18 in transient expression analysis as well as in transgenic plants (FIG. 3G and FIG. 12K, L). The effects appeared to be additive, suggesting that R-motif and uORFs control translation through distinct mechanisms.

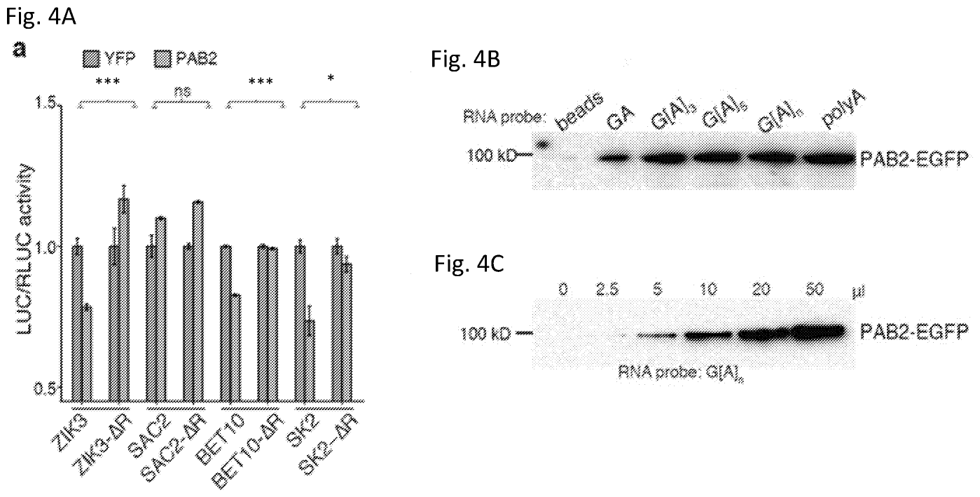

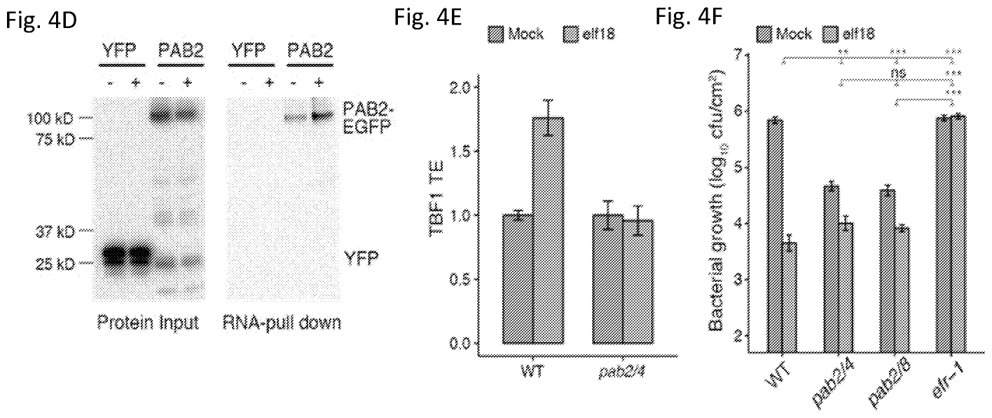

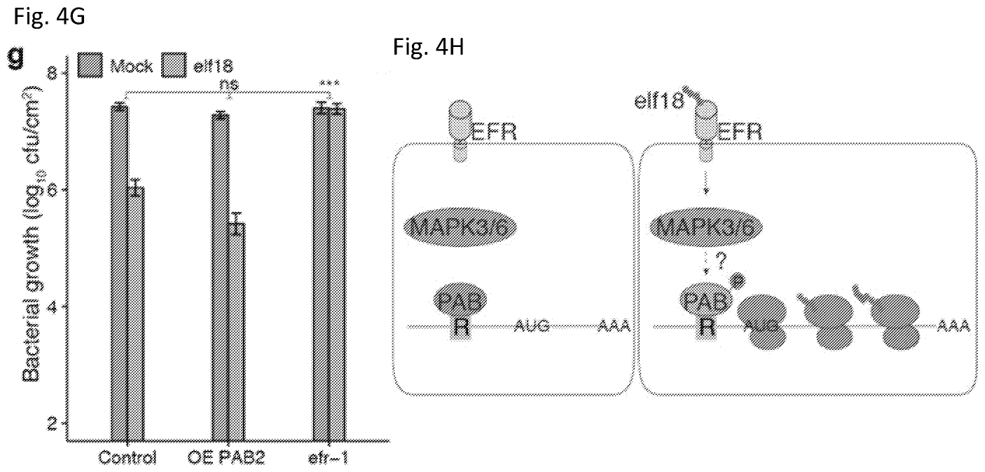

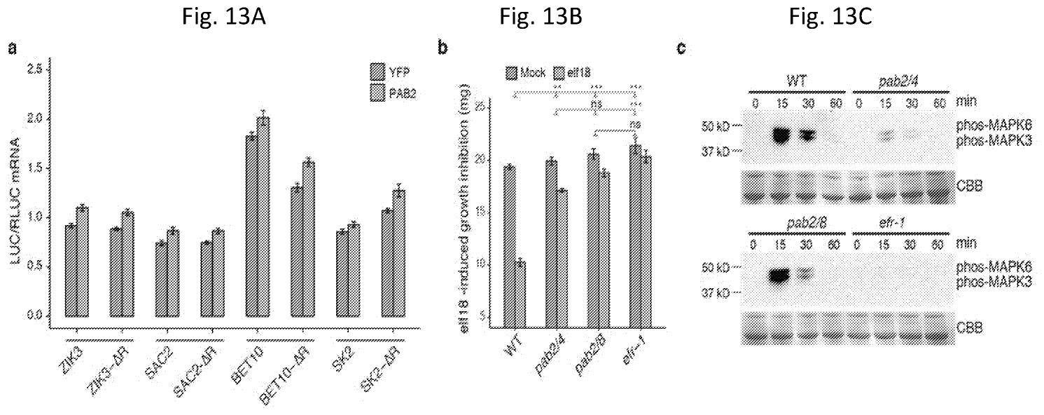

We hypothesize that the mechanism by which R-motif affects translation is likely through association with poly(A)-binding proteins (PABs) because these proteins have been shown to bind to not only poly(A) tails of transcripts to enhance translation, but also A-rich sequences located in their own 5′ leader sequences to inhibit translation^{25, 26}. To test our hypothesis, we examined the role of class II PABs (i.e., PAB2, PAB4 and PAB8), which are major PABs in plants based on genetic data²⁷. We co-expressed PAB2 with three individual R-motif-dependent genes, ZIK3, BET10, and SK2 and one R-motif-independent gene, SAC2, as a control. We found that all three R-motif-dependent genes, but not the control, had lower TE when PAB2 was co-expressed, and that this inhibition could be overcome by deleting the R-motif (FIG. 4A and FIG. 13A). This PAB2 effect is likely through a direct physical interaction with R-motif because in an in vitro binding assay, PAB2 displayed comparable affinities to G[A]₃, G[A]₆ and G[A]ₙ repeats as to poly(A) (FIGS. 4B and 4C). Moreover, plant-synthesized PAB2 could be pulled down using a G[A]ₙ RNA probe (FIG. 4D). Surprisingly, PAB2 from the elf18-induced plants appeared to bind the probe more tightly than the mock-treated control, suggesting elf18-triggered derepression was unlikely through dissociation of PAB2. PAB2 is known to switch its activity through phosphorylation²⁸, which might have occurred upon elf18 treatment.

We next examined the phenotypes of the pab2 pab4 and pab2 pab8 double mutants (the triple mutant is non-viable)²⁹. To separate the mutant effects on general translation, we focused our characterization on sensitivity to elf18. We first showed that the elf18-triggered TE increase in the endogenous TBF1 was compromised in the pab2 pab4 double mutant as measured by polysome fractionation (FIG. 4E). We then performed a test of resistance test to Psm ES4326 with and without elf18 pre-treatment. In comparison to WT, the double mutants had significantly elevated basal resistance to Psm ES4326, but reduced resistance to the pathogen after elf18 treatment (FIG. 4F). This insensitivity to elf18 was rescued by transformation of PAB2 into the pab2 pab8 double mutant background (FIG. 4G). PABs are not only essential for elf18-induced resistance against Psm ES4326 but also critical for the growth-to-defense transition because in the pab2 pab4 and pab2 pab8 mutants, the inhibitory effect of elf18 on plant growth was diminished (FIG. 13B). These data support our hypothesis that PABs play a negative role in background translation, but a positive role in elf18-induced translation (FIG. 4H). Whether the activities of PABs are regulated by components of the known PTI signalling pathway, such as MAPK3/6 remains to be tested. Detection of MAPK3/6 activity in the pab2 pab4 and pab2 pab8 mutants, albeit lower in pab2 pab4 (FIG. 13C), suggests that PABs could function downstream of MAPK3/6, possibly as substrates, or in an independent pathway.

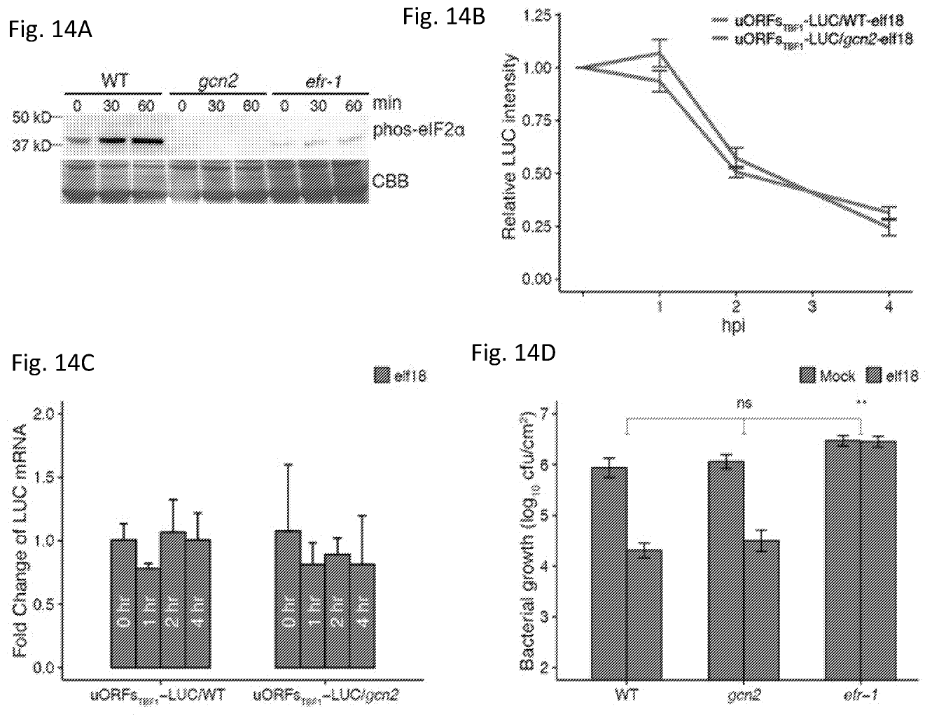

The molecular mechanisms by which any host, including Arabidopsis, activate immune-related translation are largely unknown. Besides uORF-mediated translation of key immune TFs, such as TBF1 in Arabidopsis¹ and ZIP-2 in C. elegans⁸, we identified the R-motif in the elf18-mediated TE-up transcripts. Both uORFs and R-motif normally inhibit translation of PTI-associated genes (FIG. 3 all parts). Upon immune induction, the inhibition is alleviated allowing rapid accumulation of defense proteins. In yeast, uORF inhibition on GCN4 translation is removed during starvation, when accumulation of uncharged tRNA activates GCN2 to phosphorylate and inactivate the translation initiation factor eIF2α³⁰. Surprisingly, we found that the only known eIF2α kinase in plants, GCN2³¹, is required for elf18-induced eIF2α phosphorylation, but not for elf18-induced TBF1 translation or resistance to bacteria (FIGS. 14A-14D), suggesting an alternative mechanism in immune-induced translational reprogramming in plants.

The inhibitory effect of R-motifs on translation is likely mediated by PAB proteins, since mutating either R-motif or PABs resulted in a reduction in responsiveness to elf18 induction (FIGS. 3 and 4 all parts). It has been reported that PABs can be post-translationally modified and regulated by interactors, which influence activities of PABs in translation²⁸. Further investigation will be required to dissect the regulatory mechanisms of R-motifs and understand the roles of PABs in different translation mechanisms, such as the internal ribosome entry site (IRES)-mediated translational activity observed in yeast³². Intriguingly, R-motif is also prevalent in mRNAs from other organisms, including the human p53 mRNA, suggesting a conserved regulatory mechanism may be shared across species.

### Methods

### Plant Growth, Transformation, and Treatment

Plants were grown on soil (Metro Mix 360) at 22° C. under 12/12-h light/dark cycles with 55% relative humidity. efr-1⁵, ers1-10 (a weak gain-of-function mutant)³³, ein4-1 (a gain-of-function mutant)¹⁸, wei7-4 (a loss-of-function mutant)¹⁹, eicbp.b (camta 1-3; SALK_108806)³⁴, pab2 pab4²⁹ and pab2 pab8²⁹ were previously described. efr7 (SALK_205018) and gcn2 (GABI_862B02) were from the Arabidopsis Biological Resource Center (ABRC). Transgenic plants were generated using the floral dip method³⁵.

### Ribo-Seq Library Construction

Leaves from ˜24 3-week-old plants (2 leaves/plant; ˜1.0 g) were collected. Tissue was fast frozen and ground in liquid nitrogen. 5 ml cold polysome extraction buffer [PEB; 200 mM Tris pH 9.0, 200 mM KCl, 35 mM MgCl₂, 25 mM EGTA, 5 mM DTT, 1 mM phenylmethanesulfonylfluoride (PMSF), 50 μg/ml cycloheximide, 50 μg/ml chloramphenicol, 1% (v/v) Brij-35, 1% (v/v) Igepal CA630, 1% (v/v) Tween 20, 1% (v/v) Triton X-100, 1% Sodium deoxycholate (DOC), 1% (v/v) polyoxyethylene 10 tridecyl ether (PTE)] was added. After thawing on ice for 10 min, lysate was centrifuged at 4° C./16,000 g for 2 min. Supernatant was transferred to 40 μm filter falcon tube and centrifuged at 4° C./7,000 g for 1 min. Supernatant was then transferred into a 2-ml tube and centrifuged at 4° C./16,000 g for 15 min and this step was repeated once. 0.25 ml lysate was saved for total RNA extraction for making the RNA-seq library. Another 1 ml lysate was layered on top of 0.9 ml sucrose cushion [400 mM Tris·HCl pH 9.0, 200 mM KCl, 35 mM MgCl₂, 1.75 M sucrose, 5 mM DTT, 50 μg/ml chloramphenicol, 50 μg/ml cycloheximide] in an ultracentrifuge tube (#349623, Beckman). The samples were then centrifuged at 4° C./70,000 rpm for 4 h in a TLA100.1 rotor. The pellet was washed twice with cold water, resuspended in 300 μl RNase I digestion buffer [20 mM Tris·HCl pH 7.4, 140 mM KCl, 35 mM MgCl₂, 50 μg/ml cycloheximide, 50 μg/ml chloramphenicol]¹¹ and then transferred to a new tube for brief centrifugation. The supernatant was then transferred to another new tube where 10 μl RNase I (100 U/μl) was added before 60 min incubation at 25° C. 15 μl SUPERase-In (20 U/μl) was then added to stop the reaction. The subsequent steps including ribosome recovery, footprint fragment purification, PNK treatment and linker ligation were performed as previously reported¹⁰. 2.5 μl of 5′ deadenylase (NEB) was then added to the ligation system and incubated at 30° C. for 1 h. 2.5 μl of RecJ_{f} exonuclease (NEB) was subsequently added for 1 h incubation at 37° C. The enzymes were inactivated at 70° C. for 20 min and 10 μl of the samples were taken as template for reverse transcription. The rest of the steps for the library construction were performed as in the reported protocol¹⁰, with the exception of using biotinylated oligos, rRNA1 and rRNA2, for Arabidopsis according to another reported method¹¹.

### RNA-Seq Library Construction

0.75 ml TRIzol® LS (Ambion) was added to the 0.25 ml lysate saved from the Ribo-seq library construction, from which total RNA was extracted, quantified and qualified using Nanodrop (Thermo Fisher Scientific Inc). 50-75 μg total RNA was used for mRNA purification with Dynabeads® Oligo (dT)₂₅ (Invitrogen). 20 μl of the purified poly (A) mRNA was mixed with 20 μl 2× fragmentation buffer (2 mM EDTA, 10 mM Na₂CO₃, 90 mM NaHCO₃) and incubated for 40 min at 95° C. before cooling on ice. 500 μl of cold water, 1.5 μl of GlycoBlue and 60 μl of cold 3 M sodium acetate were then added to the samples and mixed. Subsequently, 600 μl isopropanol was added before precipitation at −80° C. for at least 30 min. Samples were then centrifuged at 4° C./15,000 g for 30 min to remove all liquid and air dried for 10 min before resuspension in 5 μl of 10 mM Tris pH 8. The rest of the steps were the same as Ribo-seq library preparation.

### Plasmids

To construct the 35S:uORFs_{TBG1}-LUC reporter, the 35S promoter and the TBF1 exon1, including the R-motif, uORF1-uORF2 and the coding sequence of the first 73 amino acids of TBF1, were amplified from p35S:uORF1-uORF2-GUS¹ using Reporter-F/R primers, and ligated into pGWB235³⁶ via Gateway recombination. The 35S:ccdB cassette-LUC-NOS construct was generated by fusing PCR fragments of the 35S promoter from pMDC140³⁷, the ccdB cassette and the NOS terminator from pRNAi-LIC³⁸ and LUC from pGWB235³⁶. The 35S:ccdB cassette-LUC-NOS was then inserted into pCAMBIA1300 via PstI and EcoRI and designated as pGX301 for cloning 5′ leader sequences through replacement of the ApaI-flanked ccdB cassette³⁸. Similarly, the 35S:RLUC-HA-rbs terminator construct was made through fusion of PCR fragments of 35S from pMDC140³⁷, RLUC from pmirGLO (Promega, E1330) and rbs terminator from pCRG3301³⁹. The 35S:RLUC-HA-rbs fragment flanked with EcoRI was inserted into pTZ-57rt (Thermo fisher, K1213) via TA cloning to generate pGX125. 5′ leader sequences were amplified from the Arabidopsis (Col-0) genomic DNA or synthesized by Bio Basics (New York, USA) and inserted into pGX301 followed by transferring 35S:RLUC-HA-rbs from pGX125 via EcoRI. EFR, PAB2, PAB4 and PAB8 were amplified from U21686, C104970, U10212 and U15101 (from ABRC), respectively, and fused with the N-terminus of EGFP by PCR. Fusion fragments were then inserted between the 35S promoter and the rbs terminator to generate 35S:EFR-EGFP (pGX664), 35S:EFR (pGX665), and 35S:PAB2-EGFP (pGX694).

### LUC Reporter Assay and Dual Luciferase Assay

To record the 35S:uORFs_{TBG1}-LUC reporter activity, 3-week-old Arabidopsis plants were sprayed with 1 mM luciferin 12 h before infiltration with either 10 μM elf18 (synthesized by GenScript) or 10 mM MgCl₂ as Mock. Luciferase activity was recorded in a CCD camera-equipped box (Lightshade Company) with each exposure time of 20 min. For dual luciferase assay, N. benthamiana plants were grown at 22° C. under 12/12-h light/dark cycles. Dual luciferase constructs were transformed into the Agrobacterium strain GV3101, which was cultured overnight at 28° C. in LB supplied with kanamycin (50 mg/l), gentamycin (50 mg/l) and rifampicin (25 mg/l). Cells were then spun down at 2,600 g for 5 min, resuspended in infiltration buffer [10 mM 2-(N-morpholino) ethanesulfonic acid (MES), 10 mM MgCl₂, 200 μM acetosyringone], adjusted to OD₆₀₀ₙₘ=0.1, and incubated at room temperature for additional 4 h before infiltration using 1 ml needleless syringes. For elf18 induction, 10 mM MgCl₂ (Mock) solution or 10 μM elf18 were infiltrated 20 h after the dual luciferase construct and EFR-EGFP had been co-infiltrated at the ratio of 1:1, and samples were collected 2 h after treatment. For PAB2-EGFP co-expression assay, Agrobacterium containing a dual luciferase construct was mixed with Agrobacterium containing the PAB2-EGFP construct at the ratio of 1:5. Leaf discs were collected, ground in liquid nitrogen and lysed with the PLB buffer (Promega, E1910). Lysate was spun down at 15,000 g for 1 min, from which 10 μl was used for measuring LUC and RLUC activity using the Victor3 plate reader (PerkinElmer). At 25° C., substrates for LUC and RLUC were added using the automatic injector and after 3 s shaking and 3 s delay, the signals were captured for 3 s and recorded as CPS (counts per second).

### Elf18-Induced Growth Inhibition and Resistance to Psm ES4326

For elf18-induced growth inhibition assay, seeds were sterilized in a 2% PPM solution (Plant Cell Technology) at 4° C. for 3 d and sowed on MS media (1/2 MS basal salts, 1% sucrose, and 0.8% agar) with or without 100 nM elf18. 10-day-old seedlings were weighed with 10 seedlings per sample. For elf18-induced resistance to Psm ES4326, 1 μM elf18 or Mock (10 mM MgCl₂) was infiltrated into 3-week-old soil-grown plants 1 day prior to Psm ES4326 (OD₆₀₀ₙₘ=0.001) infection of the same leaf. Bacterial growth was scored 3 days after infection.

### Elf18-Induced MAPK Activation and Callose Deposition

For MAPK activation, 12-day-old seedlings grown on MS media were flooded with 1 μM elf18 solution and 25 seedlings were collected at indicated time points. Protein was extracted with co-IP buffer [50 mM Tris, pH 7.5, 150 mM NaCl, 0.1% (v/v) Triton X-100, 0.2% (v/v) Nonidet P-40, protease inhibitor cocktail (Roche), phos-stop phosphatase inhibitor cocktail (Roche)]. For callose deposition, 3-week-old soil-grown plants were infiltrated with 1 μM elf18. After 20 h of incubation, leaves were collected, decolorized in 100% ethanol with gentle shaking for 4 h and rehydrated in water for 30 min before stained in 0.01% (w/v) aniline blue in 0.01 M K₃PO4 pH 12 covered with aluminium foil for 24 h with gentle shaking. Callose deposition was observed with Zeiss-510 inverted confocal using 405 nm laser for excitation and 420-480 nm filter for emission.

### RNA-Pull Down of In Vitro and In Vivo Synthesized PAB Proteins

PAB2-EGFP was amplified from pGX694. GA, G[A]₃, and G[A]₆ were synthesized using Bio Basics (New York, USA) while poly(A) and G[A]ₙ were synthesized by IDT (www.idtdna.com/site). In vitro transcription and translation were performed with wheat germ translation system according to the manufacturer's instructions (BioSieg, Japan). To make biotin-labelled RNA probes, 2 μl of 10 mM biotin-16-UTP (11388908910, Roche) was added into the transcription system. DNase I was then used to remove the DNA template. 0.2 nmol biotin-labelled RNA was conjugated to 50 μl streptavidin magnetic beads (65001, Thermo Fisher) according to the manufacturer's instruction. In vitro synthesized PAB2-EGFP was incubated with biotin-labelled RNA in the glycerol-co-IP buffer [50 mM Tris, pH 7.5, 150 mM NaCl, 2.5 mM EDTA, 10% (v/v) glycerol, 1 mM PMSF, 20 U/mL Super-In RNase inhibitor, protease inhibitor cocktail (Roche)]. To perform in vivo pull down experiment, PAB2-EGFP was co-expressed with the elf18 receptor EFR (pGX665) for 40 h in N. benthamiana which was then treated with Mock or elf18 for 2 h. Protein was extracted with glycerol-co-IP buffer and used in the pull down assay at 4° C. for 4 h.

### Polysome Profiling

0.6 g Arabidopsis tissue was ground in liquid nitrogen with 2 ml cold PEB buffer. 1 ml crude lysate was loaded to 10.8 ml 15%-60% sucrose gradient and centrifuged at 4° C. for 10 h (35,000 rpm, SW 41 Ti rotor). A254 absorbance recording and fractionation were performed as described previously⁴⁰. Polysomal RNA was isolated by pelleting polysomes and TE was calculated as ratio of polysomal/total mRNA as described previously.

### Real-Time Reverse-Transcription Polymerase Chain Reaction (RT-PCR)

˜50 mg leaf tissue was used for total RNA extraction using TRIzol following the instruction (Ambion). After DNase I (Ambion) treatment, reverse transcription was performed following the instruction of SuperScript® III Reverse Transcriptase (Invitrogen) using oligo (dT). Real-time PCR was done using FastStart Universal SYBR Green Master (Roche).

### Bioinformatic and Statistical Analyses

Read processing and statistical methods were conducted following the criteria illuminated in FIG. 8 and Table 0. Generally, Bowtie2 was used to align reads to the Arabidopsis TAIR10 genome⁴¹. Read assignment was achieved using HT-seq⁴². Transcriptome and translatome changes were calculated using DESeq2⁴³. Transcriptome fold changes (RSfc) for protein-coding genes were determined using reads assigned to exon by gene. Translatome fold changes (RFfc) for protein-coding genes were measured using reads assigned to CDS by gene. TE was calculated by combining reads for all genes that passed RPKM≥1 in CDS threshold in two biological replicates and normalizing Ribo-seq RPKM to RNA-seq RPKM as reported¹⁵. The criteria used for uORF prediction are shown in FIG. 11 and performed using systemPipeR (github.com/tgirke/systemPipeR). The MEME online tool²³ was used to search strand-specific 5′ leader sequences for enriched consensuses compared to whole genome 5′ leader sequences with default parameters. Density plot was presented using IGB⁴⁴. Whole transcriptome R-motif search was performed using FIMO tool in the MEME suite²³. LUC/RLUC ratio was first tested for normal distribution using the Shapiro-Wilk test. Two-sided student's t-test was used for comparison between two samples. Two-sided one-way ANOVA or two-way ANOVA was used for more than two samples and Tukey test was used for multiple comparisons. GraphPad Prism 6 was used for all the statistical analyses. Unless specifically stated, sample size n means biological replicate and experiment has been performed three times with similar results. *P<0.05, **P<0.01, ***P<0.001, and ****P<0.0001 indicate significant increases; ns, no significance; †\\P<0.001 indicates a significant decrease.

### REFERENCES FOR EXAMPLE 1

1. Pajerowska-Mukhtar, K. M. et al. The HSF-like transcription factor TBF1 is a major molecular switch for plant growth-to-defense transition. Curr. Biol. 22, 103-112 (2012).
2. Huot, B., Yao, J., Montgomery, B. L. & He, S. Y. Growth-Defense Tradeoffs in Plants: A Balancing Act to Optimize Fitness. Mol. Plant 7, 1267-1287 (2014).
3. Couto, D. & Zipfel, C. Regulation of pattern recognition receptor signalling in plants. Nat. Rev. Immunol. 16, 537-552 (2016).
4. Wu, S. J., Shan, L. B. & He, P. Microbial signature-triggered plant defense responses and early signaling mechanisms. Plant Sci. 228, 118-126 (2014).
5. Zipfel, C. et al. Perception of the bacterial PAMP EF-Tu by the receptor EFR restricts Agrobacterium-mediated transformation. Cell 125, 749-760 (2006).
6. Zipfel, C. et al. Bacterial disease resistance in Arabidopsis through flagellin perception. Nature 428, 764-767 (2004).
7. Tintor, N. et al. Layered pattern receptor signaling via ethylene and endogenous elicitor peptides during Arabidopsis immunity to bacterial infection. Proc. Natl Acad. Sci. USA 110, 6211-6216 (2013).
8. Dunbar, T. L., Yan, Z., Balla, K. M., Smelkinson, M. G. & Troemel, E. R. C. elegans detects pathogen-induced translational inhibition to activate immune signaling. Cell Host Microbe 11, 375-386 (2012).
9. Luna, E. et al. Plant perception of beta-aminobutyric acid is mediated by an aspartyl-tRNA synthetase. Nat. Chem. Biol. 10, 450-456 (2014).
10. Ingolia, N. T., Brar, G. A., Rouskin, S., McGeachy, A. M. & Weissman, J. S. The ribosome profiling strategy for monitoring translation in vivo by deep sequencing of ribosome-protected mRNA fragments. Nat. Protoc. 7, 1534-1550 (2012).
11. Juntawong, P., Girke, T., Bazin, J. & Bailey-Serres, J. Translational dynamics revealed by genome-wide profiling of ribosome footprints in Arabidopsis. Proc. Natl Acad. Sci. USA 111, E203-212 (2014).
12. Liu, M. J. et al. Translational landscape of photomorphogenic Arabidopsis. Plant Cell 25, 3699-3710 (2013).
13. Merchante, C. et al. Gene-specific translation regulation mediated by the hormone-signaling molecule EIN2. Cell 163, 684-697 (2015).
14. Lei, L. et al. Ribosome profiling reveals dynamic translational landscape in maize seedlings under drought stress. Plant J. 84, 1206-1218 (2015).
15. Ingolia, N. T., Ghaemmaghami, S., Newman, J. R. S. & Weissman, J. S. Genome-wide analysis in vivo of translation with nucleotide resolution using ribosome profiling. Science 324, 218-223 (2009).
16. Liu, Z. X. et al. BIK1 interacts with PEPRs to mediate ethylene-induced immunity. Proc. Natl Acad. Sci. USA 110, 6205-6210 (2013).
17. Zipfel, C. Combined roles of ethylene and endogenous peptides in regulating plant immunity and growth. Proc. Natl Acad. Sci. USA 110, 5748-5749 (2013).
18. Hua, J. et al. EIN4 and ERS2 are members of the putative ethylene receptor gene family in Arabidopsis. Plant Cell 10, 1321-1332 (1998).
19. Stepanova, A. N., Hoyt, J. M., Hamilton, A. A. & Alonso, J. M. A Link between Ethylene and Auxin Uncovered by the Characterization of Two Root-Specific Ethylene-Insensitive Mutants in Arabidopsis. Plant Cell 17, 2230-2242 (2005).
20. Nakano, T., Suzuki, K., Fujimura, T. & Shinshi, H. Genome-wide analysis of the ERF gene family in Arabidopsis and rice. Plant Physiol. 140, 411-432 (2006).
21. von Arnim, A. G., Jia, Q. & Vaughn, J. N. Regulation of plant translation by upstream open reading frames. Plant Sci. 214, 1-12 (2014).
22. Barbosa, C., Peixeiro, I. & Romao, L. Gene expression regulation by upstream open reading frames and human disease. PLoS Genet. 9, e1003529 (2013).
23. Bailey, T. L. et al. MEME SUITE: tools for motif discovery and searching. Nucleic Acids Res. 37, W202-208 (2009).
24. Hinnebusch, A. G., Ivanov, I. P. & Sonenberg, N. Translational control by 5′-untranslated regions of eukaryotic mRNAs. Science 352, 1413-1416 (2016).
25. Eliseeva, I. A., Lyabin, D. N. & Ovchinnikov, L. P. Poly(A)-binding proteins: Structure, domain organization, and activity regulation. Biochemistry (Mosc) 78, 1377-1391 (2013).
26. Patel, G. P., Ma, S. & Bag, J. The autoregulatory translational control element of poly(A)-binding protein mRNA forms a heteromeric ribonucleoprotein complex. Nucleic Acids Res. 33, 7074-7089 (2005).
27. Belostotsky, D. A. Unexpected complexity of poly(A)-binding protein gene families in flowering plants: Three conserved lineages that are at least 200 million years old and possible auto- and cross-regulation. Genetics 163, 311-319 (2003).
28. Gallie, D. R. The role of the poly(A) binding protein in the assembly of the Cap-binding complex during translation initiation in plants. Translation (Austin) 2, e959378 (2014).
29. Dufresne, P. J., Ubalijoro, E., Fortin, M. G. & Laliberte, J. F. Arabidopsis thaliana class II poly(A)-binding proteins are required for efficient multiplication of turnip mosaic virus. J. Gen. Virol. 89, 2339-2348 (2008).
30. Hinnebusch, A. G. Translational regulation of GCN4 and the general amino acid control of yeast. Annu. Rev. Microbiol. 59, 407-450 (2005).
31. Browning, K. S. & Bailey-Serres, J. Mechanism of cytoplasmic mRNA translation. Arabidopsis Book 13, e0176 (2015).
32. Gilbert, W. V., Zhou, K. H., Butler, T. K. & Doudna, J. A. Cap-independent translation is required for starvation-induced differentiation in yeast. Science 317, 1224-1227 (2007).
33. Alonso, J. M. et al. Five components of the ethylene-response pathway identified in a screen for weak ethylene-insensitive mutants in Arabidopsis. Proc. Natl Acad. Sci. USA 100, 2992-2997 (2003).
34. Galon, Y. et al. Calmodulin-binding transcription activator 1 mediates auxin signaling and responds to stresses in Arabidopsis. Planta 232, 165-178 (2010).
35. Clough, S. J. & Bent, A.F. Floral dip: a simplified method for Agrobacterium-mediated transformation of Arabidopsis thaliana. Plant J. 16, 735-743 (1998).
36. Nakagawa, T. et al. Development of series of gateway binary vectors, pGWBs, for realizing efficient construction of fusion genes for plant transformation. J. Biosci. Bioeng. 104, 34-41 (2007).
37. Curtis, M. D. & Grossniklaus, U. A gateway cloning vector set for high-throughput functional analysis of genes in planta. Plant Physiol. 133, 462-469 (2003).
38. Xu, G. Y. et al. One-step, zero-background ligation-independent cloning intron-containing hairpin RNA constructs for RNAi in plants. New Phytol. 187, 240-250 (2010).
39. Li, J. T. et al. Modification of vectors for functional genomic analysis in plants. Genet. Mol. Res. 13, 7815-7825 (2014).
40. Mustroph, A., Juntawong, P. & Bailey-Serres, J. Isolation of plant polysomal mRNA by differential centrifugation and ribosome immunopurification methods. Methods Mol. Biol. 553, 109-126 (2009).
41. Langmead, B. & Salzberg, S. L. Fast gapped-read alignment with Bowtie 2. Nat. Methods 9, 357-359 (2012).
42. Anders, S., Pyl, P. T. & Huber, W. HTSeq—a Python framework to work with high-throughput sequencing data. Bioinformatics 31, 166-169 (2015).
43. Love, M. I., Huber, W. & Anders, S. Moderated estimation of fold change and dispersion for RNA-seq data with DESeq2. Genome Biol. 15, 550 (2014).
44. Nicol, J. W., Helt, G. A., Blanchard, S. G., Raja, A. & Loraine, A. E. The Integrated Genome Browser: free software for distribution and exploration of genome-scale datasets. Bioinformatics 25, 2730-2731 (2009).

### Example 2

### A Broadly Applicable Strategy For Enhancing Plant Disease Resistance With Minimal Fitness Penalty Using uORF-Mediated Translational Control

Controlling plant disease has been a struggle for mankind since the advent of agriculture^{1, 2}. Studies of plant immune mechanisms have led to strategies of engineering resistant crops through ectopic transcription of plants' own defense genes, such as the master immune regulatory gene NPR1³. However, enhanced resistance obtained through such strategies is often associated with significant penalties to fitness⁴⁻⁹, making the resulting products undesirable for agricultural applications. To remedy this problem, we sought more stringent mechanisms of expressing defense proteins. Based on our latest finding that translation of key immune regulators, such as TBF1¹⁰, is rapidly and transiently induced upon pathogen challenge (accompanying manuscript), we developed “TBF1-cassette” consisting of not only the immune-inducible promoter but also two pathogen-responsive upstream open reading frames (uORFs_{TBH1}) of the TBF1 gene. We demonstrate that inclusion of the uORFs_{TBF1}-mediated translational control over the production of snc1 (an autoactivated immune receptor) in Arabidopsis and AtNPR1 in rice enables us to engineer broad-spectrum disease resistance without compromising plant fitness in the laboratory or in the field. This broadly applicable new strategy may lead to reduced use of pesticides and lightening of selective pressure for resistant pathogens.

To meet the demand for food production caused by the explosion in world population while at the same time limiting pesticide pollution, new strategies must be developed to control crop diseases². As an alternative to the traditional chemical and breeding methods, studies of plant immune mechanisms have made it possible to engineer resistance through ectopic expression of plants' own resistance-conferring genes^{11, 12}. The first line of active defense in plants involves recognition of microbial/damage-associated molecular patterns (M/DAMPs) by host pattern-recognizing receptors (PRRs), and is known as pattern-triggered immunity (PTI)¹³. Ectopic expression of PRRs for MAMPs^{14, 15} and the DAMP signal eATP⁵, as well as in vivo release of the DAMP molecules, oligogalacturonides¹⁶, have all been shown to enhance resistance in transgenic plants. Besides PRR-mediated basal resistance, plant genomes encode hundreds of intracellular nucleotide-binding and leucine-rich repeat (NB-LRR) immune receptors (also known as “R proteins”) to detect the presence of pathogen effectors delivered inside plant cells¹⁷. Individual or stacked R genes have been transformed into plants to confer effector-triggered immunity (ETI)^{18, 19}. Besides PRR and R genes, NPR1 is another favourite gene used in engineering plant resistance¹¹. Unlike immune receptors that are activated by specific MAMPs and pathogen effectors, NPR1 is a positive regulator of broad-spectrum resistance induced by a general plant immune signal, salicylic acid³. Overexpression of the Arabidopsis NPR1 (AtNPR1) could enhance resistance in diverse plant families such as rice²⁰⁻²², wheat²³, tomato²⁴, and cotton²⁵ against a variety of pathogens.

A major challenge in engineering disease resistance, however, is to overcome the associated fitness costs⁴⁻⁹. In the absence of specialized immune cells, immune induction in plants involves switching from growth-related activities to defense^{10, 26}. Plants normally avoid autoimmunity by tightly controlling transcription, mRNA nuclear export and degradation of defense proteins²⁷. However, only transcriptional control has been used prevalently so far in engineering disease resistance^{4, 28}. Based on our global translatome analysis (accompanying manuscript), we discovered translation to be a fundamental layer of regulation during immune induction which can be explored to allow more stringent pathogen-inducible expression of defense proteins.

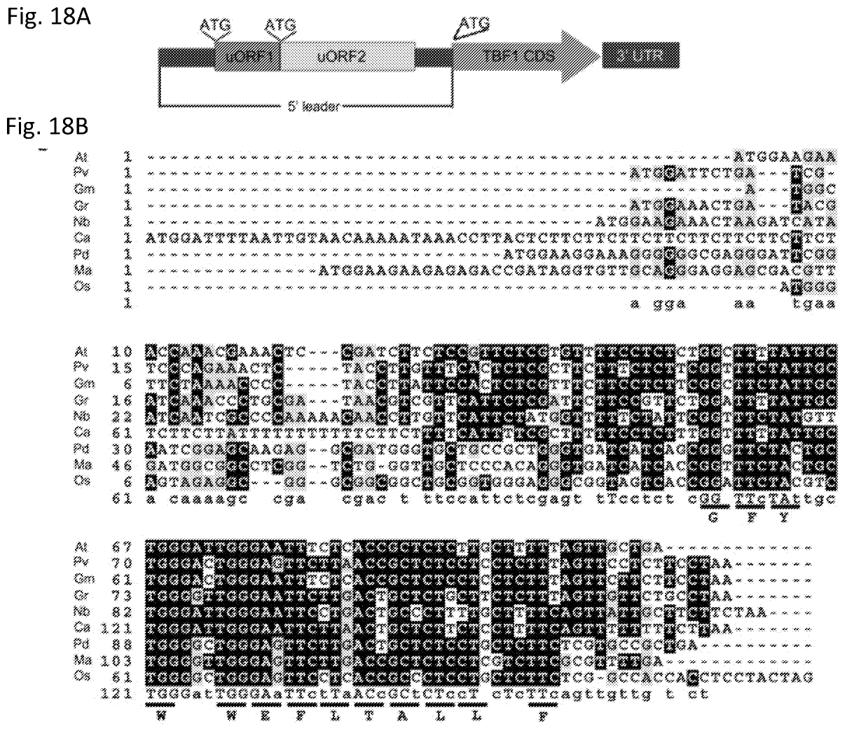

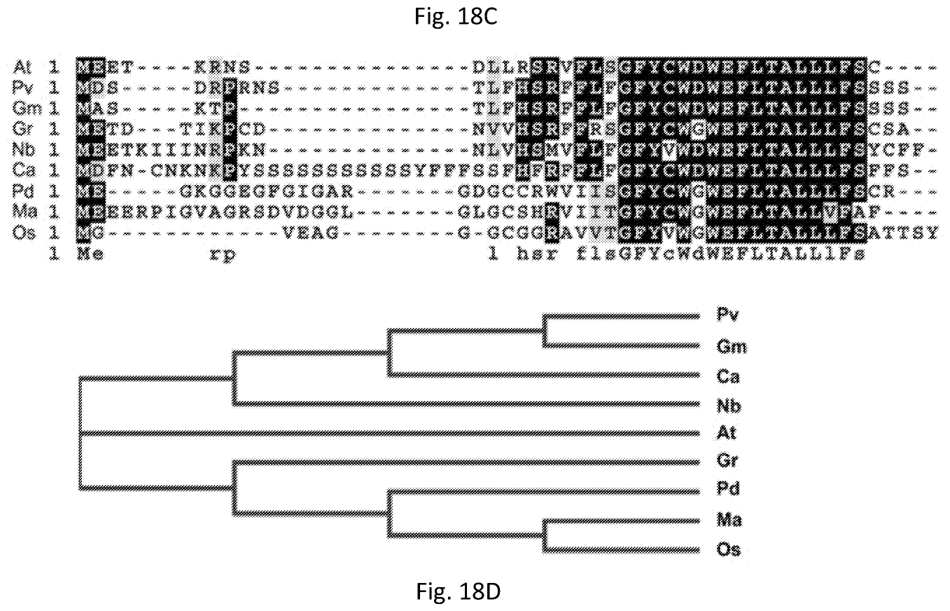

To test our hypothesis that tighter control of defense protein translation can minimize the fitness penalties associated with enhanced disease resistance, we used the TBF1 promoter (TBF1p) and the 5′ leader sequence (before the start codon for TBF1), which we designated as “TBF1-cassette”. TBF1 is an important transcription factor for the plant growth-to-defense switch upon immune induction¹⁰. Translation of TBF1 is normally suppressed by two uORFs within the 5′ leader sequence¹⁰. BLAST analysis showed that uORF2_{TBF1}, the major mRNA feature conferring the translational suppression (accompanying manuscript and ref¹⁰), is conserved across several plant species (>50% identity) (FIGS. 18A-D), suggesting an evolutionarily conserved control mechanism and a potential use of TBF1-cassette to regulate defense protein production in plant species other than Arabidopsis.

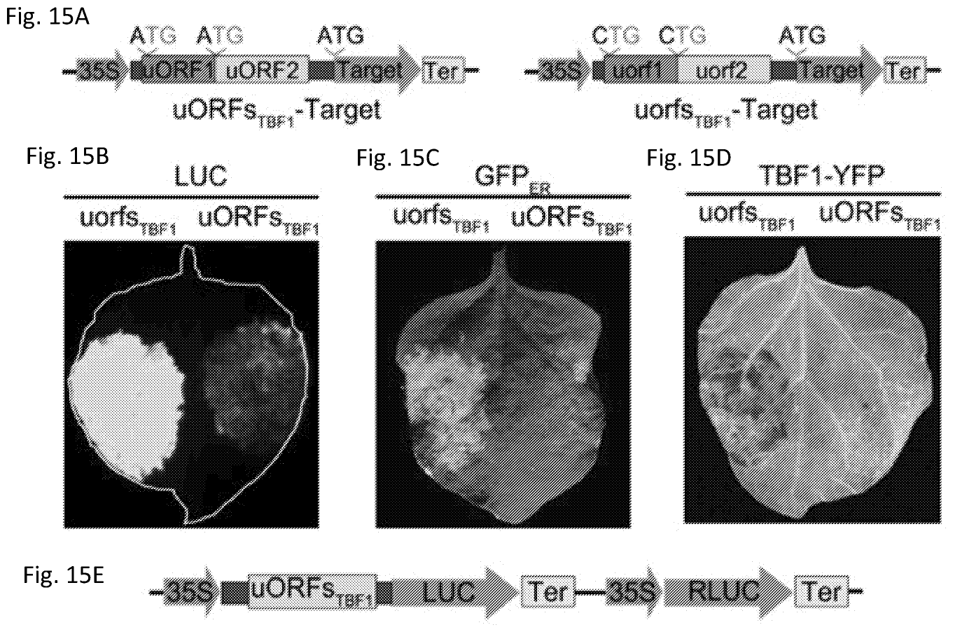

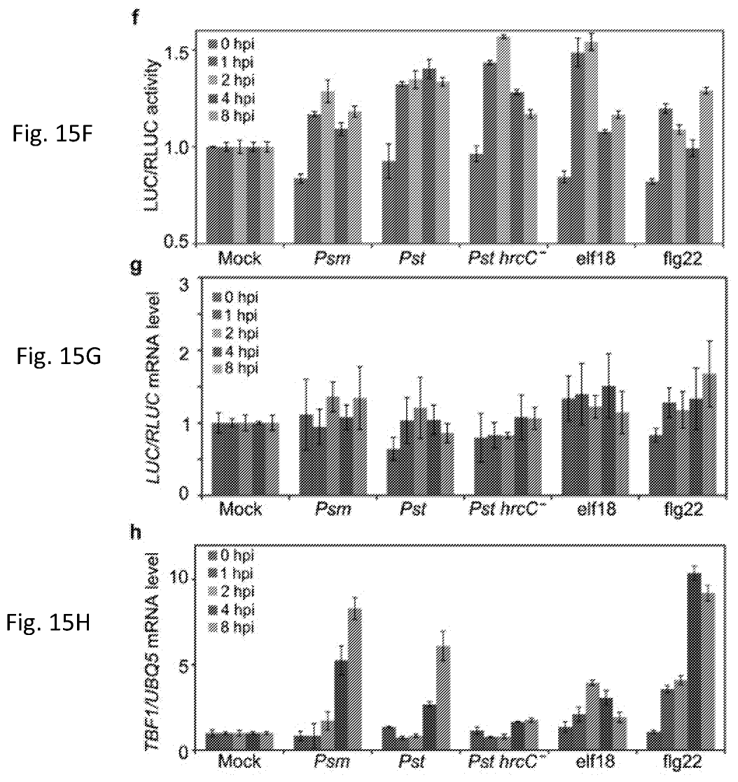

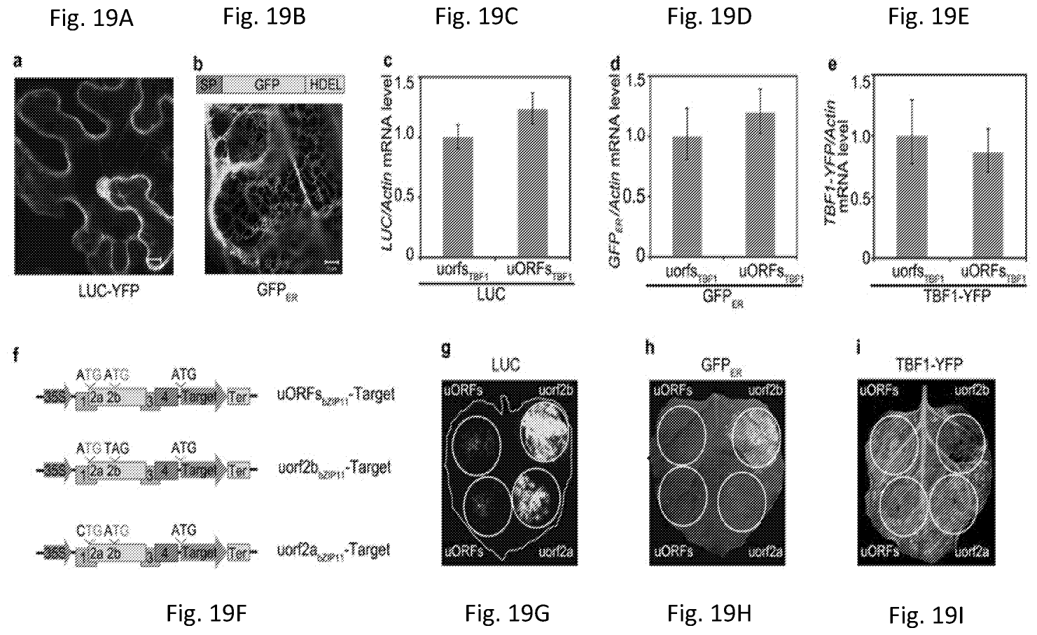

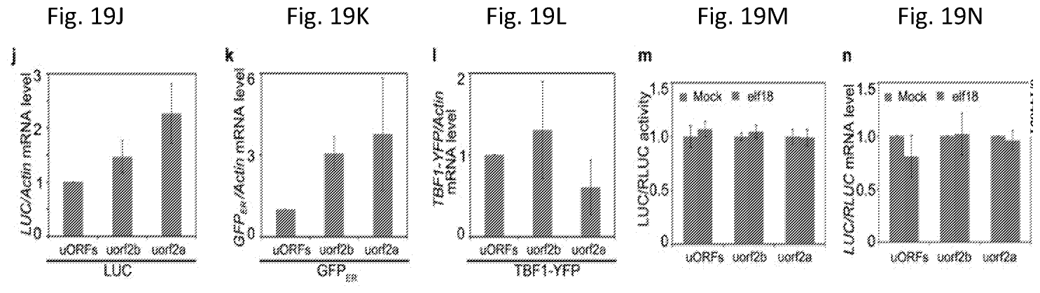

To explore the application of uORFs_{TBF1}, we first tested its capacity to control both cytosol- and ER-synthesized proteins (“Target”) using the firefly luciferase (LUC, FIG. 19A) and GFP_{ER} (FIG. 19B), respectively, as proxies under the control of wild-type (WT) uORFs_{TBF1} (35S:uORFs_{TBF1}-LUC/GFP_{ER}) or a mutant uorfs_{TBF1} (35S:uorfs_{TBF1}-LUC/GFP_{ER}) in which the ATG start codons for both uORFs were changed to CTG (FIG. 15A). Transient expression in Nicotiana benthamiana (N. benthamiana) showed that uORFs_{TBF1} could largely suppress both the cytosol-synthesized LUC and the ER-synthesized GFP_{ER} without significantly affecting mRNA levels (FIGS. 15B, 15C and FIGS. 19C, 19D). This uORFs_{TBF1}-mediated translational suppression was tight enough to prevent cell death induced by overexpression of TBF1 (TBF1-YFP) observed in 35S:uorfs_{TBF1}-TBF1-YFP (FIG. 15D and FIG. 19E). A similar repression activity was observed for another conserved uORF, uORF2b_{bZIP11} of the sucrose-responsive bZIP11 gene²⁹ (FIGS. 19F-L). However, unlike uORFs_{TBF1}, the uORF2b_{bZIP11}-mediated repression could not be alleviated by the MAMP signal elf18 (FIGS. 19M, 19N). These results support the potential utility of uORFs_{TBF1} in providing stringent control of cytosol- and ER-synthesized defense proteins specifically for engineering disease resistance.

To monitor the effect of uORFs_{TBF1} on translational efficiency (TE), a dual-luciferase system was constructed to calculate the ratio of LUC activity to the control renilla luciferase (RLUC) activity (FIG. 15E). We subjected transgenic plants harbouring this dual luciferase reporter to infection by the bacterial pathogens Pseudomonas syringae pv. maculicola ES4326 (Psm ES4326), Ps pv. tomato (Pst) DC3000, and the corresponding mutant of the type III secretion system Pst DC3000 hrcC⁻, as well as to treatments by the MAMP signals, elf18 and flg22. The rapid induction in the reporter TE within 1 h of both pathogen challenges and MAMP treatments suggests that it is likely a part of PTI, which does not involve bacterial type III effectors (FIG. 15F). The transient increases in translation were not correlated with significant changes in mRNA levels (FIG. 15G). In parallel, we examined the endogenous TBF1 mRNA levels from the TBF1p and found them to be elevated at later time points than the translational increases observed using the reporter (FIG. 15H). This suggests that in response to pathogen challenge, translational induction may precede transcriptional reprogramming in plants.

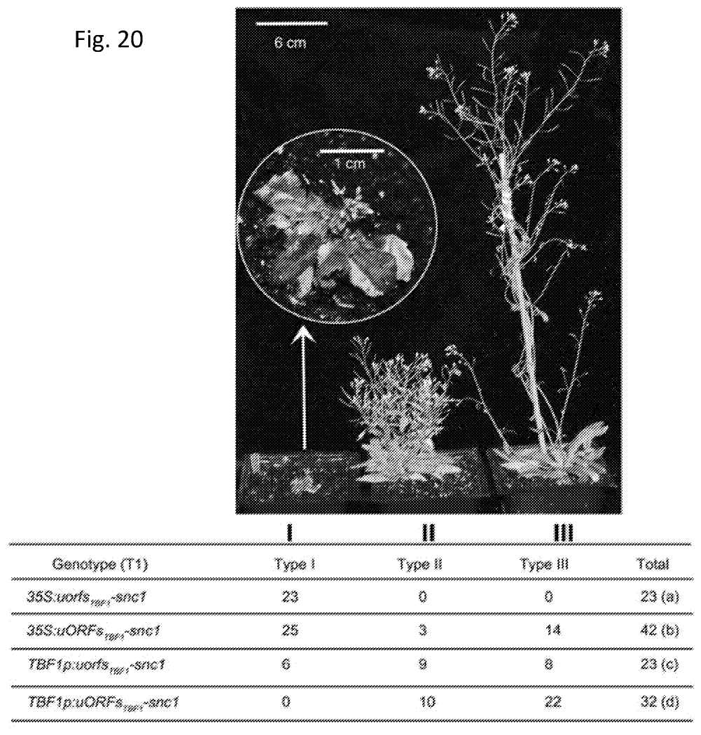

To engineer resistant plants using TBF1-cassette we picked two candidates from Arabidopsis, snc1-1³⁰ and NPR1²⁰. The Arabidopsis snc1-1 (for simplicity, snc1 from here on) is an autoactivated point mutant of the NB-LRR immune receptor SNC1. Even though the snc1 mutant plants have constitutively elevated resistance to various pathogens, their growth is significantly retarded³⁰. Such a growth defect is also prevalent in transgenic plants ectopically expressing the WT SNC1 by either the 35S promoter or its native promoter^{31, 32}, limiting the utility of SNC1, and perhaps other R genes, in engineering resistant plants. To overcome the fitness penalty associated with the snc1 mutant, we put it under the control of uORFs_{TBF1} driven by either the 35S promoter or TBF1p to create 35S:uORFs_{TBF1}-snc1 and TBF1p:uORFs_{TBF1}-snc1, respectively. As controls, we also generated 35S:uorfs_{TBF1}-snc1 and TBF1p:uorfs_{TBF1}-snc1, in which the start codons of the uORFs were mutated. The first generation of transgenic Arabidopsis (T1) with these four constructs displayed three distinct developmental phenotypes: Type I plants were small in rosette diameter, dwarf and with chlorosis (yellowing); Type II plants were healthier but still dwarf and with more branches; and Type III plants were indistinguishable from WT (FIG. 20). We found that regulating either transcription or translation of snc1 significantly improved plant growth as judged by the increased percentage of Type III plants. The highest percentage of Type III plants were found in TBF1p:uORFs_{TBF1}-snc1 transformants, in which snc1 was regulated by TBF1-cassette at both transcriptional and translational levels. The absence of Type I plants in these transformants clearly demonstrated the stringency of TBF1-cassette (FIG. 20).

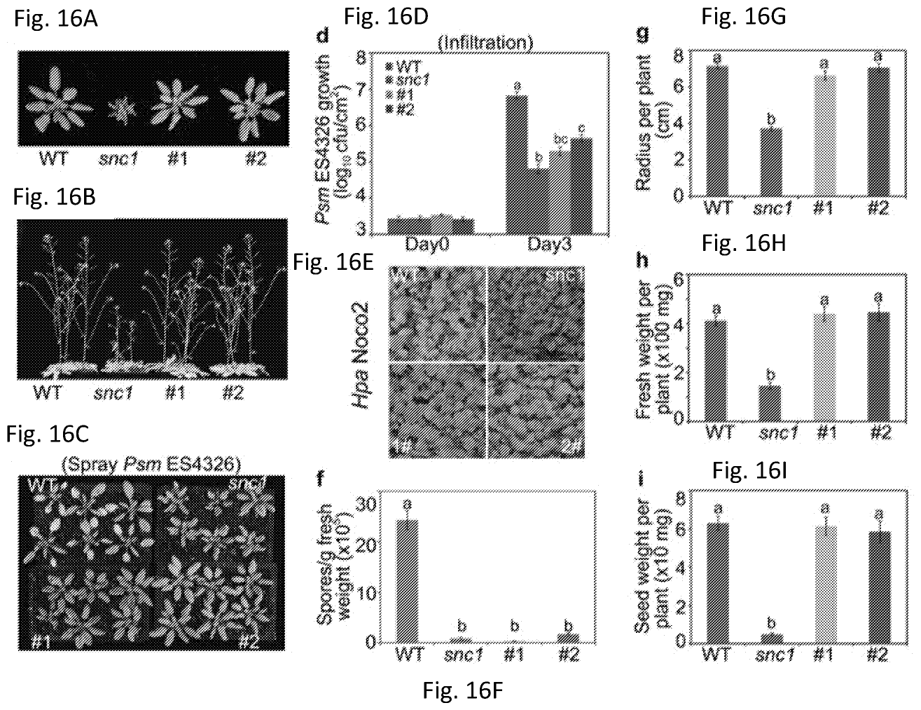

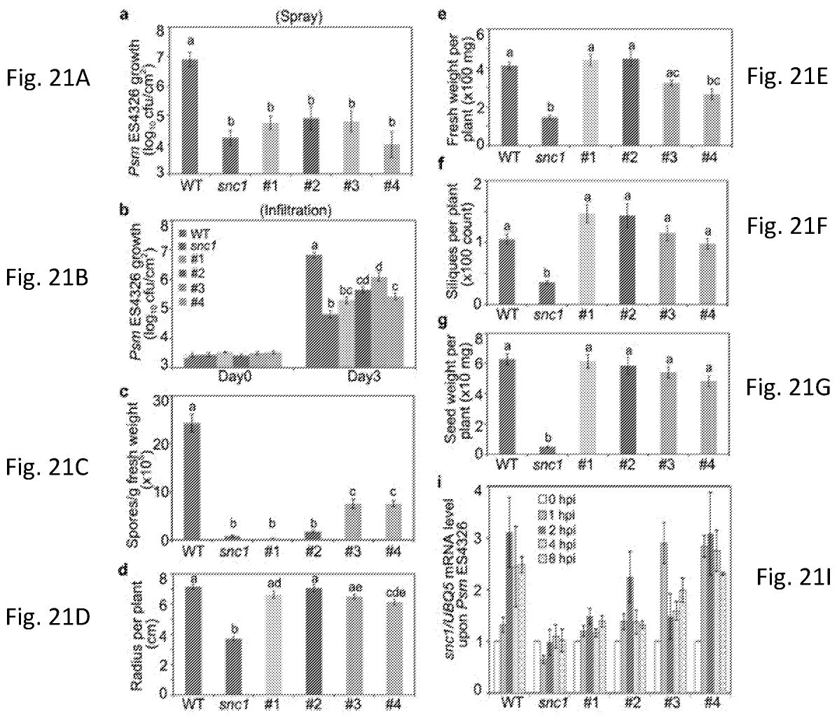

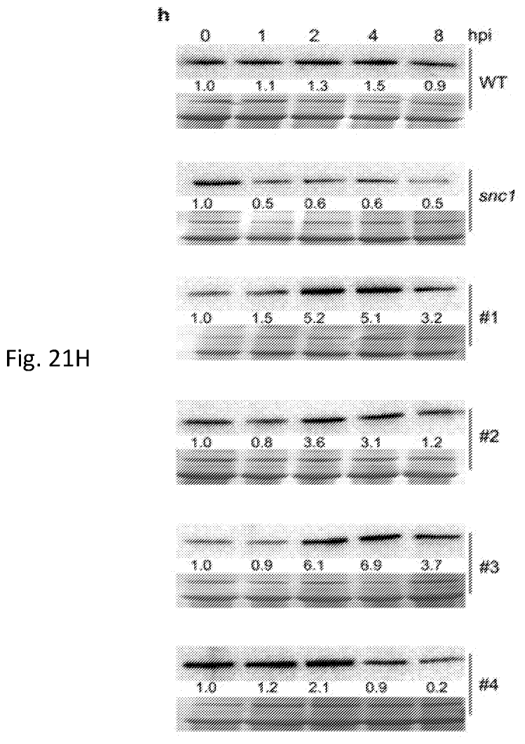

We propagated the transformants to obtain homozygotes for the transgene. For the TBF1p:uorfs_{TBF1}-snc1 and 35S:uORFs_{TBF1}-snc1 lines, most of the Type III plants in T1 showed the Type II phenotype as homozygotes, probably due to doubling of the transgene dosage. In contrast, most of the type III plants collected from the TBF1p:uORFs_{TBF1}-snc1 transformants maintained their normal growth phenotype as homozygotes. We then picked four independent TBF1p:uORFs_{TBF1}-snc1 lines for further disease resistance and fitness tests based on their similar appearance to WT plants (FIGS. 16A, 16B). We first showed that these transgenic lines indeed had elevated resistance to Psm ES4326, close to the level observed in the snc1 mutant by either spray inoculation or infiltration (FIGS. 16C, 16D and FIGS. 21A, 21B). They also displayed enhanced resistance to Hyaloperonospora arabidopsidis Noco2 (Hpa Noco2), an oomycete pathogen which causes downy mildew in Arabidopsis (FIGS. 16E, 16F and FIG. 21C). However, in contrast to snc1, these transgenic lines showed almost the same fitness as WT, as determined by rosette radius, fresh weight, silique (seed pod) number and total seed weight per plant (FIGS. 16G-I and FIGS. 21D-G). Upon Psm ES4326 challenge, we detected significant increases in the snc1 protein within 2 hpi in all four TBF1p:uORFs_{TBF1}-snc1 transgenic lines, but not in WT or snc1 (FIG. 21H). Comparison to the relatively modest changes in snc1 mRNA levels (FIG. 21I) suggests that these increases in the snc1 protein were most likely due to translational induction. These data provide a proof of concept that adding pathogen-inducible translational control is an effective way to enhance plant resistance without fitness costs.

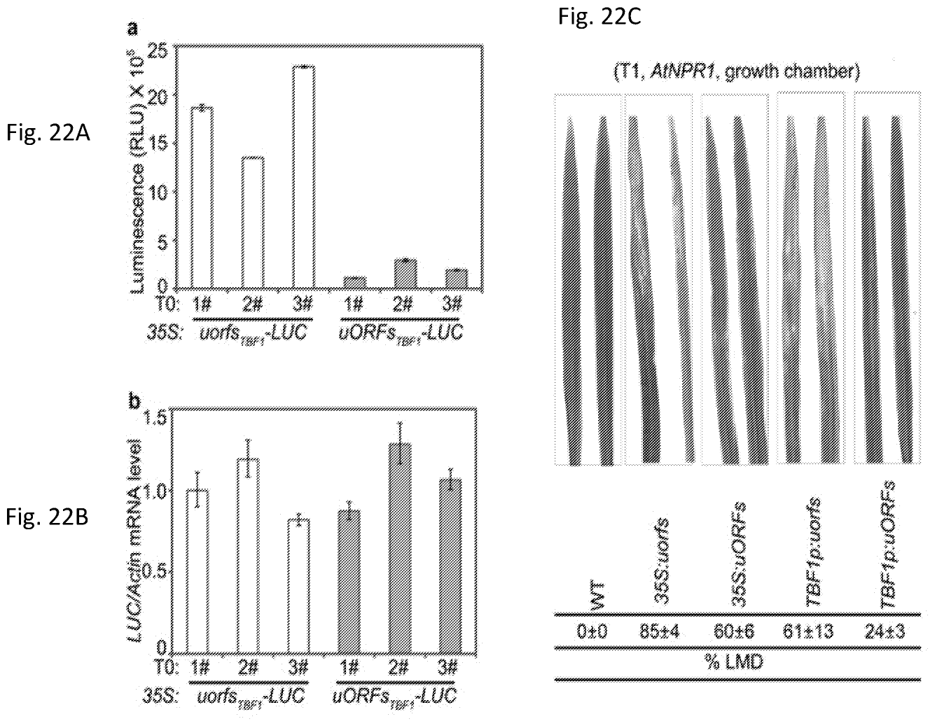

This result in Arabidopsis encouraged us to apply TBF1-cassette to engineering resistance in rice, which is not only a model organism for monocots but also one of the most important staple crops in the world. We first showed that the Arabidopsis uORFs_{TBF1}-mediated translational control is functional in rice by transforming 35S:uORFs_{TBF1}-LUC and 35S:uorfs_{TBF1}-LUC used in FIG. 15B into the rice (Oryza sativa) cultivar ZH11. The results clearly demonstrated that the Arabidopsis uORFs_{TBF1} could suppress translation of the reporter in rice without significantly influencing mRNA levels (FIGS. 22A, 22B).

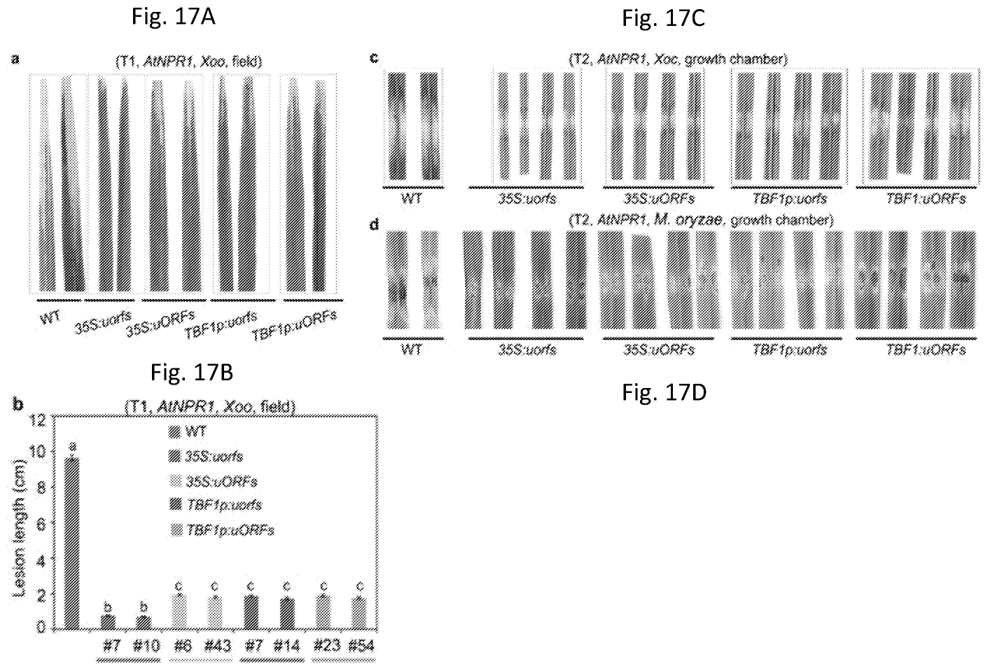

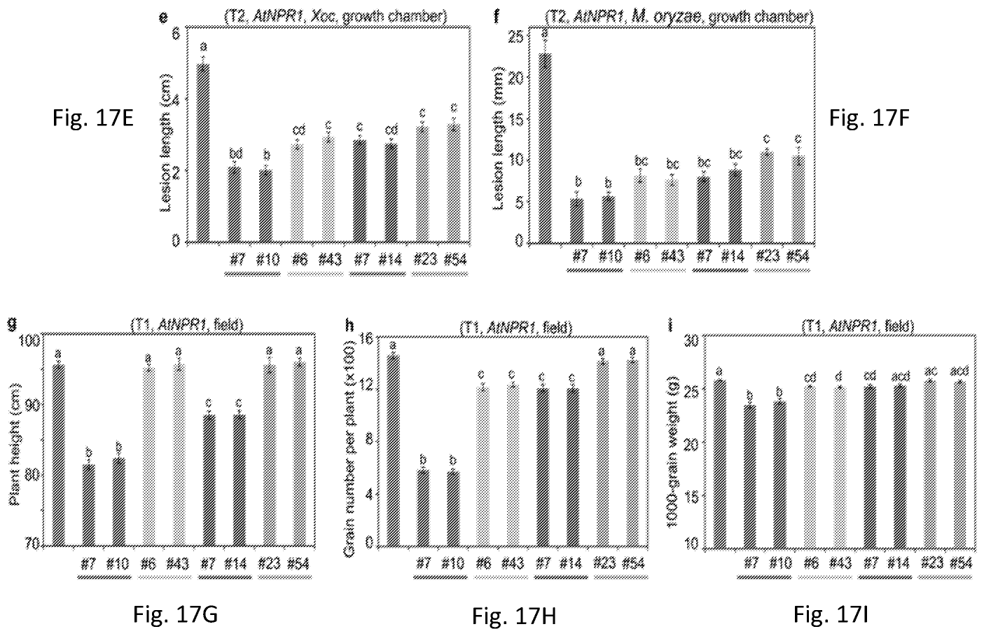

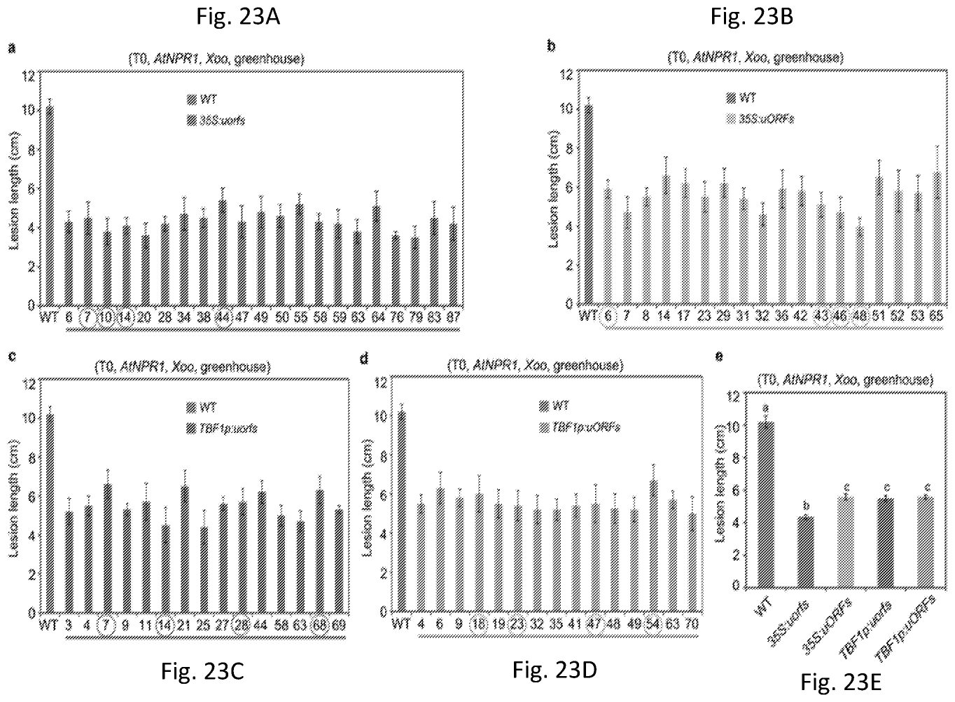

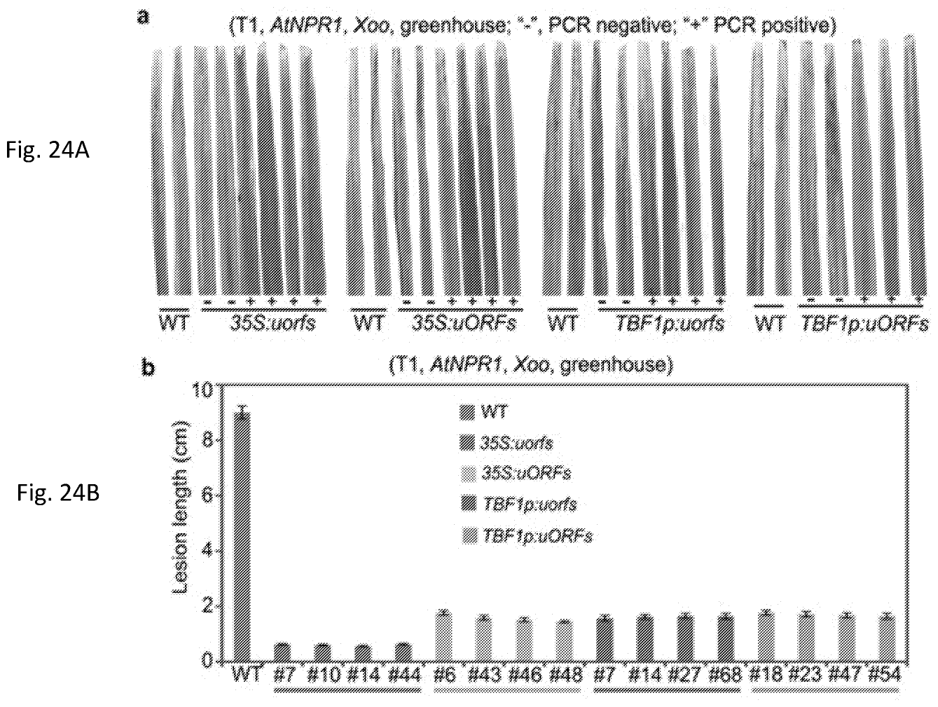

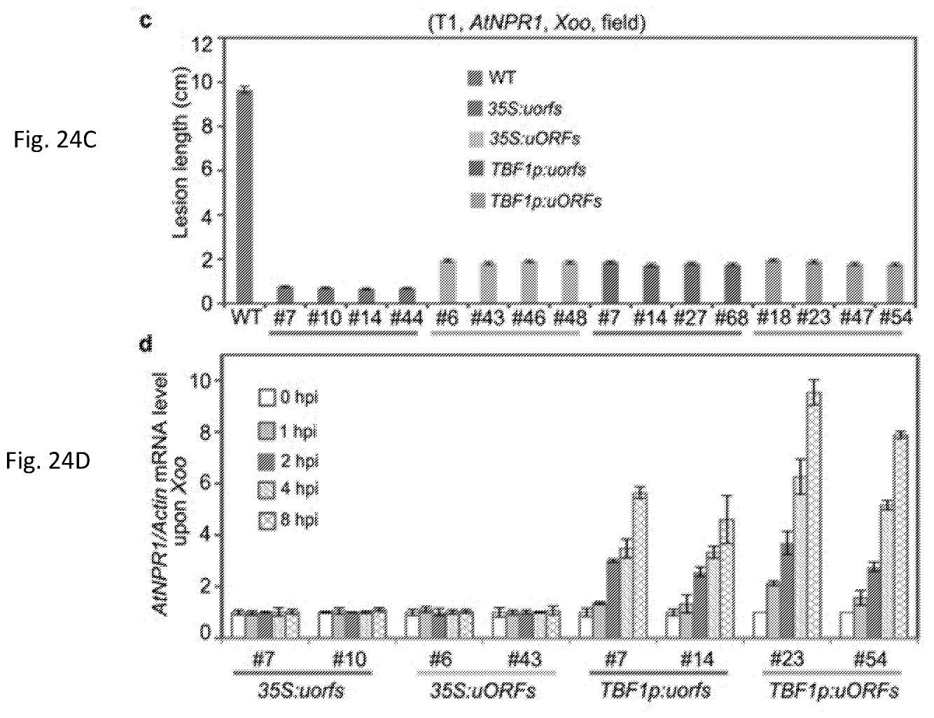

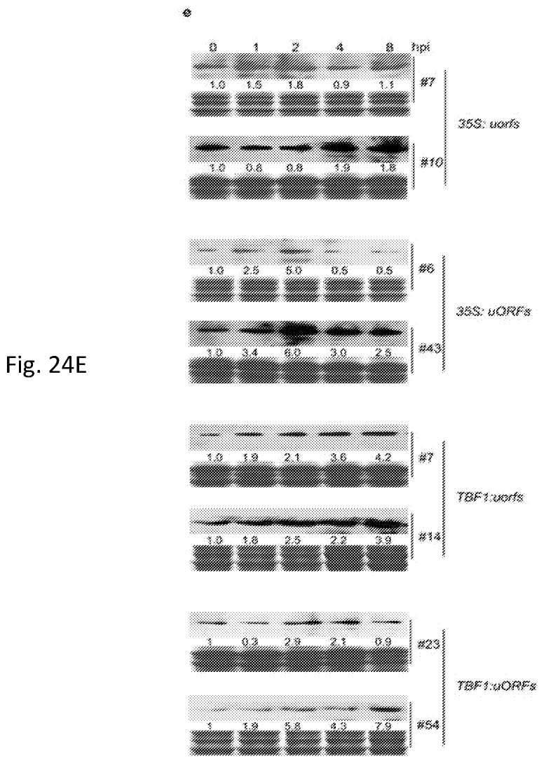

To engineer enhanced resistance in rice, we chose the Arabidopsis NPR1 (AtNPR1) gene³, which has been shown to confer broad-spectrum disease resistance in a variety of plants, including rice²⁰⁻²². However, rice plants overexpressing AtNPR1 by the maize ubiquitin promoter have been shown to have retarded growth and decreased seed size when grown in the greenhouse²¹. Additionally, they also developed the so-called lesion mimic disease (LMD) phenotype under certain environmental conditions, such as low light in the growth chamber^{8, 21}. To remedy the fitness problem, we expressed the AtNPR1-EGFP fusion gene under the following four regulatory systems: 35S:uorfs_{TBF1}-AtNPR1-EGFP, 35S:uORFs_{TBF1}-AtNPR1-EGFP, TBF1p:uorfs_{TBF1}-AtNPR1-EGFP and TBF1p:uORFs_{TBF1}-AtNPR1-EGFP. These four constructs were assigned different codes for blind testing of resistance and fitness phenotypes. Under growth chamber conditions, either the TBF1p-mediated transcriptional or the uORFs_{TBF1}-mediated translational control largely decreased the ratio and the severity of rice plants with LMD (FIG. 22C). However, the best results were obtained using TBF1-cassette with both transcriptional and translational control. Next, we tested resistance to the bacterial pathogen Xanthomonas oryzae pv. oryzae (Xoo), the causal agent for rice blight, in the first (T0 in rice research; FIGS. 23a-e) and the second (T1; FIGS. 24A, 24B) generations of transformants under the greenhouse conditions where LMD was not observed even for 35S:uorfs_{TBF1}-AtNPR1. Unsurprisingly, the 35S:uorfs_{TBF1}-AtNPR1 plants displayed the highest level of resistance to Xoo, due to the constitutive transcription and translation of AtNPR1. However, similar levels of resistance were also observed in plants with either transcriptional or translational control or with both (FIGS. 24A, 24B). Excitingly, these resistance results were faithfully reproduced in the field (FIGS. 17A, 17B and FIG. 24C). In response to Xoo challenge, transgenic lines with functional uORFs_{TBF1} displayed transient AtNPR1 protein increases which peaked around 2 hpi, even in the absence of significant changes in mRNA levels (e.g., 35S:uORFs_{TBF1}-AtNPR1 in FIG. 24d, e).

To determine the spectrum of AtNPR1-mediated resistance, we inoculated the third generation of transgenic rice plants (T2) with Xanthomonas oryzae pv. oryzicola (Xoc) and Magnaporthe oryzae (M. oryzae), the causal pathogens for rice bacterial leaf streak and fungal blast, respectively. We observed similar patterns of enhanced resistance against Xoc and M. oryzae in growth chambers designated for these controlled pathogens (FIGS. 17C-F) as for Xoo, confirming the broad spectrum of AtNPR1-mediated resistance. The lack of significant variation among the different transgenic lines suggests that they all have saturating levels of AtNPR1 in conferring resistance.

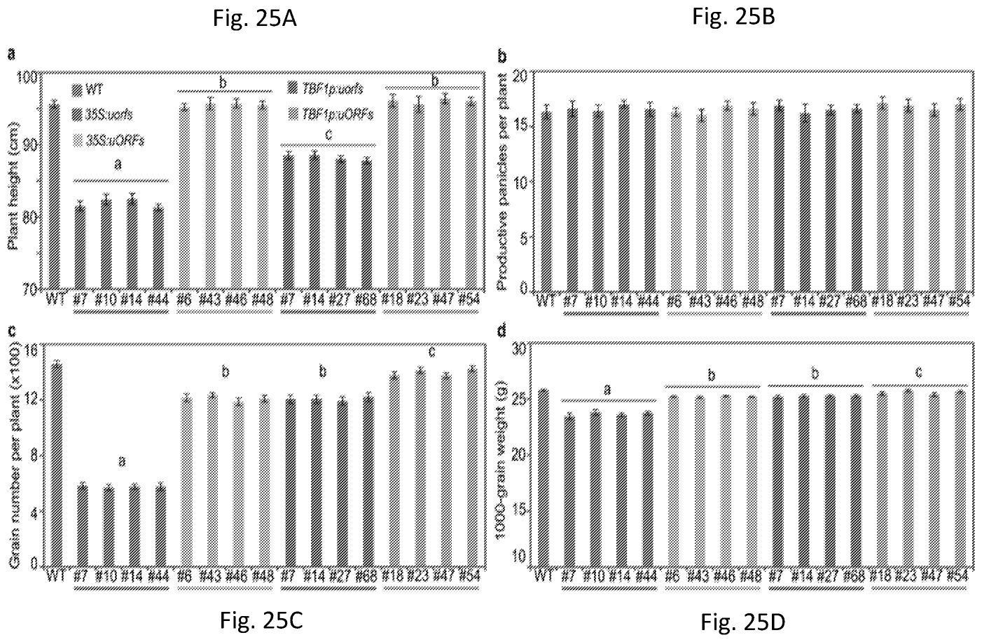

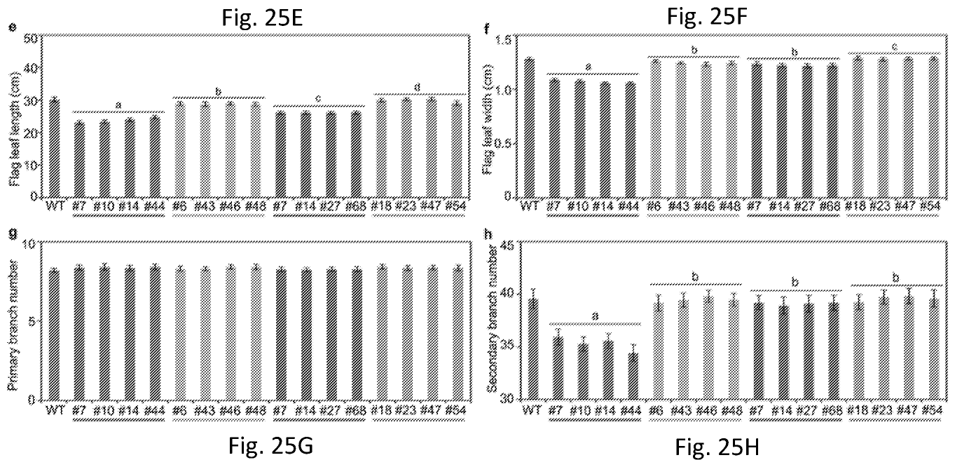

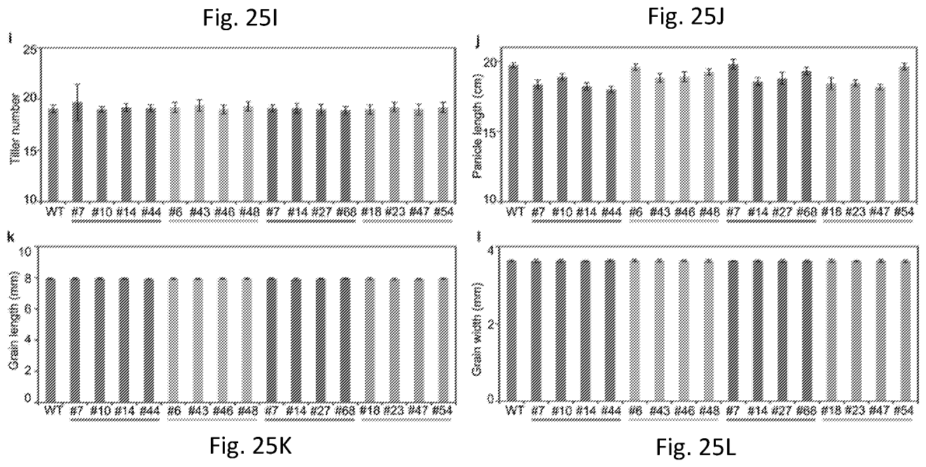

We then performed detailed fitness tests on these transgenic plants in the field. Consistent with a previous report on ectopic expression of the rice NPR1 homologue (OsNH1) by the 35S promoter³³, no obvious LMD was observed in any of the field-grown AtNPR1 transgenic rice plants. However, constitutive transcription and translation of AtNPR1 in 35S:uorfs_{TBF1}-AtNPR1 plants clearly had fitness penalties in flag leaf length and width, secondary branch number, plant height, and grain number and weight (FIGS. 17G-I and FIG. 25). Addition of transcriptional or/and translational control of AtNPR1 significantly reduced costs to these agronomically important traits, with the benefits of uORFs_{TBF1} highlighted in plant height, flag leaf length/width, and grain number per plant (FIGS. 17G, 17H and FIGS. 25E, 25F). As already observed in greenhouse experiments, combination of both transcriptional and translational control performed best in eliminating any fitness cost on yield as determined by two traits: number of grains per plant, and 1000-grain weight (FIGS. 17H, 17I), even though these plants had similar levels of disease resistance.

Using TBF1-cassette, we established a new strategy of controlling plant diseases, which cause 26% loss in crop production each year worldwide¹ and 30-40% loss in developing countries². Besides TBF1, more immune-responsive mRNA cis-elements as well as trans-acting regulators will become available through global translatome analyses. Our own ribosome footprint study of the PTI response has already revealed the functions of mRNA features such as uORFs and an mRNA consensus sequence “R-motif” in conferring translational responsiveness to PTI induction (accompanying manuscript). This translatome study also showed that translational activities are in general more stringently controlled than transcription, further emphasizing the importance of regulating translation in balancing defense and fitness. Using immune-inducible transcriptional and translational regulatory mechanisms to control defense protein expression can not only minimize the adverse effects of enhanced resistance on plant growth and development, but also help protect the environment through reduced demand for pesticides, a major source of pollution. Moreover, this inducible broad-spectrum resistance may be more difficult to overcome by a pathogen than constitutively expressed “gene-for-gene” resistance. The ubiquitous presence of uORFs in mRNAs of organisms ranging from yeast (13% of all mRNA)³⁴ to humans (49% of all mRNA)³⁵ suggests the potentially broad utility of these mRNA features for the precise control of transgene expression.

### Methods

### Arabidopsis Growth, Transformation, and Pathogen Infection

The Arabidopsis Col-0 accession was used for all experiments. Plants were grown on soil (Metro Mix 360) at 22° C. with 55% relative humidity (RH) and under 12/12-h light/dark cycles for bacterial growth assay and measurements of plant radius and fresh weight or 16/8-h light/dark cycles for seed weight and silique number measurements. Floral dip method³⁶ was used to generate transgenic plants. The BGL2:GUS reporter line³⁰ was used for snc1-related transformation. For infection, bacteria were first grown on the King's Broth medium plate at 28° C. for 2 d before resuspended in 10 mM MgCl₂ solution for infiltration. The antibiotic selection for Psm ES4326 was 100 μg/ml streptomycin, for Pst DC3000 25 μg/ml rifampicin, and for Pst DC3000 hrcC⁻ 25 μg/ml rifampicin and 30 μg/ml chloramphenicol. For spray inoculation, Psm ES4326 was transferred to liquid King's Broth with 100 μg/ml streptomycin, grown for another 8 to 12 h to OD₆₀₀ₙₘ=0.6 to 1.0 and sprayed at OD₆₀₀ₙₘ=0.4 in 10 mM MgCl₂ with 0.02% Silwet L-77. Infected leaf samples were collected on day 0 (4 biological replicates with 3 leaf discs each) and day 3 (8 replicates with 3 leaf discs each). For Hpa Noco2 infection, 12-day-old plants grown under 12/12-h light/dark cycles with 95% RH were sprayed with 4×10⁴ spores/ml and incubated for 7 d. Spores were collected by suspending infected plants in 1 ml water and counted in a hemocytometer under a microscopy.

Transient Expression in N. benthamiana

N. benthamiana plants were grown at 22° C. under 12/12-h light/dark cycles before used for Agrobacterium-mediated transient expression. Agrobacterium GV3101 transformed with each construct was grown in LB with kanamycin (50 μg/ml), gentamycin (50 μg/ml) and rifampicin (25 μg/ml) at 28° C. overnight. Cells were resuspended in the infiltration buffer [10 mM 2-(N-morpholino) ethanesulfonic acid (MES), 10 mM MgCl₂, 200 μM acetosyringone] at OD₆₀₀ₙₘ=0.1 and incubated at room temperature for 4 h before infiltration. For elf18 induction in N. benthamiana, the Agrobacterium harbouring the elf18 receptor-expressing construct (pGX664) was coinfiltrated with the Agrobacterium carrying the test construct at 1:1 ratio. 20 h later, the same leaves were infiltrated with 10 mM MgCl₂ (Mock) solution or 10 μM elf18 before leaf disc collection 2 h later.

### Dual-Luciferase Assay

The MgCl₂ solution (10 mM), Psm ES4326 (OD₆₀₀ₙₘ=0.02), Pst DC3000 (OD₆₀₀ₙₘ=0.02), Pst DC3000 hrcC⁻ (OD₆₀₀ₙₘ=0.02), elf18 (10 μM) or flg22 (10 μM), was infiltrated. Leaf discs were collected at the indicated time points. LUC and RLUC activities were measured as CPS (counts per second) using the Victor3 plate reader (PerkinElmer) according to the kit from Promega (E1910).

### Real-Time Polymerase Chain Reaction (PCR)

˜100 mg leaf tissue was collected for total RNA extraction with TRIzol (Ambion). DNase I (Ambion) treatment was performed before reverse transcription with SuperScript® III Reverse Transcriptase (Invitrogen) using oligo (dT). Real-time PCR was done using FastStart Universal SYBR Green Master (Roche).

### Rice Growth, Transformation, and Pathogen Infection

For LMD phenotype observation, rice was grown in greenhouse for 6 weeks and moved to a growth chamber for 3 weeks (12/12-h light/dark cycles, 28° C. and 90% RH). For fitness test, rice was grown during the normal rice growing season (From November 2015 to May 2016) under field conditions in Lingshui, Hainan (18° N latitude). Agrobacterium-mediated transformation into the Oryza sativa cultivar ZH11 was used to obtain transgenic rice plants³⁷. For Xoo infection in the greenhouse (performed in year 2016), rice was grown for 3 weeks from Feburary 2 and inoculated on Feburary 23 with data collection on March 8. For Xoo infection in the field (performed in year 2016), rice was grown on May 10 in the Experimental Stations of Huazhong Agricultural University, Wuhan, China (31° N latitude) and inoculated on July 20 with data collection on August 4. Xoo strains PXO347 and PXO99 were grown on nutrient agar medium (0.1% yeast extract, 0.3% beef extract, 0.5% polypeptone, and 1% sucrose) at 28° C. for 2 d before resuspension in sterile water and dilution to OD₆₀₀ₙₘ=0.5 for inoculation. 5 to 10 leaves of each plant were inoculated by the leaf-clipping method at the booting (panicle development) stage³⁸. Disease was scored by measuring the lesion length at 14 d post inoculation (dpi). PCR was performed using primer rice-F and rice-R for identification of AtNPR1 transgenic plants. Both PCR positive and negative T1 plants were scored. For Xoc infection in the growth chamber (performed in year 2016), rice was grown on October 20 and inoculated on November 15 with data collection on November 29. Xoc strain RH3 was grown on nutrient agar medium (0.1% yeast extract, 0.3% beef extract, 0.5% polypeptone, and 1% sucrose) at 28° C. for 2 d before resuspension in sterile water and dilution to OD₆₀₀ₙₘ=0.5 for inoculation. 5 to 10 leaves of each plant were inoculated by the penetration method using a needleless syringe at the tillering stage³⁸. Disease was scored by measuring the lesion length at 14 dpi. For M. oryzae infection in the growth chamber (performed in year 2016), rice was grown on October 15 and inoculated on November 16 with data collection on November 23. M. oryzae isolate RB22 was cultured on oatmeal tomato agar (OTA) medium (40 g oat, 150 ml tomato juice, 20 g agar for 1 L culture medium) at 28° C. 10 μl of the conidia suspension (5.0×10⁵ spores/ml) containing 0.05% Tween-20 was dropped to the press-injured spots on 5 to 10 fully expanded rice leaves and then wrapped with cellophane tape. Plants were maintained in darkness at 90% RH for one day and were grown under 12/12-h light/dark cycles with 90% RH. Disease was scored by measuring the lesion length at 7 dpi. For Xoc and M. oryzae, 3 independent transgenic lines for each construct were tested, with data from 2 lines shown in FIG. 17. For Xoo infection and fitness, 4 independent transgenic lines for each construct were tested, with data from 2 lines shown in FIG. 17 and from all four lines in FIGS. 24 and 25 all parts.

### Immunoblot

Arabidopsis tissue (100 mg) infected by Psm ES4326 (OD₆₀₀ₙₘ=0.02) was collected and lysed in 200 μl lysis buffer [50 mM Tris, pH 7.5, 150 mM NaCl, 0.1% Triton X-100, 0.2% Nonidet P-40, protease inhibitor cocktail (Roche, 1 tablet for 10 mL)] before centrifugation at 12,000 rpm for the supernatant. The same protocol was used to extract proteins from rice infected by Xoo (PXO99, at OD₆₀₀ₙₘ=0.5) using a slightly different lysis buffer [50 mM Tris-HCl, pH 7.5, 150 mM NaCl, 1 mM DTT, 1 mM PMSF, 2 mM EDTA, 0.1% Triton X-100, protease inhibitor cocktail (Roche, 1 tablet for 10 mL)].

### Plasmid Construction

The 35S promoter with duplicated enhancers was amplified from pRNAi-LIC³⁹ and flanked with PstI and XbaI sites using primers P1/P2. The NOS terminator was amplified from pRNAi-LIC and flanked with KpnI and EcoRI sites using primers P3/P4. Gateway cassette with LIC adapter sequences was amplified and flanked with KpnI and AflII sites using primers P5/P6/P7 (the PCR fragment by P5/P6 was used as template for P5/P7) from pDEST375 (GenBank: KC614689.1). The NOS terminator, the 35S promoter, and the Gateway cassette were sequentially ligated into pCAMBIA1300 (GenBank: AF234296.1) via KpnI/EcoRI, PstI/XbaI and KpnI/AflII, respectively. The resultant plasmid was used as an intermediate plasmid. The 5′ leader sequences of TBF1 (upstream of the ATG start codon of TBF1) with WT uORFs and mutant uorfs were amplified with P8/P9 and P8/P10 from the previously published plasmids¹⁰ carrying uORF1-uORF2-GUS and uorf1-uorf2-GUS, respectively, and cloned into the intermediate plasmid via XbaI/KpnI. The resultant plasmids were designated as pGX179 (35S:uORFs_{TBF1}-Gateway-NOS) and pGX180 (35S:uorfs_{TBF1}-Gateway-NOS). TBF1p was amplified from the Arabidopsis genomic DNA and flanked with HindIII/AscI using primers P11/P1, and the TBF1 5′ leader sequence was amplified from pGX180 and flanked with AscI/KpnI using primers P8/P13. The TBF1 promoter (P11/P12) and the TBF1 5′ leader sequence (P8/P13) were digested with AscI, ligated, and used as template for PCR and introduction of HindIII/KpnI using primer P11/P8. The 35S promoter in pGX179 was replaced by the TBF1 promoter to produce pGX1 (TBF1p:uORFs_{TBF1}-Gateway-NOS). The TBF1 promoter was amplified from the Arabidopsis genomic DNA and flanked with HindIII/SpeI using primers P14/P15 and ligated into pGX179, which was cut with HindIII/XbaI, to generate pGX181 (TBF1p:uorfs_{TBF1}-Gateway-NOS). LUC, GFP_{ER} and snc1 were amplified from pGWB235⁴⁰, GFP-HDEL⁴¹ and the snc1 mutant genomic DNA, respectively. TBF1-YFP and NPR1-EGFP were fused together through PCR, cloned via ligation independent cloning³⁹. EFR was amplified from U21686 (TAIR), fused with EGFP and controlled by the 35S promoter. The 5′ leader sequence of bZIP11 (containing uORFs_{bZIP11}) was amplified from the Arabidopsis genomic DNA with G904/G905. The start codons (ATG) for uORF2a and uORF2b in the 5′ leader sequence were mutated to CTG and TAG, respectively, to generate uorf2a_{bZIP11} and uorf2b_{bZIP11} by PCR using primers containing point mutations.

### Statistical Analyses

Normal distribution was tested using the Shapiro-Wilk test. Two-sided one-way ANOVA together with Tukey test was used for multiple comparisons. Unless specifically stated, sample size n means biological replicate. Experiments have been done three times with similar results for Arabidopsis study. GraphPad Prism 6 was used for all the statistical analyses.

### REFERENCES FOR EXAMPLE 2

1. Oerke, E. C. Crop losses to pests. J. Agric. Sci. 144, 31-43 (2006).
2. Flood, J. The importance of plant health to food security. Food Secur. 2, 215-231 (2010).
3. Fu, Z. Q. & Dong, X. N. Systemic acquired resistance: turning local infection into global defense. Annu. Rev. Plant Biol. 64, 839-863 (2013).
4. Gun, S. J. & Rushton, P. J. Engineering plants with increased disease resistance: how are we going to express it? Trends Biotechnol. 23, 283-290 (2005).
5. Bouwmeester, K. et al. The Arabidopsis lectin receptor kinase LecRK-I.9 enhances resistance to Phytophthora infestans in Solanaceous plants. Plant Biotechnol. J. 12, 10-16 (2014).
6. Tian, D., Traw, M. B., Chen, J. Q., Kreitman, M. & Bergelson, J. Fitness costs of R-gene-mediated resistance in Arabidopsis thaliana. Nature 423, 74-77 (2003).
7. Risk, J. M. et al. Functional variability of the Lr34 durable resistance gene in transgenic wheat. Plant Biotechnol. J. 10, 477-487 (2012).
8. Fitzgerald, H. A., Chern, M. S., Navarre, R. & Ronald, P. C. Overexpression of (At)NPR1 in rice leads to a BTH- and environment-induced lesion-mimic/cell death phenotype. Mol. Plant Microbe Interact. 17, 140-151 (2004).
9. Belbahri, L. et al. A local accumulation of the Ralstonia solanacearum PopA protein in transgenic tobacco renders a compatible plant-pathogen interaction incompatible. Plant J. 28, 419-430 (2001).
10. Pajerowska-Mukhtar, K. M. et al. The HSF-like transcription factor TBF1 is a major molecular switch for plant growth-to-defense transition. Curr. Biol. 22, 103-112 (2012).
11. Gun, S. J. & Rushton, P. J. Engineering plants with increased disease resistance: what are we going to express? Trends Biotechnol. 23, 275-282 (2005).
12. Piquerez, S. J. M., Harvey, S. E., Beynon, J. L. & Ntoukakis, V. Improving crop disease resistance: lessons from research on Arabidopsis and tomato. Front. Plant Sci. 5 (2014).
13. Boller, T. & Felix, G. A renaissance of elicitors: perception of microbe-associated molecular patterns and danger signals by pattern-recognition receptors. Annu. Rev. Plant Biol. 60, 379-406 (2009).
14. Schwessinger, B. et al. Transgenic expression of the dicotyledonous pattern recognition receptor EFR in rice leads to ligand-dependent activation of defense responses. Plos Pathog. 11 (2015).
15. Lacombe, S. et al. Interfamily transfer of a plant pattern-recognition receptor confers broad-spectrum bacterial resistance. Nat. Biotechnol. 28, 365-369 (2010).
16. Benedetti, M. et al. Plant immunity triggered by engineered in vivo release of oligogalacturonides, damage-associated molecular patterns. Proc. Natl Acad Sci. USA 112, 5533-5538 (2015).
17. Jones, J. D. G. & Dangl, J. L. The plant immune system. Nature 444, 323-329 (2006).
18. Dangl, J. L., Horvath, D. M. & Staskawicz, B. J. Pivoting the plant immune system from dissection to deployment. Science 341, 746-751 (2013).
19. Kim, S. H., Qi, D., Ashfield, T., Helm, M. & Innes, R. W. Using decoys to expand the recognition specificity of a plant disease resistance protein. Science 351, 684-687 (2016).
20. Chern, M. S. et al. Evidence for a disease-resistance pathway in rice similar to the NPR1-mediated signaling pathway in Arabidopsis. Plant J. 27, 101-113 (2001).
21. Quilis, J., Penas, G., Messeguer, J., Brugidou, C. & Segundo, B. S. The Arabidopsis AtNPR1 inversely modulates defense responses against fungal, bacterial, or viral pathogens while conferring hypersensitivity to abiotic stresses in transgenic rice. Mol. Plant Microbe Interact. 21, 1215-1231 (2008).
22. Molla, K. A. et al. Tissue-specific expression of Arabidopsis NPR1 gene in rice for sheath blight resistance without compromising phenotypic cost. Plant Sci. 250, 105-114 (2016).
23. Makandar, R., Essig, J. S., Schapaugh, M. A., Trick, H. N. & Shah, J. Genetically engineered resistance to Fusarium head blight in wheat by expression of Arabidopsis NPR1. Mol. Plant Microbe Interact. 19, 123-129 (2006).
24. Lin, W. C. et al. Transgenic tomato plants expressing the Arabidopsis NPR1 gene display enhanced resistance to a spectrum of fungal and bacterial diseases. Transgenic Res. 13, 567-581 (2004).
25. Kumar, V., Joshi, S. G., Bell, A. A. & Rathore, K. S. Enhanced resistance against Thielaviopsis basicola in transgenic cotton plants expressing Arabidopsis NPR1 gene. Transgenic Res. 22, 359-368 (2013).
26. Huot, B., Yao, J., Montgomery, B. L. & He, S. Y. Growth-defense tradeoffs in plants: a balancing act to optimize fitness. Mol. Plant 7, 1267-1287 (2014).
27. Johnson, K. C. M., Dong, O. X., Huang, Y. & Li, X. A rolling stone gathers no moss, but resistant plants must gather their moses. Cold Spring Harb. Symp. Quant. Biol. 77, 259-268 (2012).
28. Liu, W. & Stewart, C. N., Jr. Plant synthetic promoters and transcription factors. Curr. Opin. Biotechnol. 37, 36-44 (2015).
29. Rahmani, F. et al. Sucrose control of translation mediated by an upstream open reading frame-encoded peptide. Plant Physiol. 150, 1356-1367 (2009).
30. Li, X., Clarke, J. D., Zhang, Y. L. & Dong, X. N. Activation of an EDS1-mediated R-gene pathway in the snc1 mutant leads to constitutive, NPR1-independent pathogen resistance. Mol. Plant Microbe Interact. 14, 1131-1139 (2001).
31. Li, Y. Q., Yang, S. H., Yang, H. J. & Hua, J. The TIR-NB-LRR gene SNC1 is regulated at the transcript level by multiple factors. Mol. Plant Microbe Interact. 20, 1449-1456 (2007).
32. Yi, H. & Richards, E. J. A cluster of disease resistance genes in Arabidopsis is coordinately regulated by transcriptional activation and RNA silencing. Plant Cell 19, 2929-2939 (2007).
33. Yuan, Y. X. et al. Functional analysis of rice NPR1-like genes reveals that OsNPR1/NH1 is the rice orthologue conferring disease resistance with enhanced herbivore susceptibility. Plant Biotechnol. J. 5, 313-324 (2007).
34. Lawless, C. et al. Upstream sequence elements direct post-transcriptional regulation of gene expression under stress conditions in yeast. BMC Genomics 10, 7 (2009).
35. Calvo, S. E., Pagliarini, D. J. & Mootha, V. K. Upstream open reading frames cause widespread reduction of protein expression and are polymorphic among humans. Proc. Natl Acad Sci. USA 106, 7507-7512 (2009).
36. Clough, S. J. & Bent, A. F. Floral dip: a simplified method for Agrobacterium-mediated transformation of Arabidopsis thaliana. Plant J. 16, 735-743 (1998).
37. Lin, Y. J. & Zhang, Q. Optimising the tissue culture conditions for high efficiency transformation of indica rice. Plant Cell Rep. 23, 540-547 (2005).
38. Yuan, M. et al. A host basal transcription factor is a key component for infection of rice by TALE-carrying bacteria. Elife 5 (2016).
39. Xu, G. Y. et al. One-step, zero-background ligation-independent cloning intron-containing hairpin RNA constructs for RNAi in plants. New Phytol. 187, 240-250 (2010).
40. Nakagawa, T. et al. Development of series of gateway binary vectors, pGWBs, for realizing efficient construction of fusion genes for plant transformation. J. Biosci. Bioeng. 104, 34-41 (2007).
41. Xu, G. et al. Plant ERD2-like proteins function as endoplasmic reticulum luminal protein receptors and participate in programmed cell death during innate immunity. Plant J. 72, 57-69 (2012).

We claim:

1. A DNA construct comprising a heterologous promoter operably connected to a DNA polynucleotide encoding a RNA transcript comprising a 5′ regulatory sequence located 5′ to an insert site, wherein the 5′ regulatory sequence comprises an R-motif sequence.

2. The DNA construct of claim 1, wherein the 5′ regulatory sequence lacks a TBF1 uORF sequence.

3. The DNA construct of any one of the preceding claims, wherein the 5′ regulatory sequence comprises at least two R-motif sequences.

4. The DNA construct of any one of the preceding claims, wherein the 5′ regulatory sequence comprises between 5 and 25 R-motif sequences.

5. The DNA construct of any one of the preceding claims, wherein the R-motif sequences are separated by 0 nucleotides.

6. The DNA construct of any one of the preceding claims, wherein the R-motif comprises any one of the sequences of SEQ ID NOs: 113-293, a polynucleotide 15 nucleotides in length comprising G and A nucleotides in any ratio from 1G:1A to 1G:14A, or a variant thereof.

7. The DNA construct of any one of the preceding claims, wherein the 5′ regulatory sequence further comprises a uORF polynucleotide encoding any one of the uORF polypeptides of SEQ ID NOs: 1-38, or a variant thereof.

8. The DNA construct of any one of the preceding claims, wherein the 5′ regulatory sequence comprises any one of the polynucleotides of SEQ ID NOs: 39-76 or a variant thereof

9. The DNA construct of any one of the preceding claims, wherein the 5′ regulatory sequence comprises any one of the polynucleotides of SEQ ID NOs: 77-112, SEQ ID NOs: 294-474, or a variant thereof.

10. A DNA construct comprising a heterologous promoter operably connected to a DNA polynucleotide encoding a RNA transcript comprising a 5′ regulatory sequence located 5′ to an insert site, wherein the 5′ regulatory sequence comprises a uORF polynucleotide encoding any one of the uORF polypeptides of SEQ ID NOs: 1-38 or a variant thereof.

11. The DNA construct of claim 10, wherein the 5′ regulatory sequence comprises any one of the polynucleotides of SEQ ID NOS: 39-76, or a variant thereof.

12. The DNA construct of claim 10 or 11, wherein the 5′ regulatory sequence comprises any one of the polynucleotides of SEQ ID NOs: 77-112, SEQ ID NOs: 294-474, or a variant thereof.

13. The DNA construct of any one of the preceding claims, wherein the insert site comprises a heterologous coding sequence encoding a heterologous polypeptide.

14. The DNA construct of any one of the preceding claims, wherein the heterologous polypeptide comprises a plant pathogen resistance polypeptide.

15. The DNA construct of claim 13, wherein the plant pathogen resistance polypeptide is selected from the group consisting of snc-1 and NPR1.

16. The DNA construct of any one of the preceding claims, wherein the heterologous promoter comprises a plant promoter.

17. The DNA construct of any one of the preceding claims, wherein the heterologous promoter comprises a plant promoter inducible by a plant pathogen or chemical inducer.

18. A vector comprising the DNA construct of any one of claims 1-17.

19. The vector of claim 18, wherein the vector comprises a plasmid.

20. A cell comprising the DNA construct of any one of claims 1-17 or the vector of any one of claims 18-19.

21. The cell of claim 20, wherein the cell is a plant cell.

22. The cell of claim 21, wherein the cell is selected from the group consisting of a corn plant cell, a bean plant cell, a rice plant cell, a soybean plant cell, a cotton plant cell, a tobacco plant cell, a date palm cell, a wheat cell, a tomato cell, a banana plant cell, a potato plant cell, a pepper plant cell, a moss plant cell, a parsley plant cell, a citrus plant cell, an apple plant cell, a strawberry plant cell, a rapeseed plant cell, a cabbage plant cell, a cassava plant cell, and a coffee plant cell.

23. A plant comprising any one of the DNA constructs, vectors, or cells of claims 1-22.

24. The plant of claim 23, wherein the plant is selected from the group consisting of a corn plant, a bean plant, a rice plant, a soybean plant, a cotton plant, a tobacco plant, a date palm plant, a wheat plant, a tomato plant, a banana plant, a potato plant, a pepper plant, a moss plant, a parsley plant, a citrus plant, an apple plant, a strawberry plant, a rapeseed plant, a cabbage plant, a cassava plant, and a coffee plant.

25. A method for controlling the expression of a heterologous polypeptide in a cell comprising introducing the construct of any one of claims 13-17 or the vector of claims 18-19 into the cell.

26. The method of claim 25, wherein the cell is a plant cell.

27. The method of claim 26, wherein the cell is selected from the group consisting of a corn plant cell, a bean plant cell, a rice plant cell, a soybean plant cell, a cotton plant cell, a tobacco plant cell, a date palm cell, a wheat cell, a tomato cell, a banana plant cell, a potato plant cell, a pepper plant cell, a moss plant cell, a parsley plant cell, a citrus plant cell, an apple plant cell, a strawberry plant cell, a rapeseed plant cell, a cabbage plant cell, a cassava plant cell, and a coffee plant cell.

28. The method of any one of claims 25-27, further comprising purifying the heterologous polypeptide from the cell.

29. The method of claim 28, further comprising formulating the heterologous polypeptide into a therapeutic for administration to a subject.

30. A DNA construct comprising a heterologous promoter operably connected to a DNA polynucleotide encoding a RNA transcript comprising a 5′ regulatory sequence located 5′ to a heterologous coding sequence encoding an AtNPR polypeptide comprising SEQ ID NO: 475, wherein the 5′ regulatory sequence comprises SEQ ID NO: 476 (uORFs_{TBF1}).

31. The DNA construct of claim 30, wherein the heterologous promoter comprises SEQ ID NO: 477 (35S promoter) or SEQ ID NO: 478 (TBF1p).

32. The DNA construct of any one of claims 30-32, wherein the DNA construct comprises SEQ ID NO: 479 (35S:uORFs_{TBF1}-AtNPR1) or SEQ ID NO: 480 (TBF1p:uORFs_{TBF1}-AtNPR1).

33. A plant comprising any one of the DNA constructs of claims 30-32.

34. The plant of claim 34, wherein the plant is selected from the group consisting of a corn plant, a bean plant, a rice plant, a soybean plant, a cotton plant, a tobacco plant, a date palm plant, a wheat plant, a tomato plant, a banana plant, a potato plant, a pepper plant, a moss plant, a parsley plant, a citrus plant, an apple plant, a strawberry plant, a rapeseed plant, a cabbage plant, a cassava plant, and a coffee plant.
